SEQUENCE LISTING (1185 sequences)

SEQ ID NO: 1 - DNA, 40 bp, Artificial sequence
  synthetic sequence
tttcccttgctcttcatgattttaacaacatgatggattt

SEQ ID NO: 2 - DNA, 40 bp, Artificial sequence
  synthetic sequence
ccctgccccccattaactcacatcctgaattttatgttta

SEQ ID NO: 3 - DNA, 40 bp, Artificial sequence
  synthetic sequence
gcacttcatcatcgtctttgaagtccccttcttgtcctcc

SEQ ID NO: 4 - DNA, 40 bp, Artificial sequence
  synthetic sequence
tatgatgaacaccatgcaccacatgcaggttctggtgaag

SEQ ID NO: 5 - DNA, 40 bp, Artificial sequence
  synthetic sequence
gatacaaaagaatattggtatgtatgttgcacagactcat

SEQ ID NO: 6 - DNA, 40 bp, Artificial sequence
  synthetic sequence
cctatttcccccacacagccttcccacattggccaaccct

SEQ ID NO: 7 - DNA, 40 bp, Artificial sequence
  synthetic sequence
tacaaagggcttctctggccagagagagccggtgtctgct

SEQ ID NO: 8 - DNA, 40 bp, Artificial sequence
  synthetic sequence
tgggggggttaatggagttatggactgggatgggcagcct

SEQ ID NO: 9 - DNA, 40 bp, Artificial sequence
  synthetic sequence
acctacctagggaactctttctccctggcactaggctagt

SEQ ID NO: 10 - DNA, 40 bp, Artificial sequence
  synthetic sequence
actgactgagctgacctccagtacagggcctgaggccact

SEQ ID NO: 11 - DNA, 40 bp, Artificial sequence
  synthetic sequence
ctgggagctaaatagaagcaaatatccccaggcctgggtg

SEQ ID NO: 12 - DNA, 40 bp, Artificial sequence
  synthetic sequence
atgcgtcaagcaactacactcccacagtaaactgggaacc

SEQ ID NO: 13 - DNA, 40 bp, Artificial sequence
  synthetic sequence
cagctccttggcagcctaggctctagctcaacatctgctt

SEQ ID NO: 14 - DNA, 40 bp, Artificial sequence
  synthetic sequence
tgctggagtcgcaccaacctggctctgcctatctccagca

SEQ ID NO: 15 - DNA, 40 bp, Artificial sequence
  synthetic sequence
ctctgtaggctgcacaacgtggaacagatgaaaggaacca

SEQ ID NO: 16 - DNA, 40 bp, Artificial sequence
  synthetic sequence
tggggtaaattataatcatgaaattccgtcaagcttgaat

SEQ ID NO: 17 - DNA, 40 bp, Artificial sequence
  synthetic sequence
aacatatttaatatggcatattcaaatgacagaaagtacg

SEQ ID NO: 18 - DNA, 40 bp, Artificial sequence
  synthetic sequence
ctttattcttgctaatgttgactccttagcaaagataatt

SEQ ID NO: 19 - DNA, 40 bp, Artificial sequence
  synthetic sequence
tgatctttgctaaactcttcaggaataaatgaacatttcc

SEQ ID NO: 20 - DNA, 40 bp, Artificial sequence
  synthetic sequence
ttttcaagcagttaagaagcaagaattaatgactcgaata

SEQ ID NO: 21 - DNA, 40 bp, Artificial sequence
  synthetic sequence
atgagagtgttgactgatgaagggctcctatacgcgggtt

SEQ ID NO: 22 - DNA, 40 bp, Artificial sequence
  synthetic sequence
tctttcccatctgtttcccggcccctaccagaaataagtg

SEQ ID NO: 23 - DNA, 40 bp, Artificial sequence
  synthetic sequence
atgaacctccctcgctccaagaccagagctcctaggaagt

SEQ ID NO: 24 - DNA, 40 bp, Artificial sequence
  synthetic sequence
tctttattttattggccacaattgaacataggtataattt

SEQ ID NO: 25 - DNA, 40 bp, Artificial sequence
  synthetic sequence
cagaagcaagccctgatcaaggaaaccattcacacttgat

SEQ ID NO: 26 - DNA, 40 bp, Artificial sequence
  synthetic sequence
gtggcttttgctcaaagtgaggacgttatcagctctgccc

SEQ ID NO: 27 - DNA, 40 bp, Artificial sequence
  synthetic sequence
ctttaaacaaaaactaaaggcgtaaggaaagataactact

SEQ ID NO: 28 - DNA, 40 bp, Artificial sequence
  synthetic sequence
cagttgccacactttttttcactgctaaagttcgtaatga

SEQ ID NO: 29 - DNA, 40 bp, Artificial sequence
  synthetic sequence
ggcaatcagaagtattttggttgcttctaggtcagaatga

SEQ ID NO: 30 - DNA, 40 bp, Artificial sequence
  synthetic sequence
ggcagcaaacttgtttaggtatgattcatcattgtctgct

SEQ ID NO: 31 - DNA, 40 bp, Artificial sequence
  synthetic sequence
ctacaaaacaatgagtctgattacgacccacagaaatgaa

SEQ ID NO: 32 - DNA, 40 bp, Artificial sequence
  synthetic sequence
cctcccacagacccaaacatgctgctgcaaatgtctcact

SEQ ID NO: 33 - DNA, 40 bp, Artificial sequence
  synthetic sequence
ggacaagcacacacatcgctgggaagatctgcaagcctcc

SEQ ID NO: 34 - DNA, 40 bp, Artificial sequence
  synthetic sequence
taaacctggataacaagaacactgtttccactgcgctagt

SEQ ID NO: 35 - DNA, 40 bp, Artificial sequence
  synthetic sequence
tcatcacgatgacaatggacaagccatatccctaacaggg

SEQ ID NO: 36 - DNA, 40 bp, Artificial sequence
  synthetic sequence
tttccatgacaccaggaccgtaaagcaccttttacaccgt

SEQ ID NO: 37 - DNA, 40 bp, Artificial sequence
  synthetic sequence
aattgggatgtgcaaaacctcttaacttgtagcaccaagt

SEQ ID NO: 38 - DNA, 40 bp, Artificial sequence
  synthetic sequence
tcttgtgttattcgcctgcattgaaatcccatcccaatcc

SEQ ID NO: 39 - DNA, 40 bp, Artificial sequence
  synthetic sequence
tgagtgatctctttgctgatcataaacatattcctccatc

SEQ ID NO: 40 - DNA, 40 bp, Artificial sequence
  synthetic sequence
tgcattcattactaaatacacagggcatagcacatagtaa

SEQ ID NO: 41 - DNA, 40 bp, Artificial sequence
  synthetic sequence
cttcaatgttgccaggaaaatccttgcaggaatcacaccc

SEQ ID NO: 42 - DNA, 40 bp, Artificial sequence
  synthetic sequence
atttttttctaaagctttaggaaatacacacgtttcccct

SEQ ID NO: 43 - DNA, 40 bp, Artificial sequence
  synthetic sequence
agagtaatcttcaacaatccttggtctaaacacacacaag

SEQ ID NO: 44 - DNA, 40 bp, Artificial sequence
  synthetic sequence
cccagggacccacgccaagctcaccgcaccttccaccaaa

SEQ ID NO: 45 - DNA, 40 bp, Artificial sequence
  synthetic sequence
agctcctgtactagctggtggggtgtggagcacacagccc

SEQ ID NO: 46 - DNA, 40 bp, Artificial sequence
  synthetic sequence
tcacacagggaaagtgaggcttggtggttgatttgagcaa

SEQ ID NO: 47 - DNA, 40 bp, Artificial sequence
  synthetic sequence
ccttccaacagccgtgtgagacaagaggtcttatcctctt

SEQ ID NO: 48 - DNA, 40 bp, Artificial sequence
  synthetic sequence
acaagggtcactgagcacatgccatgtgttgggcacagtg

SEQ ID NO: 49 - DNA, 40 bp, Artificial sequence
  synthetic sequence
gtctcctaagtctcattcttttcttaggattcttcagatc

SEQ ID NO: 50 - DNA, 40 bp, Artificial sequence
  synthetic sequence
tccgcctaagtaaaacataaaattacttaagctgcgtaaa

SEQ ID NO: 51 - DNA, 40 bp, Artificial sequence
  synthetic sequence
cattttgacctgattatctttgtctataagtcttaagcca

SEQ ID NO: 52 - DNA, 40 bp, Artificial sequence
  synthetic sequence
ccggttcctccaccctcactgccccaacaactgaaagaag

SEQ ID NO: 53 - DNA, 40 bp, Artificial sequence
  synthetic sequence
acagtgtgttgaaagaatccataactctttctttccagcc

SEQ ID NO: 54 - DNA, 40 bp, Artificial sequence
  synthetic sequence
gaagtttcatctttatcaaaatctccattcccaggcggac

SEQ ID NO: 55 - DNA, 40 bp, Artificial sequence
  synthetic sequence
aagtccatttttttaagctttgcgcttcagctccagaaca

SEQ ID NO: 56 - DNA, 40 bp, Artificial sequence
  synthetic sequence
tcttcgttatgaatacaaataggaaaacaatcagacccaa

SEQ ID NO: 57 - DNA, 40 bp, Artificial sequence
  synthetic sequence
tcctcggggcattctagaaccgtagcagacctgcttacat

SEQ ID NO: 58 - DNA, 40 bp, Artificial sequence
  synthetic sequence
tccttatgtgggaaaataaagaggatagacagatttgatt

SEQ ID NO: 59 - DNA, 40 bp, Artificial sequence
  synthetic sequence
agctgcgagtccctaacagacttccaggacagctgaaaaa

SEQ ID NO: 60 - DNA, 40 bp, Artificial sequence
  synthetic sequence
aggacaagggagagacgcccacccgcctctgtcagggata

SEQ ID NO: 61 - DNA, 40 bp, Artificial sequence
  synthetic sequence
aatccatgagggtgacatacacatccttactgttcccaca

SEQ ID NO: 62 - DNA, 40 bp, Artificial sequence
  synthetic sequence
acttccttccctgagatgcccatcctttgattctgggatt

SEQ ID NO: 63 - DNA, 40 bp, Artificial sequence
  synthetic sequence
gctcccggataaattaattaccgtgaccctgagctgcttc

SEQ ID NO: 64 - DNA, 40 bp, Artificial sequence
  synthetic sequence
tagactaagagaatctaatttgtggcaaagatcttgagtg

SEQ ID NO: 65 - DNA, 40 bp, Artificial sequence
  synthetic sequence
tgaaggatgactaagagcttccctataaaccccatactgg

SEQ ID NO: 66 - DNA, 40 bp, Artificial sequence
  synthetic sequence
agccaggactatagagtttcagaaaagggagaaaattcta

SEQ ID NO: 67 - DNA, 40 bp, Artificial sequence
  synthetic sequence
tgctgctaatttaagtttctggcaagtcaaaataaatctc

SEQ ID NO: 68 - DNA, 40 bp, Artificial sequence
  synthetic sequence
cgaaaaccatcaattaactagaatgatcaggaaattgcgt

SEQ ID NO: 69 - DNA, 40 bp, Artificial sequence
  synthetic sequence
tttatttagtccccagggtgtatgaagtgctcttccaggc

SEQ ID NO: 70 - DNA, 40 bp, Artificial sequence
  synthetic sequence
ggtccttcttggtaccgatattgccatattggctggacat

SEQ ID NO: 71 - DNA, 40 bp, Artificial sequence
  synthetic sequence
tggcttggtaggatgcactcacatgggctgtagtaatact

SEQ ID NO: 72 - DNA, 40 bp, Artificial sequence
  synthetic sequence
tatcaccagcataacttgtggttcttcagccagtaatttc

SEQ ID NO: 73 - DNA, 40 bp, Artificial sequence
  synthetic sequence
gaacaactgggtatctacaggcaaagaaatgaaccttgac

SEQ ID NO: 74 - DNA, 40 bp, Artificial sequence
  synthetic sequence
taggtactgttgtgtccctatatatttgacttggtaataa

SEQ ID NO: 75 - DNA, 40 bp, Artificial sequence
  synthetic sequence
tatgtgaacatcggtgaatatcataatttattatgcaaac

SEQ ID NO: 76 - DNA, 40 bp, Artificial sequence
  synthetic sequence
agctgaacactctttgtggtcctcttgaagcctagaatta

SEQ ID NO: 77 - DNA, 40 bp, Artificial sequence
  synthetic sequence
ccccacctcactgccccccagttctgactcacggtgtccc

SEQ ID NO: 78 - DNA, 40 bp, Artificial sequence
  synthetic sequence
actcccatcacctggccagcttggctgtcccctgacccac

SEQ ID NO: 79 - DNA, 40 bp, Artificial sequence
  synthetic sequence
ggctgcccagctgcccagcagcaaaactgcataggaactc

SEQ ID NO: 80 - DNA, 40 bp, Artificial sequence
  synthetic sequence
gcccaggacgccaagtgtcaccaccctctccccaggcagg

SEQ ID NO: 81 - DNA, 40 bp, Artificial sequence
  synthetic sequence
cacaaggtcagctccacccgtgggtcagtgtgccccagat

SEQ ID NO: 82 - DNA, 40 bp, Artificial sequence
  synthetic sequence
ggagacaaaacgggcacccagcccagtcatgcccgtgcct

SEQ ID NO: 83 - DNA, 40 bp, Artificial sequence
  synthetic sequence
ctgaaatcagtcagcagtttcggtgagtctgcagctgaca

SEQ ID NO: 84 - DNA, 40 bp, Artificial sequence
  synthetic sequence
cgccacatttggggctgggagagatgtcacaggggctgac

SEQ ID NO: 85 - DNA, 40 bp, Artificial sequence
  synthetic sequence
cacatgttctctgcataggtttttaagcagccagcagctg

SEQ ID NO: 86 - DNA, 40 bp, Artificial sequence
  synthetic sequence
tttaaaatgaaaacccacacttccaaaatagcacttgagt

SEQ ID NO: 87 - DNA, 40 bp, Artificial sequence
  synthetic sequence
aacatgtttgtgtaattaagcattttaaaatcataaccat

SEQ ID NO: 88 - DNA, 40 bp, Artificial sequence
  synthetic sequence
tgcttatctgtgctttttatgttccacccccccaccacca

SEQ ID NO: 89 - DNA, 40 bp, Artificial sequence
  synthetic sequence
attaataataattctgtgtttatggggattgcagatacat

SEQ ID NO: 90 - DNA, 40 bp, Artificial sequence
  synthetic sequence
ccagctttgtgtcttcatgacccaactggagtaagaatgg

SEQ ID NO: 91 - DNA, 40 bp, Artificial sequence
  synthetic sequence
aaagacctcatttgcagcatggttagcagtgtcaaacatt

SEQ ID NO: 92 - DNA, 40 bp, Artificial sequence
  synthetic sequence
tctcgtagcactggctgcagccggcctgtgtgtgcccacc

SEQ ID NO: 93 - DNA, 40 bp, Artificial sequence
  synthetic sequence
gccttcatcctgaacggctgaccagcggaaacaaaagatc

SEQ ID NO: 94 - DNA, 40 bp, Artificial sequence
  synthetic sequence
atggccagataacagtgtttagacatgtctttgatgtttt

SEQ ID NO: 95 - DNA, 40 bp, Artificial sequence
  synthetic sequence
ccctgactgtgtaaggggtctctctccatggggaatagag

SEQ ID NO: 96 - DNA, 40 bp, Artificial sequence
  synthetic sequence
ctgagcttagcttctactgtgctgttaatttcaggcaaga

SEQ ID NO: 97 - DNA, 40 bp, Artificial sequence
  synthetic sequence
agatcaataatatttgcattagctacttacatcagtctct

SEQ ID NO: 98 - DNA, 40 bp, Artificial sequence
  synthetic sequence
taattgcagaaaacttataaagcatggaagaatacaaaac

SEQ ID NO: 99 - DNA, 40 bp, Artificial sequence
  synthetic sequence
aaacaaattcctctacctggacatgactgttgttagcatt

SEQ ID NO: 100 - DNA, 40 bp, Artificial sequence
  synthetic sequence
gggagattcttcatatccttttaatgtagatatgcacatt

SEQ ID NO: 101 - DNA, 40 bp, Artificial sequence
  synthetic sequence
acaaaaaaggctatcatattgtacatataactttgctgta

SEQ ID NO: 102 - DNA, 40 bp, Artificial sequence
  synthetic sequence
tctgctaggaacctgtacccatgtcattactgtaagcatt

SEQ ID NO: 103 - DNA, 40 bp, Artificial sequence
  synthetic sequence
actactcaaattttagtatctgcagatatcagatatcctt

SEQ ID NO: 104 - DNA, 40 bp, Artificial sequence
  synthetic sequence
tgaaatggtattgttgccctttctgattagtaaagtatac

SEQ ID NO: 105 - DNA, 40 bp, Artificial sequence
  synthetic sequence
ttataatctagcaaggttagagatcatggatcactttcag

SEQ ID NO: 106 - DNA, 40 bp, Artificial sequence
  synthetic sequence
acagcttgcctccgataagccagaattccagagcttctgg

SEQ ID NO: 107 - DNA, 40 bp, Artificial sequence
  synthetic sequence
tcaatcaacctgatagcttaggggataaactaatttgaag

SEQ ID NO: 108 - DNA, 40 bp, Artificial sequence
  synthetic sequence
gatcatgaaggatgaaagaatttcaccaatattataataa

SEQ ID NO: 109 - DNA, 40 bp, Artificial sequence
  synthetic sequence
tttagccatctgtatcaatgagcagatataagctttacac

SEQ ID NO: 110 - DNA, 40 bp, Artificial sequence
  synthetic sequence
aggggtagattatttatgctgcccatttttagaccataaa

SEQ ID NO: 111 - DNA, 40 bp, Artificial sequence
  synthetic sequence
cactaccatttcacaattcgcactttctttctttgtcctt

SEQ ID NO: 112 - DNA, 40 bp, Artificial sequence
  synthetic sequence
gctccatcaaatcataaaggacccacttcaaatgccatca

SEQ ID NO: 113 - DNA, 40 bp, Artificial sequence
  synthetic sequence
tcctactttcaggaacttctttctccaaacgtcttctgcc

SEQ ID NO: 114 - DNA, 40 bp, Artificial sequence
  synthetic sequence
aattctattttttcttcaacgtactttaggcttgtaatgt

SEQ ID NO: 115 - DNA, 40 bp, Artificial sequence
  synthetic sequence
taagatgcaaatagtaagcctgagcccttctgtctaactt

SEQ ID NO: 116 - DNA, 40 bp, Artificial sequence
  synthetic sequence
ctgtgtttcagaataaaataccaactctactactctcatc

SEQ ID NO: 117 - DNA, 40 bp, Artificial sequence
  synthetic sequence
gaaaccatgtttatctcaggtttacaaatctccacttgtc

SEQ ID NO: 118 - DNA, 40 bp, Artificial sequence
  synthetic sequence
ctttggaaaagtaatcaggtttagaggagctcatgagagc

SEQ ID NO: 119 - DNA, 40 bp, Artificial sequence
  synthetic sequence
gctgaatccccaactcccaattggctccatttgtggggga

SEQ ID NO: 120 - DNA, 40 bp, Artificial sequence
  synthetic sequence
ggtgttatgaacttaacgcttgtgtctccagaaaattcac

SEQ ID NO: 121 - DNA, 40 bp, Artificial sequence
  synthetic sequence
agttaatgcacgttaataagcaagagtttagtttaatgtg

SEQ ID NO: 122 - DNA, 40 bp, Artificial sequence
  synthetic sequence
taattgagaaggcagattcactggagttcttatataattg

SEQ ID NO: 123 - DNA, 40 bp, Artificial sequence
  synthetic sequence
cacggtcagatgaaaatatagtgtgaagaatttgtataac

SEQ ID NO: 124 - DNA, 40 bp, Artificial sequence
  synthetic sequence
cacaagtcagcatcagcgtgtcatgtctcagcagcagaac

SEQ ID NO: 125 - DNA, 40 bp, Artificial sequence
  synthetic sequence
ggaggtggggacttaggtgaaggaaatgagccagcagaag

SEQ ID NO: 126 - DNA, 40 bp, Artificial sequence
  synthetic sequence
gtcacagcatttcaaggaggagacctcattgtaagcttct

SEQ ID NO: 127 - DNA, 40 bp, Artificial sequence
  synthetic sequence
aaagaggtgaaattaatcccatacccttaagtctacagac

SEQ ID NO: 128 - DNA, 40 bp, Artificial sequence
  synthetic sequence
ctttactaaggaacttttcattttaagtgttgacgcatgc

SEQ ID NO: 129 - DNA, 40 bp, Artificial sequence
  synthetic sequence
caggtttttctttccacggtaactacaatgaagtgatcct

SEQ ID NO: 130 - DNA, 40 bp, Artificial sequence
  synthetic sequence
gctctacagggaggttgaggtgttagagatcagagcagga

SEQ ID NO: 131 - DNA, 40 bp, Artificial sequence
  synthetic sequence
tactatttccaacggcatctggcttttctcagcccttgtg

SEQ ID NO: 132 - DNA, 40 bp, Artificial sequence
  synthetic sequence
aaggtttaggcagggatagccattctattttattaggggc

SEQ ID NO: 133 - DNA, 40 bp, Artificial sequence
  synthetic sequence
aggggctcaacgaagaaaaagtgttccaagctttaggaag

SEQ ID NO: 134 - DNA, 40 bp, Artificial sequence
  synthetic sequence
gggctgaacccccttccctggattgcagcacagcagcgag

SEQ ID NO: 135 - DNA, 40 bp, Artificial sequence
  synthetic sequence
ctgacgtcataatctaccaaggtcatggatcgagttcaga

SEQ ID NO: 136 - DNA, 40 bp, Artificial sequence
  synthetic sequence
gaaggtagagctctcctccaataagccagatttccagagt

SEQ ID NO: 137 - DNA, 40 bp, Artificial sequence
  synthetic sequence
caccaatattattataattcctatcaacctgataggttag

SEQ ID NO: 138 - DNA, 40 bp, Artificial sequence
  synthetic sequence
agatataagccttacacaggattatgaagtctgaaaggat

SEQ ID NO: 139 - DNA, 40 bp, Artificial sequence
  synthetic sequence
acatgtatctttctggtcttttagccgcctaacactttga

SEQ ID NO: 140 - DNA, 40 bp, Artificial sequence
  synthetic sequence
caaagaacaagtgcaatatgtgcagctttgttgcgcaggt

SEQ ID NO: 141 - DNA, 40 bp, Artificial sequence
  synthetic sequence
tattattatgtgagtaactggaagatactgataagttgac

SEQ ID NO: 142 - DNA, 40 bp, Artificial sequence
  synthetic sequence
taaaaatctttctcacccatccttagattgagagaagtca

SEQ ID NO: 143 - DNA, 40 bp, Artificial sequence
  synthetic sequence
ttgggttcacctcagtctctataatctgtaccagcatacc

SEQ ID NO: 144 - DNA, 40 bp, Artificial sequence
  synthetic sequence
cacacccatctcacagatcccctatcttaaagagacccta

SEQ ID NO: 145 - DNA, 40 bp, Artificial sequence
  synthetic sequence
atggaacccaaccagactctcagatatggccaaagatcta

SEQ ID NO: 146 - DNA, 40 bp, Artificial sequence
  synthetic sequence
gacaccagtctctgacacattcttaaaggtcaggctctac

SEQ ID NO: 147 - DNA, 40 bp, Artificial sequence
  synthetic sequence
agagattcaaaagattcacttgtttaggccttagcgggct

SEQ ID NO: 148 - DNA, 40 bp, Artificial sequence
  synthetic sequence
tccttagtctgaggaggagcaattaagattcacttgttta

SEQ ID NO: 149 - DNA, 40 bp, Artificial sequence
  synthetic sequence
taaatggggaagttgtttgaaaacaggagggatcctagat

SEQ ID NO: 150 - DNA, 40 bp, Artificial sequence
  synthetic sequence
gggtttatacatgacttttagaacactgccttggtttttg

SEQ ID NO: 151 - DNA, 40 bp, Artificial sequence
  synthetic sequence
aactcttaaaagatattgcctcaaaagcataagaggaaat

SEQ ID NO: 152 - DNA, 40 bp, Artificial sequence
  synthetic sequence
aaatcgaggaataagacagttatggataaggagaaatcaa

SEQ ID NO: 153 - DNA, 40 bp, Artificial sequence
  synthetic sequence
tcagttaggatttaatcaatgtcagaagcaatgatatagg

SEQ ID NO: 154 - DNA, 40 bp, Artificial sequence
  synthetic sequence
cttgaaaacacttgaaattgcttgtgtaaagaaacagttt

SEQ ID NO: 155 - DNA, 40 bp, Artificial sequence
  synthetic sequence
ataatcttcagaggaaagttttattctctgacttatttaa

SEQ ID NO: 156 - DNA, 40 bp, Artificial sequence
  synthetic sequence
agattccttctgtcattttgcctctgttcgaatactttct

SEQ ID NO: 157 - DNA, 40 bp, Artificial sequence
  synthetic sequence
atttcagcttctaaactttatttggcaatgccttcccatg

SEQ ID NO: 158 - DNA, 40 bp, Artificial sequence
  synthetic sequence
gcaggagtttgttttcttctgcttcagagctttgaattta

SEQ ID NO: 159 - DNA, 40 bp, Artificial sequence
  synthetic sequence
acatatcaacggcactggttctttatctaactctctggca

SEQ ID NO: 160 - DNA, 40 bp, Artificial sequence
  synthetic sequence
ttatgcttccctgaaacaataccacctgctattctccact

SEQ ID NO: 161 - DNA, 40 bp, Artificial sequence
  synthetic sequence
ttctcactccctaccactgaggacaagtttatgtccttag

SEQ ID NO: 162 - DNA, 40 bp, Artificial sequence
  synthetic sequence
ttagagattatgtcattaccagagttaaaattctataatg

SEQ ID NO: 163 - DNA, 40 bp, Artificial sequence
  synthetic sequence
ggtcattcttagaatagtaatccagccaatagtacaggtt

SEQ ID NO: 164 - DNA, 40 bp, Artificial sequence
  synthetic sequence
caggcaataagggctttttaagcaaaacagttgtgataaa

SEQ ID NO: 165 - DNA, 40 bp, Artificial sequence
  synthetic sequence
atgatgggcactgaaggttaaaacttgagtctgtcaactt

SEQ ID NO: 166 - DNA, 40 bp, Artificial sequence
  synthetic sequence
aactcataaatatcccattttccgctgaaatatagcttta

SEQ ID NO: 167 - DNA, 40 bp, Artificial sequence
  synthetic sequence
cctggtttctttgaccttttgggaccttgagtaagtaaag

SEQ ID NO: 168 - DNA, 40 bp, Artificial sequence
  synthetic sequence
cttcatttattttcatgattaaaattctaagaaattcttg

SEQ ID NO: 169 - DNA, 40 bp, Artificial sequence
  synthetic sequence
tttttaattaaattgcattgcctaatgtatttatgaacta

SEQ ID NO: 170 - DNA, 40 bp, Artificial sequence
  synthetic sequence
catagaaataaaacaatactctgaagtagttcagaatgtg

SEQ ID NO: 171 - DNA, 40 bp, Artificial sequence
  synthetic sequence
caatttatataaagagttaattcaaatgagactattttaa

SEQ ID NO: 172 - DNA, 40 bp, Artificial sequence
  synthetic sequence
agggctttgaatcttatgtctagaaattttgaaaaacctc

SEQ ID NO: 173 - DNA, 40 bp, Artificial sequence
  synthetic sequence
tatatgctaagattccacctctagtgctagaactgagaag

SEQ ID NO: 174 - DNA, 40 bp, Artificial sequence
  synthetic sequence
tgacttggtgatcttttttaaattctgaaacaacagcaac

SEQ ID NO: 175 - DNA, 40 bp, Artificial sequence
  synthetic sequence
agctaaggactttttcttgcctatgcatgctatcttcagt

SEQ ID NO: 176 - DNA, 40 bp, Artificial sequence
  synthetic sequence
tgattatttagtattgaaactataacatagtatgtttcct

SEQ ID NO: 177 - DNA, 40 bp, Artificial sequence
  synthetic sequence
aaaaaatgtgtatttctctggagaaggttaaaactgagga

SEQ ID NO: 178 - DNA, 40 bp, Artificial sequence
  synthetic sequence
caagtgagcaaggcttaaatggaagaagcaatgatctcgt

SEQ ID NO: 179 - DNA, 40 bp, Artificial sequence
  synthetic sequence
ccaccttcattaacgagatcatccatcatgaggaaatatg

SEQ ID NO: 180 - DNA, 40 bp, Artificial sequence
  synthetic sequence
accaggccccctctgttttgtgtcactaagggtgaggatg

SEQ ID NO: 181 - DNA, 40 bp, Artificial sequence
  synthetic sequence
atgatttttccctcccccgggcttcttttagccatcaata

SEQ ID NO: 182 - DNA, 40 bp, Artificial sequence
  synthetic sequence
tagccccacaggagtttgttctgaaagtaaacttccacaa

SEQ ID NO: 183 - DNA, 40 bp, Artificial sequence
  synthetic sequence
aagcttattgaggctaaggcatctgtgaaggaaagaaaca

SEQ ID NO: 184 - DNA, 40 bp, Artificial sequence
  synthetic sequence
ctctaaaccactatgctgctagagcctcttttctgtactc

SEQ ID NO: 185 - DNA, 40 bp, Artificial sequence
  synthetic sequence
ctcattcagacactagtgtcaccagtctcctcatatacct

SEQ ID NO: 186 - DNA, 40 bp, Artificial sequence
  synthetic sequence
tattttcttcttcttgctggtttagtcatgttttctggga

SEQ ID NO: 187 - DNA, 40 bp, Artificial sequence
  synthetic sequence
ggcaaacccattatttttttctttagacttgggatggtga

SEQ ID NO: 188 - DNA, 40 bp, Artificial sequence
  synthetic sequence
tgggcagcgtcagaaactgtgtgtggatatagataagagc

SEQ ID NO: 189 - DNA, 40 bp, Artificial sequence
  synthetic sequence
gactatgctgagctgtgatgagggaggggcctagctaaag

SEQ ID NO: 190 - DNA, 40 bp, Artificial sequence
  synthetic sequence
tgagagtcagaatgctcctgctattgccttctcagtcccc

SEQ ID NO: 191 - DNA, 40 bp, Artificial sequence
  synthetic sequence
ttggtttctacacaagtagatacatagaaaaggctatagg

SEQ ID NO: 192 - DNA, 40 bp, Artificial sequence
  synthetic sequence
tgtttgagagtcctgcatgattagttgctcagaaatgccc

SEQ ID NO: 193 - DNA, 40 bp, Artificial sequence
  synthetic sequence
ttacaaatatgtgattatcatcaaaacgtgagggctaaag

SEQ ID NO: 194 - DNA, 40 bp, Artificial sequence
  synthetic sequence
cagataacttgcaagtcctaggataccaggaaaataaatt

SEQ ID NO: 195 - DNA, 40 bp, Artificial sequence
  synthetic sequence
agcattatgtctgtctgtcattgtttttcatcctcttgta

SEQ ID NO: 196 - DNA, 40 bp, Artificial sequence
  synthetic sequence
ttcacagttacccacacaggtgaacccttttagctctcct

SEQ ID NO: 197 - DNA, 40 bp, Artificial sequence
  synthetic sequence
gaatgtttctttcctctcaggatcagagttgcctacatct

SEQ ID NO: 198 - DNA, 40 bp, Artificial sequence
  synthetic sequence
aatgcaccaagactggcctgagatgtatccttaagatgag

SEQ ID NO: 199 - DNA, 40 bp, Artificial sequence
  synthetic sequence
tcccagtagcaccccaagtcagatctgaccccgtatgtga

SEQ ID NO: 200 - DNA, 40 bp, Artificial sequence
  synthetic sequence
gtgtcctctaacagcacaggccttttgccacctagctgtc

SEQ ID NO: 201 - DNA, 40 bp, Artificial sequence
  synthetic sequence
ggcaaacaaggtttgttttcttttcctgttttcatgcctt

SEQ ID NO: 202 - DNA, 40 bp, Artificial sequence
  synthetic sequence
ttccatatccttgtttcatattaatacatgtgtatagatc

SEQ ID NO: 203 - DNA, 40 bp, Artificial sequence
  synthetic sequence
aaatctatacacatgtattaataaagcctgattctgccgc

SEQ ID NO: 204 - DNA, 40 bp, Artificial sequence
  synthetic sequence
aggtatagaggccacctgcaagataaatatttgattcaca

SEQ ID NO: 205 - DNA, 40 bp, Artificial sequence
  synthetic sequence
ctaatcattctatggcaattgataacaacaaatatatata

SEQ ID NO: 206 - DNA, 40 bp, Artificial sequence
  synthetic sequence
ataatatattctagaatatgtcacattctgtctcaggcat

SEQ ID NO: 207 - DNA, 40 bp, Artificial sequence
  synthetic sequence
tttctttatgatgccgtttgaggtggagttttagtcaggt

SEQ ID NO: 208 - DNA, 40 bp, Artificial sequence
  synthetic sequence
agcttctccttttttttgccatctgccctgtaagcatcct

SEQ ID NO: 209 - DNA, 40 bp, Artificial sequence
  synthetic sequence
gggacccagataggagtcatcactctaggctgagaacatc

SEQ ID NO: 210 - DNA, 40 bp, Artificial sequence
  synthetic sequence
cacacaccctaagcctcagcatgactcatcatgactcagc

SEQ ID NO: 211 - DNA, 40 bp, Artificial sequence
  synthetic sequence
ctgtgcttgagccagaaggtttgcttagaaggttacacag

SEQ ID NO: 212 - DNA, 40 bp, Artificial sequence
  synthetic sequence
aactgctcatgcttggactatgggaggtcactaatggaga

SEQ ID NO: 213 - DNA, 40 bp, Artificial sequence
  synthetic sequence
cagaaatgtaacaggaactaaggaaaaactgaagcttatt

SEQ ID NO: 214 - DNA, 40 bp, Artificial sequence
  synthetic sequence
cagagatgaggatgctggaagggatagagggagctgagct

SEQ ID NO: 215 - DNA, 40 bp, Artificial sequence
  synthetic sequence
aaaagtatagtaatcattcagcaaatggttttgaagcacc

SEQ ID NO: 216 - DNA, 40 bp, Artificial sequence
  synthetic sequence
gtatcttattccccacaagagtccaagtaaaaaataacag

SEQ ID NO: 217 - DNA, 40 bp, Artificial sequence
  synthetic sequence
gaaaagaatgtttctctcactgtggattattttagagagt

SEQ ID NO: 218 - DNA, 40 bp, Artificial sequence
  synthetic sequence
aatggtcaagatttttttaaaaattaagaaaacataagtt

SEQ ID NO: 219 - DNA, 40 bp, Artificial sequence
  synthetic sequence
cttgagaaatgaaaatttatttttttgttggaggataccc

SEQ ID NO: 220 - DNA, 40 bp, Artificial sequence
  synthetic sequence
tctatctcccatcagggcaagctgtaaggaactggctaag

SEQ ID NO: 221 - DNA, 40 bp, Artificial sequence
  synthetic sequence
agtgagacagagtgacttagtcttagaggccccactggta

SEQ ID NO: 222 - DNA, 40 bp, Artificial sequence
  synthetic sequence
gatgagaaggcaccttcatcactcatcacagtcagctctg

SEQ ID NO: 223 - DNA, 40 bp, Artificial sequence
  synthetic sequence
tctcctctctcctttctcatcagaaatttcataagtctac

SEQ ID NO: 224 - DNA, 40 bp, Artificial sequence
  synthetic sequence
gtcaggcagatcacataagaaaagaggatgccagttaagg

SEQ ID NO: 225 - DNA, 40 bp, Artificial sequence
  synthetic sequence
gttgctgttagacaatttcatctgtgccctgcttaggagc

SEQ ID NO: 226 - DNA, 40 bp, Artificial sequence
  synthetic sequence
tctttaatgaaagctaagctttcattaaaaaaagtctaac

SEQ ID NO: 227 - DNA, 40 bp, Artificial sequence
  synthetic sequence
tgcattcgactttgactgcagcagctggttagaaggttct

SEQ ID NO: 228 - DNA, 40 bp, Artificial sequence
  synthetic sequence
gaggagggtcccagcccattgctaaattaacatcaggctc

SEQ ID NO: 229 - DNA, 40 bp, Artificial sequence
  synthetic sequence
actggcagtatatctctaacagtggttgatgctatcttct

SEQ ID NO: 230 - DNA, 40 bp, Artificial sequence
  synthetic sequence
cttgcctgctacattgagaccactgacccatacataggaa

SEQ ID NO: 231 - DNA, 40 bp, Artificial sequence
  synthetic sequence
atagctctgtcctgaactgttaggccactggtccagagag

SEQ ID NO: 232 - DNA, 40 bp, Artificial sequence
  synthetic sequence
catctcctttgatcctcataataaccctatgagatagaca

SEQ ID NO: 233 - DNA, 40 bp, Artificial sequence
  synthetic sequence
tattactcttactttatagatgatgatcctgaaaacatag

SEQ ID NO: 234 - DNA, 40 bp, Artificial sequence
  synthetic sequence
caaggcacttgcccctagctgggggtataggggagcagtc

SEQ ID NO: 235 - DNA, 40 bp, Artificial sequence
  synthetic sequence
gtagtagtagaatgaaaaatgctgctatgctgtgcctccc

SEQ ID NO: 236 - DNA, 40 bp, Artificial sequence
  synthetic sequence
ctttcccatgtctgccctctactcatggtctatctctcct

SEQ ID NO: 237 - DNA, 40 bp, Artificial sequence
  synthetic sequence
cctgggagtcatggactccacccagcaccaccaacctgac

SEQ ID NO: 238 - DNA, 40 bp, Artificial sequence
  synthetic sequence
ccacctatctgagcctgccagcctataacccatctgggcc

SEQ ID NO: 239 - DNA, 40 bp, Artificial sequence
  synthetic sequence
tagctggtggccagccctgaccccaccccaccctccctgg

SEQ ID NO: 240 - DNA, 40 bp, Artificial sequence
  synthetic sequence
tctgatagacacatctggcacaccagctcgcaaagtcacc

SEQ ID NO: 241 - DNA, 40 bp, Artificial sequence
  synthetic sequence
gggtcttgtgtttgctgagtcaaaattccttgaaatccaa

SEQ ID NO: 242 - DNA, 40 bp, Artificial sequence
  synthetic sequence
ttagagactcctgctcccaaatttacagtcatagacttct

SEQ ID NO: 243 - DNA, 40 bp, Artificial sequence
  synthetic sequence
ggctgtctcctttatccacagaatgattcctttgcttcat

SEQ ID NO: 244 - DNA, 40 bp, Artificial sequence
  synthetic sequence
ccatccatctgatcctcctcatcagtgcagcacagggccc

SEQ ID NO: 245 - DNA, 40 bp, Artificial sequence
  synthetic sequence
gcagtagctgcagagtctcacataggtctggcactgcctc

SEQ ID NO: 246 - DNA, 40 bp, Artificial sequence
  synthetic sequence
atgtccgaccttaggcaaatgcttgactcttctgagctca

SEQ ID NO: 247 - DNA, 40 bp, Artificial sequence
  synthetic sequence
tgtcatggcaaaataaagataataatagtgtttttttatg

SEQ ID NO: 248 - DNA, 40 bp, Artificial sequence
  synthetic sequence
tagcgtgaggatggaaaacaatagcaaaattgattagact

SEQ ID NO: 249 - DNA, 40 bp, Artificial sequence
  synthetic sequence
aaggtctcaacaaatagtagtagattttatcgtccattaa

SEQ ID NO: 250 - DNA, 40 bp, Artificial sequence
  synthetic sequence
tccctctcctctcttactcatcccatcacgtatgcctctt

SEQ ID NO: 251 - DNA, 40 bp, Artificial sequence
  synthetic sequence
ttcccttacctataataagagttattcctcttattatatt

SEQ ID NO: 252 - DNA, 40 bp, Artificial sequence
  synthetic sequence
ttatagtgattctggatattaaagtgggaatgaggggcag

SEQ ID NO: 253 - DNA, 40 bp, Artificial sequence
  synthetic sequence
ctaacgaagaagatgtttctcaaagaagccattctcccca

SEQ ID NO: 254 - DNA, 40 bp, Artificial sequence
  synthetic sequence
gatcatctcagcagggttcaggaagataaaggaggatcaa

SEQ ID NO: 255 - DNA, 40 bp, Artificial sequence
  synthetic sequence
tgttgaggtgggaggaccgcttgagcctgggaagtgcaag

SEQ ID NO: 256 - DNA, 40 bp, Artificial sequence
  synthetic sequence
agtgagccgagattttgccactacactcccatttgggtga

SEQ ID NO: 257 - DNA, 40 bp, Artificial sequence
  synthetic sequence
gtgagaccctttctcaaaaacaaactaattaaaaaaccct

SEQ ID NO: 258 - DNA, 40 bp, Artificial sequence
  synthetic sequence
tttacagatgaagaaactgagtcatacaactactaagaga

SEQ ID NO: 259 - DNA, 40 bp, Artificial sequence
  synthetic sequence
gagtcactaatcactcaggtggtctggctccagcatctgt

SEQ ID NO: 260 - DNA, 40 bp, Artificial sequence
  synthetic sequence
ttaatctctgctctatactgcccaagacttttataaagtc

SEQ ID NO: 261 - DNA, 40 bp, Artificial sequence
  synthetic sequence
gttgagtcactgaaatgagttattgggatggctgtgtggg

SEQ ID NO: 262 - DNA, 40 bp, Artificial sequence
  synthetic sequence
gtgctaagttctttcctaaaggtatgtgagaatacaaagg

SEQ ID NO: 263 - DNA, 40 bp, Artificial sequence
  synthetic sequence
aagcatcctcctttttacacacgtgaactagtgcatgcaa

SEQ ID NO: 264 - DNA, 40 bp, Artificial sequence
  synthetic sequence
gacactcagtgggcctgggtgaaggtgagaattttattgc

SEQ ID NO: 265 - DNA, 40 bp, Artificial sequence
  synthetic sequence
tgagagcctctggggacatcttgccagtcaatgagtctca

SEQ ID NO: 266 - DNA, 40 bp, Artificial sequence
  synthetic sequence
caatttccttctcagtcttggagtaacagaagctcatgca

SEQ ID NO: 267 - DNA, 40 bp, Artificial sequence
  synthetic sequence
ataaacggaaattttgtattgaaatgagagccattggaaa

SEQ ID NO: 268 - DNA, 40 bp, Artificial sequence
  synthetic sequence
ttactccagactcctacttataaaaagagaaactgaggct

SEQ ID NO: 269 - DNA, 40 bp, Artificial sequence
  synthetic sequence
gaagggtggggactttctcagtatgacatggaaatgatca

SEQ ID NO: 270 - DNA, 40 bp, Artificial sequence
  synthetic sequence
tggattcaaagctcctgactttctgtctagtgtatgtgca

SEQ ID NO: 271 - DNA, 40 bp, Artificial sequence
  synthetic sequence
gccccttttcctctaactgaaagaaggaaaaaaaaatgga

SEQ ID NO: 272 - DNA, 40 bp, Artificial sequence
  synthetic sequence
aaaatattctacatagtttccatgtcacagccagggctgg

SEQ ID NO: 273 - DNA, 40 bp, Artificial sequence
  synthetic sequence
tctcctgttatttcttttaaaataaatatatcatttaaat

SEQ ID NO: 274 - DNA, 40 bp, Artificial sequence
  synthetic sequence
aaataagcaaaccctgctcgggaatgggagggagagtctc

SEQ ID NO: 275 - DNA, 40 bp, Artificial sequence
  synthetic sequence
gtccaccccttctcggccctggctctgcagatagtgctat

SEQ ID NO: 276 - DNA, 40 bp, Artificial sequence
  synthetic sequence
gccctgacagagccctgcccattgctgggccttggagtga

SEQ ID NO: 277 - DNA, 40 bp, Artificial sequence
  synthetic sequence
gcctagtagagaggcagggcaagccatctcatagctgctg

SEQ ID NO: 278 - DNA, 40 bp, Artificial sequence
  synthetic sequence
ggagagagaaaagggctcattgtctataaactcaggtcat

SEQ ID NO: 279 - DNA, 40 bp, Artificial sequence
  synthetic sequence
attcttattctcacactaagaaaaagaatgagatgtctac

SEQ ID NO: 280 - DNA, 40 bp, Artificial sequence
  synthetic sequence
accctgcgtcccctcttgtgtactggggtccccaagagct

SEQ ID NO: 281 - DNA, 40 bp, Artificial sequence
  synthetic sequence
aaaagtgatggcaaagtcattgcgctagatgccatcccat

SEQ ID NO: 282 - DNA, 40 bp, Artificial sequence
  synthetic sequence
tataaacctgcatttgtctccacacaccagtcatggacaa

SEQ ID NO: 283 - DNA, 40 bp, Artificial sequence
  synthetic sequence
cctcctcccaggtccacgtgcttgtctttgtataatactc

SEQ ID NO: 284 - DNA, 40 bp, Artificial sequence
  synthetic sequence
aatttcggaaaatgtattctttcaatcttgttctgttatt

SEQ ID NO: 285 - DNA, 40 bp, Artificial sequence
  synthetic sequence
tttcaatggcttagtagaaaaagtacatacttgttttccc

SEQ ID NO: 286 - DNA, 40 bp, Artificial sequence
  synthetic sequence
attgacaatagacaatttcacatcaatgtctatatgggtc

SEQ ID NO: 287 - DNA, 40 bp, Artificial sequence
  synthetic sequence
tgtttgctgtgtttgcaaaaactcacaataactttatatt

SEQ ID NO: 288 - DNA, 40 bp, Artificial sequence
  synthetic sequence
ctactctaagaaagttacaacatggtgaatacaagagaaa

SEQ ID NO: 289 - DNA, 40 bp, Artificial sequence
  synthetic sequence
ttacaagtccagaaaataaaagttatcatcttgaggcctc

SEQ ID NO: 290 - DNA, 40 bp, Artificial sequence
  synthetic sequence
ttctaggaataatatcaatattacaaaattaatctaacaa

SEQ ID NO: 291 - DNA, 40 bp, Artificial sequence
  synthetic sequence
gaacagcaatgagataatgtgtacaaagtacccagaccta

SEQ ID NO: 292 - DNA, 40 bp, Artificial sequence
  synthetic sequence
gtagagcatcaaggaagcgcattgcggagcagttttttgt

SEQ ID NO: 293 - DNA, 40 bp, Artificial sequence
  synthetic sequence
ttgtttttgtattctgtttcgtgaggcaaggtttcactct

SEQ ID NO: 294 - DNA, 40 bp, Artificial sequence
  synthetic sequence
tccaggctggagtgcagtggcaagatcatgtctcactgca

SEQ ID NO: 295 - DNA, 40 bp, Artificial sequence
  synthetic sequence
tgacctcctgagctcaagggatcctcccatttcggcctcc

SEQ ID NO: 296 - DNA, 40 bp, Artificial sequence
  synthetic sequence
tagctgggactacaggtgtacatcacatgcctggctaatt

SEQ ID NO: 297 - DNA, 40 bp, Artificial sequence
  synthetic sequence
ttttttttttaagtagagacgaggtcttgctatgttgtcc

SEQ ID NO: 298 - DNA, 40 bp, Artificial sequence
  synthetic sequence
taatatcaaactcttgagctcaagcagtcctcccacttct

SEQ ID NO: 299 - DNA, 40 bp, Artificial sequence
  synthetic sequence
tggaggtatccagtatgaaatttagataatacctgccttc

SEQ ID NO: 300 - DNA, 40 bp, Artificial sequence
  synthetic sequence
gttgaaattagaacttaatgatataatgcatcaatgaact

SEQ ID NO: 301 - DNA, 40 bp, Artificial sequence
  synthetic sequence
atagttcctagcacaaagtaagaatcctttcaatgtgtgt

SEQ ID NO: 302 - DNA, 40 bp, Artificial sequence
  synthetic sequence
gtgtatgtatttatctgttattaataggaatcttatgggc

SEQ ID NO: 303 - DNA, 40 bp, Artificial sequence
  synthetic sequence
tctcacttaatccttattaataactatgaagcaggtattt

SEQ ID NO: 304 - DNA, 40 bp, Artificial sequence
  synthetic sequence
gagttttccaagtgagttaagtatagcttgtaatacttaa

SEQ ID NO: 305 - DNA, 40 bp, Artificial sequence
  synthetic sequence
atatccacaggttacatagctagtatataactgagaaata

SEQ ID NO: 306 - DNA, 40 bp, Artificial sequence
  synthetic sequence
tatttatattataaaacattctaacaatacagatgtatat

SEQ ID NO: 307 - DNA, 40 bp, Artificial sequence
  synthetic sequence
taaaaaactgaaagggctcatgcaaccctaccttctcaat

SEQ ID NO: 308 - DNA, 40 bp, Artificial sequence
  synthetic sequence
cttcttcacttagaaaaaaccagccttagctgtctgctat

SEQ ID NO: 309 - DNA, 40 bp, Artificial sequence
  synthetic sequence
cctttcaaaatatacttctgagaaatgagagagagaaatg

SEQ ID NO: 310 - DNA, 40 bp, Artificial sequence
  synthetic sequence
gggtagaaggaaggaagatagggtaagagacagggaagga

SEQ ID NO: 311 - DNA, 40 bp, Artificial sequence
  synthetic sequence
tggggaaagaaattaaattattcttttctctgtctcttga

SEQ ID NO: 312 - DNA, 40 bp, Artificial sequence
  synthetic sequence
gctctttccattacattgaatcaaaggtaatgttgccatt

SEQ ID NO: 313 - DNA, 40 bp, Artificial sequence
  synthetic sequence
gactcttgaaataaagaaagaccgatgtatgaaataattt

SEQ ID NO: 314 - DNA, 40 bp, Artificial sequence
  synthetic sequence
agtctatggcattttcaaaatgcaaggtgatgtcttacta

SEQ ID NO: 315 - DNA, 40 bp, Artificial sequence
  synthetic sequence
gcctttgctttattattagaaatggggaagtgagtataga

SEQ ID NO: 316 - DNA, 40 bp, Artificial sequence
  synthetic sequence
ttatcaggagatatattaggaaaaagggaaactggagaaa

SEQ ID NO: 317 - DNA, 40 bp, Artificial sequence
  synthetic sequence
gaggagtatccagatgtcctgtccctgtaaggtgggggca

SEQ ID NO: 318 - DNA, 40 bp, Artificial sequence
  synthetic sequence
ccttcaatcaaaagggctccttaacaacttccttgcttgg

SEQ ID NO: 319 - DNA, 40 bp, Artificial sequence
  synthetic sequence
ccaccatcttggaccattagctccacaggtatcttcttcc

SEQ ID NO: 320 - DNA, 40 bp, Artificial sequence
  synthetic sequence
agtggtcataacagcagcttcagctacctctctaaagagt

SEQ ID NO: 321 - DNA, 40 bp, Artificial sequence
  synthetic sequence
ccagatataggtcaggaaatataatccactaataaaaaga

SEQ ID NO: 322 - DNA, 40 bp, Artificial sequence
  synthetic sequence
cattttgactgtagttgtttgttttttgtcattgtgacta

SEQ ID NO: 323 - DNA, 40 bp, Artificial sequence
  synthetic sequence
taacattctcactctttcatcagtaatcactcaggttatt

SEQ ID NO: 324 - DNA, 40 bp, Artificial sequence
  synthetic sequence
gaccaacagactgtgggaaaaatcagagaaggaggcatcc

SEQ ID NO: 325 - DNA, 40 bp, Artificial sequence
  synthetic sequence
gcttactagcctaaactgaaattgctatagcagagtgaac

SEQ ID NO: 326 - DNA, 40 bp, Artificial sequence
  synthetic sequence
aggtttacagatattttccacaaagagtaaaaggattgaa

SEQ ID NO: 327 - DNA, 40 bp, Artificial sequence
  synthetic sequence
tctccagatcaatgcataggaaataataatggaccataaa

SEQ ID NO: 328 - DNA, 40 bp, Artificial sequence
  synthetic sequence
atattatgacgaacaacattaggataagtccatatcaatt

SEQ ID NO: 329 - DNA, 40 bp, Artificial sequence
  synthetic sequence
atccagtcataagcacagactacgtgaagcacgtccaagt

SEQ ID NO: 330 - DNA, 40 bp, Artificial sequence
  synthetic sequence
gcaggagaaatgagaggagcaagaaagaggagccatttga

SEQ ID NO: 331 - DNA, 40 bp, Artificial sequence
  synthetic sequence
gaatagcagaaaaaggaaaggcaagtcatattaacaaatg

SEQ ID NO: 332 - DNA, 40 bp, Artificial sequence
  synthetic sequence
tcatgccaacagtacagataactctgctaataaaggtaga

SEQ ID NO: 333 - DNA, 40 bp, Artificial sequence
  synthetic sequence
taatacaggtagtagcagatatctacatagtagttaaagg

SEQ ID NO: 334 - DNA, 40 bp, Artificial sequence
  synthetic sequence
ggccatcagtacagaagattccataaaggagaacctaaag

SEQ ID NO: 335 - DNA, 40 bp, Artificial sequence
  synthetic sequence
agaataatttgtcagaagcttaaaagctgaactctgaggc

SEQ ID NO: 336 - DNA, 40 bp, Artificial sequence
  synthetic sequence
aactacaatatccttttgactgtggaaagggtggtgaaag

SEQ ID NO: 337 - DNA, 40 bp, Artificial sequence
  synthetic sequence
gttcaaggacatttgagccaacatagagaggaacattggc

SEQ ID NO: 338 - DNA, 40 bp, Artificial sequence
  synthetic sequence
tgagggatatctgtcctgatgttgtccaggatggtgatga

SEQ ID NO: 339 - DNA, 40 bp, Artificial sequence
  synthetic sequence
catataaataacgtagagaaaacaggaggggatagagatc

SEQ ID NO: 340 - DNA, 40 bp, Artificial sequence
  synthetic sequence
caaagaggcatcaaagatagggatgtttgtaaggatgaaa

SEQ ID NO: 341 - DNA, 40 bp, Artificial sequence
  synthetic sequence
ctgttcttctctgagtagccaagctcagcttggttcaagc

SEQ ID NO: 342 - DNA, 40 bp, Artificial sequence
  synthetic sequence
catactgtggatctgtagcaaattccccctgaaaacccag

SEQ ID NO: 343 - DNA, 40 bp, Artificial sequence
  synthetic sequence
tctgaccctcacattcaagttctgaggaagggccactgcc

SEQ ID NO: 344 - DNA, 40 bp, Artificial sequence
  synthetic sequence
gccttgagatacctggtccttattccttggactttggcaa

SEQ ID NO: 345 - DNA, 40 bp, Artificial sequence
  synthetic sequence
atagggcttgttttagggagaaacctgttctccaaactct

SEQ ID NO: 346 - DNA, 40 bp, Artificial sequence
  synthetic sequence
ctggtgtccatactctgaatgggaagaatgatgggattac

SEQ ID NO: 347 - DNA, 40 bp, Artificial sequence
  synthetic sequence
agcaggagaggatcaaccccatactctgaatctaagagaa

SEQ ID NO: 348 - DNA, 40 bp, Artificial sequence
  synthetic sequence
tcagatccctggatgcaagccaggtctggaaccataggca

SEQ ID NO: 349 - DNA, 40 bp, Artificial sequence
  synthetic sequence
ctcctccctaccacctttagccataaggaaacatggaatg

SEQ ID NO: 350 - DNA, 40 bp, Artificial sequence
  synthetic sequence
gacacaaacctgggcctttcaatgctataacctttcttga

SEQ ID NO: 351 - DNA, 40 bp, Artificial sequence
  synthetic sequence
ctacctgacttctgagtcaggatttataagccttgttact

SEQ ID NO: 352 - DNA, 40 bp, Artificial sequence
  synthetic sequence
tgaaccaacaagcatcgaagcaataatgagactgcccgca

SEQ ID NO: 353 - DNA, 40 bp, Artificial sequence
  synthetic sequence
gaaaagcaataatccatttttcatggtatctcatatgata

SEQ ID NO: 354 - DNA, 40 bp, Artificial sequence
  synthetic sequence
taacacttatctctctgaactttgggcttttaatatagga

SEQ ID NO: 355 - DNA, 40 bp, Artificial sequence
  synthetic sequence
ttttctgactgtctaatctttctgatctatcctggatggc

SEQ ID NO: 356 - DNA, 40 bp, Artificial sequence
  synthetic sequence
atcttcatcgaatttgggtgtttctttctaaaagtccttt

SEQ ID NO: 357 - DNA, 40 bp, Artificial sequence
  synthetic sequence
gaaattacaaatgctaaagcaaacccaaacaggcaggaat

SEQ ID NO: 358 - DNA, 40 bp, Artificial sequence
  synthetic sequence
attaggcatcttacagtttttagaatcctgcatagaactt

SEQ ID NO: 359 - DNA, 40 bp, Artificial sequence
  synthetic sequence
tacaatatttgactcttcaggttaaacatatgtcataaat

SEQ ID NO: 360 - DNA, 40 bp, Artificial sequence
  synthetic sequence
aacattcagtgaagtgaagggcctactttacttaacaaga

SEQ ID NO: 361 - DNA, 40 bp, Artificial sequence
  synthetic sequence
tcttttcctatcagtggtttacaagccttgtttatatttt

SEQ ID NO: 362 - DNA, 40 bp, Artificial sequence
  synthetic sequence
tatttttgttctgagaatatagatttagatacataatgga

SEQ ID NO: 363 - DNA, 40 bp, Artificial sequence
  synthetic sequence
caaaatctaacacaaaatctagtagaatcatttgcttaca

SEQ ID NO: 364 - DNA, 40 bp, Artificial sequence
  synthetic sequence
agaatttatgacttgtgatatccaagtcattcctggataa

SEQ ID NO: 365 - DNA, 40 bp, Artificial sequence
  synthetic sequence
ttacactagaaaatagccacaggcttcctgcaaggcagcc

SEQ ID NO: 366 - DNA, 40 bp, Artificial sequence
  synthetic sequence
agtttgaacacttgttatggtctattctctcattctttac

SEQ ID NO: 367 - DNA, 40 bp, Artificial sequence
  synthetic sequence
acttcgtgagagatgaggcagaggtacactacgaaagcaa

SEQ ID NO: 368 - DNA, 40 bp, Artificial sequence
  synthetic sequence
tcttgagaatgagcctcagccctggctcaaactcacctgc

SEQ ID NO: 369 - DNA, 40 bp, Artificial sequence
  synthetic sequence
aataggatgtctgtgctccaagttgccagagagagagatt

SEQ ID NO: 370 - DNA, 40 bp, Artificial sequence
  synthetic sequence
attaaagatccctcctgcttaattaacattcacaagtaac

SEQ ID NO: 371 - DNA, 40 bp, Artificial sequence
  synthetic sequence
acttaaagtagcgataccctttcaccctgtcctaatcaca

SEQ ID NO: 372 - DNA, 40 bp, Artificial sequence
  synthetic sequence
tctcaggtgttaactttatagtgaggactttcctgccata

SEQ ID NO: 373 - DNA, 40 bp, Artificial sequence
  synthetic sequence
atagtttcatataaatgggttcctcatcatctatgggtac

SEQ ID NO: 374 - DNA, 40 bp, Artificial sequence
  synthetic sequence
ggtatttacatttgccattccctatgccctaaatatttaa

SEQ ID NO: 375 - DNA, 40 bp, Artificial sequence
  synthetic sequence
tattgatattccttgaaaattctaagcatcttacatcttt

SEQ ID NO: 376 - DNA, 40 bp, Artificial sequence
  synthetic sequence
cttttattctccccttcaccgaatctcatcctacattggc

SEQ ID NO: 377 - DNA, 40 bp, Artificial sequence
  synthetic sequence
tagtgtcccaaattttataatttaggacttctatgatctc

SEQ ID NO: 378 - DNA, 40 bp, Artificial sequence
  synthetic sequence
atatggtcacctctttgttcaaagtcttctgatagtttcc

SEQ ID NO: 379 - DNA, 40 bp, Artificial sequence
  synthetic sequence
acaatcttcctgcttctaccactgccccactacaatttct

SEQ ID NO: 380 - DNA, 40 bp, Artificial sequence
  synthetic sequence
agtcactgtcaccaccacctaaattatagctgttgactca

SEQ ID NO: 381 - DNA, 40 bp, Artificial sequence
  synthetic sequence
ctgaccccttgccttcacctccaatgctaccactctggtc

SEQ ID NO: 382 - DNA, 40 bp, Artificial sequence
  synthetic sequence
agaaaatcctgttggtttttcgtgaaaggatgttttcaga

SEQ ID NO: 383 - DNA, 40 bp, Artificial sequence
  synthetic sequence
acatatactcacagccagaaattagcatgcactagagtgt

SEQ ID NO: 384 - DNA, 40 bp, Artificial sequence
  synthetic sequence
acccaaagactcactttgcctagcttcaaaatccttactc

SEQ ID NO: 385 - DNA, 40 bp, Artificial sequence
  synthetic sequence
tgaggtagagactgtgatgaacaaacaccttgacaaaatt

SEQ ID NO: 386 - DNA, 40 bp, Artificial sequence
  synthetic sequence
tccatatccacccacccagctttccaattttaaagccaat

SEQ ID NO: 387 - DNA, 40 bp, Artificial sequence
  synthetic sequence
aaggtatgatgtgtagacaagctccagagatggtttctca

SEQ ID NO: 388 - DNA, 40 bp, Artificial sequence
  synthetic sequence
ctctggtcagcatccaagaaatacttgatgtcactttggc

SEQ ID NO: 389 - DNA, 40 bp, Artificial sequence
  synthetic sequence
aactgtgaacttccttcagctagaggggcctggctcagaa

SEQ ID NO: 390 - DNA, 40 bp, Artificial sequence
  synthetic sequence
tgattgttctctgacttatctaccattttccctccttaaa

SEQ ID NO: 391 - DNA, 40 bp, Artificial sequence
  synthetic sequence
aaacaaaacccatcaaattccctgaccgaacagaattctg

SEQ ID NO: 392 - DNA, 40 bp, Artificial sequence
  synthetic sequence
cagaggtcacagcctaaacatcaaattccttgaggtgcgg

SEQ ID NO: 393 - DNA, 40 bp, Artificial sequence
  synthetic sequence
gaaggcaggtgtggctctgcagtgtgattgggtacttgca

SEQ ID NO: 394 - DNA, 40 bp, Artificial sequence
  synthetic sequence
catggaggaaaaactcatcagggatggaggcacgcctcta

SEQ ID NO: 395 - DNA, 40 bp, Artificial sequence
  synthetic sequence
agcttgttaaattgaattctatccttcttattcaattcta

SEQ ID NO: 396 - DNA, 40 bp, Artificial sequence
  synthetic sequence
catagttgtcagcacaatgcctaggctataggaagtactc

SEQ ID NO: 397 - DNA, 40 bp, Artificial sequence
  synthetic sequence
gcagatatagcttgatggccccatgcttggtttaacatcc

SEQ ID NO: 398 - DNA, 40 bp, Artificial sequence
  synthetic sequence
ctaaataactagaatactctttattttttcgtatcatgaa

SEQ ID NO: 399 - DNA, 40 bp, Artificial sequence
  synthetic sequence
agtgtttaaagggtgatatcagactaaacttgaaatatgt

SEQ ID NO: 400 - DNA, 40 bp, Artificial sequence
  synthetic sequence
ggatgggtctagaaagactagcattgttttaggttgagtg

SEQ ID NO: 401 - DNA, 40 bp, Artificial sequence
  synthetic sequence
tgctgccaacattaacagtcaagaaatacctccgaataac

SEQ ID NO: 402 - DNA, 40 bp, Artificial sequence
  synthetic sequence
tattgtgagaggtctgaatagtgttgtaaaataagctgaa

SEQ ID NO: 403 - DNA, 40 bp, Artificial sequence
  synthetic sequence
ttacaacatgatggcttgttgtctaaatatctcctaggga

SEQ ID NO: 404 - DNA, 40 bp, Artificial sequence
  synthetic sequence
ctaagtagaagggtactttcacaggaacagagagcaaaag

SEQ ID NO: 405 - DNA, 40 bp, Artificial sequence
  synthetic sequence
gtcttgtattgcccagtgacatgcacactggtcaaaagta

SEQ ID NO: 406 - DNA, 40 bp, Artificial sequence
  synthetic sequence
ccctatgtcttccctgatgggctagagttcctctttctca

SEQ ID NO: 407 - DNA, 40 bp, Artificial sequence
  synthetic sequence
aaagtttccccaaattttaccaatgcaagccatttctcca

SEQ ID NO: 408 - DNA, 40 bp, Artificial sequence
  synthetic sequence
aactgcagattctctgcatctccctttgccgggtctgaca

SEQ ID NO: 409 - DNA, 40 bp, Artificial sequence
  synthetic sequence
tagtgctgtggtgctgtgataggtacacaagaaatgagaa

SEQ ID NO: 410 - DNA, 40 bp, Artificial sequence
  synthetic sequence
taactagcgtcaagaactgagggccctaaactatgctagg

SEQ ID NO: 411 - DNA, 40 bp, Artificial sequence
  synthetic sequence
cattggctccgtcttcatcctgcagtgacctcagtgcctc

SEQ ID NO: 412 - DNA, 40 bp, Artificial sequence
  synthetic sequence
tgtttatgtgttatagtgttcatttactcttctggtctaa

SEQ ID NO: 413 - DNA, 40 bp, Artificial sequence
  synthetic sequence
cctttgaccccttggtcaagctgcaactttggttaaaggg

SEQ ID NO: 414 - DNA, 40 bp, Artificial sequence
  synthetic sequence
ttctcttgggttacagagattgtcatatgacaaattataa

SEQ ID NO: 415 - DNA, 40 bp, Artificial sequence
  synthetic sequence
tggaagttgtggtccaagccacagttgcagaccatacttc

SEQ ID NO: 416 - DNA, 40 bp, Artificial sequence
  synthetic sequence
ctgccctgtggcccttgcttcttacttttacttcttgtcg

SEQ ID NO: 417 - DNA, 40 bp, Artificial sequence
  synthetic sequence
aactcagatattgtggatgcgagaaattagaagtagatat

SEQ ID NO: 418 - DNA, 40 bp, Artificial sequence
  synthetic sequence
tacagaaccaccaagtagtaaggctaggatgtagacccag

SEQ ID NO: 419 - DNA, 40 bp, Artificial sequence
  synthetic sequence
tgagctctcctactgtctacattacatgagctcttattaa

SEQ ID NO: 420 - DNA, 40 bp, Artificial sequence
  synthetic sequence
aagctaataagtagacaattagtaattagaagtcagatgg

SEQ ID NO: 421 - DNA, 40 bp, Artificial sequence
  synthetic sequence
agcccaatgtacttgtagtgtagatcaacttattgaaagc

SEQ ID NO: 422 - DNA, 40 bp, Artificial sequence
  synthetic sequence
ccaatactcagaagtagattattacctcatttattgatga

SEQ ID NO: 423 - DNA, 40 bp, Artificial sequence
  synthetic sequence
gctagaatcaaatttaagtttatcatatgaggccgggcac

SEQ ID NO: 424 - DNA, 40 bp, Artificial sequence
  synthetic sequence
taatactaatgataagtaacacctcttgagtacttagtat

SEQ ID NO: 425 - DNA, 40 bp, Artificial sequence
  synthetic sequence
atggtaattctgtgagatatgtattattgaacatactata

SEQ ID NO: 426 - DNA, 40 bp, Artificial sequence
  synthetic sequence
tgaaagagaagtgggaattaatacttactgaaatctttct

SEQ ID NO: 427 - DNA, 40 bp, Artificial sequence
  synthetic sequence
gagagacacgaggaaatagtgtagatttaggctggaggta

SEQ ID NO: 428 - DNA, 40 bp, Artificial sequence
  synthetic sequence
gttgagagggaaacaagatggtgaagggactagaaaccac

SEQ ID NO: 429 - DNA, 40 bp, Artificial sequence
  synthetic sequence
caaggttctgaacatgagaaatttttaggaatctgcacag

SEQ ID NO: 430 - DNA, 40 bp, Artificial sequence
  synthetic sequence
tgccatctaaaaaaatctgacttcactggaaacatggaag

SEQ ID NO: 431 - DNA, 40 bp, Artificial sequence
  synthetic sequence
gggatcctctcttaagtgtttcctgctggaatctcctcac

SEQ ID NO: 432 - DNA, 40 bp, Artificial sequence
  synthetic sequence
gtttccttcatgtgacagggagcctcctgccccgaacttc

SEQ ID NO: 433 - DNA, 40 bp, Artificial sequence
  synthetic sequence
ttggataagagtagggaagaacctagagcctacgctgagc

SEQ ID NO: 434 - DNA, 40 bp, Artificial sequence
  synthetic sequence
atctggggctttgtgaagactggcttaaaatcagaagccc

SEQ ID NO: 435 - DNA, 40 bp, Artificial sequence
  synthetic sequence
accgcaatgcttcctgcccattcagggctccagcatgtag

SEQ ID NO: 436 - DNA, 40 bp, Artificial sequence
  synthetic sequence
tatggggaagcagggtatgaaagagctctgaatgaaatgg

SEQ ID NO: 437 - DNA, 40 bp, Artificial sequence
  synthetic sequence
ggttgcatgaatcagattatcaacagaaatgttgagacaa

SEQ ID NO: 438 - DNA, 40 bp, Artificial sequence
  synthetic sequence
aatgcaggcctaggcatgactgaaggctctctcataattc

SEQ ID NO: 439 - DNA, 40 bp, Artificial sequence
  synthetic sequence
taacgttttcttgtctgctaccccatcatatgcacaacaa

SEQ ID NO: 440 - DNA, 40 bp, Artificial sequence
  synthetic sequence
ttaattcccaaactcatatagctctgagaaagtctatgct

SEQ ID NO: 441 - DNA, 40 bp, Artificial sequence
  synthetic sequence
ccctataggggatttctaccctgagcaaaaggctggtctt

SEQ ID NO: 442 - DNA, 40 bp, Artificial sequence
  synthetic sequence
tcctcaccatatagaaagcttttaacccatcattgaataa

SEQ ID NO: 443 - DNA, 40 bp, Artificial sequence
  synthetic sequence
taagctgtctagcaaaagcaagggcttggaaaatctgtga

SEQ ID NO: 444 - DNA, 40 bp, Artificial sequence
  synthetic sequence
aggattagaagattcttctgtgtgtaagaatttcataaac

SEQ ID NO: 445 - DNA, 40 bp, Artificial sequence
  synthetic sequence
attatcttctggaatagggaatcaagttatattatgtaac

SEQ ID NO: 446 - DNA, 40 bp, Artificial sequence
  synthetic sequence
ctctctggttgactgttagagttctggcacttgtcactat

SEQ ID NO: 447 - DNA, 40 bp, Artificial sequence
  synthetic sequence
tcttcagttagatggttaactttgtgaagttgaaaactgt

SEQ ID NO: 448 - DNA, 40 bp, Artificial sequence
  synthetic sequence
ctacaccatgtggagaaggggtggtggttttgattgctgc

SEQ ID NO: 449 - DNA, 40 bp, Artificial sequence
  synthetic sequence
actttcctaacctgagcctaacatccctgacatcaggaaa

SEQ ID NO: 450 - DNA, 40 bp, Artificial sequence
  synthetic sequence
tacactttattcgtctgtgtcctgctctgggatgatagtc

SEQ ID NO: 451 - DNA, 40 bp, Artificial sequence
  synthetic sequence
tactctttgcattccactgtttttcctaagtgactaaaaa

SEQ ID NO: 452 - DNA, 40 bp, Artificial sequence
  synthetic sequence
aaaggcctcccaggccaagttatccattcagaaagcattt

SEQ ID NO: 453 - DNA, 40 bp, Artificial sequence
  synthetic sequence
tattgacatgtacttcttggcagtctgtatgctggatgct

SEQ ID NO: 454 - DNA, 40 bp, Artificial sequence
  synthetic sequence
tttggtcctaattatgtctttgctcactatccaataaata

SEQ ID NO: 455 - DNA, 40 bp, Artificial sequence
  synthetic sequence
gttaaaaaaactacctctcaacttgctcaagcatacactc

SEQ ID NO: 456 - DNA, 40 bp, Artificial sequence
  synthetic sequence
taattagtgctttgcataattaatcatatttaatactctt

SEQ ID NO: 457 - DNA, 40 bp, Artificial sequence
  synthetic sequence
actagtgttctgtactttatgcccattcatctttaactgt

SEQ ID NO: 458 - DNA, 40 bp, Artificial sequence
  synthetic sequence
gtattttttgtttaactgcaatcattcttgctgcaggtga

SEQ ID NO: 459 - DNA, 40 bp, Artificial sequence
  synthetic sequence
gcagtgacttataaatgctaactactctagaaatgtttgc

SEQ ID NO: 460 - DNA, 40 bp, Artificial sequence
  synthetic sequence
ttataagcatgattacaggagttttaacaggctcataaga

SEQ ID NO: 461 - DNA, 40 bp, Artificial sequence
  synthetic sequence
agtatccctcaagtagtgtcaggaattagtcatttaaata

SEQ ID NO: 462 - DNA, 40 bp, Artificial sequence
  synthetic sequence
agtcacccatttggtatattaaagatgtgttgtctactgt

SEQ ID NO: 463 - DNA, 40 bp, Artificial sequence
  synthetic sequence
tggtcataaaacattgaattctaatctccctctcaaccct

SEQ ID NO: 464 - DNA, 40 bp, Artificial sequence
  synthetic sequence
acagttgaaaagacctaagcttgtgcctgatttaagcctt

SEQ ID NO: 465 - DNA, 40 bp, Artificial sequence
  synthetic sequence
caactacagggccttgaactgcacactttcagtccggtcc

SEQ ID NO: 466 - DNA, 40 bp, Artificial sequence
  synthetic sequence
gtggttctttgaagagacttccacctgggaacagttaaac

SEQ ID NO: 467 - DNA, 40 bp, Artificial sequence
  synthetic sequence
tggaggaaatatttatccccaggtagttccctttttgcac

SEQ ID NO: 468 - DNA, 40 bp, Artificial sequence
  synthetic sequence
gcctggtgcttttggtaggggagcttgcactttccccctt

SEQ ID NO: 469 - DNA, 40 bp, Artificial sequence
  synthetic sequence
tctcatttctttgagaacttcagggaaaatagacaaggac

SEQ ID NO: 470 - DNA, 40 bp, Artificial sequence
  synthetic sequence
caaacttttcaagccttctctaatcttaaaggtaaacaag

SEQ ID NO: 471 - DNA, 40 bp, Artificial sequence
  synthetic sequence
tcaacaaaggagaaaagtttgttggcctccaaaggcacag

SEQ ID NO: 472 - DNA, 40 bp, Artificial sequence
  synthetic sequence
gatgcaacagaccttggaagcatacaggagagctgaactt

SEQ ID NO: 473 - DNA, 40 bp, Artificial sequence
  synthetic sequence
catctgagatcccagcttctaagaccttcaattctcactc

SEQ ID NO: 474 - DNA, 40 bp, Artificial sequence
  synthetic sequence
tatcttaacagtgagtgaacaggaaatctcctcttttccc

SEQ ID NO: 475 - DNA, 40 bp, Artificial sequence
  synthetic sequence
aactcatgctttgtagatgactagatcaaaaaatttcagc

SEQ ID NO: 476 - DNA, 40 bp, Artificial sequence
  synthetic sequence
tcaaaggaagtcaaaagatgtgaaaaacaatttctgaccc

SEQ ID NO: 477 - DNA, 40 bp, Artificial sequence
  synthetic sequence
tgccttcacttaagtaatcaattcctaggttatattctga

SEQ ID NO: 478 - DNA, 40 bp, Artificial sequence
  synthetic sequence
ccctaccttgttcaaaatgttcctgtccagaccaaagtac

SEQ ID NO: 479 - DNA, 40 bp, Artificial sequence
  synthetic sequence
gcacttacaaattatactacgctctatactttttgtttaa

SEQ ID NO: 480 - DNA, 40 bp, Artificial sequence
  synthetic sequence
ctttagtttcatttcaaacaatccatacacacacagccct

SEQ ID NO: 481 - DNA, 40 bp, Artificial sequence
  synthetic sequence
tagggaccacagggttaagggggcagtagaattatactcc

SEQ ID NO: 482 - DNA, 40 bp, Artificial sequence
  synthetic sequence
ctcacaattaagctaagcagctaagagtcttgcagggtag

SEQ ID NO: 483 - DNA, 40 bp, Artificial sequence
  synthetic sequence
gttgaaagacagagaggatggggtgctatgccccaaatca

SEQ ID NO: 484 - DNA, 40 bp, Artificial sequence
  synthetic sequence
gcttgtctaattttatatatcaccctactgaacatgaccc

SEQ ID NO: 485 - DNA, 40 bp, Artificial sequence
  synthetic sequence
aatattgtacacgtacaccaaagcatcatgttgtacccca

SEQ ID NO: 486 - DNA, 40 bp, Artificial sequence
  synthetic sequence
tgtgaagtggtggatttgttaattagccttatttaaccat

SEQ ID NO: 487 - DNA, 40 bp, Artificial sequence
  synthetic sequence
tgacacatatgacattttaactatgttccagatttttgaa

SEQ ID NO: 488 - DNA, 40 bp, Artificial sequence
  synthetic sequence
gcaaggaatcattcaatgttttctaaatctattactgcat

SEQ ID NO: 489 - DNA, 40 bp, Artificial sequence
  synthetic sequence
cattttcataggttttcctcgattgatcattattcatgat

SEQ ID NO: 490 - DNA, 40 bp, Artificial sequence
  synthetic sequence
aaagtgatcaagatatttttagttcaggctccaaaatttt

SEQ ID NO: 491 - DNA, 40 bp, Artificial sequence
  synthetic sequence
ctttacaggccgagaaaaatgaatctgaattcctgacctc

SEQ ID NO: 492 - DNA, 40 bp, Artificial sequence
  synthetic sequence
tccactcaaggcctacattctgctataatgcaatttcaag

SEQ ID NO: 493 - DNA, 40 bp, Artificial sequence
  synthetic sequence
aactgcttaaaattaatggcacaagtcatgtttttgatgt

SEQ ID NO: 494 - DNA, 40 bp, Artificial sequence
  synthetic sequence
ctgactgtgacgtagcaataaagaaacccacgtttcatat

SEQ ID NO: 495 - DNA, 40 bp, Artificial sequence
  synthetic sequence
ctggcccactgcttggaggagagcactcaggaccatgaac

SEQ ID NO: 496 - DNA, 40 bp, Artificial sequence
  synthetic sequence
ttctgaaatgataaagtcaatcacaggaaggcacctggac

SEQ ID NO: 497 - DNA, 40 bp, Artificial sequence
  synthetic sequence
atcattctctttcccttcctctatgtggcagaaagtaaaa

SEQ ID NO: 498 - DNA, 40 bp, Artificial sequence
  synthetic sequence
ggagataataatgtgttactccctaaggcagagtgccctt

SEQ ID NO: 499 - DNA, 40 bp, Artificial sequence
  synthetic sequence
caattaacttggccatgtgactggttgtgactaaaataat

SEQ ID NO: 500 - DNA, 40 bp, Artificial sequence
  synthetic sequence
cactaaatcaatatacttctcaacaatttccaacagccct

SEQ ID NO: 501 - DNA, 40 bp, Artificial sequence
  synthetic sequence
ctaggctcctgagtttgctggggatgcgaagaacccttat

SEQ ID NO: 502 - DNA, 40 bp, Artificial sequence
  synthetic sequence
ccgaggaccccgcactcggagccgccagccggccccaccg

SEQ ID NO: 503 - DNA, 40 bp, Artificial sequence
  synthetic sequence
ttggaagcacagggtgtgggataatgctaattactagtga

SEQ ID NO: 504 - DNA, 40 bp, Artificial sequence
  synthetic sequence
gttcagtatgcctttgattttacaataatattcctgttat

SEQ ID NO: 505 - DNA, 40 bp, Artificial sequence
  synthetic sequence
agattccatgaagtattacagcatttggtagtctttttgc

SEQ ID NO: 506 - DNA, 40 bp, Artificial sequence
  synthetic sequence
tatttgctctgaaataagacataatttggggtgagaaagc

SEQ ID NO: 507 - DNA, 40 bp, Artificial sequence
  synthetic sequence
actcatgatatttggctctagaatacatgctctgaatcat

SEQ ID NO: 508 - DNA, 40 bp, Artificial sequence
  synthetic sequence
tccaagatgaagtggctactaactgacagagggcataatt

SEQ ID NO: 509 - DNA, 40 bp, Artificial sequence
  synthetic sequence
tattcacagtaactctgtgcctcaagtactattgtaatac

SEQ ID NO: 510 - DNA, 40 bp, Artificial sequence
  synthetic sequence
acatcctcaatctacacactaggatagtataaaagtaata

SEQ ID NO: 511 - DNA, 40 bp, Artificial sequence
  synthetic sequence
gtctacccatatgtgaccttcatgtctttgctctaagccc

SEQ ID NO: 512 - DNA, 40 bp, Artificial sequence
  synthetic sequence
cgtgtaatccttgacaatgtcatctcatctatttattccc

SEQ ID NO: 513 - DNA, 40 bp, Artificial sequence
  synthetic sequence
tctgaaagagactaaccttccctcgctttgcagagaaaga

SEQ ID NO: 514 - DNA, 40 bp, Artificial sequence
  synthetic sequence
atgcatggattctcttgaaaaaatgtttctgccatgatgt

SEQ ID NO: 515 - DNA, 40 bp, Artificial sequence
  synthetic sequence
tagttgaagacctactgtgttcagggccgtgagccagggc

SEQ ID NO: 516 - DNA, 40 bp, Artificial sequence
  synthetic sequence
caacgtggagagctgtcctggcaccatttcttcctgctgt

SEQ ID NO: 517 - DNA, 40 bp, Artificial sequence
  synthetic sequence
atcctcaaaggagcctggcttgggctaacaaggaagaact

SEQ ID NO: 518 - DNA, 40 bp, Artificial sequence
  synthetic sequence
tgcctgggaccctgccccaagcaaagtaataatctgaatg

SEQ ID NO: 519 - DNA, 40 bp, Artificial sequence
  synthetic sequence
ctggtgtgtccagtgtgatccctgcacccatgcccggagc

SEQ ID NO: 520 - DNA, 40 bp, Artificial sequence
  synthetic sequence
ctgccccctgcagcagggaaggggctctggaagggtctga

SEQ ID NO: 521 - DNA, 40 bp, Artificial sequence
  synthetic sequence
tagctgctgccccactatgcaccatcgcttatctgttctt

SEQ ID NO: 522 - DNA, 40 bp, Artificial sequence
  synthetic sequence
gaaacccgaaaaatgtcctggtcctcttcttaagtctggg

SEQ ID NO: 523 - DNA, 40 bp, Artificial sequence
  synthetic sequence
gctgagaacatgactctgcttggcgttccatttaattgac

SEQ ID NO: 524 - DNA, 40 bp, Artificial sequence
  synthetic sequence
gagagggtgtgcatttgaagtatagatttgttaaacatag

SEQ ID NO: 525 - DNA, 40 bp, Artificial sequence
  synthetic sequence
catcaggcaaaaatacttcgatgggactgtgttctttcag

SEQ ID NO: 526 - DNA, 40 bp, Artificial sequence
  synthetic sequence
tctaaagtgatgtaatgttgccacggaaattctaatccct

SEQ ID NO: 527 - DNA, 40 bp, Artificial sequence
  synthetic sequence
cgtgcagaaccagctctgtcttcccagacactgtcgcttt

SEQ ID NO: 528 - DNA, 40 bp, Artificial sequence
  synthetic sequence
acccctgagcacctcagtgtccgtgactgtggagcggagg

SEQ ID NO: 529 - DNA, 40 bp, Artificial sequence
  synthetic sequence
ctgcctgggacacgtacggctgcccagtgatcctgagcgc

SEQ ID NO: 530 - DNA, 40 bp, Artificial sequence
  synthetic sequence
cacagccggatggtgtgggagctggcactgccggggctcc

SEQ ID NO: 531 - DNA, 40 bp, Artificial sequence
  synthetic sequence
cgtcttggcagaggctccctgtcatcaaggacctgaggtt

SEQ ID NO: 532 - DNA, 40 bp, Artificial sequence
  synthetic sequence
gaccccacaaagatgagcgggtccccttcccaattttcgg

SEQ ID NO: 533 - DNA, 40 bp, Artificial sequence
  synthetic sequence
tcaggaagccggtgctcagcaaacttatctgaagctcttg

SEQ ID NO: 534 - DNA, 40 bp, Artificial sequence
  synthetic sequence
gaggctgcagaggaacatcgtttggtcaaatgtgaaatgt

SEQ ID NO: 535 - DNA, 40 bp, Artificial sequence
  synthetic sequence
ctagcttctagaaagtgctgccaatttggggaccaaggga

SEQ ID NO: 536 - DNA, 40 bp, Artificial sequence
  synthetic sequence
ggaaacacttctttttcccttgacaaaggacatcctctgc

SEQ ID NO: 537 - DNA, 40 bp, Artificial sequence
  synthetic sequence
gcatgtgcataaacactcgtgtgtgtgtccttttatccca

SEQ ID NO: 538 - DNA, 40 bp, Artificial sequence
  synthetic sequence
ccaaatctctatacatgtccatagagagaggcagacgtat

SEQ ID NO: 539 - DNA, 40 bp, Artificial sequence
  synthetic sequence
gggttgaagacaaggggctcagagcttgctttttatacac

SEQ ID NO: 540 - DNA, 40 bp, Artificial sequence
  synthetic sequence
agattcatcttcatggcaggacttcaggcaagagaggccc

SEQ ID NO: 541 - DNA, 40 bp, Artificial sequence
  synthetic sequence
ctcaccccttagcaggaccctgacggaactgggtacaggc

SEQ ID NO: 542 - DNA, 40 bp, Artificial sequence
  synthetic sequence
ggttgggagacaatgggtggcccctcggtgtggtgtcctc

SEQ ID NO: 543 - DNA, 40 bp, Artificial sequence
  synthetic sequence
agagtctagagggcccgtggggacgggagtcctgggaacc

SEQ ID NO: 544 - DNA, 40 bp, Artificial sequence
  synthetic sequence
gcggcatgtccggcttcaccctgcccagaatcacagcctc

SEQ ID NO: 545 - DNA, 40 bp, Artificial sequence
  synthetic sequence
atggttaaaaaattctcctacttaagactcccagacccct

SEQ ID NO: 546 - DNA, 40 bp, Artificial sequence
  synthetic sequence
tgagattccagggctggttccacaacggccggcatcggcc

SEQ ID NO: 547 - DNA, 40 bp, Artificial sequence
  synthetic sequence
ctgagtcactaacaaagctcaggcctgaccacaggacatt

SEQ ID NO: 548 - DNA, 40 bp, Artificial sequence
  synthetic sequence
ggctggcctacctgccacggggccagggctgggtgctttc

SEQ ID NO: 549 - DNA, 40 bp, Artificial sequence
  synthetic sequence
gggctctggacgctggaggcctgaggctgcaccccaggtt

SEQ ID NO: 550 - DNA, 40 bp, Artificial sequence
  synthetic sequence
acagtggccactcacccactgggcccacatccccacaggc

SEQ ID NO: 551 - DNA, 40 bp, Artificial sequence
  synthetic sequence
actctgccagcctttgatgcctcgctgagacagagggtct

SEQ ID NO: 552 - DNA, 40 bp, Artificial sequence
  synthetic sequence
agccggggctctggccccatccaggggctcccccagcagc

SEQ ID NO: 553 - DNA, 40 bp, Artificial sequence
  synthetic sequence
ccttggaagtcagtcagcaggtcaggacacagttcagccc

SEQ ID NO: 554 - DNA, 40 bp, Artificial sequence
  synthetic sequence
ttacatgcagttggtcttctcctgtgaatggggaaactga

SEQ ID NO: 555 - DNA, 40 bp, Artificial sequence
  synthetic sequence
ctgcatcacagaacagctgcatttctaatgtcaggcttct

SEQ ID NO: 556 - DNA, 40 bp, Artificial sequence
  synthetic sequence
cagcctgggaggcttgtcaacctcctttgacaagcacgcc

SEQ ID NO: 557 - DNA, 40 bp, Artificial sequence
  synthetic sequence
agaaactggggctccagggcatggaggctgcctgtggcca

SEQ ID NO: 558 - DNA, 40 bp, Artificial sequence
  synthetic sequence
tcccggcctggaggaagtcttattagcctcatttcatgga

SEQ ID NO: 559 - DNA, 40 bp, Artificial sequence
  synthetic sequence
tcctgccagccccctcacgctcacgaattcagtcccaggg

SEQ ID NO: 560 - DNA, 40 bp, Artificial sequence
  synthetic sequence
aattctaaaggtgaagggacgtctacacccccaacaaaac

SEQ ID NO: 561 - DNA, 40 bp, Artificial sequence
  synthetic sequence
ggaaatattagtcccctctgcctgggacaagaccaccgaa

SEQ ID NO: 562 - DNA, 40 bp, Artificial sequence
  synthetic sequence
aaacacacctctgaatggaaagctgagaaacagtgatctc

SEQ ID NO: 563 - DNA, 40 bp, Artificial sequence
  synthetic sequence
actgcaccccctcccttcccgtgccggcaatttaaccggg

SEQ ID NO: 564 - DNA, 40 bp, Artificial sequence
  synthetic sequence
tgccttcctaccttgaccagtcggtccttgcgggggtccc

SEQ ID NO: 565 - DNA, 40 bp, Artificial sequence
  synthetic sequence
atttccttcatcttgtccttctagcctggagactcttcgg

SEQ ID NO: 566 - DNA, 40 bp, Artificial sequence
  synthetic sequence
aatgcccgaaaattccagcagcagcccaagatggtggcca

SEQ ID NO: 567 - DNA, 40 bp, Artificial sequence
  synthetic sequence
cgttgcaaatgcccaagggggtaaccctaaaagttaaagg

SEQ ID NO: 568 - DNA, 40 bp, Artificial sequence
  synthetic sequence
acacaacccctgtgcaagtttcattccggcgcacaggggc

SEQ ID NO: 569 - DNA, 40 bp, Artificial sequence
  synthetic sequence
tgcaagaactaatttagcatgcaaggacggggaggaccgg

SEQ ID NO: 570 - DNA, 40 bp, Artificial sequence
  synthetic sequence
gccacgagggcacccacgggcggacagacggccaaagaat

SEQ ID NO: 571 - DNA, 40 bp, Artificial sequence
  synthetic sequence
accccatatccaagccggcagaatgggcgcatttccaaga

SEQ ID NO: 572 - DNA, 40 bp, Artificial sequence
  synthetic sequence
gcctggggagaccacgagaaggggtgactggggcgcggcg

SEQ ID NO: 573 - DNA, 40 bp, Artificial sequence
  synthetic sequence
ctgcagtaggggacaactaggaaggccggcaggccacacg

SEQ ID NO: 574 - DNA, 40 bp, Artificial sequence
  synthetic sequence
gagtgggtcccccgggatttagggggtgaggtggaggtgg

SEQ ID NO: 575 - DNA, 40 bp, Artificial sequence
  synthetic sequence
tccccgccagggaagaggggtgcagggggccccgtccgcc

SEQ ID NO: 576 - DNA, 40 bp, Artificial sequence
  synthetic sequence
tgaggcgccgcgcctgccctgcggcggagttgcccctgta

SEQ ID NO: 577 - DNA, 40 bp, Artificial sequence
  synthetic sequence
aaacgccgggagcagcgaggggcagagcccaaaagccatc

SEQ ID NO: 578 - DNA, 40 bp, Artificial sequence
  synthetic sequence
ttgttaagcaaagatcaaagcccggcagagaatgggagcg

SEQ ID NO: 579 - DNA, 40 bp, Artificial sequence
  synthetic sequence
caacttcaacaaaactcccctgtagtccgtgtgacgttac

SEQ ID NO: 580 - DNA, 40 bp, Artificial sequence
  synthetic sequence
ctgctactgcgccgacagccctctggaggctccaggactt

SEQ ID NO: 581 - DNA, 40 bp, Artificial sequence
  synthetic sequence
gctcttctgcccctcgccggagcgtgcggactctgctgct

SEQ ID NO: 582 - DNA, 40 bp, Artificial sequence
  synthetic sequence
tccgcgctcggctctcgcttctgctgccccgcgctccctc

SEQ ID NO: 583 - DNA, 40 bp, Artificial sequence
  synthetic sequence
tttccacttcgcagcacaggagctggtgttccatggctgg

SEQ ID NO: 584 - DNA, 40 bp, Artificial sequence
  synthetic sequence
ggtcgttgaggaggttggcatcggggtacgcgcggcggat

SEQ ID NO: 585 - DNA, 40 bp, Artificial sequence
  synthetic sequence
tgtcctacttcaaatgtgtgcagaaggaggtcctgccgtc

SEQ ID NO: 586 - DNA, 40 bp, Artificial sequence
  synthetic sequence
tcgggcggctctcttaagacttccctgcaacttgttgccc

SEQ ID NO: 587 - DNA, 40 bp, Artificial sequence
  synthetic sequence
acccacgtttctttgctactcacccccctcccttctctcc

SEQ ID NO: 588 - DNA, 40 bp, Artificial sequence
  synthetic sequence
ctagaactttgaagtttgccgtggtgtttctagggatccg

SEQ ID NO: 589 - DNA, 40 bp, Artificial sequence
  synthetic sequence
agaagggggtccgggaggggtgccttcgggagaagccagt

SEQ ID NO: 590 - DNA, 40 bp, Artificial sequence
  synthetic sequence
caggggcaccccaatgggcccgagggtgcgggctggcagg

SEQ ID NO: 591 - DNA, 40 bp, Artificial sequence
  synthetic sequence
gggtgcgctttgtgtcccccgcctgcgccccagcccggct

SEQ ID NO: 592 - DNA, 40 bp, Artificial sequence
  synthetic sequence
gcctcagcggccgggagccgccaactccggggggaggggg

SEQ ID NO: 593 - DNA, 40 bp, Artificial sequence
  synthetic sequence
aaagtgcagtaatacccttgatcagagttgatgacttgaa

SEQ ID NO: 594 - DNA, 40 bp, Artificial sequence
  synthetic sequence
gagagaaataaagtagttgctctatttgtaaattgaaaag

SEQ ID NO: 595 - DNA, 40 bp, Artificial sequence
  synthetic sequence
ggtagcagtgattgctgtatatttgtgaaaaggaggcaag

SEQ ID NO: 596 - DNA, 40 bp, Artificial sequence
  synthetic sequence
tgctgataatggaagtgcagtgggttagctttgtttccat

SEQ ID NO: 597 - DNA, 40 bp, Artificial sequence
  synthetic sequence
ccgttctaccgtgactagtatggaattgtgggaaccagaa

SEQ ID NO: 598 - DNA, 40 bp, Artificial sequence
  synthetic sequence
ttaacatcagtgtcaactgcagtgttgtttctgagtaata

SEQ ID NO: 599 - DNA, 40 bp, Artificial sequence
  synthetic sequence
cataactccatgctctcaaaccaatcactccttcattcat

SEQ ID NO: 600 - DNA, 40 bp, Artificial sequence
  synthetic sequence
ttctcctatgctgcaccagaaagggttttgtgggttatca

SEQ ID NO: 601 - DNA, 40 bp, Artificial sequence
  synthetic sequence
atcgttcagcatctttaggaaatatccagagactgcattg

SEQ ID NO: 602 - DNA, 40 bp, Artificial sequence
  synthetic sequence
tttattaagagcaaaaaaagcctgtttcgttagccagtca

SEQ ID NO: 603 - DNA, 40 bp, Artificial sequence
  synthetic sequence
ttgttcatatgcctaacttaataaattcttcatacagaaa

SEQ ID NO: 604 - DNA, 40 bp, Artificial sequence
  synthetic sequence
ataacttttaaacccaaacacctagagatttcattatgta

SEQ ID NO: 605 - DNA, 40 bp, Artificial sequence
  synthetic sequence
ttcttaccattaagtcttccaaatgataatttattataaa

SEQ ID NO: 606 - DNA, 40 bp, Artificial sequence
  synthetic sequence
tatgtaaggacaacttcattatatgcttgaagaaattgtt

SEQ ID NO: 607 - DNA, 40 bp, Artificial sequence
  synthetic sequence
aatcttaaaagtgacactagtcacattccacacggttaaa

SEQ ID NO: 608 - DNA, 40 bp, Artificial sequence
  synthetic sequence
attttgaaaactattcctttatctggaatgaatgtaaacc

SEQ ID NO: 609 - DNA, 40 bp, Artificial sequence
  synthetic sequence
ttgcattaagggcaccagaaacttatagaaaaccaaaaag

SEQ ID NO: 610 - DNA, 40 bp, Artificial sequence
  synthetic sequence
taaaagacagtgaactgaacagtaattaacattacatcca

SEQ ID NO: 611 - DNA, 40 bp, Artificial sequence
  synthetic sequence
caaaaaactgtgtttatcatataccaaacattttcaagtt

SEQ ID NO: 612 - DNA, 40 bp, Artificial sequence
  synthetic sequence
tctcaggatattttgttctctgacacaaatacaccagtca

SEQ ID NO: 613 - DNA, 40 bp, Artificial sequence
  synthetic sequence
tagctttacatctcagaatgaatcaatgtgggggcagaaa

SEQ ID NO: 614 - DNA, 40 bp, Artificial sequence
  synthetic sequence
agacctatatacctatagtgcctaatagacaataagccac

SEQ ID NO: 615 - DNA, 40 bp, Artificial sequence
  synthetic sequence
tctctcccctgcctagactaaggtaagtgggtcttacctt

SEQ ID NO: 616 - DNA, 40 bp, Artificial sequence
  synthetic sequence
catcctgcttttaaaacccttagtgctcagcggcttgtct

SEQ ID NO: 617 - DNA, 40 bp, Artificial sequence
  synthetic sequence
agcttataaacttcagagtaatgtagcacaaatgtctgtc

SEQ ID NO: 618 - DNA, 40 bp, Artificial sequence
  synthetic sequence
aacttgaaataaaactttaaacgttgattgattctttccc

SEQ ID NO: 619 - DNA, 40 bp, Artificial sequence
  synthetic sequence
gacaggcttagagtccataacaaacaatcttagctggaaa

SEQ ID NO: 620 - DNA, 40 bp, Artificial sequence
  synthetic sequence
tgctcaacaacacttgtggaagagcagggcaagctatttc

SEQ ID NO: 621 - DNA, 40 bp, Artificial sequence
  synthetic sequence
ttacaacatcactgtagacattacttttacccacagtgcc

SEQ ID NO: 622 - DNA, 40 bp, Artificial sequence
  synthetic sequence
atcctagttgtatatacttcttggataaagtatcttcgta

SEQ ID NO: 623 - DNA, 40 bp, Artificial sequence
  synthetic sequence
atttttggggagtgccattcctgcaggtcttgaagacagg

SEQ ID NO: 624 - DNA, 40 bp, Artificial sequence
  synthetic sequence
cacacagccaatgaaactgacagagccaatgcaaccaaaa

SEQ ID NO: 625 - DNA, 40 bp, Artificial sequence
  synthetic sequence
acgacttcaatcaagagaaacaggcaggtcagagtgtgaa

SEQ ID NO: 626 - DNA, 40 bp, Artificial sequence
  synthetic sequence
ctggttatcagggttcatagcacataggtttgacaaccac

SEQ ID NO: 627 - DNA, 40 bp, Artificial sequence
  synthetic sequence
tttattattcagctgggtaagccaagtgacagtcttcccc

SEQ ID NO: 628 - DNA, 40 bp, Artificial sequence
  synthetic sequence
gttttattctaggaatcaactgctttctaaaaatgtctaa

SEQ ID NO: 629 - DNA, 40 bp, Artificial sequence
  synthetic sequence
tttactgatggtacttattcccccaattattgattattga

SEQ ID NO: 630 - DNA, 40 bp, Artificial sequence
  synthetic sequence
gcatttaggaatattcaatattgatactaaggtcatcttt

SEQ ID NO: 631 - DNA, 40 bp, Artificial sequence
  synthetic sequence
tactctgtaatgtagtaatctttatgaagaaataaatttg

SEQ ID NO: 632 - DNA, 40 bp, Artificial sequence
  synthetic sequence
attttgaaaaaatgtttcactgcattttactatacaagct

SEQ ID NO: 633 - DNA, 40 bp, Artificial sequence
  synthetic sequence
accacacattcatcaaaaaatacctcaaagaaaattctgc

SEQ ID NO: 634 - DNA, 40 bp, Artificial sequence
  synthetic sequence
gttgtcacaataaactcagtactgagtaaaatatcacaaa

SEQ ID NO: 635 - DNA, 40 bp, Artificial sequence
  synthetic sequence
gagtatatattgtattacttacctgatgcgcaaagaccca

SEQ ID NO: 636 - DNA, 40 bp, Artificial sequence
  synthetic sequence
aaaatgacagcaacataggtgccacctgaggtccacatct

SEQ ID NO: 637 - DNA, 40 bp, Artificial sequence
  synthetic sequence
tggagagagtggggttaatctgttactacactttgctact

SEQ ID NO: 638 - DNA, 40 bp, Artificial sequence
  synthetic sequence
atttccatcattttgtctttcagtaagcatgtacgaagta

SEQ ID NO: 639 - DNA, 40 bp, Artificial sequence
  synthetic sequence
gagatgaagatggtacatcagtagggagcccctctactgg

SEQ ID NO: 640 - DNA, 40 bp, Artificial sequence
  synthetic sequence
tctaattcatcaaagtattctgggttgattccaggtacgt

SEQ ID NO: 641 - DNA, 40 bp, Artificial sequence
  synthetic sequence
acaaactcgttttgtacagagaggaaaatattaaaacacc

SEQ ID NO: 642 - DNA, 40 bp, Artificial sequence
  synthetic sequence
atgttaattataaacactgttataagttttacaaatgtaa

SEQ ID NO: 643 - DNA, 40 bp, Artificial sequence
  synthetic sequence
tccactggcagagagaatatatgtttccattacggtccca

SEQ ID NO: 644 - DNA, 40 bp, Artificial sequence
  synthetic sequence
tcaaaggttttctatcacgttttctattatttactcacat

SEQ ID NO: 645 - DNA, 40 bp, Artificial sequence
  synthetic sequence
aaaaacaagagtcacacaacctatgctccacaatatctgc

SEQ ID NO: 646 - DNA, 40 bp, Artificial sequence
  synthetic sequence
ataggttattctacaatcgacaccaactatcagcggcttt

SEQ ID NO: 647 - DNA, 40 bp, Artificial sequence
  synthetic sequence
attgaattaaatgatggcttgattatccaggaatcagcca

SEQ ID NO: 648 - DNA, 40 bp, Artificial sequence
  synthetic sequence
cttaccataacagagtaatctctagcttattccaaggata

SEQ ID NO: 649 - DNA, 40 bp, Artificial sequence
  synthetic sequence
acctaaaatttaactagaatcacttttcaatgaagctgct

SEQ ID NO: 650 - DNA, 40 bp, Artificial sequence
  synthetic sequence
taaactaagagcctttgatcttgccttattctgataaaat

SEQ ID NO: 651 - DNA, 40 bp, Artificial sequence
  synthetic sequence
aaataataattcacaaggaaatccttattgtttatttaaa

SEQ ID NO: 652 - DNA, 40 bp, Artificial sequence
  synthetic sequence
gtaatatgtaggttaaacagaaatgttggttgaatcatgt

SEQ ID NO: 653 - DNA, 40 bp, Artificial sequence
  synthetic sequence
tgcagacactaatcaaaccaaacagggccaattaaaattg

SEQ ID NO: 654 - DNA, 40 bp, Artificial sequence
  synthetic sequence
taaagtgcaatgggacagagcaacttcattttcacaaaca

SEQ ID NO: 655 - DNA, 40 bp, Artificial sequence
  synthetic sequence
taatctaattgccagaaatgcttgcccattgcaatgggag

SEQ ID NO: 656 - DNA, 40 bp, Artificial sequence
  synthetic sequence
agttgacaatgactgcttagtttagggttttgaagtaaac

SEQ ID NO: 657 - DNA, 40 bp, Artificial sequence
  synthetic sequence
cagatggcaggtattctgtgaattaacactgatgcttctg

SEQ ID NO: 658 - DNA, 40 bp, Artificial sequence
  synthetic sequence
agtcaagttcagaaatgatctgttatgaccccatgaaacg

SEQ ID NO: 659 - DNA, 40 bp, Artificial sequence
  synthetic sequence
gggatgctctgatacatcattcagtaaaatgatagaaaaa

SEQ ID NO: 660 - DNA, 40 bp, Artificial sequence
  synthetic sequence
tagctgtattgcttgatagcttcatagcttgataaccatt

SEQ ID NO: 661 - DNA, 40 bp, Artificial sequence
  synthetic sequence
ttttagcagggaattaacacaggtatataaatgaagaaaa

SEQ ID NO: 662 - DNA, 40 bp, Artificial sequence
  synthetic sequence
ttgattgtttatgaagctgagattgtttactggtttcgag

SEQ ID NO: 663 - DNA, 40 bp, Artificial sequence
  synthetic sequence
tctgtgtttttatgtttgggaacatgagggaatcagttct

SEQ ID NO: 664 - DNA, 40 bp, Artificial sequence
  synthetic sequence
ttcttaagctttcatttttccagtggtgaatgtagagaga

SEQ ID NO: 665 - DNA, 40 bp, Artificial sequence
  synthetic sequence
acggtaactgaataaacttaagaactgaggtaaagttttc

SEQ ID NO: 666 - DNA, 40 bp, Artificial sequence
  synthetic sequence
tcaatatgtaaaattgatcaattcagacacctttatatgg

SEQ ID NO: 667 - DNA, 40 bp, Artificial sequence
  synthetic sequence
tgtctctttcatgctgtaaatagagcattgcatgaaagat

SEQ ID NO: 668 - DNA, 40 bp, Artificial sequence
  synthetic sequence
ttcatagcacagtttataaacctaagaaagcaaagatgaa

SEQ ID NO: 669 - DNA, 40 bp, Artificial sequence
  synthetic sequence
aaccaagcaggattctatgactaaaaaagtgtatttgtat

SEQ ID NO: 670 - DNA, 40 bp, Artificial sequence
  synthetic sequence
agatagagaatttcaaagaaaccatctttatcagctgcac

SEQ ID NO: 671 - DNA, 40 bp, Artificial sequence
  synthetic sequence
ccaagaatgaaaagatgcactaattcgactgaaagccaag

SEQ ID NO: 672 - DNA, 40 bp, Artificial sequence
  synthetic sequence
tcatagttgagacatataacaaccataaaggtccgcatat

SEQ ID NO: 673 - DNA, 40 bp, Artificial sequence
  synthetic sequence
aggaaagggtggaaaggcaagcagcggggagtgttggctg

SEQ ID NO: 674 - DNA, 40 bp, Artificial sequence
  synthetic sequence
ctataaattgacctatcctgtaaaaaaggatgtcacagca

SEQ ID NO: 675 - DNA, 40 bp, Artificial sequence
  synthetic sequence
acaattgacctaagactgtaaattgtaaattgactataaa

SEQ ID NO: 676 - DNA, 40 bp, Artificial sequence
  synthetic sequence
gcaagactgggtatactattaataggaaaaaatgaacttc

SEQ ID NO: 677 - DNA, 40 bp, Artificial sequence
  synthetic sequence
attgctttgatattgattgaatcacagagaaaatcctaag

SEQ ID NO: 678 - DNA, 40 bp, Artificial sequence
  synthetic sequence
tagattatgctggcaaatctcagtgatcagagaattatat

SEQ ID NO: 679 - DNA, 40 bp, Artificial sequence
  synthetic sequence
attcagaaatggaataggaagatatttatgtgccatcctg

SEQ ID NO: 680 - DNA, 40 bp, Artificial sequence
  synthetic sequence
gtttgaattattattcaaacagtgtatgtttgtttgtact

SEQ ID NO: 681 - DNA, 40 bp, Artificial sequence
  synthetic sequence
aatgcaacagagacaggtatttatagcatctgttttccat

SEQ ID NO: 682 - DNA, 40 bp, Artificial sequence
  synthetic sequence
tttaatatccaaatatgtatggacacatacaattgtacat

SEQ ID NO: 683 - DNA, 40 bp, Artificial sequence
  synthetic sequence
acgtctaccgtcattttcgtaattattcggtttccctgtc

SEQ ID NO: 684 - DNA, 40 bp, Artificial sequence
  synthetic sequence
ggagcgctcctgcgcgccttgttcgttaggatttattttt

SEQ ID NO: 685 - DNA, 40 bp, Artificial sequence
  synthetic sequence
ggtggctccctaatgcctgctcgtttcaggtctcagctct

SEQ ID NO: 686 - DNA, 40 bp, Artificial sequence
  synthetic sequence
ccttagtgtgttgaggacgctgcagaaggtacagaggaga

SEQ ID NO: 687 - DNA, 40 bp, Artificial sequence
  synthetic sequence
gaccagatggtaggacagtcattctcctctgcgtctccgc

SEQ ID NO: 688 - DNA, 40 bp, Artificial sequence
  synthetic sequence
cgtgaggcatggagtttttgtcctgcccctgcctggttag

SEQ ID NO: 689 - DNA, 40 bp, Artificial sequence
  synthetic sequence
tttaagtctctggcaccgtgcatagcagaattggttggga

SEQ ID NO: 690 - DNA, 40 bp, Artificial sequence
  synthetic sequence
tctttctccaagtgcctctatgttggcacatctctgaaat

SEQ ID NO: 691 - DNA, 40 bp, Artificial sequence
  synthetic sequence
tgcgtcccggccaggtaagcagcttccctctcagctgcct

SEQ ID NO: 692 - DNA, 40 bp, Artificial sequence
  synthetic sequence
gggtgtatgtagctggcagaagtgggacttggtcgcaacc

SEQ ID NO: 693 - DNA, 40 bp, Artificial sequence
  synthetic sequence
cgtggcgagtgggcggtagctgctcgtagagcgtgtgaaa

SEQ ID NO: 694 - DNA, 40 bp, Artificial sequence
  synthetic sequence
gttggccctaaaagttatcattcatgctagtttgaccaat

SEQ ID NO: 695 - DNA, 40 bp, Artificial sequence
  synthetic sequence
aagtgggaggagctgggcaagaaagtccacccctttttct

SEQ ID NO: 696 - DNA, 40 bp, Artificial sequence
  synthetic sequence
gccgagccgaagtcatctgccaatcaaaacagccacaggg

SEQ ID NO: 697 - DNA, 40 bp, Artificial sequence
  synthetic sequence
cgcgtacctaatgggagacagacaggtgcctttaaagcgg

SEQ ID NO: 698 - DNA, 40 bp, Artificial sequence
  synthetic sequence
tggggaaagcggaggaaggcatggagtgtgggcgttaggg

SEQ ID NO: 699 - DNA, 40 bp, Artificial sequence
  synthetic sequence
gcatattctgccttgaagtcattggttggtcctggaagtg

SEQ ID NO: 700 - DNA, 40 bp, Artificial sequence
  synthetic sequence
aattggtctgggggaggagctacgacagtccaggggcggg

SEQ ID NO: 701 - DNA, 40 bp, Artificial sequence
  synthetic sequence
gtgtcgtgctgattggatgtatccgcccccctctcttaaa

SEQ ID NO: 702 - DNA, 40 bp, Artificial sequence
  synthetic sequence
caacacgccagcgcgaggacccgaacgtcaatcaagagac

SEQ ID NO: 703 - DNA, 40 bp, Artificial sequence
  synthetic sequence
gcgttcgattggcctcccgcgcaggctgctaggattggct

SEQ ID NO: 704 - DNA, 40 bp, Artificial sequence
  synthetic sequence
ccctgccccctttcgcggattgggtgatcgctccaaggcg

SEQ ID NO: 705 - DNA, 40 bp, Artificial sequence
  synthetic sequence
ctgacccttggaggctttctattggttcctggcagggatg

SEQ ID NO: 706 - DNA, 40 bp, Artificial sequence
  synthetic sequence
tcccgaatataggccagtcattgctcctgctgaacgtcgc

SEQ ID NO: 707 - DNA, 40 bp, Artificial sequence
  synthetic sequence
cccctcctctcttctcgtctctggcgccgacccgcccccg

SEQ ID NO: 708 - DNA, 40 bp, Artificial sequence
  synthetic sequence
gctcaagggaggccgcggcgtctgccgatggctccgcgga

SEQ ID NO: 709 - DNA, 40 bp, Artificial sequence
  synthetic sequence
tgggggagtgggcccggggttgttctgacgacgggggtcg

SEQ ID NO: 710 - DNA, 40 bp, Artificial sequence
  synthetic sequence
cccgggcgctatcgcgatagcggcgcgaagcggaagtggg

SEQ ID NO: 711 - DNA, 40 bp, Artificial sequence
  synthetic sequence
cgggggaggcgagcgcccgccgcctttttctcgcgccccg

SEQ ID NO: 712 - DNA, 40 bp, Artificial sequence
  synthetic sequence
cacaggagctggcgccgccgctgaggagcgtatcgcgaca

SEQ ID NO: 713 - DNA, 40 bp, Artificial sequence
  synthetic sequence
gttgccgactcgcgctctcggcttctgctccggggcttct

SEQ ID NO: 714 - DNA, 40 bp, Artificial sequence
  synthetic sequence
actcggagctcggatcccagtgtggacctggactcgaatc

SEQ ID NO: 715 - DNA, 40 bp, Artificial sequence
  synthetic sequence
ggctcctccttgttccgagcccgaaggcccgccccttcac

SEQ ID NO: 716 - DNA, 40 bp, Artificial sequence
  synthetic sequence
ctttccggagcccgtctgttccccttcgggtccaaagctt

SEQ ID NO: 717 - DNA, 40 bp, Artificial sequence
  synthetic sequence
gaccccgcctcattcctcacggcgagctccagaccccgcc

SEQ ID NO: 718 - DNA, 40 bp, Artificial sequence
  synthetic sequence
agaactcaagctcccgattgtgcccgaaggaacccgaagg

SEQ ID NO: 719 - DNA, 40 bp, Artificial sequence
  synthetic sequence
actattgccgaagtgagccgaagtttgtggccccgcttcc

SEQ ID NO: 720 - DNA, 40 bp, Artificial sequence
  synthetic sequence
acatgtggctccgcccacactggcctcagctctccgttct

SEQ ID NO: 721 - DNA, 40 bp, Artificial sequence
  synthetic sequence
acagtgaccctaaggactcgactacctccgaagaaagccg

SEQ ID NO: 722 - DNA, 40 bp, Artificial sequence
  synthetic sequence
cttgtacccaactatctacgaagtaaaccgaagcttgtgg

SEQ ID NO: 723 - DNA, 40 bp, Artificial sequence
  synthetic sequence
tatctggcgaacctgttgactccgcctatcatcctagcgt

SEQ ID NO: 724 - DNA, 40 bp, Artificial sequence
  synthetic sequence
ggcaagtcgctttcgccccgcccccttgtaaatactcatg

SEQ ID NO: 725 - DNA, 40 bp, Artificial sequence
  synthetic sequence
ctcctctacttgggaacttgaggatcgtcaccctggcccg

SEQ ID NO: 726 - DNA, 40 bp, Artificial sequence
  synthetic sequence
ttggctccgccccactgagcgcacctccctctgccgcttc

SEQ ID NO: 727 - DNA, 40 bp, Artificial sequence
  synthetic sequence
tccttgctccaccccctcatgccgacaccctcgtcaactt

SEQ ID NO: 728 - DNA, 40 bp, Artificial sequence
  synthetic sequence
tccaccgatagaaccagcgagtcacctcataaacagtaat

SEQ ID NO: 729 - DNA, 40 bp, Artificial sequence
  synthetic sequence
cgctcagtccgcctccttgcctcccttcagaatgtcccac

SEQ ID NO: 730 - DNA, 40 bp, Artificial sequence
  synthetic sequence
gccgtccactctccgctcgggcgggctcaccccaattggg

SEQ ID NO: 731 - DNA, 40 bp, Artificial sequence
  synthetic sequence
cgaccgaaccccacagccgaaagccccgccccctggacac

SEQ ID NO: 732 - DNA, 40 bp, Artificial sequence
  synthetic sequence
ctccgagcgccagcgcaccccagttggggagttcccgccc

SEQ ID NO: 733 - DNA, 40 bp, Artificial sequence
  synthetic sequence
agccccgcctcctcccggacgcaataggttcggcgttcgg

SEQ ID NO: 734 - DNA, 40 bp, Artificial sequence
  synthetic sequence
agcaatttgacgttcgggtgttctcggctcggccgaatcc

SEQ ID NO: 735 - DNA, 40 bp, Artificial sequence
  synthetic sequence
tgccccctcccgagcacaggaagttcggcgttcgggcgtc

SEQ ID NO: 736 - DNA, 40 bp, Artificial sequence
  synthetic sequence
tttcggacctcctcgctctcagactcccacagtacaaaac

SEQ ID NO: 737 - DNA, 40 bp, Artificial sequence
  synthetic sequence
cgagccttcgctcctcctctttccgaacgactgtgattcg

SEQ ID NO: 738 - DNA, 40 bp, Artificial sequence
  synthetic sequence
gaggctaaggcaccgccgaggccacaccctcttccggacg

SEQ ID NO: 739 - DNA, 40 bp, Artificial sequence
  synthetic sequence
gcgtcccccttcgggtgttcccgtcagcggtcagaagctc

SEQ ID NO: 740 - DNA, 40 bp, Artificial sequence
  synthetic sequence
ccttacaaaggtccattttggcaccaccctcttgcaaagt

SEQ ID NO: 741 - DNA, 40 bp, Artificial sequence
  synthetic sequence
ggagcgtgaaaaacaaacctccgcaagcgcggcgacacgc

SEQ ID NO: 742 - DNA, 40 bp, Artificial sequence
  synthetic sequence
acccgctctgtgcccgcactgccgtacctaccattgcgcc

SEQ ID NO: 743 - DNA, 40 bp, Artificial sequence
  synthetic sequence
ggtcctcagcatctgcatatgtagcccctcccgctggtca

SEQ ID NO: 744 - DNA, 40 bp, Artificial sequence
  synthetic sequence
cccaacccctacccccaatccatcttagagctgattctct

SEQ ID NO: 745 - DNA, 40 bp, Artificial sequence
  synthetic sequence
actccagtgattcttccttatgctagggactcgaggaccc

SEQ ID NO: 746 - DNA, 40 bp, Artificial sequence
  synthetic sequence
gagaattgagaagtcagtgtgggaggggatgtcccagtac

SEQ ID NO: 747 - DNA, 40 bp, Artificial sequence
  synthetic sequence
tttctggttcgcgttggctgcattgtggagctgagggatg

SEQ ID NO: 748 - DNA, 40 bp, Artificial sequence
  synthetic sequence
tagcttcttaatctccttctttaggtcagcctcatacttt

SEQ ID NO: 749 - DNA, 40 bp, Artificial sequence
  synthetic sequence
ttctccctgggacccagcagtccactctcccagttccctc

SEQ ID NO: 750 - DNA, 40 bp, Artificial sequence
  synthetic sequence
aaagtcagacctcaggacccaggaactggggcccacagct

SEQ ID NO: 751 - DNA, 40 bp, Artificial sequence
  synthetic sequence
tcttgatttggtccctcagccgctgcagatgggaaaagca

SEQ ID NO: 752 - DNA, 40 bp, Artificial sequence
  synthetic sequence
taagctgcctcttgtccttgatctcgttggacgctaccca

SEQ ID NO: 753 - DNA, 40 bp, Artificial sequence
  synthetic sequence
ggctctgggctcctaccgtctcaatgagcttgcggttgtc

SEQ ID NO: 754 - DNA, 40 bp, Artificial sequence
  synthetic sequence
tgaggacctctggggtctggccgctctgcctccgcccctt

SEQ ID NO: 755 - DNA, 40 bp, Artificial sequence
  synthetic sequence
ctgcctcttcacttcccttaggtgcagaaaccttacttct

SEQ ID NO: 756 - DNA, 40 bp, Artificial sequence
  synthetic sequence
cgacctgagcctcgtgaccctactttctgagctctgagtc

SEQ ID NO: 757 - DNA, 40 bp, Artificial sequence
  synthetic sequence
tcaaaggtgggaaaggagctgactaagggccagcagacac

SEQ ID NO: 758 - DNA, 40 bp, Artificial sequence
  synthetic sequence
ccgttccatttgctgtagagagtgcagttggcaggggggc

SEQ ID NO: 759 - DNA, 40 bp, Artificial sequence
  synthetic sequence
gctgtaagctttggttttggtctctcgttccacaactttg

SEQ ID NO: 760 - DNA, 40 bp, Artificial sequence
  synthetic sequence
ccaactcaccgtgagccactggccaacctcttccttctcc

SEQ ID NO: 761 - DNA, 40 bp, Artificial sequence
  synthetic sequence
ccagggctcaggatcctcagagttcacctcctcttctcta

SEQ ID NO: 762 - DNA, 40 bp, Artificial sequence
  synthetic sequence
gtccacctgcatgttgagcgtgtcgatggtattctagggg

SEQ ID NO: 763 - DNA, 40 bp, Artificial sequence
  synthetic sequence
gcgtgtctgcactgacagtgactccacttcactctcaaac

SEQ ID NO: 764 - DNA, 40 bp, Artificial sequence
  synthetic sequence
tgtcgggtctccctcactcacatccttgtcgcccttcttc

SEQ ID NO: 765 - DNA, 40 bp, Artificial sequence
  synthetic sequence
ctgctggccagcccattcccatgcccatccccatcccaaa

SEQ ID NO: 766 - DNA, 40 bp, Artificial sequence
  synthetic sequence
gaatccaggccccaactcccaggagcataaatgactggcc

SEQ ID NO: 767 - DNA, 40 bp, Artificial sequence
  synthetic sequence
tctcaaatccctaatcccggctgttggccctgtccgcctg

SEQ ID NO: 768 - DNA, 40 bp, Artificial sequence
  synthetic sequence
cctgccccacgcgtgcagctgctaagccctcccaatcctg

SEQ ID NO: 769 - DNA, 40 bp, Artificial sequence
  synthetic sequence
cccagacacccaggggaccctgagattctgtctgacctcc

SEQ ID NO: 770 - DNA, 40 bp, Artificial sequence
  synthetic sequence
cttcccccaagtcgctcctcttcacaaaggccccacggtc

SEQ ID NO: 771 - DNA, 40 bp, Artificial sequence
  synthetic sequence
cctctgggtgccaggaggcctcttgccatgggtgtccttc

SEQ ID NO: 772 - DNA, 40 bp, Artificial sequence
  synthetic sequence
ctgccttgtctctacccactgtgctctccctaggaccagg

SEQ ID NO: 773 - DNA, 40 bp, Artificial sequence
  synthetic sequence
ggcgagggggaggtcctgcagctgctcgcgtgggctgccc

SEQ ID NO: 774 - DNA, 40 bp, Artificial sequence
  synthetic sequence
tgcgctcgatctcatccttcagttcgtagcccacctgggg

SEQ ID NO: 775 - DNA, 40 bp, Artificial sequence
  synthetic sequence
tcacctgcttcacaggcggcggctcctgccacttgtcgaa

SEQ ID NO: 776 - DNA, 40 bp, Artificial sequence
  synthetic sequence
ctcgcttcttccgctgtccatccaggggcgcaggcagcgg

SEQ ID NO: 777 - DNA, 40 bp, Artificial sequence
  synthetic sequence
cccatgcctaccggacccccagggcccctcacctgcggcc

SEQ ID NO: 778 - DNA, 40 bp, Artificial sequence
  synthetic sequence
agtcggctgggaggaggacgccggcttctcccctccatga

SEQ ID NO: 779 - DNA, 40 bp, Artificial sequence
  synthetic sequence
atcttgcggtacctggggacgggtgggtgggcggcgccag

SEQ ID NO: 780 - DNA, 40 bp, Artificial sequence
  synthetic sequence
ttggcctgcttccggatctccgtcagccccagccgctcct

SEQ ID NO: 781 - DNA, 40 bp, Artificial sequence
  synthetic sequence
ggagggcgctctgggagtctgacctctccgaagctcatac

SEQ ID NO: 782 - DNA, 40 bp, Artificial sequence
  synthetic sequence
aggaggcagagggcggtggcggctggctggctgtggggtt

SEQ ID NO: 783 - DNA, 40 bp, Artificial sequence
  synthetic sequence
agacatgagccagggccacaggacgagaggaggggcggtg

SEQ ID NO: 784 - DNA, 40 bp, Artificial sequence
  synthetic sequence
ccaagggccgcgagggtcgctttggggctgaatggatgga

SEQ ID NO: 785 - DNA, 40 bp, Artificial sequence
  synthetic sequence
gatgggaagccgcgggggctctaagcagcggagacacagg

SEQ ID NO: 786 - DNA, 40 bp, Artificial sequence
  synthetic sequence
ggagcctctgggcagggaggaaccggccaaggagcccggg

SEQ ID NO: 787 - DNA, 40 bp, Artificial sequence
  synthetic sequence
ggcggggcccagggacggggcggccgtgcagcagggcact

SEQ ID NO: 788 - DNA, 40 bp, Artificial sequence
  synthetic sequence
ctgcaggaccaaggggatgacgctgggataacagaggaga

SEQ ID NO: 789 - DNA, 40 bp, Artificial sequence
  synthetic sequence
cagaacaggtttaataggatgaggtggcctctgagttcgg

SEQ ID NO: 790 - DNA, 40 bp, Artificial sequence
  synthetic sequence
ccattccttccttactcgtgtgggtcgggggatgtcagga

SEQ ID NO: 791 - DNA, 40 bp, Artificial sequence
  synthetic sequence
ggcccggtcccagcactgctctgtgagctcagagttggga

SEQ ID NO: 792 - DNA, 40 bp, Artificial sequence
  synthetic sequence
tgggggcccacacacgcgggggatgccggggagcctgaga

SEQ ID NO: 793 - DNA, 40 bp, Artificial sequence
  synthetic sequence
cacgggcacctgctccggtacccactcggcccggctgagg

SEQ ID NO: 794 - DNA, 40 bp, Artificial sequence
  synthetic sequence
ctccaccagccggaagcccagcggtcaccagccggccggt

SEQ ID NO: 795 - DNA, 40 bp, Artificial sequence
  synthetic sequence
aggcgtcctcctcgatctagggggaagaggaggcgccctg

SEQ ID NO: 796 - DNA, 40 bp, Artificial sequence
  synthetic sequence
acttgcccaggtggcccaggctgaatcccaggtcctcctg

SEQ ID NO: 797 - DNA, 40 bp, Artificial sequence
  synthetic sequence
tggcctcgtttacctgtgtctgccgcacacgcccactgcc

SEQ ID NO: 798 - DNA, 40 bp, Artificial sequence
  synthetic sequence
gtctggcccatacctgcagcgtcttggagatcctggcctt

SEQ ID NO: 799 - DNA, 40 bp, Artificial sequence
  synthetic sequence
gctccccccaccttgtgtccctcggtccccagccccacct

SEQ ID NO: 800 - DNA, 40 bp, Artificial sequence
  synthetic sequence
tgcagggtccgctgtggggaggacagggaggctgcgatct

SEQ ID NO: 801 - DNA, 40 bp, Artificial sequence
  synthetic sequence
tcgcggatggtggacttcccgccatatacgacgctctgct

SEQ ID NO: 802 - DNA, 40 bp, Artificial sequence
  synthetic sequence
agtggggtgaaggccacgctggaggccgtgcccgaggagc

SEQ ID NO: 803 - DNA, 40 bp, Artificial sequence
  synthetic sequence
cggctgctgagcctaaccacctcctgggcttctttccagc

SEQ ID NO: 804 - DNA, 40 bp, Artificial sequence
  synthetic sequence
gctcatggtatccctaccgcaggcaatctgtggacagcac

SEQ ID NO: 805 - DNA, 40 bp, Artificial sequence
  synthetic sequence
ctgaatgtcacctgaagggtcacagaagctactcacaggg

SEQ ID NO: 806 - DNA, 40 bp, Artificial sequence
  synthetic sequence
ttaagtgttctcaatatgagattagctggagccgcctaat

SEQ ID NO: 807 - DNA, 40 bp, Artificial sequence
  synthetic sequence
gaagatccatctgttggaagccagaggactagtgggaaac

SEQ ID NO: 808 - DNA, 40 bp, Artificial sequence
  synthetic sequence
cccccacagggatctgacacacaacttaggttgtcagcca

SEQ ID NO: 809 - DNA, 40 bp, Artificial sequence
  synthetic sequence
gcccagcttcccaagtcctgcctggacaccgccccatgga

SEQ ID NO: 810 - DNA, 40 bp, Artificial sequence
  synthetic sequence
aatcaccttcatgcttaaaacactcacactgatttccagc

SEQ ID NO: 811 - DNA, 40 bp, Artificial sequence
  synthetic sequence
cctcttggggacctgggtgaccttactcaccctcatggct

SEQ ID NO: 812 - DNA, 40 bp, Artificial sequence
  synthetic sequence
gttgctgtggacaggcttggagccgtttttggctggagac

SEQ ID NO: 813 - DNA, 40 bp, Artificial sequence
  synthetic sequence
ggaggggtaggtgggcggcacagctggggactgagggtgc

SEQ ID NO: 814 - DNA, 40 bp, Artificial sequence
  synthetic sequence
gccaggagtggtgctcaaggcagaggcagcaggcgggggg

SEQ ID NO: 815 - DNA, 40 bp, Artificial sequence
  synthetic sequence
cagggcacttgggggtgctgcgggggcggggaccccattg

SEQ ID NO: 816 - DNA, 40 bp, Artificial sequence
  synthetic sequence
ggtgcccgagttgtggctgggagctggactggccttgggg

SEQ ID NO: 817 - DNA, 40 bp, Artificial sequence
  synthetic sequence
ctgcttgccagcccctccaccggcactgctgttactactg

SEQ ID NO: 818 - DNA, 40 bp, Artificial sequence
  synthetic sequence
gccccccaccccgctgcctcctcactcactggtggcgcca

SEQ ID NO: 819 - DNA, 40 bp, Artificial sequence
  synthetic sequence
cgggctgtctgccacaactgagctgtaacctgggaacaaa

SEQ ID NO: 820 - DNA, 40 bp, Artificial sequence
  synthetic sequence
gctggcattgttgcccccactgctgctcaaagccacctct

SEQ ID NO: 821 - DNA, 40 bp, Artificial sequence
  synthetic sequence
aggtgggttgtgggggccggaaggggggcccaaggcctgg

SEQ ID NO: 822 - DNA, 40 bp, Artificial sequence
  synthetic sequence
tcccaaccctgccgatggccgagacactcacgaggtgctg

SEQ ID NO: 823 - DNA, 40 bp, Artificial sequence
  synthetic sequence
gggggtgaggcgcctgcgcctctctgtttcaaaaggctgc

SEQ ID NO: 824 - DNA, 40 bp, Artificial sequence
  synthetic sequence
attcccagcagcaagggcggggggttcagaacccaccgat

SEQ ID NO: 825 - DNA, 40 bp, Artificial sequence
  synthetic sequence
gggggtgtaacacccgagggagatggaggatagcgcttgg

SEQ ID NO: 826 - DNA, 40 bp, Artificial sequence
  synthetic sequence
caaagcagggaggctgatgtagtttccttgctggaaagaa

SEQ ID NO: 827 - DNA, 40 bp, Artificial sequence
  synthetic sequence
cttccacttagatgagaacgtattttagaatgttctgaag

SEQ ID NO: 828 - DNA, 40 bp, Artificial sequence
  synthetic sequence
taacagaaatggggaggaaagggtatggggctcttgagaa

SEQ ID NO: 829 - DNA, 40 bp, Artificial sequence
  synthetic sequence
aaacagtgaccctccggtggcagtcaattggcctcaggca

SEQ ID NO: 830 - DNA, 40 bp, Artificial sequence
  synthetic sequence
gcagaggaataaggacttcgggacaattcactttgaaaag

SEQ ID NO: 831 - DNA, 40 bp, Artificial sequence
  synthetic sequence
gacccagtggaatggtctgagctaagatttgaaggagtgg

SEQ ID NO: 832 - DNA, 40 bp, Artificial sequence
  synthetic sequence
tgcacactgatctttcttagggcattcttcgggaaacagg

SEQ ID NO: 833 - DNA, 40 bp, Artificial sequence
  synthetic sequence
ggctcaggatgaacagcaacaggggttgggatgatcactg

SEQ ID NO: 834 - DNA, 40 bp, Artificial sequence
  synthetic sequence
gatcatggagatgtgatctagggaacaaagccagagaagg

SEQ ID NO: 835 - DNA, 40 bp, Artificial sequence
  synthetic sequence
aggcattcccacggtgtgaggtcagattgggcagggccta

SEQ ID NO: 836 - DNA, 40 bp, Artificial sequence
  synthetic sequence
agagccagcacttgctgttccacacatactagatcagtct

SEQ ID NO: 837 - DNA, 40 bp, Artificial sequence
  synthetic sequence
tggacaaccccctcccacacccagagctgtggaaggggag

SEQ ID NO: 838 - DNA, 40 bp, Artificial sequence
  synthetic sequence
cacctagatgctgaccaaggccctccccatgctgctggag

SEQ ID NO: 839 - DNA, 40 bp, Artificial sequence
  synthetic sequence
ataaagccttcattctccaggaccccgcccttgccctgtt

SEQ ID NO: 840 - DNA, 40 bp, Artificial sequence
  synthetic sequence
aggtggtgagtttggggctggggggcctccctgaggagcc

SEQ ID NO: 841 - DNA, 40 bp, Artificial sequence
  synthetic sequence
gagagaaccaggtcccacatgctgacacaggtgtccacgg

SEQ ID NO: 842 - DNA, 40 bp, Artificial sequence
  synthetic sequence
atccccccaatctcaccagtgcaccccacagacaaggcga

SEQ ID NO: 843 - DNA, 40 bp, Artificial sequence
  synthetic sequence
aagggcttcagcataagagtcagaacccgccccccttcct

SEQ ID NO: 844 - DNA, 40 bp, Artificial sequence
  synthetic sequence
tgtgggctgaagggacgaggctggggcactgggtgggagg

SEQ ID NO: 845 - DNA, 40 bp, Artificial sequence
  synthetic sequence
ttgcaatgtggaagagtcaggggcacattgtctgggctga

SEQ ID NO: 846 - DNA, 40 bp, Artificial sequence
  synthetic sequence
taagtgggagggagcggggacctagtgtgggcatgaggac

SEQ ID NO: 847 - DNA, 40 bp, Artificial sequence
  synthetic sequence
ggagcagggatttggctgggcaatggagagaaaggtctga

SEQ ID NO: 848 - DNA, 40 bp, Artificial sequence
  synthetic sequence
acacagagatgcccaggaacttgctctttagtaaagcagc

SEQ ID NO: 849 - DNA, 40 bp, Artificial sequence
  synthetic sequence
tggagagaggtccttgaaaggttttgaaccccataaagag

SEQ ID NO: 850 - DNA, 40 bp, Artificial sequence
  synthetic sequence
tcaggaggcagcccagtgatagggtccaaggaaccagtgg

SEQ ID NO: 851 - DNA, 40 bp, Artificial sequence
  synthetic sequence
acagtctactgacttttcctattcagctgtgagcattcaa

SEQ ID NO: 852 - DNA, 40 bp, Artificial sequence
  synthetic sequence
ctgtcccctggaccttgacacctggctccccaaccctgtc

SEQ ID NO: 853 - DNA, 40 bp, Artificial sequence
  synthetic sequence
aggaaacccagattccaccagacacttccttcttcccccc

SEQ ID NO: 854 - DNA, 40 bp, Artificial sequence
  synthetic sequence
ggctatctggcctgagacaacaaatgctgcctcccaccct

SEQ ID NO: 855 - DNA, 40 bp, Artificial sequence
  synthetic sequence
gtctggcactgggactttcagaactcctccttccctgact

SEQ ID NO: 856 - DNA, 40 bp, Artificial sequence
  synthetic sequence
ttgccccagacccgtcattcaatggctagctttttccatg

SEQ ID NO: 857 - DNA, 40 bp, Artificial sequence
  synthetic sequence
aaaaacacgagcacccccaaccacaacggccagttctctg

SEQ ID NO: 858 - DNA, 40 bp, Artificial sequence
  synthetic sequence
ttaaccttggacatggtaaaccatccaaaaccttcctctc

SEQ ID NO: 859 - DNA, 40 bp, Artificial sequence
  synthetic sequence
agcaactaaacctctccactgggcacttatccttggtttc

SEQ ID NO: 860 - DNA, 40 bp, Artificial sequence
  synthetic sequence
gaacctcttattctcttagaacccacagctgccaccacag

SEQ ID NO: 861 - DNA, 40 bp, Artificial sequence
  synthetic sequence
tcccttctcccagtgtaagaccccaaatcactccaaatga

SEQ ID NO: 862 - DNA, 40 bp, Artificial sequence
  synthetic sequence
caacccccaacccgatgcctgcttcagatgtttcccatgt

SEQ ID NO: 863 - DNA, 40 bp, Artificial sequence
  synthetic sequence
cataaacctggctcctaaaggctaaatattttgttggaga

SEQ ID NO: 864 - DNA, 40 bp, Artificial sequence
  synthetic sequence
ctgctgacctgccctcccaggtcagaatcatcctcatgca

SEQ ID NO: 865 - DNA, 40 bp, Artificial sequence
  synthetic sequence
tgttctccagacctgtgcactctatctgtgcaacagagat

SEQ ID NO: 866 - DNA, 40 bp, Artificial sequence
  synthetic sequence
cgtgcagcaaacaatgtggaattccaataaccccccactc

SEQ ID NO: 867 - DNA, 40 bp, Artificial sequence
  synthetic sequence
aaatatgagtctcccaaagttccctagcatttcaaaatcc

SEQ ID NO: 868 - DNA, 40 bp, Artificial sequence
  synthetic sequence
catcataaaaagatcttgtggtccacagatcctctagccc

SEQ ID NO: 869 - DNA, 40 bp, Artificial sequence
  synthetic sequence
ctcccaacccagaatccagctccacagatacattgctact

SEQ ID NO: 870 - DNA, 40 bp, Artificial sequence
  synthetic sequence
cactctgagaccagaaactagaacttttattcctcatgct

SEQ ID NO: 871 - DNA, 40 bp, Artificial sequence
  synthetic sequence
caccagcactcaggagattgtgagactccctgatccctgc

SEQ ID NO: 872 - DNA, 40 bp, Artificial sequence
  synthetic sequence
tgcctagatcctttgcactccaagacccagtgtgccctaa

SEQ ID NO: 873 - DNA, 40 bp, Artificial sequence
  synthetic sequence
gggggtgggtacgatccccgattcttcatacaaagcctca

SEQ ID NO: 874 - DNA, 40 bp, Artificial sequence
  synthetic sequence
ggacaaaggcagaggagacacgcccaggatgaaacagaaa

SEQ ID NO: 875 - DNA, 40 bp, Artificial sequence
  synthetic sequence
tggatgcaccaggccctgtagctcatggagacttcatcta

SEQ ID NO: 876 - DNA, 40 bp, Artificial sequence
  synthetic sequence
gggagagctagcacttgctgttctgcaattactagatcac

SEQ ID NO: 877 - DNA, 40 bp, Artificial sequence
  synthetic sequence
ggctggacaaccccctcccacacccagagctgtggaaggg

SEQ ID NO: 878 - DNA, 40 bp, Artificial sequence
  synthetic sequence
tggcacccagaggctgaccaaggccctccccatgctgctg

SEQ ID NO: 879 - DNA, 40 bp, Artificial sequence
  synthetic sequence
cctataaaaccttcattccccaggactccgcccctgccct

SEQ ID NO: 880 - DNA, 40 bp, Artificial sequence
  synthetic sequence
tgcaggtggtaagcttggggctggggagcctcccccagga

SEQ ID NO: 881 - DNA, 40 bp, Artificial sequence
  synthetic sequence
aggaagacaaccgggacccacatggtgacacagctctccg

SEQ ID NO: 882 - DNA, 40 bp, Artificial sequence
  synthetic sequence
caaccatggcccctctcaccaatccacgtcacggacaggg

SEQ ID NO: 883 - DNA, 40 bp, Artificial sequence
  synthetic sequence
tcagcttgacagtcagggctggctccctctcctgcatccc

SEQ ID NO: 884 - DNA, 40 bp, Artificial sequence
  synthetic sequence
tccctgtctgggctggggtgctgggttgggggggaaagag

SEQ ID NO: 885 - DNA, 40 bp, Artificial sequence
  synthetic sequence
tgtgggagtgaggactgttgcaatatggaggggctggggg

SEQ ID NO: 886 - DNA, 40 bp, Artificial sequence
  synthetic sequence
gggagaaagttctggggtaagtgggagggagcggggacct

SEQ ID NO: 887 - DNA, 40 bp, Artificial sequence
  synthetic sequence
ttgtggggctcaaaacctccaaggacctctctcaatgcca

SEQ ID NO: 888 - DNA, 40 bp, Artificial sequence
  synthetic sequence
tgcccaaccctatcccagagaccttgatgcttggcctccc

SEQ ID NO: 889 - DNA, 40 bp, Artificial sequence
  synthetic sequence
tcttgccctaggatacccagatgccaaccagacacctcct

SEQ ID NO: 890 - DNA, 40 bp, Artificial sequence
  synthetic sequence
ttcctagccaggctatctggcctgagacaacaaatgggtc

SEQ ID NO: 891 - DNA, 40 bp, Artificial sequence
  synthetic sequence
tcttagccccagactcttcattcagtggcccacattttcc

SEQ ID NO: 892 - DNA, 40 bp, Artificial sequence
  synthetic sequence
aggaaaaacatgagcatccccagccacaactgccagctct

SEQ ID NO: 893 - DNA, 40 bp, Artificial sequence
  synthetic sequence
ccccttcagagttactgacaaacaggtgggcactgagact

SEQ ID NO: 894 - DNA, 40 bp, Artificial sequence
  synthetic sequence
tggaaagttagcttatttgtttgcaagtcagtaaaatgtc

SEQ ID NO: 895 - DNA, 40 bp, Artificial sequence
  synthetic sequence
gactcaggagtctcatggactctgccagcattcacaaaac

SEQ ID NO: 896 - DNA, 40 bp, Artificial sequence
  synthetic sequence
atgctgtctgctaagctgtgagcagtaaaagcctttgcct

SEQ ID NO: 897 - DNA, 40 bp, Artificial sequence
  synthetic sequence
gatttggggggggcaaggtgtactaatgtgaacatgaacc

SEQ ID NO: 898 - DNA, 40 bp, Artificial sequence
  synthetic sequence
gtgtgcacagcatccacctagactgctctggtcaccctac

SEQ ID NO: 899 - DNA, 40 bp, Artificial sequence
  synthetic sequence
aggattcctaatctcaggtttctcaccagtggcacaaacc

SEQ ID NO: 900 - DNA, 40 bp, Artificial sequence
  synthetic sequence
caaaggctgagcaggtttgcaagttgtcccagtataagat

SEQ ID NO: 901 - DNA, 40 bp, Artificial sequence
  synthetic sequence
gtcaaggacaatcgatacaatatgttcctccagagtaggt

SEQ ID NO: 902 - DNA, 40 bp, Artificial sequence
  synthetic sequence
gcaagatgatatctctctcagatccaggcttgcttactgt

SEQ ID NO: 903 - DNA, 40 bp, Artificial sequence
  synthetic sequence
tctgtgtgtcttctgagcaaagacagcaacaccttttttt

SEQ ID NO: 904 - DNA, 40 bp, Artificial sequence
  synthetic sequence
aacgttgagactgtcctgcagacaagggtggaaggctctg

SEQ ID NO: 905 - DNA, 40 bp, Artificial sequence
  synthetic sequence
cataaataagcaggatgtgacagaagaagtatttaatggt

SEQ ID NO: 906 - DNA, 40 bp, Artificial sequence
  synthetic sequence
gctgccagacacagtcgatcgggacctagaaccttggtta

SEQ ID NO: 907 - DNA, 40 bp, Artificial sequence
  synthetic sequence
gggatcctgagcgctgccttattctgggtttggcagtgga

SEQ ID NO: 908 - DNA, 40 bp, Artificial sequence
  synthetic sequence
tcactcaaacccagaagttctgatccccagccatgcccct

SEQ ID NO: 909 - DNA, 40 bp, Artificial sequence
  synthetic sequence
agcctcttcctcctttgaaattcaagagggtggacccact

SEQ ID NO: 910 - DNA, 40 bp, Artificial sequence
  synthetic sequence
ggagctgggaccttaccagtctcctccctcattgacctaa

SEQ ID NO: 911 - DNA, 40 bp, Artificial sequence
  synthetic sequence
gaggatatgagattcttaggccattcccacatcagtacct

SEQ ID NO: 912 - DNA, 40 bp, Artificial sequence
  synthetic sequence
tacccagaactctacccctcaggattccagcaccttcttc

SEQ ID NO: 913 - DNA, 40 bp, Artificial sequence
  synthetic sequence
gcctctgcccttcaggggccaaagagccttaagccacaaa

SEQ ID NO: 914 - DNA, 40 bp, Artificial sequence
  synthetic sequence
atcccattactatcaccccaaaccctggacctaatggttc

SEQ ID NO: 915 - DNA, 40 bp, Artificial sequence
  synthetic sequence
aatgggcaaccctcgatcctcagactcttgaggaatcaag

SEQ ID NO: 916 - DNA, 40 bp, Artificial sequence
  synthetic sequence
gataccctcaagtggagtaaggattaggtggcaagatgga

SEQ ID NO: 917 - DNA, 40 bp, Artificial sequence
  synthetic sequence
gtgcttgcccaggggcaccttcatggagctagaagggctg

SEQ ID NO: 918 - DNA, 40 bp, Artificial sequence
  synthetic sequence
gatgacacccaaggcctctggggcatctttcatgctcaga

SEQ ID NO: 919 - DNA, 40 bp, Artificial sequence
  synthetic sequence
tgctggccacaccctcagagtgtggatgctggatgatgag

SEQ ID NO: 920 - DNA, 40 bp, Artificial sequence
  synthetic sequence
gaggcacgctgcagggatagtcacagcaacatgacgtcat

SEQ ID NO: 921 - DNA, 40 bp, Artificial sequence
  synthetic sequence
agaggaggatgtcggcagctctacggttggcaggtggctg

SEQ ID NO: 922 - DNA, 40 bp, Artificial sequence
  synthetic sequence
gacactaggcctcagcctggcaccatgcaggccactccca

SEQ ID NO: 923 - DNA, 40 bp, Artificial sequence
  synthetic sequence
acttttgagtcctggatccctatgattccaggctccctgt

SEQ ID NO: 924 - DNA, 40 bp, Artificial sequence
  synthetic sequence
ccttgagatttcatggatggtgacatatggccattctcta

SEQ ID NO: 925 - DNA, 40 bp, Artificial sequence
  synthetic sequence
aaaacccataagttcaggtccctgtgccctccacccagaa

SEQ ID NO: 926 - DNA, 40 bp, Artificial sequence
  synthetic sequence
tcgtatctgggagactcacttgggagagcaatagacttgg

SEQ ID NO: 927 - DNA, 40 bp, Artificial sequence
  synthetic sequence
tacaagatgtggtggagataaggctgatgctggcacagtg

SEQ ID NO: 928 - DNA, 40 bp, Artificial sequence
  synthetic sequence
gtacacaccatggtgttcatcagggccctgggtagtccct

SEQ ID NO: 929 - DNA, 40 bp, Artificial sequence
  synthetic sequence
gctgtgacctcacaggagtccgtgcctccaccccctactc

SEQ ID NO: 930 - DNA, 40 bp, Artificial sequence
  synthetic sequence
ttggctgacctgattgctgtgtcctgtgtcagctgctgct

SEQ ID NO: 931 - DNA, 40 bp, Artificial sequence
  synthetic sequence
atgtaccatttgcccctggatgttctgcactatagggtaa

SEQ ID NO: 932 - DNA, 40 bp, Artificial sequence
  synthetic sequence
tacttttacccatgcatttaaagttctaggtgatatggcc

SEQ ID NO: 933 - DNA, 40 bp, Artificial sequence
  synthetic sequence
aaacatgggtatcacttctgggctgaaagccttctcttct

SEQ ID NO: 934 - DNA, 40 bp, Artificial sequence
  synthetic sequence
ggtgtttaaatcttgtggggtggctccttctgataatgct

SEQ ID NO: 935 - DNA, 40 bp, Artificial sequence
  synthetic sequence
catttgcatggctgcttgatgtccccccactgtgtttagc

SEQ ID NO: 936 - DNA, 40 bp, Artificial sequence
  synthetic sequence
catctggcctggtgcaataggccctgcatgcactggatgc

SEQ ID NO: 937 - DNA, 40 bp, Artificial sequence
  synthetic sequence
ggtactagtagttcctgctatgtcacttccccttggttct

SEQ ID NO: 938 - DNA, 40 bp, Artificial sequence
  synthetic sequence
gataggtggattatttgtcatccatcctatttgttcctga

SEQ ID NO: 939 - DNA, 40 bp, Artificial sequence
  synthetic sequence
gtccagaatgctggtagggctatacattcttactatttta

SEQ ID NO: 940 - DNA, 40 bp, Artificial sequence
  synthetic sequence
gtctacatagtctctaaagggttcctttggtccttgtctt

SEQ ID NO: 941 - DNA, 40 bp, Artificial sequence
  synthetic sequence
ctcctgtgaagcttgctcggctcttagagttttatagaac

SEQ ID NO: 942 - DNA, 40 bp, Artificial sequence
  synthetic sequence
cgcattttggaccaacaaggtttctgtcatccaatttttt

SEQ ID NO: 943 - DNA, 40 bp, Artificial sequence
  synthetic sequence
tcctactccctgacatgctgtcatcatttcttctagtgta

SEQ ID NO: 944 - DNA, 40 bp, Artificial sequence
  synthetic sequence
gctcattgcttcagccaaaactcttgccttatggccgggt

SEQ ID NO: 945 - DNA, 40 bp, Artificial sequence
  synthetic sequence
attgcctctctgcatcattatggtagctgaatttgttact

SEQ ID NO: 946 - DNA, 40 bp, Artificial sequence
  synthetic sequence
gccacaattgaaacacttaacaatctttctttggttccta

SEQ ID NO: 947 - DNA, 40 bp, Artificial sequence
  synthetic sequence
tttcctaggggccctgcaatttctggctgtgtgcccttct

SEQ ID NO: 948 - DNA, 40 bp, Artificial sequence
  synthetic sequence
cccagacctgaagctctcttctggtggggctgttggctct

SEQ ID NO: 949 - DNA, 40 bp, Artificial sequence
  synthetic sequence
gtctatcggctcctgcttctgagggggagttgttgtctct

SEQ ID NO: 950 - DNA, 40 bp, Artificial sequence
  synthetic sequence
gccaaagagtgacctgagggaagttaaaggatacagttcc

SEQ ID NO: 951 - DNA, 40 bp, Artificial sequence
  synthetic sequence
cctttagttgcccccctatctttattgtgacgaggggtcg

SEQ ID NO: 952 - DNA, 40 bp, Artificial sequence
  synthetic sequence
cttctaatactgtatcatctgctcctgtatctaatagagc

SEQ ID NO: 953 - DNA, 40 bp, Artificial sequence
  synthetic sequence
gtatctgatcatactgtcttactttgataaaacctccaat

SEQ ID NO: 954 - DNA, 40 bp, Artificial sequence
  synthetic sequence
ctaatactgtacctatagctttatgtccacagatttctat

SEQ ID NO: 955 - DNA, 40 bp, Artificial sequence
  synthetic sequence
tcaacagatttcttccaattatgttgacaggtgtaggtcc

SEQ ID NO: 956 - DNA, 40 bp, Artificial sequence
  synthetic sequence
ttgggccatccattcctggctttaattttactggtacagt

SEQ ID NO: 957 - DNA, 40 bp, Artificial sequence
  synthetic sequence
caaatactggagtattgtatggattttcaggcccaatttt

SEQ ID NO: 958 - DNA, 40 bp, Artificial sequence
  synthetic sequence
cttcccagaagtcttgagttctcttattaagttctctgaa

SEQ ID NO: 959 - DNA, 40 bp, Artificial sequence
  synthetic sequence
ctgaaaaatatgcatcacccacatccagtactgttactga

SEQ ID NO: 960 - DNA, 40 bp, Artificial sequence
  synthetic sequence
tggtaaatgcagtatacttcctgaagtcttcatctaaggg

SEQ ID NO: 961 - DNA, 40 bp, Artificial sequence
  synthetic sequence
actgatatctaatccctggtgtctcattgtttatactagg

SEQ ID NO: 962 - DNA, 40 bp, Artificial sequence
  synthetic sequence
atattgctggtgatcctttccatccctgtggaagcacatt

SEQ ID NO: 963 - DNA, 40 bp, Artificial sequence
  synthetic sequence
gttttctaaaaggctctaagatttttgtcatgctactttg

SEQ ID NO: 964 - DNA, 40 bp, Artificial sequence
  synthetic sequence
acaaatcatccatgtattgatagataactatgtctggatt

SEQ ID NO: 965 - DNA, 40 bp, Artificial sequence
  synthetic sequence
tttttgttctatgctgccctatttctaagtcagatcctac

SEQ ID NO: 966 - DNA, 40 bp, Artificial sequence
  synthetic sequence
tggtaagtccccacctcaacagatgttgtctcagctcctc

SEQ ID NO: 967 - DNA, 40 bp, Artificial sequence
  synthetic sequence
taggctgtactgtccatttatcaggatggagttcataacc

SEQ ID NO: 968 - DNA, 40 bp, Artificial sequence
  synthetic sequence
gtatgtcattgacagtccagctgtctttttctggcagcac

SEQ ID NO: 969 - DNA, 40 bp, Artificial sequence
  synthetic sequence
ggtaaatctgacttgcccaattcaatttccccactaactt

SEQ ID NO: 970 - DNA, 40 bp, Artificial sequence
  synthetic sequence
ttcctctaaggagtttacataattgccttactttaatccc

SEQ ID NO: 971 - DNA, 40 bp, Artificial sequence
  synthetic sequence
ctgcttcttctgttagtggtattacttctgttagtgcttt

SEQ ID NO: 972 - DNA, 40 bp, Artificial sequence
  synthetic sequence
ctgctattaagtcttttgatgggtcataatacactccatg

SEQ ID NO: 973 - DNA, 40 bp, Artificial sequence
  synthetic sequence
aaatttgatatgtccattggccttgcccctgcttctgtat

SEQ ID NO: 974 - DNA, 40 bp, Artificial sequence
  synthetic sequence
ctgttaattgttttacatcattagtgtgggcacccctcat

SEQ ID NO: 975 - DNA, 40 bp, Artificial sequence
  synthetic sequence
atgtttccttttgtatgggcagtttaaatttaggagtctt

SEQ ID NO: 976 - DNA, 40 bp, Artificial sequence
  synthetic sequence
gaatccaggtggcttgccaatactctgtccaccatgtttc

SEQ ID NO: 977 - DNA, 40 bp, Artificial sequence
  synthetic sequence
ataatttcactaagggaggggtattaacaaactcccactc

SEQ ID NO: 978 - DNA, 40 bp, Artificial sequence
  synthetic sequence
aggtttctgctcctactatgggttctttctctaactggta

SEQ ID NO: 979 - DNA, 40 bp, Artificial sequence
  synthetic sequence
ttcctaatttagtctccctgttagctgccccatctacata

SEQ ID NO: 980 - DNA, 40 bp, Artificial sequence
  synthetic sequence
ttgcttgtaactcagtcttctgatttgttgtgtcagttag

SEQ ID NO: 981 - DNA, 40 bp, Artificial sequence
  synthetic sequence
ctatgtttacttctaatcccgaatcctgcaaagctagata

SEQ ID NO: 982 - DNA, 40 bp, Artificial sequence
  synthetic sequence
gttgtgcttgaatgattcctaatgcatattgtgagtctgt

SEQ ID NO: 983 - DNA, 40 bp, Artificial sequence
  synthetic sequence
gctctattatttgattgactaactctgattcactttgatc

SEQ ID NO: 984 - DNA, 40 bp, Artificial sequence
  synthetic sequence
tccaattactgtgatatttctcatgttcatcttgggcctt

SEQ ID NO: 985 - DNA, 40 bp, Artificial sequence
  synthetic sequence
ttgctactacaggtggcaggttaaaatcactagccattgc

SEQ ID NO: 986 - DNA, 40 bp, Artificial sequence
  synthetic sequence
ctccttttagctgacatttatcacagctggctactatttc

SEQ ID NO: 987 - DNA, 40 bp, Artificial sequence
  synthetic sequence
ctaccaggataacttttccttctaaatgtgtacaatctag

SEQ ID NO: 988 - DNA, 40 bp, Artificial sequence
  synthetic sequence
gaataacttctgcttctatatatccactggctacatgaac

SEQ ID NO: 989 - DNA, 40 bp, Artificial sequence
  synthetic sequence
accaacaggcggccctaaccgtagcaccggtgaaattgct

SEQ ID NO: 990 - DNA, 40 bp, Artificial sequence
  synthetic sequence
ggggattgtagggaattccaaattcctgcttgattcccgc

SEQ ID NO: 991 - DNA, 40 bp, Artificial sequence
  synthetic sequence
tcttaagatgttcagcctgatctcttacctgtcctataat

SEQ ID NO: 992 - DNA, 40 bp, Artificial sequence
  synthetic sequence
ctactattctttcccctgcactgtaccccccaatcccccc

SEQ ID NO: 993 - DNA, 40 bp, Artificial sequence
  synthetic sequence
tccagaggagctttgctggtcctttccaaagtggatttct

SEQ ID NO: 994 - DNA, 40 bp, Artificial sequence
  synthetic sequence
ttatgtcactattatcttgtattactactgccccttcacc

SEQ ID NO: 995 - DNA, 40 bp, Artificial sequence
  synthetic sequence
cctgtctacttgccacacaatcatcacctgccatctgttt

SEQ ID NO: 996 - DNA, 40 bp, Artificial sequence
  synthetic sequence
catatggtgttttactaaacttttccatgttctaatcctc

SEQ ID NO: 997 - DNA, 40 bp, Artificial sequence
  synthetic sequence
gtgatgtctataaaaccatcccctagctttccctgaaaca

SEQ ID NO: 998 - DNA, 40 bp, Artificial sequence
  synthetic sequence
gatgtgtacttctgaacttattcttggatgagggctttca

SEQ ID NO: 999 - DNA, 40 bp, Artificial sequence
  synthetic sequence
accccaatatgttgttattaccaatctagcatcccctagt

SEQ ID NO: 1000 - DNA, 40 bp, Artificial sequence
  synthetic sequence
gtcaaagtaatacagatgaattagttggtctgctagttca

SEQ ID NO: 1001 - DNA, 40 bp, Artificial sequence
  synthetic sequence
gtgtcctaataaggcctttcttatagcagagtctgaaaaa

SEQ ID NO: 1002 - DNA, 40 bp, Artificial sequence
  synthetic sequence
cttgttatgtcctgcttgatattcacacctagggctaact

SEQ ID NO: 1003 - DNA, 40 bp, Artificial sequence
  synthetic sequence
tgttattaatgctgctagtgccaagtattgtagagatcct

SEQ ID NO: 1004 - DNA, 40 bp, Artificial sequence
  synthetic sequence
cagtttcgtaacactaggcaaaggtggctttatctttttt

SEQ ID NO: 1005 - DNA, 40 bp, Artificial sequence
  synthetic sequence
gtggcccttggtcttctggggcttgttccatctatcctct

SEQ ID NO: 1006 - DNA, 40 bp, Artificial sequence
  synthetic sequence
cctctaaaagctctagtgtccattcattgtgtggctccct

SEQ ID NO: 1007 - DNA, 40 bp, Artificial sequence
  synthetic sequence
gccaaatcctaggaaaatgtctaacagcttcattcttaag

SEQ ID NO: 1008 - DNA, 40 bp, Artificial sequence
  synthetic sequence
tatccccataagtttcatagatatgttgccctaagccatg

SEQ ID NO: 1009 - DNA, 40 bp, Artificial sequence
  synthetic sequence
gttgttgcagaattcttattatggcttccactcctgccca

SEQ ID NO: 1010 - DNA, 40 bp, Artificial sequence
  synthetic sequence
tctgctatgtcgacacccaattctgaaaatggataaacag

SEQ ID NO: 1011 - DNA, 40 bp, Artificial sequence
  synthetic sequence
actggctccatttcttgctctcctctgtcgagtaacgcct

SEQ ID NO: 1012 - DNA, 40 bp, Artificial sequence
  synthetic sequence
ggctgacttcctggatgcttccagggctctagtctaggat

SEQ ID NO: 1013 - DNA, 40 bp, Artificial sequence
  synthetic sequence
gagatgcctaaggcttttgttatgaaacaaacttggcaat

SEQ ID NO: 1014 - DNA, 40 bp, Artificial sequence
  synthetic sequence
tgatgagctcttcgtcgctgtctccgcttcttcctgccat

SEQ ID NO: 1015 - DNA, 40 bp, Artificial sequence
  synthetic sequence
acttactgctttgatagagaagcttgatgagtctgactgt

SEQ ID NO: 1016 - DNA, 40 bp, Artificial sequence
  synthetic sequence
gctactattgctactattggtataggttgcattacatgta

SEQ ID NO: 1017 - DNA, 40 bp, Artificial sequence
  synthetic sequence
ctgtcttctgctctttctattagtctatcaattaacctgt

SEQ ID NO: 1018 - DNA, 40 bp, Artificial sequence
  synthetic sequence
tcatcaacatcccaaggagcatggtgccccatctccaccc

SEQ ID NO: 1019 - DNA, 40 bp, Artificial sequence
  synthetic sequence
cataatagactgtgacccacaatttttctgtagcactaca

SEQ ID NO: 1020 - DNA, 40 bp, Artificial sequence
  synthetic sequence
cacaaaatagagtggtggttgcttccttccacacaggtac

SEQ ID NO: 1021 - DNA, 40 bp, Artificial sequence
  synthetic sequence
aaacattatgtacctctgtatcatatgctttagcatctga

SEQ ID NO: 1022 - DNA, 40 bp, Artificial sequence
  synthetic sequence
cttgtgggttggggtctgtgggtacacaggcatgtgtggc

SEQ ID NO: 1023 - DNA, 40 bp, Artificial sequence
  synthetic sequence
aactgattatatcctcatgcatctgttctaccatgtcatt

SEQ ID NO: 1024 - DNA, 40 bp, Artificial sequence
  synthetic sequence
gtggggttaattttacacatggctttaggctttgatccca

SEQ ID NO: 1025 - DNA, 40 bp, Artificial sequence
  synthetic sequence
tagtatcattcttcaaatcagtgcactttaaactaacaca

SEQ ID NO: 1026 - DNA, 40 bp, Artificial sequence
  synthetic sequence
ctcctttctccattatcattctcccgctactactattggt

SEQ ID NO: 1027 - DNA, 40 bp, Artificial sequence
  synthetic sequence
ttgtcaacttatagctggtagtatcattatctattggtat

SEQ ID NO: 1028 - DNA, 40 bp, Artificial sequence
  synthetic sequence
atacctttggacaggcctgtgtaatgactgaggtgttaca

SEQ ID NO: 1029 - DNA, 40 bp, Artificial sequence
  synthetic sequence
ttccatgtgtacattgtactgtgctgacatttgtacatgg

SEQ ID NO: 1030 - DNA, 40 bp, Artificial sequence
  synthetic sequence
gactgccatttaacagcagttgagttgatactactggcct

SEQ ID NO: 1031 - DNA, 40 bp, Artificial sequence
  synthetic sequence
ccgtgaaattgacagatctaattactacctcttcttctgc

SEQ ID NO: 1032 - DNA, 40 bp, Artificial sequence
  synthetic sequence
ctacagatgtgttcagctgtactattatggttttagcatt

SEQ ID NO: 1033 - DNA, 40 bp, Artificial sequence
  synthetic sequence
ctattgtaacaaatgctctccctggtcctctctggatacg

SEQ ID NO: 1034 - DNA, 40 bp, Artificial sequence
  synthetic sequence
tactaatgttacaatgtgcttgtctcatatttcctatttt

SEQ ID NO: 1035 - DNA, 40 bp, Artificial sequence
  synthetic sequence
atttgctagctatctgttttaaagtgttattccattttgc

SEQ ID NO: 1036 - DNA, 40 bp, Artificial sequence
  synthetic sequence
taaaactgtgcgttacaatttctgggtcccctcctgagga

SEQ ID NO: 1037 - DNA, 40 bp, Artificial sequence
  synthetic sequence
acagttgtgttgaattacagtagaaaaattcccctccaca

SEQ ID NO: 1038 - DNA, 40 bp, Artificial sequence
  synthetic sequence
acccttcagtactccaagtactattaaaccaagtactatt

SEQ ID NO: 1039 - DNA, 40 bp, Artificial sequence
  synthetic sequence
tgcatgggagggtgattgtgtcacttccttcagtgttatt

SEQ ID NO: 1040 - DNA, 40 bp, Artificial sequence
  synthetic sequence
atgaacatctaatttgtccactgatgggaggggcatacat

SEQ ID NO: 1041 - DNA, 40 bp, Artificial sequence
  synthetic sequence
tattaccaccatctcttgttaatagcagccctgtaatatt

SEQ ID NO: 1042 - DNA, 40 bp, Artificial sequence
  synthetic sequence
tatctcctcctccaggtctgaagatctcggactcattgtt

SEQ ID NO: 1043 - DNA, 40 bp, Artificial sequence
  synthetic sequence
gtggtagctgaagaggcacaggctccgcagatcgtcccag

SEQ ID NO: 1044 - DNA, 40 bp, Artificial sequence
  synthetic sequence
ttccacaatcctcgttacaatcaagagtaagtctctcaag

SEQ ID NO: 1045 - DNA, 40 bp, Artificial sequence
  synthetic sequence
ccaccaatatttgagggcttcccaccccctgcgtcccaga

SEQ ID NO: 1046 - DNA, 40 bp, Artificial sequence
  synthetic sequence
agcactattctttagttcctgactccaatactgtaggaga

SEQ ID NO: 1047 - DNA, 40 bp, Artificial sequence
  synthetic sequence
cccctcagctactgctatggctgtggcattgagcaagcta

SEQ ID NO: 1048 - DNA, 40 bp, Artificial sequence
  synthetic sequence
agctctacaagctccttgtactacttctataaccctatct

SEQ ID NO: 1049 - DNA, 40 bp, Artificial sequence
  synthetic sequence
acactactttttgaccacttgccacccatcttatagcaaa

SEQ ID NO: 1050 - DNA, 40 bp, Artificial sequence
  synthetic sequence
tcagctcgtctcattctttcccttacagtaggccatccaa

SEQ ID NO: 1051 - DNA, 40 bp, Artificial sequence
  synthetic sequence
tccaggtctcgagatgctgctcccaccctatctgctgctg

SEQ ID NO: 1052 - DNA, 40 bp, Artificial sequence
  synthetic sequence
ttggtagctgctgtattgctacttgtgattgctccatgtt

SEQ ID NO: 1053 - DNA, 40 bp, Artificial sequence
  synthetic sequence
gtcattggtcttaaaggtacctgaggtgtgactggaaaac

SEQ ID NO: 1054 - DNA, 40 bp, Artificial sequence
  synthetic sequence
tcttgtcttctttgggagtgaattagcccttccagtcccc

SEQ ID NO: 1055 - DNA, 40 bp, Artificial sequence
  synthetic sequence
gggaagtagccttgtgtgtggtagatccacagatcaagga

SEQ ID NO: 1056 - DNA, 40 bp, Artificial sequence
  synthetic sequence
ggatatctgacccctggccctggtgtgtagttctgctaat

SEQ ID NO: 1057 - DNA, 40 bp, Artificial sequence
  synthetic sequence
ggctcaactggtactagcttgtagcaccatccaaaggtca

SEQ ID NO: 1058 - DNA, 40 bp, Artificial sequence
  synthetic sequence
aagctggtgttctctcctttattggcctcttctatcttat

SEQ ID NO: 1059 - DNA, 40 bp, Artificial sequence
  synthetic sequence
ctctccgggtcatccatcccatgcaggctcacagggtgta

SEQ ID NO: 1060 - DNA, 40 bp, Artificial sequence
  synthetic sequence
tgaaatgctaggcggctgtcaaacctccactctaacactt

SEQ ID NO: 1061 - DNA, 40 bp, Artificial sequence
  synthetic sequence
cagttcttgaagtactccggatgcagctctcgggccacgt

SEQ ID NO: 1062 - protein, 196 aa, Artificial sequence
  synthetic sequence
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 1063 - DNA, 53 bp, Artificial sequence
  synthetic sequence
tgtgtaacaatgcctgtggctctctgatgacagtgcatggctgcaatgtgtga

SEQ ID NO: 1064 - DNA, 57 bp, Artificial sequence
  synthetic sequence
tgctcagcccagctcagcctgcagccctgtgggaaatggtagagaatgagaggggga

SEQ ID NO: 1065 - DNA, 50 bp, Artificial sequence
  synthetic sequence
tcccctccaccccacagtgtccctagtggccccaggattggtgacagaaa

SEQ ID NO: 1066 - protein, 198 aa, Artificial sequence
  synthetic sequence
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFRS

SEQ ID NO: 1067 - protein, 198 aa, Artificial sequence
  synthetic sequence
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFRS

SEQ ID NO: 1068 - DNA, 53 bp, Artificial sequence; identical to SEQ ID NO: 1063
  synthetic sequence

SEQ ID NO: 1069 - DNA, 53 bp, Artificial sequence; identical to SEQ ID NO: 1063
  synthetic sequence

SEQ ID NO: 1070 - DNA, 58 bp, Artificial sequence
  synthetic sequence
tggagtgtgtaacaatgcctgtggctctctgatgacagtgcatggctgcaatgtgtga

SEQ ID NO: 1071 - DNA, 40 bp, Artificial sequence
  synthetic sequence
tgcaagaaccaaaacccgttcctcctggctcaggccggaa

SEQ ID NO: 1072 - DNA, 40 bp, Artificial sequence
  synthetic sequence
tctggcccagtcgactcaggggctgaatcgggcatgactc

SEQ ID NO: 1073 - DNA, 40 bp, Artificial sequence
  synthetic sequence
tcgtggcctggagccaccgctccctccaacaccgcaaagt

SEQ ID NO: 1074 - DNA, 40 bp, Artificial sequence
  synthetic sequence
ctggggttcagtgagagcacgtgatctgctcagccagtca

SEQ ID NO: 1075 - DNA, 40 bp, Artificial sequence
  synthetic sequence
ttcgctttccctggcttacttgctgttttcctctctctgg

SEQ ID NO: 1076 - DNA, 40 bp, Artificial sequence
  synthetic sequence
gctgggagagaagacagaccggcctcaggcacgaccatcc

SEQ ID NO: 1077 - DNA, 40 bp, Artificial sequence
  synthetic sequence
gctctggccatagtgtggccctggcagccactcacaggca

SEQ ID NO: 1078 - DNA, 40 bp, Artificial sequence
  synthetic sequence
ccacatgatgcagaattccccgaggtgctggcatccagac

SEQ ID NO: 1079 - DNA, 40 bp, Artificial sequence
  synthetic sequence
ctctaaggagggcgggtcttttgcaccccctgcaggacac

SEQ ID NO: 1080 - DNA, 40 bp, Artificial sequence
  synthetic sequence
gggctgcagtgcgcaggacctggatcacaggctgcacccc

SEQ ID NO: 1081 - DNA, 40 bp, Artificial sequence
  synthetic sequence
gtgacaccctgtgacacccggctccacacaggagcctcag

SEQ ID NO: 1082 - DNA, 40 bp, Artificial sequence
  synthetic sequence
cggggtgggactctgcggccccaaatcacaaggcgactgc

SEQ ID NO: 1083 - DNA, 40 bp, Artificial sequence
  synthetic sequence
aagaccactggggccactggaaagaccctcagccgtgctg

SEQ ID NO: 1084 - DNA, 40 bp, Artificial sequence
  synthetic sequence
acattggtgggggatattggcttgtaggatcagccaggaa

SEQ ID NO: 1085 - DNA, 40 bp, Artificial sequence
  synthetic sequence
gaaattgctcataacttgcatcagcttctcagagggggcc

SEQ ID NO: 1086 - DNA, 40 bp, Artificial sequence
  synthetic sequence
tccagggggtctgtgaactttctgacgttgtattttcctg

SEQ ID NO: 1087 - DNA, 40 bp, Artificial sequence
  synthetic sequence
ggatccagatctgggtgatttaggctccctctgtctggat

SEQ ID NO: 1088 - DNA, 40 bp, Artificial sequence
  synthetic sequence
attctttgtagcctctcccgctctggttcagggcccagct

SEQ ID NO: 1089 - DNA, 40 bp, Artificial sequence
  synthetic sequence
accaaccttgatgctacactgttgcctgcgtttctccttg

SEQ ID NO: 1090 - DNA, 40 bp, Artificial sequence
  synthetic sequence
cacccaccgcaccaaccttgatgctacactctcacccact

SEQ ID NO: 1091 - DNA, 40 bp, Artificial sequence
  synthetic sequence
gctacactctcacccaccgcaccaaccttgatgctacact

SEQ ID NO: 1092 - DNA, 40 bp, Artificial sequence
  synthetic sequence
caaccttgatgctacactctcacccaccgcaccaaccttg

SEQ ID NO: 1093 - DNA, 40 bp, Artificial sequence
  synthetic sequence
cccaccgcaccaaccttgatgctacactctcacccaccgc

SEQ ID NO: 1094 - DNA, 40 bp, Artificial sequence
  synthetic sequence
caacacgctaccccctgtgttgaccttgatgctacactct

SEQ ID NO: 1095 - DNA, 40 bp, Artificial sequence
  synthetic sequence
cctgccacaaggaaaacctcctgcagaaccacagtaggga

SEQ ID NO: 1096 - DNA, 40 bp, Artificial sequence
  synthetic sequence
tgcaggcattgtacatcttcgcctgatgcacagcaggtat

SEQ ID NO: 1097 - DNA, 40 bp, Artificial sequence
  synthetic sequence
gatctcttcccaggtatagacataaacacattttttccta

SEQ ID NO: 1098 - DNA, 40 bp, Artificial sequence
  synthetic sequence
tcatcatcccccaacgaaaccctgcaaccgcttagccatc

SEQ ID NO: 1099 - DNA, 40 bp, Artificial sequence
  synthetic sequence
acggggtcgggcatttatgaccacattggttgtagaacat

SEQ ID NO: 1100 - DNA, 40 bp, Artificial sequence
  synthetic sequence
aattcacccaaagtgcacacttcagtgctttttagtctat

SEQ ID NO: 1101 - DNA, 40 bp, Artificial sequence
  synthetic sequence
tttacagaaaagttgaagcaatagcatgtgactacccata

SEQ ID NO: 1102 - DNA, 40 bp, Artificial sequence
  synthetic sequence
gaaatggggagtgggtcaaatcagccctggacctggattc

SEQ ID NO: 1103 - DNA, 40 bp, Artificial sequence
  synthetic sequence
cgtgacggcggagatctgaggttcgggagcccctctttgg

SEQ ID NO: 1104 - DNA, 40 bp, Artificial sequence
  synthetic sequence
ggggtccacgagagccatgcgggaggactagctagtggga

SEQ ID NO: 1105 - DNA, 40 bp, Artificial sequence
  synthetic sequence
gccgctggccaggctgaaaggataggattccgcgtgggtt

SEQ ID NO: 1106 - DNA, 40 bp, Artificial sequence
  synthetic sequence
accggcagcctccgagacttctgacgcggctgtcctgacg

SEQ ID NO: 1107 - DNA, 40 bp, Artificial sequence
  synthetic sequence
ggaccgtgtggaaggaaagggagactgacgaggaaatgag

SEQ ID NO: 1108 - DNA, 40 bp, Artificial sequence
  synthetic sequence
tggagtggaagggtgtgagcatggttcccggcagactcca

SEQ ID NO: 1109 - DNA, 40 bp, Artificial sequence
  synthetic sequence
ctggtgccgcttcatggggtggttgtcagggtctggctgg

SEQ ID NO: 1110 - DNA, 40 bp, Artificial sequence
  synthetic sequence
cgtccctgaagcttgcttccctgatttcctaaaacaggac

SEQ ID NO: 1111 - DNA, 40 bp, Artificial sequence
  synthetic sequence
ggcttgcctcccagctctgcctgtgactggtgactccagg

SEQ ID NO: 1112 - DNA, 40 bp, Artificial sequence
  synthetic sequence
acacaggatccctgggtccccagcatgtcttctaaagtcc

SEQ ID NO: 1113 - DNA, 40 bp, Artificial sequence
  synthetic sequence
ttctagggaaggggtgttgcttctagcaggtgtgtgatgg

SEQ ID NO: 1114 - DNA, 40 bp, Artificial sequence
  synthetic sequence
gggtccaggagcccctgaaactgtgtctggccaggttcat

SEQ ID NO: 1115 - DNA, 40 bp, Artificial sequence
  synthetic sequence
cctgtcctctgagactcatcgtaccccaggagccttcata

SEQ ID NO: 1116 - DNA, 40 bp, Artificial sequence
  synthetic sequence
ggggggagtaggggcatgcaggggttgccagggactggtc

SEQ ID NO: 1117 - DNA, 40 bp, Artificial sequence
  synthetic sequence
aaccctgccgcaggtctttctgggaggggatgcgtttact

SEQ ID NO: 1118 - DNA, 40 bp, Artificial sequence
  synthetic sequence
gtggagggactcacccaggagtgcgttaggtaggattgct

SEQ ID NO: 1119 - DNA, 40 bp, Artificial sequence
  synthetic sequence
tgagtaactgaggggattggaatgccggggcggggtgggt

SEQ ID NO: 1120 - DNA, 40 bp, Artificial sequence
  synthetic sequence
atgagaactcaaacccctaccaactgggactgtcaatccc

SEQ ID NO: 1121 - DNA, 40 bp, Artificial sequence
  synthetic sequence
ggcctgcctccaggattgcttggagcccagcacacgcaca

SEQ ID NO: 1122 - DNA, 40 bp, Artificial sequence
  synthetic sequence
gcctgggcaccgaggctgaccctgcttcctaggattgtct

SEQ ID NO: 1123 - DNA, 40 bp, Artificial sequence
  synthetic sequence
acctcctcacccgtggtctccaggctgagagctttagagg

SEQ ID NO: 1124 - DNA, 40 bp, Artificial sequence
  synthetic sequence
gagtcggacgccatggaggggctgctgaaggcggagatcg

SEQ ID NO: 1125 - DNA, 40 bp, Artificial sequence
  synthetic sequence
gccgccgtcaacagtgacggggacctgcccctggacctgg

SEQ ID NO: 1126 - DNA, 40 bp, Artificial sequence
  synthetic sequence
gcccccacccccaggtacctcctgagccacggggccaaca

SEQ ID NO: 1127 - DNA, 40 bp, Artificial sequence
  synthetic sequence
ggacctggtcggggtgggggcctggaccctcagccctgac

SEQ ID NO: 1128 - DNA, 40 bp, Artificial sequence
  synthetic sequence
gctacctagatatcgccaggtgaggcaagggagggccggg

SEQ ID NO: 1129 - DNA, 40 bp, Artificial sequence
  synthetic sequence
acaacgagggctggacgccactgcacgtggccgcctcctg

SEQ ID NO: 1130 - DNA, 40 bp, Artificial sequence
  synthetic sequence
tgcgcttcttggtggagcagggcgccactgtgaaccaggc

SEQ ID NO: 1131 - DNA, 40 bp, Artificial sequence
  synthetic sequence
tttcccacccccaggcctgcattgatgagaacctggaggt

SEQ ID NO: 1132 - DNA, 40 bp, Artificial sequence
  synthetic sequence
ttgctgggacaccgtggctggggtaggtgcggctgacggc

SEQ ID NO: 1133 - DNA, 40 bp, Artificial sequence
  synthetic sequence
tgtccctggatctgttttcgtggctccctctggagtcccg

SEQ ID NO: 1134 - DNA, 40 bp, Artificial sequence
  synthetic sequence
gccagaggctgttgggtcattttccccactgtcctagcac

SEQ ID NO: 1135 - DNA, 40 bp, Artificial sequence
  synthetic sequence
gcctgaccactgggcaaccaggcgtatcttaaacagccag

SEQ ID NO: 1136 - DNA, 40 bp, Artificial sequence
  synthetic sequence
gagtcctttcgtggtttccactgagcactgaaggcctggc

SEQ ID NO: 1137 - DNA, 40 bp, Artificial sequence
  synthetic sequence
ccccctcccttccccgttcacttcctgtttgcagatagcc

SEQ ID NO: 1138 - DNA, 40 bp, Artificial sequence
  synthetic sequence
tctaacaggtaccatgtggggttcccgcacccagatgaga

SEQ ID NO: 1139 - DNA, 40 bp, Artificial sequence
  synthetic sequence
ctggaagcgccacctgtgggtggtgacgggggttttgccg

SEQ ID NO: 1140 - DNA, 40 bp, Artificial sequence
  synthetic sequence
ctgctggggtggtttccgagcttgacccttggaaggacct

SEQ ID NO: 1141 - DNA, 40 bp, Artificial sequence
  synthetic sequence
cctgcatagccctgggcccacggcttcgttcctgcagagt

SEQ ID NO: 1142 - DNA, 40 bp, Artificial sequence
  synthetic sequence
aggcccctgagtctgtcccagcacagggtggccttcctcc

SEQ ID NO: 1143 - DNA, 40 bp, Artificial sequence
  synthetic sequence
acacaggtgtgcagctgtctcacccctctgggagtcccgc

SEQ ID NO: 1144 - DNA, 40 bp, Artificial sequence
  synthetic sequence
ggggcctcagtgaactggagtgtgacagcctggggcccag

SEQ ID NO: 1145 - DNA, 40 bp, Artificial sequence
  synthetic sequence
ggtggcccgtgtcagcccctggctgcagggccccgtgcag

SEQ ID NO: 1146 - DNA, 40 bp, Artificial sequence
  synthetic sequence
tgtccccccaagttttggacccctaagggaagaatgagaa

SEQ ID NO: 1147 - DNA, 40 bp, Artificial sequence
  synthetic sequence
cctggggcaagtccctcctccgaccccctggacttcggct

SEQ ID NO: 1148 - DNA, 40 bp, Artificial sequence
  synthetic sequence
agctccagttcaggtcccggagcccacccagtgtccacaa

SEQ ID NO: 1149 - DNA, 40 bp, Artificial sequence
  synthetic sequence
atttatcccgtggatctaggagtttagcttcactccttcc

SEQ ID NO: 1150 - DNA, 40 bp, Artificial sequence
  synthetic sequence
tccagatgggcagctttggagaggtgagggacttgggggg

SEQ ID NO: 1151 - DNA, 40 bp, Artificial sequence
  synthetic sequence
atgacctcatgctcttggccctcgtagctccctcccgcct

SEQ ID NO: 1152 - DNA, 40 bp, Artificial sequence
  synthetic sequence
cgttcccagggcacgtgcggccccttcacagcccgagttt

SEQ ID NO: 1153 - DNA, 40 bp, Artificial sequence
  synthetic sequence
cgccatgacaactgggtggaaataaacgagccgagttcat

SEQ ID NO: 1154 - DNA, 40 bp, Artificial sequence
  synthetic sequence
gaaagggaaaggcccattgctctccttgcccccctcccct

SEQ ID NO: 1155 - DNA, 40 bp, Artificial sequence
  synthetic sequence
tcaggcatctttcacagggatgcctgtactgggcaggtcc

SEQ ID NO: 1156 - DNA, 40 bp, Artificial sequence
  synthetic sequence
ttgggggctagagtaggaggggctggagccaggattctta

SEQ ID NO: 1157 - DNA, 40 bp, Artificial sequence
  synthetic sequence
tgcccccattcctgcaccccaattgccttagtggctaggg

SEQ ID NO: 1158 - DNA, 40 bp, Artificial sequence
  synthetic sequence
accccacgtgggtttatcaaccacttggtgaggctggtac

SEQ ID NO: 1159 - DNA, 40 bp, Artificial sequence
  synthetic sequence
agcatcgcccccctgctgtggctgttcccaagttcttagg

SEQ ID NO: 1160 - DNA, 40 bp, Artificial sequence
  synthetic sequence
gctgtgtttctcgtcctgcatccttctccaggcaggtccc

SEQ ID NO: 1161 - DNA, 40 bp, Artificial sequence
  synthetic sequence
ctctgggtgactcttgattcccggccagtttctccacctg

SEQ ID NO: 1162 - DNA, 40 bp, Artificial sequence
  synthetic sequence
gaaaccctcagtcctaggaaaacagggatggttggtcact

SEQ ID NO: 1163 - DNA, 40 bp, Artificial sequence
  synthetic sequence
ccagcttatgctgtttgcccaggacagcctagttttagca

SEQ ID NO: 1164 - DNA, 40 bp, Artificial sequence
  synthetic sequence
agcaggggagctgggtttgggtcaggtctgggtgtggggt

SEQ ID NO: 1165 - DNA, 40 bp, Artificial sequence
  synthetic sequence
ttcagagaggagggattcccttctcaggttacgtggccaa

SEQ ID NO: 1166 - DNA, 40 bp, Artificial sequence
  synthetic sequence
cggggtatcccaggaggcctggagcattggggtgggctgg

SEQ ID NO: 1167 - DNA, 40 bp, Artificial sequence
  synthetic sequence
tctcctccaactgtggggtgactgcttggcaaactcactc

SEQ ID NO: 1168 - DNA, 40 bp, Artificial sequence
  synthetic sequence
ggccaccccagccctgtctaccaggctgccttttgggtgg

SEQ ID NO: 1169 - DNA, 40 bp, Artificial sequence
  synthetic sequence
ccagaggccccaggccacctacttggcctggaccccacga

SEQ ID NO: 1170 - DNA, 40 bp, Artificial sequence
  synthetic sequence
cctgcatccccgttcccctgcatcccccttcccctgcatc

SEQ ID NO: 1171 - DNA, 40 bp, Artificial sequence
  synthetic sequence
acaggggttcctggctctgctcttcagactgagccccgtt

SEQ ID NO: 1172 - DNA, 40 bp, Artificial sequence
  synthetic sequence
tcgtccaccatctcatgcccctggctctcctgccccttcc

SEQ ID NO: 1173 - DNA, 40 bp, Artificial sequence
  synthetic sequence
gcaagcccaggagaggcgctcaggcttccctgtccccctt

SEQ ID NO: 1174 - DNA, 40 bp, Artificial sequence
  synthetic sequence
ttccctaaggccctgctctgggcttctgggtttgagtcct

SEQ ID NO: 1175 - DNA, 40 bp, Artificial sequence
  synthetic sequence
tgctatctgggacatattcctccgcccagagcagggtccc

SEQ ID NO: 1176 - DNA, 40 bp, Artificial sequence
  synthetic sequence
ggtgcgtcctaggtgttcaccaggtcgtggccgcctctac

SEQ ID NO: 1177 - DNA, 40 bp, Artificial sequence
  synthetic sequence
gaggaggggggtgtccgtgtggaaaactccctttgtgaga

SEQ ID NO: 1178 - DNA, 40 bp, Artificial sequence
  synthetic sequence
agataaggccagtagccagccccgtcctggcagggctgtg

SEQ ID NO: 1179 - DNA, 40 bp, Artificial sequence
  synthetic sequence
ccccaatttatattgttcctccgtgcgtcagttttacctg

SEQ ID NO: 1180 - DNA, 40 bp, Artificial sequence
  synthetic sequence
agttggtcctgagttctaactttggctcttcacctttcta

SEQ ID NO: 1181 - DNA, 40 bp, Artificial sequence
  synthetic sequence
ctggtgcgtttcactgatcctggtgctgcagcttccttac

SEQ ID NO: 1182 - DNA, 40 bp, Artificial sequence
  synthetic sequence
cgctaccctctcccagaacctgagctgctctgacgcggcc

SEQ ID NO: 1183 - DNA, 40 bp, Artificial sequence
  synthetic sequence
gggggggatgcgtgacctgcccggttctcagtggccaccc

SEQ ID NO: 1184 - DNA, 40 bp, Artificial sequence
  synthetic sequence
tccttgccagaacctctaaggtttgcttacgatggagcca

SEQ ID NO: 1185 - DNA, 40 bp, Artificial sequence
  synthetic sequence
ccttatctggtgacacacccccatttcctggagccatctc

(19) United States
(12) Patent Application Publication
(10) Pub. No.: US 2021/0147922 A1
(43) Pub. Date: May 20, 2021

(54) METHODS FOR ASSESSING SPECIFICITY OF CELL ENGINEERING TOOLS

(71) Applicant: ALTIUS INSTITUTE FOR BIOMEDICAL SCIENCES, Seattle, WA (US)

(72) Inventors: Fyodor Urnov, Seattle, WA (US); John A. Stamatoyannopoulos, Seattle, WA (US); Vivek Nandakumar, Seattle, WA (US); Pavel Zrazhevskiy, Seattle, WA (US); Shreeram Akilesh, Seattle, WA (US)

(21) Appl. No.: 17/047,456

(22) PCT Filed: Apr. 18, 2019
(86) PCT No.: PCT/US2019/028200

§ 371 Date: Oct. 14, 2020

Related U.S. Application Data
(60) Provisional application No. 62/690,908, filed on Jun. 27, 2018.
(60) Provisional application No. 62/659,664, filed on Apr. 18, 2018.

(51) Int. Cl. C12Q 1/6841; C12N 15/10

(52) U.S. Cl. CPC C12Q 1/6841; C12N 2800/80; C12N 2310/20; C12N 15/1024

(57) ABSTRACT

The present disclosure provides methods and compositions for image based analysis and quantification of a protein load from protein (e.g., p53BP1) accumulation, induced by a cellular perturbation, such as administration of a genome editing tool comprising a DNA binding domain and a nuclease domain, a gene repressor, or a gene activator.

### CROSS-REFERENCE TO RELATED APPLICATIONS

This application claims priority to U.S. Provisional Application Ser. No. 62/659,664 filed Apr. 18, 2018 and U.S. Provisional Application Ser. No. 62/690,908 filed Jun. 27, 2018, the disclosures of which are herein incorporated by reference in their entirety.

### INTRODUCTION

Current tools to assess off-target activity of nucleases such as transcription activator-like effector nucleases (TALENs), Zinc Finger Nucleases (ZFNs), Cas nucleases are predominantly bulk-cell based, and thus only provide population-averaged estimates. Furthermore, these techniques necessitate costly deep sequencing and complex computational strategies to obtain the required results. All current techniques preclude information about the cell-cell variability in the (1) the extent of off-target nuclease activity, (2) nuclear localization of nuclease activity, (3) cell transfection efficiency, (4) levels of nuclease expression, (5) nuclease induced cytotoxicity. Thus, there is a need torr a quantitative imaging-based assay to overcome these limitations, which could be applied to all nuclease classes in primary cells and immortalized cells.

### SUMMARY

Methods to assess the specificity of cell engineering tools disclosed herein measure the differential response of a cell to a cellular perturbation by a cell engineering tool by quantifying the change in the load of protein relevant to such a response, relative to the background load of the same protein in untreated reference cells, and, in some cases, normalized by the predicted magnitude of response to perturbation by a target-specific cell engineering tool. Degree of deviation of the change in protein load beyond that expected for a target-specific cell engineering tool is used as an indicator of additional off-target activity by cell engineering tool, which might be undesirable. The cell engineering tool might be optimized to achieve an increased target-specific response using the analytical workflow disclosed herein.

In various aspects, the present disclosure provides a method of quantifying a protein load, the method comprising quantifying a protein that accumulates in a primary cell in response to a cellular perturbation on a per allele per cell basis.

In various aspects, the present disclosure provides a method of quantifying a protein load, the method comprising quantifying a protein that accumulates in a plurality of cells in response to a cellular perturbation in less than 24 hours on a per allele per cell basis.

In various aspects, the present disclosure provides a method of screening a plurality of cell engineering tools for specificity, the method comprising quantifying a protein load in an intact cell in less than 24 hours and determining the specificity of the cell engineering tool for a target genomic locus based on the protein load.

In various aspects, the present disclosure provides a method of producing a potent and specific cell engineering tool, the method comprising: a) administering a cell engineering tool to a cell; b) determining specificity, activity, or a combination thereof of the cell engineering tool for a target genomic locus by quantifying a protein load; c) quantifying potency of the cell engineering tool by measuring gene editing efficiency, activation of gene expression, or repression of gene expression; and d) adjusting a parameter of the cell engineering tool to increase specificity for the target genomic locus.

In some aspects, the protein accumulates in response to a cellular perturbation. In further aspects, the method further comprises quantifying the protein load on a per allele per cell basis. In some aspects, the intact cell comprises an intact primary cell. In some aspects, the cell comprises an intact primary cell. In further aspects, the cellular perturbation comprises administering a cell engineering tool.

In some aspects, the method further comprises determining specificity of the cell engineering tool for a target genomic locus. In some aspects, the method further comprises quantifying gene editing efficiency, activation of gene expression, or repression or gene expression. In some aspects, the plurality of cells comprises at least 5 cells, at least 10 cells, at least 20 cells, at least 50 cells, at least 100 cells, at least 200 cells, at least 500 cells, or at least 1000 cells.

In some aspects, the protein indicates a cellular response. In some aspects, the cellular response comprises a double strand break, activation of transcription, repression of transcription, or chromosome translocation.

In other aspects, the cell or intact cell comprises an immortalized cell. In some aspects, the cell engineering tool comprises a genome editing complex or a gene regulator. In some aspects, the gene regulator comprises a gene activator or a gene repressor. In some aspects, the protein comprises phosphorylated p53BP1 (p53BP1), γH2AX, 53BP1, H3K4me1, H3K4me2, H3K27ac, KAP1, H3K9me3, H3K27me3, or HP1. In further aspects, the protein comprises p53BP1.

In some aspects, the method further comprises staining the cell for the protein. In some aspects, the staining the cell for the protein comprises labeling with a primary antibody against the protein and a secondary antibody conjugated to a first fluorophore. In other aspects, the staining the cell for the protein comprises direct labeling with a primary antibody conjugated to a first fluorophore. In some aspects, the method further comprises imaging the cell for one or more protein foci comprising the first fluorophore. In some aspects, the method further comprises image analysis of the cell for the one or more protein foci comprising the first fluorophore.

In some aspects, the method further comprises quantifying the protein load from the one or more protein foci comprising the first fluorophore. In some aspects, the protein load comprises a number of protein foci, total protein content within the nucleus, spatial localization pattern, or any combination thereof. In further aspects, the cell engineering tool further comprises a polypeptide tag. In still further aspects, the polypeptide tag is a FLAG tag.

In some aspects, the method further comprises staining the cell for the cell engineering tool. In some aspects, the staining the cell for the cell engineering tool comprises staining with a primary antibody against the polypeptide tag and a secondary antibody conjugated to a second fluorophore. In other aspects, the staining the cell for the cell engineering tool comprises direct labeling with a primary antibody conjugated to a second fluorophore. In some aspects, the staining of the cell for the cell engineering tool comprises staining with a primary antibody against the nuclease and a secondary antibody conjugated to a second fluorophore. In other aspects, the staining the cell for the cell engineering tool comprises direct labeling with a primary antibody conjugated to a second fluorophore.

In some aspects, the method further comprises imaging the cell for one or more cell engineering tool foci comprising the second fluorophore. In some aspects, the method further comprises image analysis of the cell for the one or more cell engineering tool foci comprising the second fluorophore. In some aspects, the method further comprises quantifying cell engineering tool load from the one or more cell engineering tool foci comprising the second fluorophore. In some aspects, the cell engineering tool load comprises a number of cell engineering tool foci, total content of the cell engineering tool within the nucleus, spatial localization pattern, or any combination thereof.

In some aspects, the method further comprises hybridizing a probe set comprising a plurality of probes to the cell, wherein the probe set targets and binds to a target genomic locus. In some aspects, each probe of the plurality of probes comprises a third fluorophore. In some aspects, the probe set comprises an oligonucleotide probe set. In some aspects, the method further comprises imaging the cell for one or more Nano-FISH foci comprising the third fluorophore. In some aspects, the method further comprises image analysis of the cell for the one or more Nano-FISH foci comprising the third fluorophore. In some aspects, co-localization of signal from the first fluorophore and the third fluorophore indicates that the cellular perturbation occurs at the target genomic locus.

In some aspects, the method further comprises hybridizing a second probe set comprising a second plurality of probes to the cell, wherein the second probe set targets and binds to an off-target genomic locus. In some aspects, each probe of the second plurality of probes comprises a fourth fluorophore. In further aspects, the second probe set comprises a second oligonucleotide probe set. In further aspects, the method further comprises imaging the cell for one or more Nano-FISH foci comprising the fourth fluorophore. In some aspects, the method further comprises image analysis of the cell for the one or more Nano-FISH foci comprising the fourth fluorophore. In some aspects, co-localization of signal from the first fluorophore, the third fluorophore, and the fourth fluorophore indicates a chromosome translocation.

In some aspects, imaging the cell comprises acquiring images of the cell by a microscopy mode selected from the group consisting of epifluorescence, widefield, confocal, selective plane illumination, tomography, holography, super-resolution, and synthetic aperture optics (SAO). In further aspects, the method further comprises processing the acquired images to identify regions of interest (ROIs) comprising cell nuclei, protein marker foci, sites of cell engineering tool localization, or a combination thereof.

In some aspects, the method further comprises processing the ROIs to extract a plurality of features selected from the group consisting of count, spatial location, size (area/volume), shape (circularity/sphericity, eccentricity, irregularity (concavity/convexity), diameter, perimeter/surface area, quantitative measures of image texture that are pixel-based or region-based over a tunable length scale, nuclear diameter, nuclear area, nuclear volume, perimeter, surface area, DNA content, DNA texture measures, number of protein marker foci, size of protein marker foci, shape of protein marker foci, amount of protein marker per cell, spatial location and localization pattern of protein marker foci, number of nuclease per cell, amount of nuclease per cell, nuclease localization or texture, number of cell engineering tool foci, size of cell engineering tool foci, shape of cell engineering tool foci, amount of cell engineering tool foci per cell, spatial location and localization pattern of cell engineering tool foci, number of Nano-FISH foci, size of Nano-FISH foci, shape of Nano-FISH foci, amount of Nano-FISH foci, spatial location of Nano-FISH foci, and localization pattern of Nano-FISH foci.

In some aspects, the method further comprises processing the extracted plurality of features to measure a degree of co-localization between the one or more Nano-FISH foci and the one or more protein marker foci, thereby determining specificity of the genome editing complex or the gene regulator. In some aspects, the method further comprises applying a machine learning predictor to the extracted plurality of features to evaluate performance of cell engineering tools by predicting a distinction capability of nucleases.

In some aspects, the method further comprises the genome editing complex comprises a DNA binding domain and a nuclease. In further aspects, the genome editing complex further comprises a linker. In some aspects, the gene activator comprises a DNA binding domain and an activation domain. In further aspects, the gene activator further comprises a linker. In some aspects, the gene repressor comprises a DNA binding domain and a repressor domain. In further aspects, the gene repressor further comprises a linker.

In some aspects, the DNA binding domain comprises a transcription activator-like effector (TALE) protein, a zinc finger protein (ZFP), or a single guide RNA (sgRNA). In further aspects, the genome editing complex is a TALEN, a ZRN, a CRISPR/Cas9, a megaTAL, or a meganuclease. In some aspects, the nuclease comprises FokI. In further aspects, FokI has at least 70%, at least 75%, at least 80%, at least 85%, at least 90%, at least 92%, at least 95%, at least 97%, or at least 99% sequence identity to SEQ ID NO: 1062. In some aspects, the linker comprises the naturally occurring C-terminus of a TALE protein or any truncation thereof. In some aspects, the linker comprises 0-15 residues of glycine, methionine, aspartic acid, alanine, lysine, serine, leucine, threonine, tryptophan, or any combination thereof.

In some aspects, the activation domain comprises VP16, VP64, p65, p300 catalytic domain, TET1 catalytic domain, TDG, Ldb1 self-associated domain, SAM activator (VP64, p65, HSF1), VPR (VP64, p65, Rta). In other aspects, the repressor domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

In some aspects, a parameter of the genome editing complex or the gene regulator is adjusted improve specificity. In some aspects, the parameter is a sequence of the DNA binding domain or length of the DNA binding domain. In some aspects, the protein load is quantified in at least 50 to 100,000 cells. In some aspects, the protein load is quantified in no more than 1000, no more than 500, no more than 100, or no more than 50 cells. In some aspects, the cell comprises a hematopoietic stem cells (HSC), a T cell, a chimeric antigen receptor T cell (CAR T cell). In other aspects, the cell is from a normal solid tissue or a tumorigenic solid tissue. In some aspects, the target genomic locus is within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, an IL2RG gene, or a combination thereof. In some aspects, a chimeric antigen receptor (CAR), alpha-L iduronidase (IDUA), iduronate-2-sulfatase (IDS), or Factor 9 (F9) is inserted upon cleavage of a region of the target nucleic acid sequence.

In certain aspects, a method for determining specificity of a protein engineering tool comprises contacting a live cell with a cell engineering tool comprising a DNA binding domain and a nuclease domain, a gene repressor, or a gene activator, wherein the live cell comprises genomic DNA comprising a target genomic locus for the DNA binding domain of the cell engineering tool; fixing the cell and contacting the fixed cell with a plurality of nucleic acid probes complementary to the target genomic locus and assaying for presence of a protein indicative of cellular response to the contacting; and assaying for colocalization of the probes and the protein, wherein detection of the colocalization indicates activity of the cell engineering tool at the target genomic locus and absence of the colocalization indicates activity of the cell engineering tool at an off-target site.

In certain aspects, assaying for colocalization comprises imaging the cell at 40× or higher magnification. In certain aspects, the fixing of the cell is performed within 24 hours or less of the contacting. The cell engineering tool may include a DNA binding domain and a nuclease domain. The nuclease domain induces a double strand break in the genomic DNA and where the protein indicative of cellular response to the contacting comprises a DNA repair protein. The DNA repair protein may be p53BP1, γH2AX, MRE-11, BRCA1, RAD-51, phospho-ATM or MDC1.

The cell engineering tool may include a DNA binding domain and a gene repressor. The gene repressor may be KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

The cell engineering tool may include a DNA binding domain and a gene activator. The gene activator may be VP16, VP64, p65, p300 catalytic domain, IET1 catalytic domain, TDG, Ldb1 self-associated domain, SAM activator (VP64, p65, HSF1), VPR (VP64, p65, Rta).

The DNA binding domain may be a transcription activator-like effector (TALE) protein, a zinc finger protein (ZFP), or a single guide RNA (sgRNA).

The cell may be any cell of interest, including the cells as provided herein, e.g., primary cells. The cell may be hematopoietic stem cell (HSC), a T cell, or a chimeric antigen receptor T cell (CAR T cell). The cell may be from a normal solid tissue or a tumorigenic solid tissue. The cell may be an immortalized cell.

The target genomic locus may be within a PDCD1 gene, a CTLA4 gene, a LAG3 gene, a IET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, or an IL2RG gene, e.g., in the open reading frame, intron, promoter, regulatory elements, and the like of the gene.

The assaying for the colocalization comprises imaging the cell by a microscopy mode selected epifluorescence, widefield, confocal, selective plane illumination, tomography, holography, super-resolution, and synthetic aperture optics (SAO).

The plurality of nucleic acid probes may be 30-60 bases in length and may include 20-200 probes having distinct sequences. The plurality of nucleic acid probes may bind to a 1 kilobase (kb) to 5 kb region comprising the target genomic locus.

In certain aspects, when the absence of colocalization is detected, the method further comprises adjusting a parameter of the genome editing tool to improve specificity. The parameter may be a sequence of the DNA binding domain or length of the DNA binding domain. The parameter may be an amount of the genome editing tool introduced into the cell.

Also provided is a method for measuring total activity of a cell engineering tool in a cell (for example, activity at the target genomic locus, as well as, at an off-target location(s)). The method may include contacting a live cell with a cell engineering tool comprising a DNA binding domain and a nuclease domain, a gene repressor, or a gene activator, wherein the live cell comprises genomic DNA comprising a target genomic locus for the DNA binding domain of the cell engineering tool; fixing the cell and assaying for presence of a measurable change in nuclear protein load of a protein indicative of cellular response to the contacting, wherein the measurement reflects the total activity of the cell engineering tool. In certain aspects, the method may further include contacting the fixed cell with a plurality of nucleic acid probes complementary to the target genomic locus; and assaying for colocalization of the probes and the protein indicative of cellular response, wherein detection of the colocalization indicates activity of the cell engineering tool at the target genomic locus and absence of the colocalization indicates activity of the cell engineering tool at an off-target site.

Assaying for the change in nuclear protein load comprises imaging the cell by a microscopy mode selected from the group consisting of epifluorescence, widefield, confocal, selective plane illumination, tomography, holography, super-resolution, and synthetic aperture optics (SAO) and comparing to nuclear protein load in a reference cell not contacted with the cell engineering tool.

In certain aspects, when the measured change in protein load above an application-specific baseline level is detected, the method further comprises adjusting a parameter of the genome editing tool to improve specificity.

Details of the type of genome engineering tools that can be assessed, types of cells, probes, and imaging are provided herein.

### BRIEF DESCRIPTION OF THE DRAWINGS

FIG. 1 shows a brief summary of the assay workflow including the steps of nuclease transfection in cells, immunolabeling imaging, processing raw images by deconvolution, optional enhancement, deconvolution or reconstruction and segmentation, feature computation (e.g., count, amount, size, location of signal from immunolabel), and informatics and analysis (e.g., determining nuclease load and/or specificity, cytotoxicity, and/or heterogeneity).

FIG. 2 shows further details on image analysis including the steps of obtaining a microscopy image, deconvolution, delineation/segmentation of nuclei, p53BP1 foci, and nuclease protein, morphological data estimation, and informatics/analysis as described in FIG. 1.

FIGS. 3A and 3B illustrate dose response assessments of GA7 TALENs (XXX) in primary CD34+ hematopoietic stem cells.

FIG. 3A shows the number of p53BP1 foci per cell for CD34+ primary cells treated with a blank transfection control, 0.5 μg GA7 per TALEN monomer, 1 μg GA7 per TALEN monomer, 2 μg GA7 per TALEN monomer, and 4 μg GA7 per TALEN monomer.

FIG. 3B shows the total p53BP1 content (fluorescence intensity) per nucleus normalized by the nuclear size versus total FLAG tag content per nucleus normalized by the nuclear size indicative of a nuclease for CD34+ primary cells treated with a blank transfection control, 0.5 μg GA7 per TALEN monomer, 1 μg GA7 per TALEN monomer, 2 μg GA7 per TALEN monomer, and 4 μg GA7 per TALEN monomer.

FIGS. 4A and 4B illustrate dose response assessments of GA6 TALENs in immortalized K562 cells.

FIG. 4A shows the number of p53BP1 foci per cell for immortalized K562 cells treated with a blank transfection control, 0.5 μg GA6 per TALEN monomer, 1 μg GA6 per TALEN monomer, 2 μg GA6 per TALEN monomer, and 4 μg GA6 per TALEN monomer.

FIG. 4B shows the total p53BP1 content (fluorescence intensity) per nucleus normalized by the nuclear size versus total FLAG tag content per nucleus normalized by the nuclear size indicative of a nuclease for immortalized K562 cells treated with a blank transfection control, 0.5 μg GA6 per TALEN monomer, 1 μg GA6 per TALEN monomer, 2 μg GA6 per TALEN monomer, and 4 μg GA6 per TALEN monomer.

FIGS. 5A and 5B illustrate dose response assessments of AAVS1 TALENs in immortalized K562 cells.

FIG. 5A shows the number of p53BP1 foci per cell for immortalized K562 cells treated with a blank transfection control, 0.5 μg AASV1 per TALEN monomer, 1 μg AASV1 per TALEN monomer, 2 μg AASV1 per TALEN monomer, and 4 μg AASV1 per TALEN monomer.

FIG. 5B shows the total p53BP1 content (fluorescence intensity) per nucleus normalized by the nuclear size versus total FLAG tag content per nucleus normalized by the nuclear size indicative of a nuclease for immortalized K562 cells treated with a blank transfection control, 0.5 μg AASV1 per TALEN monomer, 1 μg GA6, 2 μg AASV1 per TALEN monomer, and 4 μg AAS per TALEN monomer.

FIG. 6 shows a graph of the number of p53BP1 foci per K562 cells at 6 hours, 12 hours, 24 hours, 48 hours, and 72 hours post transfection of AASV1 as compared to a control at each time point.

FIGS. 7A-7E show the results of control transfection and AASV1-targeting TALEN transfection in various cell types.

FIG. 7A shows the number of p53BP1 foci in adherent immortalized A549 cells transfected with a control and with an AASV1-targeting TALEN 24 hours post-transfection.

FIG. 7B shows the number of p53BP1 foci in suspension immortalized K562 cells transfected with a control and with an AASV1-targeting TALEN 24 hours post-transfection.

FIG. 7C shows the number of p53BP1 foci in primary CD34+ progenitor cells transfected with a control and with an AASV1-targeting TALEN 24 hours post-transfection.

FIG. 7D shows the number of p53BP1 foci in primary CD4+ T cells transfected with a control and with an AASV1-targeting TALEN 24 hours post-transfection.

FIG. 7E shows representative images of cells treated with AAVS1 TALENs versus untreated controls. Cells were stained for p53BP1 with an antibody and are visualized in green. TALENs were stained with a FLAG tag and are visualized in red. Nuclei were stained with DAPI and are visualized in grey. The scale bar indicates a size of 5 μm.

FIGS. 8A-8B illustrate assessment of nuclease specificity in K562 cells for TALENs and Cas9 nucleases targeting the AAVS1 genomic locus.

FIG. 8A illustrates the number of p53BP1 foci per cell for K562 cells transfected with Cas9 protein along with AAVS1 guide RNAs as compared to a blank transfection control.

FIG. 8B illustrates the number of p53BP1 foci per cell for K562 cells transfected with AAVS1-targeting TALENs as compared to a blank transfection control.

FIGS. 9A-9B show the DNA damage response, as measured by p53BP1 foci quantification, in CD34+ cells and T cells with TALENs targeting various genomic loci.

FIG. 9A shows the number of p53BP1 foci per cell in primary CD34+ progenitor cells after transfection with GA6-targeting TALENs, AAVS1-targeting TALENs, GA7-targeting TALENs, GA6-EK-targeting TALENs, and GA7-targeting TALENs. Controls include blank transfection controls.

FIG. 9B shows the number of p53BP1 foci per cell in primary stimulated CD4+ T cells after transfection with TP150-targeting TALENs, AAVS1-targeting TALENs, and TP171-targeting TALENs. Controls include non-electroporated naïve T cells, non-electroporated stimulated T cells, and untreated blank transfection control stimulated T cells.

FIG. 10 shows the number of p53BP1 foci per cell in K562 cells transfected with GA6_L14, GA6_L17, and GA6_L19.

FIG. 11 shows the number of p53BP1 foci per cell in K562 cells transfected with GA6_L, GA6_R, GA6_LR versus untreated control cells.

FIG. 12 shows the number of p53BP1foci per cell in K562 cells transfected with GA6 or GA6_EK TALENs.

FIG. 13 shows fluorescence microscopy images of control cells and AAVS1-targeting TALEN treated cells. A DAPI stain (gray) was used to visualize nuclei, p53BP1 is shown in green and the AAVS1 oligonucleotide Nano-FISH probe was visualized in red. Imaging showed that in cells transfected with AAVS1-targeting TALEN, spots indicative of double stranded breaks (indicated by p53BP1 foci) co-localized with AAVS1 oligonucleotide Nano-FISH probe spots.

FIGS. 14A-14C show histograms of the proportion of pairwise distances between AAVS1 Nano-FISH spots and p53BP1 foci.

FIG. 14A shows histograms of control and AAVS1 TALEN treated cells at pairwise distances of 0.1 to 0.5.

FIG. 14B shows histograms of control and AAVS1 TALEN treated cells at pairwise distances of 0 to 0.025.

FIG. 14C shows histograms of control and AAVS1 TALEN treated cells at pairwise distances of 0-0.08.

FIGS. 15A-15C show evaluation of nuclease specificity by counting p53BP1 foci in cells transfected with AAVS1-targeting TALENs.

FIG. 15A illustrates the number of p53BP1 foci on the x axis versus the proportion of cells with p53BP1 foci on the y-axis in cells transfected with AAVS1-targeting TALENs and, in 3D, imaged on a Nikon widefield fluorescence microscope with a 60× magnification lens using oil immersion contact techniques. “Ref” samples indicate control cells that were not transfected with TALENs Biological replicates are shown for control and transfected cells (indicated by set x). The number of cells analyzed in each sample is indicated by “n.”

FIG. 15B illustrates the number of p53BP1 foci on the x axis versus the proportion of cells with p53BP1 foci on the y-axis in cells transfected with AAVS1-targeting TALENs and imaged, in 3D, on a Nikon widefield fluorescence microscope with a 40× magnification lens using non-contact techniques. ‘Ref’ samples indicate control cells that were not transfected with TALENs Biological replicates are shown for control and transfected cells. The number of cells analyzed in each sample is indicated by “n.”

FIG. 15C illustrates the number of p53BP1 foci on the x axis versus the proportion of cells with p53BP1 foci on the y-axis in cells transfected with AAVS1-targeting TALENs and imaged on a Stellar-Vision (SV) fluorescence microscope using non-contact techniques. “Ref” samples indicate control cells that were not transfected with TALENs. Biological replicates are shown for control and transfected cells. The number of cells analyzed in each sample is indicated by “n.”

FIG. 16 shows a graph of the number of p53BP1 foci per CD4+ T cell at 24 hours and 48 hours post-transfection with AASV1-targeting TALENs as compared to blank transfection controls at each time point.

FIG. 17 shows an assay workflow for microscopy on a Stellar-Vision microscope. Images are captured on the Stellar-Vision microscope, images were reconstructed, images were segmented for regions of interest such as cell nucleic, p53BP1 foci, and nuclease localization, features were computed (such as count, size, diameter, area, volume, perimeter length, circularity, irregularity, eccentricity, etc.). The measured per-cell feature information was statistically analyzed to produce quantitative specificity metrics for the tested nuclease(s).

FIG. 18 depicts a method for estimating nuclease specificity based on p53BP1 foci characteristics.

FIG. 19 depicts a method for estimating nuclease specificity based on p53BP1 foci counts.

FIG. 20 shows a comparison of off-target activity estimated using Guide-Seq vs. p53BP1 imaging assay.

FIG. 21 illustrates use of the number of p53BP1 foci as a read out for improved nuclease specificity.

FIG. 22 illustrates use of the number of p53BP1 foci as a read out for improved nuclease specificity.

FIG. 23A illustrates the use of immunoNanoFISH and p53BP1 staining for per-allele per-cell on/off-target activity estimation in K562 cells.

FIG. 23B illustrates the use of immunoNanoFISH and p53BP1 staining for per-allele per-cell on/off-target activity estimation in CD34+ cells.

FIG. 24A illustrates the use of p53BP1 imaging for identifying nucleases suitable for targeting TCR-alpha locus.

FIG. 24B illustrates the use of p53BP1 imaging for identifying nucleases suitable for targeting PDCD-1.

FIG. 25 illustrates the use of p53BP1 imaging for dose titration of a lead TALEN.

FIG. 26 illustrates the use of p53BP1 imaging for screening nucleases for specificity and potency.

FIG. 27 shows that double strand break (DSB) repair protein serve as markers for evaluating nuclease specificity.

### DETAILED DESCRIPTION

The present disclosure provides compositions and methods for image-based analysis of cells eliciting a cellular response comprising accumulation of a moiety, such as a domain or a protein, in response to a cellular perturbation. The methods disclosed herein can allow for quantification of a protein load in a cell, wherein the protein can accumulate in response to a cellular response to a cellular perturbation. In some embodiments, the cellular response can be accumulation of a protein at the site of a double strand break. Alternatively, the cellular response can be active or passive accumulation of a protein, which participates in activating or repressing translational machinery. In some embodiments, the cellular perturbation comprises administration of a cell engineering tool. Examples of cell engineering tools include genome editing complex or gene regulator (an epigenetic repressor or activator). The genome editing complex or gene regulator can be designed to edit or regulate a target genomic locus. Modification of the target genomic locus can have therapeutic value. For example, modification of the target genomic locus can include introduction of a gene encoding a functional protein, knocking out a gene encoding a protein, or repressing expression of a protein for, e.g., treatment of indications that would benefit from the modification of the target genomic locus, such as, an indication that results from aberrant protein expression.

In some embodiments, the methods and compositions disclosed herein include an image-based assay for quantitation of foci within the nucleus of the cell. For example, the image-based assay can allow for visualization of fluorescent foci within the cell nucleus. The fluorescent foci may indicate accumulation of a protein. The protein can be labeled with any detectable agent disclosed herein. Upon accumulation within the nucleus, said detectable agent-labeled protein can be visualized as agglomerations or spots, also referred to as “foci.” The present disclosure also describes foci representing other detectable agents. For example, disclosed herein are foci of fluorescently labeled cell engineering tools (e.g., genome editing complex or gene regulator such as an epigenetic repressor or activator). Cell engineering tools (e.g., genome editing complex or gene regulator such as an epigenetic repressor or activator) can be labeled with a second fluorophore, different from the fluorophore conjugated to the protein. This can allow for simultaneous imaging and image analysis of the cell engineering tool (e.g., genome editing complex or gene regulator such as an epigenetic repressor or activator) and a protein, which accumulates during a cellular response. Also disclosed herein are foci of a fluorescently labeled genomic locus, wherein the genomic locus is visualized by labeled oligonucleotide Nano-FISH probe sets, which have a third fluorophore different from the first and second fluorophore. The genomic locus can be a target or off-target genomic locus. To visualize target and off-target genomic loci of interest, two separate Nano-FISH probe sets can be used, each with a different detectable agent.

The methods and compositions disclosed herein include an image-based assay for quantifying a protein that accumulates during a cellular response to a cellular perturbation caused by a cell engineering tool (e.g., genome editing complex or gene regulator such as an epigenetic repressor or activator), thereby serving as a marker of specificity and/or activity of the cell engineering tool. Specifically, the image-based methods can quantify a protein load, wherein the protein load is number of protein foci or total protein content per nucleus. The image-based methods described herein can also quantify a cell engineering tool load, wherein the cell engineering tool load can be a number of cell engineering tool foci or total cell engineering tool content per nucleus.

In some embodiments, a cellular perturbation comprising accumulation of a protein can be induced by a genome editing complex, which includes a DNA binding domain, a nuclease, and an optional linker. Genome editing complexes can also be referred to simply as “nucleases.” Specific genome editing complexes, whose cellular activity can be monitored, can include TALENs, megaTAL, a meganuclease, CAS nuclease (e.g., CRISPR/Cas9 systems), and zinc finger nucleases (ZFNs).

In other embodiments, the cellular perturbation can be induced by a gene regulator, such as a gene repressor, which can include a DNA binding domain, a repressor domain, and, optionally, a linker. In certain embodiments, the image based analysis of this disclosure allows for quantification of spots in a cell or a subcellular compartment, such as the nucleus, which are indicative of protein accumulation in response to a cellular perturbation.

In some embodiments, the image-based assay allows for quantification of spots representing protein accumulation within the nucleus on a per allele per cell basis. For example, when cells are edited with a genome editing complex (e.g., a TALEN, CRISPR/Cas9, ZFN, megaTALs, or meganucleases) to introduce a functional gene or to knock out a gene, nucleases (e.g., FokI or Cas9) induce a double strand break at the site of modification. Upon induction of the double strand break, a protein, such as a DNA repair protein, e.g., phosphorylated (ser1778) 53BP1 (p53BP1) or γH2AX can accumulate at the site of the double strand break and is indicative of a DNA damage response. In some embodiments, p53BP1 serves as a surrogate marker of a double strand break.

The present disclosure provides methods for staining cells for p53BP1 with a detectable agent. The detectable agent can comprise a primary antibody and a secondary antibody conjugated to a fluorophore. In other embodiments, the detectable agent can comprise a direct primary antibody conjugated to a fluorophore. Thus, p53BP1 foci, including one or more p53BP1 protein moieties accumulating at the site of a double strand break, can be resolved and visualized in the nucleus of the cell. The number of p53BP1 foci can indicate the number of double strand breaks induced in a cell and image analysis can, thus, serve to quantitatively resolve the DNA damage process spatially and temporally in each cell induced by a gene editing complex (e.g., a TALEN, CRISPR/Cas9, megaTALs, or meganucleases). Staining and visualizing p53BP1 foci within the nucleus of a cell, using the staining and image analysis techniques disclosed herein, can serve as a powerful tool to probe the specificity of a genome editing complex (e.g., a TALEN, CRISPR/Cas9, Lf N, megaTALs, or meganucleases) on a per allele per cell basis.

The compositions and methods of the present disclosure can be a powerful tool for assessing the specificity and activity of cell engineering tools (e.g., genome editing complex or gene regulator such as an epigenetic repressor or activator). These methods can be used to screen at least 5, at least 10, at least 50, at least 100, at least 150, at least 200, at least 250, at least 300, at least 350, at least 400, at least 500, or at least 1000 cell engineering tools (e.g., genome editing complex or gene regulator such as an epigenetic repressor or activator). These methods can be used to screen at 5-10, 10-50, 50-100, 150-200, 200-250, 250-300, 300-350, 350-400, 400-450, 450-500, or 500-1000 (e.g., genome editing complex or gene regulator such as an epigenetic repressor or activator) for lead candidates that exhibit potency (e.g., high gene editing efficiency or heightened or dampened gene expression) and specificity (low off-target (not at the genomic locus) cellular responses). The methods of the present disclosure can also be used to produce a potent and specific cell engineering tool, by iteratively tuning a parameter of a cell engineering tool and testing for improved specificity.

The compositions and methods of the present disclosure can be used to evaluate cell engineering tools for activity and/or specificity in primary cells. In some embodiments, immortalized cells can also be used with the compositions and methods of the present disclosure. In further embodiments, the primary cells and immortalized cell lines can be intact. Thus, the image-based methods described herein allow probing of an allele in intact cells, such as, a fixed cell without requiring isolation of genomic DNA for sequencing.

### Determining Specificity of Genome Editing Complexes

In some embodiments, the present disclosure provides compositions and methods for probing the specificity of a genome editing complex (e.g., a TALEN, CRISPR/Cas9, megaTALs, or meganucleases) by imaging and analyzing p53BP1 foci. Genome editing complexes are a type of a cell engineering tool and can be referred to herein as a “nuclease.” In other words, imaging and analyzing p53BP1 foci after administration of a genome editing complex (e.g., a TALEN, CRISPR/Cas9, ZFN, megaTALs, or meganucleases) can be used to quantify off-target DNA damage induced by the nuclease. Described below are several genome editing complexes (e.g., a TALEN, CRISPR/Cas9, and/or ZFN), which can be used to introduce a functional gene or knock out a gene, via nuclease-induced double strand breaks. Genome editing complexes can be administered to a cell by electroporation, lipofection, viral transduction, or another suitable delivery method. Further described below are the types of outcomes or readouts that can be analyzed using image-based analysis of p53BP1 or γH2AX foci. In particular the methods can be used to quantify a protein (p53BP1) load, which can comprise the number of p53BP1foci and/or total p53BP1 content within the nucleus.

A. TALENs

A nuclease may comprise a Transcription Activator-Like Effector (TALE) sequence. A TALE may comprise a DNA-binding module which includes a variable number of repeat units or repeat modules having about 33-35 amino acid residues. Each acid repeat unit recognizes one nucleotide through two adjacent amino acids (such as at amino acids at positions 12 and 13 of the repeat). In general, the amino acid sequences of each repeat unit does not vary significantly outside of positions 12 and 13. The amino acids at positions 12 and 13 of a repeat may also be referred to as repeat-variable diresidue (RVD).

A TALE probe described herein may comprise between about 1 to about 50 TALE repeat modules. A TALE probe described herein may comprise between about 5 and about 45, between about 8 and about 45, between about 10 and about 40, between about 12 and about 35, between about 15 and about 30, between about 20 and about 30, between about 8 and about 40, between about 8 and about 35, between about 8 and about 30, between about 10 and about 35, between about 10 and about 30, between about 10 and about 25, between about 10 and about 20, or between about 15 and about 25 TAL effector repeat modules.

A TALE probe described herein may comprise about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 45, or about 50 TALE repeat modules. A TALE probe described herein may comprise about 5 TALE repeat modules. A TALE probe described herein may comprise about 10 TALE repeat modules. A TALE probe described herein may comprise about 11 TALE repeat modules. A TALE probe described herein may comprise about 12 TALE repeat modules. A TALE probe described herein may comprise about 13 TALE repeat modules. A TALE probe described herein may comprise about 14 TALE repeat modules. A TALE probe described herein may comprise about 15 TALE repeat modules. A TALE probe described herein may comprise about 16 TALE repeat modules. A TALE probe described herein may comprise about 17 TALE repeat modules. A TALE probe described herein may comprise about 18 TALE repeat modules. A TALE probe described herein may comprise about 19 TALE repeat modules. A TALE probe described herein may comprise about 20 TALE repeat modules. A TALE probe described herein may comprise about 21 TALE repeat modules. A TALE probe described herein may comprise about 22 TALE repeat modules. A TALE probe described herein may comprise about 23 TALE repeat modules. A TALE probe described herein may comprise about 24 TALE repeat modules. A TALE probe described herein may comprise about 25 TALE repeat modules. A TALE probe described herein may comprise about 26 TALE repeat modules. A TALE probe described herein may comprise about 27 TALE repeat modules. A TALE probe described herein may comprise about 28 TALE repeat modules. A TALE probe described herein may comprise about 29 TALE repeat modules. A TALE probe described herein may comprise about 30 TALE repeat modules. A TALE probe described herein may comprise about 35 TALE repeat modules. A TALE probe described herein may comprise about 40 TALE repeat modules. A TALE probe described herein may comprise about 45 TALE repeat modules. A TALE probe described herein may comprise about 50 TALE repeat modules.

A TAL effector repeat module may be a wild-type TALE DNA-binding module or a modified TALE DNA-binding repeat module enhanced for specific recognition of a nucleotide. A TALE probe described herein may comprise one or more wild-type TALE DNA-binding module. A TALE probe described herein may comprise one or more modified TAL effector DNA-binding repeat module enhanced for specific recognition of a nucleotide. A modified TALE DNA-binding repeat module may comprise 1, 2, 3, 4, 5, 6, 7, 8, 9, 10, 15, 20, 25 or more mutations that may enhance the repeat module for specific recognition of a nucleic acid sequence (e.g., a target sequence). In some cases, a modified TALE DNA-binding repeat module is modified at amino acid position 2, 3, 4, 11, 12, 13, 21, 23, 24, 25, 26, 27, 28, 30, 31, 32, 33, 34, or 35. In some cases, a modified TALE DNA-binding repeat module is modified at amino acid positions 12 or 13.

A TALE repeat module may be a repeat module-like domain or RVD-like domain. A RVD-like domain has a sequence different from naturally occurring polynucleotidic repeat module comprising RVD (RVD domain) but have a similar function and/or global structure. Non-limiting examples of RVD-like domains include protein domains selected from Puf RNA binding protein or Ankyrin super-family.

A TALE repeat module may comprise a RVD of TABLE 1. A TALE probe described herein may comprise one or more RVDs selected from TABLE 1. Sometimes, a TALE probe described herein may comprise up to 1, up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, up to 30, up to 31, up to 32, up to 33, up to 34, up to 35, up to 36, up to 37, up to 38, up to 39, up to 40, up to 45, up to 50, up to 60, up to 70, up to 80, up to 90, or up to 100 RVDs selected from TABLE 1.

TABLE 1

| RVD | Nucleotide |
|---|---|
| HD | C |
| NG | T |
| NI | A |
| NN | G > A |
| NS | G, A > C > T |
| NH | G |
| N* | T > C >> G, A |
| NP | T > A, C |
| HG | T |
| H* | T |
| IG | T |
| HA | C |
| ND | C |
| NK | G |
| HI | C |
| HN | G > A |
| NT | G > A |
| NA | G |
| SN | G or A |
| SH | G |
| YG | T |
| IS | — |

A RVD may recognize or interact with one type of nucleotide (e.g., the RVD HD binds only to C). A RVD may recognize or interact with more than one type of nucleotide (e.g., the RVD binds to G and A). The efficiency of a RVD domain at recognizing a nucleotide is ranked as “strong”, “intermediate” or “weak”. The ranking may be according to a ranking described in Streubel et al., “TAL effector RVD specificities and efficiencies,” Nature Biotechnology 30(7): 593-595 (2012). The ranking of RVD may be as illustrated in TABLE 2, based on the ranking provided in Streubel et al. Nature Biotechnology 30(7): 593-595 (2012).

TABLE 2

| RVD | Nucleotide | Efficiency |
|---|---|---|
| HD | C | strong |
| NG | T | weak |
| NI | A | weak |
| NN | G > A | Strong (G), intermediate (A) |
| NS | G, A > C > T | intermediate |
| NH | G | intermediate |
| N* | T > C >> G, A | weak |
| NP | T > A, C | intermediate |
| NK | G | weak |
| HN | G > A | intermediate |
| NT | G > A | intermediate |
| SN | G or A | Weak |
| SH | G | Weak |
| IS | — | weak |

*Denotes a gap in the repeat sequence corresponding to a lack of an amino acid residue at the second position of the RVD.

A TALE DNA-binding domain may further comprise a C-terminal truncated TALE DNA-binding repeat module, such as, a shortened, e.g., a half-repeat unit. A C-terminal truncated TALE DNA-binding repeat module may be between about 15 and about 34 residues in length. A C-terminal truncated TALE DNA-binding repeat module may be between about 15 and about 32, between about 18 and about 34, between about 18 and about 32, between about 24 and about 35, between about 28 and about 32, between about 25 and about 34, between about 25 and about 32, between about 25 and about 30, between about 28 and about 32, or between about 28 and about 30 residues in length. A C-terminal truncated TALE DNA-binding repeat module may be at least 18, at least 19, at least 20, at least 21, at least 22, at least 23, at least 24, at least 25, at least 26, at least 27, at least 28, at least 29, at least 30, at least 31, at least 32, at least 33, up to 34 residues in length. A C-terminal truncated TALE DNA-binding repeat module may be up to 15 residues, up to 18 residues, up to 19 residues, up to 20 residues, up to 21 residues, up to 22 residues, up to 23 residues, up to 24 residues, up to 25 residues, up to 26 residues, up to 27 residues, up to 28 residues, up to 29 residues, up to 30 residues, up to 31 residues, up to 32 residues, up to 33 residues, or up to 34 residues in length. A C-terminal truncated TALE DNA-binding repeat module may include a RVD of TABLE 1.

A TALE DNA-binding domain may further comprise an N-terminal cap. An N-terminal cap may be a polypeptide sequence flanking the DNA-binding repeat module. An N-terminal cap may be any length and may comprise from about 0 to about 136 amino acid residues in length. An N-terminal cap may be about 5, about 10, about 15, about 20, about 25, about 30, about 35, about 40, about 45, about 50, about 60, about 70, about 80, about 90, about 100, about 110, about 120, or about 130 amino acid residues in length. An N-terminal cap may modulate structural stability of the DNA-binding repeat modules. An N-terminal cap may modulate nonspecific interactions. An N-terminal cap may decrease nonspecific interaction. An N-terminal cap may reduce off-target effect. As used here, off-target effect refers to the binding of a DNA binding protein (e.g., a TALE protein) to a sequence that is not the target sequence of interest. An N-terminal cap may further comprise a wild-type N-terminal cap sequence of a TALE protein or may comprise a modified N-terminal cap sequence a TALE protein, such as a TALE protein from Xanthomonas.

A TALE DNA-binding domain may further comprise a C-terminal cap sequence. A C-terminal cap sequence may be a polypeptide portion flanking the C-terminal truncated TALE DNA-binding repeat module. A C-terminal cap may be any length and may comprise from about 0 to about 278 amino acid residues in length. A C-terminal cap may be about 5, about 10, about 15, about 20, about 25, about 30, about 35, about 40, about 45, about 50, about 60, about 80, about 100, about 150, about 200, or about 250 amino acid residues in length. A C-terminal cap may further comprise a wild-type C-terminal cap sequence of a TALE protein or may comprise a modified C-terminal cap sequence a TALE protein, such as a TALE protein from Xanthomonas.

A nuclease domain may be linked to a TALE DNA-binding domain either directly or through a linker. A linker may be between about 1 and about 50 amino acid residues in length. A linker may be from about 5 to about 45, from about 5 to about 40, from about 5 to about 35, from about 5 to about 30, from about 5 to about 25, from about 5 to about 20, from about 5 to about 15, from about 10 to about 40, from about 10 to about 35, from about 10 to about 30, from about 10 to about 25, from about 10 to about 20, from about 12 to about 40, from about 12 to about 35, from about 12 to about 30, from about 12 to about 25, from about 12 to about 20, from about 14 to about 40, from about 14 to about 35, from about 14 to about 30, from about 14 to about 25, from about 14 to about 20, from about 14 to about 16, from about 15 to about 40, from about 15 to about 35, from about 15 to about 30, from about 15 to about 25, from about 15 to about 20, from about 15 to about 18, from about 18 to about 40, from about 18 to about 35, from about 18 to about 30, from about 18 to about 25, from about 18 to about 24, from about 20 to about 40, from about 20 to about 35, from about 20 to about 30, or from about 25 to about 30 amino acid residues in length.

A nuclease domain fused to a TALE can be an endonuclease or an exonuclease. An endonuclease can include restriction endonucleases and homing endonucleases. An endonuclease can also include S1 Nuclease, mung bean nuclease, pancreatic DNase I, micrococcal nuclease, or yeast HO endonuclease. An exonuclease can include a 3′-5′ exonuclease or a 5′-3′ exonuclease. An exonuclease can also include a DNA exonuclease or an RNA exonuclease. Examples of exonuclease includes exonucleases I, II, III, IV, V, and VIII; DNA polymerase I, RNA exonuclease 2, and the like. A nuclease domain fused to a TALE can be a restriction endonuclease (or restriction enzyme). In some instances, a restriction enzyme cleaves DNA at a site removed from the recognition site and has a separate binding and cleavage domains. In some instances, such restriction enzyme is a Type IIS restriction enzyme.

A nuclease domain fused to a TALE can be a Type IIS nuclease. A Type IIS nuclease can be FokI or Bfil. In some cases, a nuclease domain fused to a TALE is FokI. In other cases, a nuclease domain fused to a TALE is Bfil.

FokI can be a wild-type FokI or can comprise one or more mutations. In some cases, FokI can comprise 1, 2, 3, 4, 5, 6, 7, 8, 9, 10, or more mutations. A mutation can enhance cleavage efficiency. A mutation can abolish cleavage activity. In some cases, a mutation can modulate homodimerization. For example, FokI can have a mutation at one or more amino acid residue positions 446, 447, 479, 483, 484, 486, 487, 490, 491, 496, 498, 499, 500, 531, 534, 537, and 538 to modulate homodimerization.

In some instances, a FokI cleavage domain is, for example, as described in Kim et al. “Hybrid restriction enzymes: Zinc finger fusions to Fok I cleavage domain,” PNAS 93: 1156-1160 (1996), which is incorporated herein by reference in its entirety. In some cases, a FokI cleavage domain described herein is a FokI of (QLVKSELEEKKSELRHKLKYVPHEYIELIEIARNSTQDRILEMKVMEFFMKVYGYRG KHLGGSRKPDGAIYTVGSPIDYGVIVDTKAYSGGYNLPIGQADEMQRYVEENQTRN KHINPNEWWKVYPSSVTEFKFLFVSGHFKGNYKAQLTRLNHITNCNGAVLSVEELLI GGEMIKAGTLTLEEVRRKFNNGEINF, SEQ ID NO: 1062). In other instances, a FokI cleavage domain described herein is a FokI, for example, as described in U.S. Pat. No. 8,586,526, which is incorporated herein by reference in its entirety.

A TALE probe can be designed to recognize each strand of a double-stranded segment of DNA by engineering the TALE to include a sequence of repeat-variable diresidue subunits that may comprise about 22, about 23, about 24, about 25, about 26, about 27, about 28, about 29, about 30, about 31, about 32, about 33, about 34, about 35, about 36, about 37, about 38, about 39, or about 40 amino acid repeats capable of associating with specific DNA sequences, such that the detectable label of the TALE probe is located at the target nucleic acid sequence.

Also described herein are megaTALs, in which a TALE DNA binding domain is fused to a monomeric meganuclease, also referred to as a “homing endonuclease” capable of binding and cleaving a target genomic locus of interest. Image-based analysis methods and compositions described herein can be used to evaluate the specificity and/or activity of a megaTAL.

Image-based analysis methods and compositions described herein can be used to evaluate the specificity and/or activity of a meganuclease. Meganucleases can include intron endonucleases and intein endonucleases. Meganucleases can be a LAGLIDADG endonuclease and can include I-CreI or I-SceI.

B. CRISPR/Cas9

Similar to TALENs and ZFNs, clustered regularly interspaced palindromic repeats-associated-Cas9 (CRISPR-Cas9) systems can also be engineered to target and edit a specific nucleic acid sequence. A CRISPR-dCas9 can comprise multiple components in a ribonucleoprotein complex, which can include the Cas9 protein that can interact with a single-guide RNA (sgRNA), an optional linker, and a repressor domain. The sgRNA can be made of a CRISPR RNA (crRNA) and a trans-activating crRNA (tracrRNA). The CRISPR-Cas9s described herein can be used to modulate transcription of a target gene to which the sgRNA binds. For example, the CRISPR-Cas9s of the present disclosure can be used to repress expression of a target gene.

The sgRNA can comprise at least 18, at least 19, at least 20, at least 21, at least 22, at least 23, at least 24, or at least 25 nucleotides that are complementary to a target sequences of interest. Thus, this portion of the sgRNA is analogous to the DNA binding domain described herein with respect to TALENs and ZFNs. The portion of the sgRNA (e.g., the about 20 nucleotides within the sgRNA that bind to a target) bind adjacent to a protospacer adjacent motif (PAM), which can comprise 2-6 nucleotides in the target sequence that is bound by Cas9.

C. ZFNs

Similar to TALEN, zinc-finger nuclease (ZFN) is a restriction enzyme that can be engineered to target and edit specific nucleic acid sequences. A Lf N can comprise a zinc-finger DNA binding domain linked either directly or indirectly to a nuclease domain.

A zinc-finger DNA binding domain of a ZFN can comprise from about 1 to about 10 zinc finger motifs. A zinc-finger DNA binding domain can comprise from about 1 to about 9, from about 2 to about 8, from about 2 to about 6 or from about 2 to about 4 zinc finger motifs. In some cases, a zinc-finger DNA binding domain can comprise at least 1, 2, 3, 4, 5, 6, 7, 8, 9, 10, or more zinc finger motifs. A zinc-finger DNA binding domain can comprise at least 1, 2, 3, 4, 5, 6, 7, 8, 9, or 10 zinc finger motifs. A zinc-finger DNA binding domain can comprise about 1 zinc finger motif. A zinc-finger DNA binding domain can comprise about 2 zinc finger motif. A zinc-finger DNA binding domain can comprise about 3 zinc finger motif. A zinc-finger DNA binding domain can comprise about 4 zinc finger motif. A zinc-finger DNA binding domain can comprise about 5 zinc finger motif. A zinc-finger DNA binding domain can comprise about 6 zinc finger motif. A zinc-finger DNA binding domain can comprise about 7 zinc finger motif. A zinc-finger DNA binding domain can comprise about 8 zinc finger motif. A zinc-finger DNA binding domain can comprise about 9 zinc finger motif. A zinc-finger DNA binding domain can comprise about 10 zinc finger moti.

A zinc finger motif can be a wild-type zinc finger motif or a modified zinc finger motif enhanced for specific recognition of a set of nucleotides. A ZFN described herein can comprise one or more wild-type zinc finger motif. A ZFN described herein can comprise one or more modified zinc finger motif enhanced for specific recognition of a set of nucleotides. A modified zinc finger motif can comprise 1, 2, 3, 4, 5, 6, 7, 8, 9, 10, 15, 20, 25, or more mutations that can enhance the motif for specific recognition of a set of nucleotides. In some cases, one or more amino acid residues within the α-helix of a zinc finger motif are modified. In some cases, one or more amino acid residues at positions −1, +1, +2, +3, +4, +5, and/or +6 relative to the N-terminus of the α-helix of a zinc finger motif can be modified.

A nuclease domain linked to a zinc-finger DNA-binding domain can be an endonuclease or an exonuclease. An endonuclease can include restriction endonucleases and homing endonucleases. An endonuclease can also include S1 Nuclease, mung bean nuclease, pancreatic DNase I, micrococcal nuclease, or yeast HO endonuclease. An exonuclease can include a 3′-5′ exonuclease or a 5′-3′ exonuclease. An exonuclease can also include a DNA exonuclease or an RNA exonuclease. Examples of exonuclease includes exonucleases I, II, III IV, V and VIII; DNA polymerase I, RNA exonuclease 2, and the like.

A nuclease domain fused to a zinc-finger DNA-binding domain can be a restriction endonuclease (or restriction enzyme). In some instances, a restriction enzyme cleaves DNA at a site removed from the recognition site and has a separate binding and cleavage domains. In some instances, such restriction enzyme is a Type ITS restriction enzyme.

A nuclease domain fused to a zinc-finger DNA-binding domain can be a Type IIS nuclease. A Type ITS nuclease can be FokI or Bfil. In some cases, a nuclease domain fused to a zinc-finger DNA-binding domain is FokI. In other cases, a nuclease domain fused to a zinc-finger DNA-binding domain is Bfil.

A nuclease domain can be linked to a zinc-finger DNA-binding domain either directly or through a linker. A linker can be between about 1 to about 50 amino acid residues in length. A linker can be from about 5 to about 45, from about 5 to about 40, from about 5 to about 35, from about 5 to about 30, from about 5 to about 25, from about 5 to about 20, from about 5 to about 15, from about 10 to about 40, from about 10 to about 35, from about 10 to about 30, from about 10 to about 25, from about 10 to about 20, from about 12 to about 40, from about 12 to about 35, from about 12 to about 30, from about 12 to about 25, from about 12 to about 20, from about 14 to about 40, from about 14 to about 35, from about 14 to about 30, from about 14 to about 25, from about 14 to about 20, from about 14 to about 16, from about 15 to about 40, from about 15 to about 35, from about 15 to about 30, from about 15 to about 25, from about 15 to about 20, from about 15 to about 18, from about 18 to about 40, from about 18 to about 35, from about 18 to about 30, from about 18 to about 25, from about 18 to about 24, from about 20 to about 40, from about 20 to about 35, from about 20 to about 30, or from about 25 to about 30 amino acid residues in length.

A linker for linking a nuclease domain to a zinc-finger DNA-binding domain can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acid residues in length.

D. Genome Editing Complex Readouts

In some embodiments, the present disclosure provides an image-based assay for quantification of protein (e.g., p53BP1 or γH2AX) load on a per cell basis after administration of any of the gene editing complexes disclosed herein (e.g., a TALEN, CRISPR/Cas9, ZFN, megaTALs, or meganucleases). Protein load can be determined, for example, by quantification of number of p53BP1 foci or total p53BP1 content per nucleus. Types of analyses that can be performed include identification of DNA damage response proteins as surrogates for nuclease activity, development of a reliable quantitative imaging assay to visualize the protein (e.g., p53BP1 or γH2AX), quantification of nuclease activity in each cell at its target genomic locus and elsewhere (for example, by measurement of indels), quantification of cell transfection efficiency and levels of nuclease expression, quantification of cytotoxicity resulting from nuclease activity, screening of nucleases in a high-throughput (96-well) format, and screening of gene editing complexes with high precision using as low as 50 cells to as high as 1000 cells or more. Image-based analysis of p53BP1 for evaluating nuclease specificity can be performed across all nucleases (e.g., a TALEN, CRISPR/Cas9, ZFN megaTALs, or meganucleases) and across all cell types including immortalized cells and primary cells.

In some embodiments, the genome editing complex can be tagged, for example with a FLAG tag. When further staining for p53BP1 foci, the image analysis methods of the present disclosure allows for co-quantification of genome editing complex amount by staining for the FLAG tag (e.g., antibody-based methods) and p53BP1 load (e.g., number of p53BP1 foci, total p53BP1 amount per nucleus), which serves as a measure of genome editing complex specificity. Additionally, genome editing complex-induced cytotoxicity can be measured by quantifying the fraction of apoptotic nuclei in transfected cells.

Genome editing complex specificity can be measured by evaluating dose response in cells using the image-based assay of the present disclosure and analyzing for p53BP1 load. In certain embodiments, genome editing complex with high specificity can induce a similar level of double strand breaks, as visualized by a similar p53BP1 load, regardless of the genome editing complex dose. In some embodiments, genome editing complex specificity can be measured over time, for example up to 3 hrs post-transfection, up to 6 hours post-transfection, up to 12 hours post transfection, up to 24 hours post-transfection, up to 48 hours post transfection, up to 60 hrs post-transfection, 0 to 6 hours post-transfection, 3 to 60 hours post transfection, 6 to 12 hours post transfection, 24 to 48 hours post transfection, 6 to 24 hours 48 hours to 5 days after transfection. 5 to 10 days after transfection, 10-15 days post transfection 15 to 20 days post transfection, 20 to 25 days post transfection, 25 to 30 days post transfection, or 6 hours to 30 days post transfection.

In some embodiments, imaging p53BP1 foci for quantification of double strand breaks can be used to determine which component of a genome editing complex drives specificity versus off target activity. For example, TALENs can be comprised of a left DNA binding domain coupled to FokI targeting a top DNA strand and a right DNA binding domain coupled to FokI targeting a bottom DNA strand. These can be referred to as a left TALEN monomer and a right TALEN monomer. Quantification of p53BP1 foci after administration of just one TALEN monomer can reveal which monomer leads to off-target enzymatic activity.

In some embodiments, genome editing complexes can be iteratively improved upon by changing a parameter of the genome editing complex, testing for specificity by image analysis of p53BP1 load after administration in cells, and, optionally, further tuning the parameter of the genome editing complex and re-testing specificity. For example, as described herein, a TALEN can include a DNA binding domain comprising a number of repeat units. As length of the DNA binding domain is increased, specificity for the target genomic locus can be increased. TALENs can be iteratively designed to increase the number of repeats within the DNA binding domain, administering said TALEN to a cell, evaluating specificity by imaging for p53BP1 foci and quantifying p53BP1 load, and if needed further increasing the number of repeats within the DNA binding domain.

In some embodiments, visualization of DNA double strand breaks, induced by a genome editing complex, via staining for p53BP1 can be further combined with imaging of the target genomic locus of interest using oligonucleotide Nano-FISH probe sets and methods described further below. For example, cells can be transfected with a genome editing complex targeting a genomic locus of interest. The nuclease enzyme (e.g., FokI) of the genome editing complex can be tagged (e.g., via a FLAG tag) and cells can be denatured and labeled with oligonucleotide Nano-FISH probes for the same genomic locus of interest. DNA double strand breaks can be further imaged via staining for p53BP1 foci. Co-localization of signal from p53BP1 foci with signal from oligonucleotide Nano-FISH probe foci indicates nuclease activity at the target genomic locus of interest, thus indicating specificity. Signal from p53BP1 foci that are spatially separated from signal from oligonucleotide Nano-FISH probe foci can indicate off-target nuclease activity that may not be at the genomic locus of interest.

Image based analysis of the specificity of genome editing complexes via visualization of p53BP1 can be done at high throughput. High throughput analysis can involve analysis of greater than 1000, greater than 10,000, or greater than 100,000 cells in less than 24 hours or less than 48 hours. In some embodiments, high throughput analysis can involve analysis of more than 1 unique sample, more than 5 unique samples, more than 10 unique samples, or more than 100 unique samples within 24 hours. In other embodiments, cell populations less than 1000, less than 500, less than 100, or 50 or less can be analyzed.

In some embodiments, image-based analysis of p53BP1 content in a cell after administration of a gene editing complex can be combined with measurements of gene editing efficiency (e.g., measuring indels at the target site). Thus, the present disclosure allows assessment of genome editing complexes for potency and specificity, wherein potency is determined by measuring gene editing efficiency and specificity is measured via quantification of p53BP1 foci either alone or in combination with oligonucleotide Nano-FISH for the genomic locus of interest.

### Gene Regulators

In some embodiments, the present disclosure provides compositions and methods for probing the specificity of a gene regulator (e.g., a TALE-TF, CRISPR/dCas9, and/or ZFP-TF) by imaging and analyzing for protein accumulation at a target genomic locus. Described below are several gene regulators (e.g., a TALE-TF, CRISPR/dCas9, and/or ZFP-TF), which can be used to activate expression of a target gene or repress expression of a target gene. In some cases, additional proteins are recruited to the target genomic locus and can serve as a marker for gene activation (e.g., H3K4me1, H3K4me2, H3K27ac) or gene repression (e.g., KAP1, H3K9me3, H3K27me3 or HP1). Further described below are the types of outcomes or readouts that can be analyzed using image-based analysis of gene repression.

A. Transcription Activator-Like Effector-Transcription Factor (TALE-TF)

The present disclosure provides for a gene regulator or an engineered transcription factor, wherein the engineered transcription factor can be a transcription activator-like effector-transcription factor (TALE-TF). A TALE-IF can include multiple components including the transcription activator-like effector (TALE) protein, an optional linker, and a repressor domain. The TALE-TFs described herein can be used to modulate transcription of a target gene to which the TALE protein binds. For example, the TALE-TFs of the present disclosure can be used to repress expression of a target gene.

In some embodiments, the TAL effector can be any TAL effector described above. A TALE-IF of the present disclosure can further include a transcription repressor domain. The repressor domain can be a Krüppel-associated box (KRAB) protein, which induces transcriptional repression of polymerases (RNA pol I, II, and/or II) by binding to other corepressors. Alternatively, the repressor domain can be any one of KOX, TGF-beta-inducible early gene (TIEG), v-erbA, SID, MBD2, MBD3, DNMT1, DNMT3A-L, or DNMT3B, Rb, and MeCP2.

In some embodiments, a TALE-TF of the present disclosure can further include a transcription activation domain. The activation domain can comprises VP16, VP64, p65, p300 catalytic domain, TET1 catalytic domain, TDG, Ldb1 self-associated domain, SAM activator (VP64, p65, HSF1), or VPR (VP64, p65, Rta)

In some embodiments, any one of the TALEs described herein can bind to a region of interest of any gene. For example, the TALEs described herein can bind upstream of the promoter region, upstream of the gene transcription start site, or downstream of the transcription start site. In certain embodiments, the TALE protein binding region is no farther than 50 base pairs downstream of the transcription start site. In some embodiments, the TALE protein is designed to bind in proximity to the transcription start site (TSS). In other embodiments, the TALE can be designed to bind in the 5′ UTR region.

B. Zinc Finger Protein—Transcription Factor (ZFP-TF)

The present disclosure provides for a engineered transcription factor, wherein the engineered transcription factor can be a zinc-finger protein-transcription factor (ZFP-TF). A ZFP-TF can include multiple components including the zinc finger protein (ZFP), an optional linker, and a repressor domain. The ZFP-TFs described herein can be used to modulate transcription of a target gene to which the ZFP binds. For example, the ZFP-TFs of the present disclosure can be used to repress expression of a target gene. The repressor domain can be a Krüppel-associated box (KRAB) protein, which induces transcriptional repression of polymerases (RNA pol I, II, and/or III) by binding to other corepressors. Alternatively, the repressor domain can be any one of Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

In some embodiments, a ZFP-TF of the present disclosure can further include a transcription activation domain. The activation domain can comprises VP16, VP64, p65, p300 catalytic domain, TET1 catalytic domain, TDG, Ldb1 self-associated domain, SAM activator (VP64, p65, HSF1), or VPR (VP64, p65, Rta)

The ZFP can also be referred to as a zinc finger DNA binding domain. The zinc-finger DNA binding domain can comprise a set of zinc finger motifs. Each zinc finger motif can be about 30 amino acids in length and can folk into a pa structure in which the α-helix can be inserted into the major groove of the DNA double helix and can engage in sequence-specific interaction with the DNA site. In some cases, the sequence-specific recognition can span over 3 base pairs. In some cases, a single zinc finger motif can interact specifically with 1, 2 or 3 nucleotides.

C. CRISPR-dCas9—Transcription Factor (CRISPR-dCas9-TF)

The present disclosure provides for a engineered transcription factor, wherein the engineered transcription factor can be a clustered regularly interspaced palindromic repeats-associated-deactivated Cas9 (CRISPR-dCas9). A CRISPR-dCas9 can comprise multiple components in a ribonucleoprotein complex, which can include the dCas9 protein that can interact with a single-guide RNA (sgRNA), an optional linker, and a repressor domain. The sgRNA can be made of a CRISPR RNA (crRNA) and a trans-activating crRNA (tracrRNA). The CRISPR-dCas9s described herein can be used to modulate transcription of a target gene to which the sgRNA binds. For example, the CRISPR-dCas9s of the present disclosure can be used to repress expression of a target gene.

The sgRNA can comprise at least 18, at least 19, at least 20, at least 21, at least 22, at least 23, at least 24, or at least 25 nucleotides that are complementary to a target sequences of interest. Thus, this portion of the sgRNA is analogous to the DNA binding domain described above with respect to ZFPs and TALEs. The portion of the sgRNA (e.g., the about 20 nucleotides within the sgRNA that bind to a target) bind adjacent to a protospacer adjacent motif (PAM), which can comprise 2-6 nucleotides in the target sequence that is bound by dCas9.

The dCas9 can be generated from a wild-type Cas9 protein by mutating 2 residues. The CRISPR-dCas9 ribonucleoprotein complex can repress a target gene by steric hindrance. The CRISPR-dCas9 ribonucleoprotein complex can be further coupled to any repressor domain described herein (e.g., KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2) to provide repression of a target gene.

In some embodiments, a CRISPR-dCas9 ribonucleoprotein complex can be further coupled to a transcription activation domain. The activation domain can comprises VP16, VP64, p65, p300 catalytic domain, TET1 catalytic domain, TDG, Ldb1 self-associated domain, SAM activator (VP64, p65, HSF1), or VPR (VP64, p65, Rta)

D. Epigenetic Regulation Readouts

In some embodiments, the present disclosure provides for imaging protein accumulation after administration of a gene regulator (e.g., TALE-TF, CRISPR-dCas9, or ZFP-TF). Types of analyses that can be performed include identification of protein for repression of translation machinery, development of a reliable quantitative imaging assay to visualize the chosen surrogate protein, quantification of gene repression activity in each cell at its target genomic locus and elsewhere, quantification of cell transfection efficiency and levels of gene regulator expression, and screening of gene regulators in a high-throughput (96-we) format. For example, a TALE-TF comprising a DNA binding domain, a KRAB repressor domain and, optionally, a linker can be transfected into a cell of interest. The cell can be an immortalized cell or a primary cell. Upon binding to the target genomic locus, the KRAB repressor domain is capable of recruiting other co-repressors (e.g., KAP1). Staining can be performed against recruited co-repressors (e.g., KAP1) for evaluating repressor activity. The staining can include a primary and secondary antibody-fluorophore conjugate or a primary antibody-fluorophore conjugate.

In another example, the TALE-TF can comprise a DNMT3a repressor domain. In another example, the TALE-TF can comprise any repressor domain or activation domain described herein. Staining can then be performed for proteins accumulating at the site gene activation (e.g., H3K4me1, H3K4me2, H3K27ac) or gene repression (e.g., KAP1, H3K9me3, H3K27me3 or HP1) to evaluate specificity of the gene regulator. These image-based analyses of proteins indicative of gene regulator activity can be performed across a gene regulators (e.g., TALE-TF, CRISPR/dCas9, ZFP-TFs) and across a cell types, including immortalized cells and primary cells.

In some embodiments, the activation or repression domain can be tagged with a detectable agent, such as a fluorescent moiety. When further staining for proteins that accumulate in response to gene activation (e.g., H3K4me1, H3K4me2, H3K27ac) or gene repression (e.g., KAP1, H3K9me3, H3K27me3 or HP1), the image analysis methods of the present disclosure allows for co-quantification of gene regulator amount and a protein (e.g., H3K4me1, H3K4me2, H3K27ac proteins for activation or KAP1, H3K9me3, H3K27me3 or HP1 proteins for repression) load, which serves as a measure of gene regulator activity. As described above, protein load can include number of protein foci or total protein content per nucleus.

Additionally, cytotoxicity induced by administration of gene regulators (e.g., TALE-TF, CRISPR-dCas9, or ZFP-TF) can be measured by quantifying the fraction of apoptotic nuclei in transfected cells. Gene regulator specificity can be measured by evaluating dose response in cells using the image-based assay of the present disclosure and analyzing for foci comprising markers of gene activation (e.g., H3K4me1, H3K4me2, H3K27ac) or gene repression (e.g., KAP1, H3K9me3, H3K27me3 or HP1). In some embodiments, gene regulator specificity can be measured over time, for example 6 hours post-transfection, 12 hours post transfection, 24 hours post-transfection, 48 hours post transfection, 0-6 hours post-transfection. 6-12 hours post transfection, 24-48 hours post transfection, 48 hours to 5 days after transfection. 5-10 days after transfection, 10-15 days post transfection. 15-20 days post transfection, 20-25 days post transfection, 25-30 days post transfection, or 6 hours-30 days post transfection.

In some embodiments, visualization of gene regulator activity, via staining for a protein that accumulates in response to gene activation (e.g., H3K4me, H3K4me2, H3K27ac) or gene repression (e.g., KAP1, H3K9me3, H3K27me3 or HP1), can be further combined with imaging of the target genomic locus of interest using oligonucleotide Nano-FISH probe sets and methods described further below. For example, cells can be transfected with a gene regulator (e.g., TALE-TF, ZFP-TF, CRISPR/dCas9) targeting a genomic locus of interest Cells can be denatured and labeled with oligonucleotide Nano-FISH probes for the same genomic locus of interest. Recruited protein that accumulates in response to gene activation (e.g., H3K4me1, H3K4me2, H3K27ac) or gene repression (e.g., KAP1, H3K9me3, H3K27me3 or HP1) can be further imaged via staining Co-localization of protein foci (e.g., H3K4me, H3K4me2, H3K27ac for activators or KAP1, H3K9me3, H3K27me3 or HP1 for repressors) with signal from oligonucleotide Nano-FISH probes indicates activity of the gene regulator at the target genomic locus of interest Signal from protein foci that are spatially separated from signal from oligonucleotide Nano-FISH probes indicates off-target gene regulator activity that may not be at the genomic locus of interest.

### Translocation

In some embodiments, the present disclosure involves imaging of a translocation event, such as chromosome translocation. For example, chromosome translocation can involve the generation of double strand breaks in two non-homologous regions of DNA, which can result in joining of the two non-homologous regions (translocation).

A genome editing complex (e.g., TALEN, ZFN, CRISPR/Cas9, megaTAL, meganuclease) can be administered to an immortalized or primary cell. Cells can be stained for p53BP1 with a first detectable agent, subsequently or concurrently contacted with a oligonucleotide Nano-FISH probe set with a second detectable agent to hybridize to a target genomic locus, and contacted with a different oligonucleotide Nano-FISH probe set with a third detectable agent to hybridize to an off-target genomic locus. Samples are imaged and analyzed using the techniques disclosed herein. Foci of p53BP1 can be visualized by signal from the first detectable agent, indicating a double strand break and gene editing with the genome editing complex. Foci of the oligonucleotide Nano-FISH probe set hybridized to a target genomic locus can be visualized by signal from the second detectable agent, indicating the target genomic locus. Foci of the oligonucleotide Nano-FISH probe set hybridized to an off-target genomic locus can be visualized by signal from the third detectable agent, indicating the off-target genomic locus.

In the absence of a translocation event, co-localization of the signal from the first detectable agent and the second detectable agent can be visualized observed, indicating co-localization of p53BP1 with the oligonucleotide Nano-FISH probe set for the target genomic locus. When chromosomal translocation occurs, co-localization of the signal from the first detectable agent, the second detectable agent, and the third detectable agent can be observed, indicating co-localization of p53BP1 with the oligonucleotide Nano-FISH probe set for the target genomic locus and the oligonucleotide Nano-FISH probe set for the off-target genomic locus.

The term “hybridization” or “hybridizes” refers to a process in which a region of nucleic acid strand anneals to and forms a stable duplex, either a homoduplex or a heteroduplex, under normal hybridization conditions with a complementary nucleic acid strand and does not form a stable duplex with unrelated (non-complementary) nucleic acid molecules under the same normal hybridization conditions. The formation of a duplex is accomplished by annealing two complementary nucleic acids under hybridization conditions. The hybridization condition can be made to be highly specific by adjustment of the conditions under which the hybridization reaction takes place, such that two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

### Genes and Indications of Interest

In some embodiments, the image-based analysis of protein (e.g., p53BP1) of cellular perturbation (e.g., genome editing with a TALEN, CRISPR/Cas9, or ZFN) and/or Nano-FISH image analysis can be used to identify a lead genome editing complex for the purposes of genetic modification of a cell. In some embodiments, genome editing can be performed by fusing a nuclease of the present disclosure with a DNA binding domain for a particular genomic locus of interest. Genetic modification can involve introducing a functional gene for therapeutic purposes, knocking out a gene for therapeutic gene, or engineering a cell ex vivo (e.g., HSCs or CAR T cells) to be administered back into a subject in need thereof. For example, the genome editing complex can have a target site within a gene such as PDCD1, CTLA4, LAG3, TET2, BTLA, HAVCR2, CCR5, CXCR4, TRA, TRB, B2M, albumin, HBB, HBA1, TTR, NR3C1, CD52, erythroid specific enhancer of the BCL11A gene, CBLB, TGFBR1, SERPINA1, HBV genomic DNA in infected cells, CEP290, DMD, CFTR, IL2RG, CS-1, or any combination thereof. A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control region.

In some embodiments, a genome editing complex can cleave double stranded DNA at a target site in order to insert a chimeric antigen receptor (CAR), alpha-L iduronidase (IDUA), iduronate-2-sulfatase (IDS), or Factor 9 (F9). Cells, such as hematopoietic stem cells (HSCs) and T cells, can be engineered ex vivo with the genome editing complex. Alternatively, genome editing complexes can be directly administered to a subject in need thereof. Image-based analysis of protein (e.g., p53BP1) of said genome editing complexes can enable the development of highly specific genome editing complexes with less than 10 off-target double strand breaks, less than 5 off-target double strand breaks, less than 4 off-target double strand breaks, less than 3 off-target double strand breaks, less than 2 off-target double strand breaks, less than 1 off-target double strand breaks, or no off-target double strand breaks.

The subject receiving treatment can be suffering from a disease such as transthyretin amyloidosis (ATTR), HIV, glioblastoma multiforme, cancer, acute lymphoblastic leukemia, acute myeloid leukemia, beta-thalassemia, sickle cell disease, MPSI, MPSII, Hemophilia B, multiple myeloma, melanoma, sarcoma, Leber congenital amaurosis (LCA10), CD19 malignancies, BCMA-related malignancies, duchenne muscular dystrophy (DMD), cystic fibrosis, alpha-1 antitrypsin deficiency, X-linked severe combined immunodeficiency (X-SCID), or Hepatitis B.

A Nano-FISH probe set, as described below, can be designed for any genomic locus of interest described herein (e.g., PDCD1, CTLA4, LAG3, TET2, BTLA, HAVCR2, CCR5, CXCR4, TRA, TRB, B2M, albumin, HBB, HBA, TTR, NR3C1, CD52, erythroid specific enhancer of the BCL11A gene, CBLB, TGFBR1, SERPINA1, HBV genomic DNA in infected cells, CEP290, DMD, CFTR, IL2RG, CS-1, or any combination thereof) to be used in combination with image-based analysis of protein (e.g., p53BP1) of cellular perturbation.

### Nano-FISH and Viral Nano-FISH Techniques

Any of the above compositions and methods for image-based analysis of a surrogate marker (e.g., a protein such as p53BP1) for a cellular response induced by a cellular perturbation can be further combined with Nano-FISH. Oligonucleotide Nano-FISH probe sets can be used to visualize a target genomic locus of interest. Thus, the specificity of a genome editing complex (e.g., a TALEN, CRISPR/Cas9, ZFN), a gene regulator (e.g., a TALE-TF, ZFP-TF, CRISPR/dCas9), or a translocation event can be visualized by combination imaging with Nano-FISH. Compositions and methods for Nano-FISH are described in further detail below.

Described herein are methods of detecting a cellular regulatory element in situ utilizing a super-resolution microscopy technique to determine the presence, absence, and/or activity of a regulatory element. Also described herein are methods of detecting different types of regulatory elements simultaneously utilizing a heterogeneous set of detection agents, and translating the molecular information from the different types of regulatory elements to determine the activity state of a cell. The activity state of a cell may correlate to a localization, expression level, and/or interaction state of a regulatory element. One or more of the methods described herein may further interpolate 2-dimensional images to generate 3-dimensional maps which enable detection of localization, interaction states, and activity of one or more regulatory elements. Intrinsic properties such as size, intensity, and location of a detection agent further may enable detection of a regulatory element Described herein are methods of determining the localization of a regulatory element and measuring the activity of a regulatory element. The methods provided herein may avoid the introduction of artifacts such as biological stressors and perturbations or destroys cellular architecture.

One or more methods described herein may detect different types of regulatory elements, distinguish between different types of regulatory elements, and/or generate a map of a regulatory element (e.g., chromatin). For example, a regulatory element may be labeled by one or more different types of detection agents. The one or more different types of detection agents may include DNA detection agents, RNA detection agents, protein detection agents, or combinations thereof. The detection agent may comprise a probe portion, which may interact (e.g., hybridize) to a target site within the regulatory element, and optionally comprise a detectable moiety. The detectable moiety may include a fluorophore, such as a fluorescent dye or a quantum dot. The detection agent may be an unlabeled probe which can be further conjugated to an additional labeled probe. Upon labeling, the regulatory element may be detected by stochastic or deterministic super-resolution microscopy method. The stochastic super-resolution microscopy method may be a synthetic aperture optics (SAO) method. The SAO method may generate a detection profile, which can encompass fluorescent signal intensity, size, shape, or localization of the detection agent. Based on the detection profile, the activity state, the localization, expression level, and/or interaction state of the regulatory element may be determined. A map based on the detection profile of the regulatory element may also be generated, and may be correlated to cell type identification (e.g., cancerous cell identification). The regulatory element may be further analyzed in the presence of an exogenous agent or condition, such as a small molecule fragment or a drug or under an environment such as a change in temperature, pH, nutrient, or a combination thereof. The perturbation of the activity state of the regulatory element in the presence of the exogenous agent or condition may be measured. A report may further be generated and provided to a user, such as a laboratory clinician or health care provider.

The systems and methods disclosed herein also relate to a novel nanoscale fluorescence in situ hybridization methodology (hereinafter referred to as “Nano-FISH”) to reliably label and detect localized small (less than 12 kb in size) DNA segments in cells. In some cases, Nano-FISH can utilize defined pools or sets of synthetic fluorescent dye-labeled oligonucleotides (probe pools or probe sets) to reliably detect small genomic regions in large numbers of adherent or suspension cells in situ. In some instances, Nano-FISH can be conducted utilizing conventional wide-field microscopic imaging. In other embodiments, Nano-FISH can be conducted using super-resolution imaging techniques.

In some cases, Nano-FISH can be coupled with an automated image informatics pipeline to enable high-throughput detection and 2D and/or 3D spatial localization of small genomic DNA elements in situ in hundreds of thousands of or more individual cells per experiment. In some instances, to facilitate rigorous statistical analyses of the resulting large image data sets, a scalable image analysis software suite can reliably identify and quantitatively annotate labeled loci on a single-cell basis.

In some cases, Nano-FISH can allow detection of the precise localization of specific regulatory genomic elements in 2D or 3D nuclear space, the identification of small-scale structural genomic variations (such as sequence gains or losses), the quantitation of spatial interactions between regulatory elements and their putative target gene(s), or the detection of genomic conformational changes that induce stimulus-dependent gene expression. In some instances, Nano-FISH can allow the visualization of the precise localization of a target nucleic acid sequence. The target nucleic acid sequence can be an endogenous nucleic acid sequence, a nucleic acid sequence derived from an exogenous source, or a combination thereof. An exogenous nucleic acid sequence can be introduced into a first cell and can be further detected in progeny of the first cen. An exogenous target nucleic acid sequence can be introduced to a cell through electroporation, lipofection, transfection, microinjection, viral transduction, or a gene gun. Non-limiting examples of vector systems that can be used to introduce a target nucleic acid sequence into a cell may include viral vector, episomal vector, naked RNA (recombinant or natural), naked DNA (recombinant or natural), bacterial artificial chromosome (BAC), and RNA/DNA hybrid systems used separately or in combination Vector systems can be used without additional reagents meant to aid in the incorporation and/or expression of desired mutations. A non-limiting list of reagents meant to aid in the incorporation and/or expression of desired mutations can include Lipofectamine, FuGENE, FuGENE HD, calcium phosphate, HeLaMONSTER, Xtreme Gene. An endogenous nucleic acid sequence can be a gene sequence or fragment thereof. An endogenous nucleic acid sequence can be a sequence in a chromosome. An endogenous nucleic acid sequence can be a nucleic acid sequence resulting from somatic chromosomal rearrangement, such as the nucleic acid sequence of a B cell receptor, T cell receptor, or fragment thereof. In some instances, Nano-FISH can allow the detection of the precise localization of exogenous nucleic acids inserted or integrated into a genome. In some embodiments, Nano-FISH can allow the detection of the precise localization of exogenous DNA inserted into a genome, as may be inserted by a genetic engineering technique or by viral infection or transduction. In some instances, Nano-FISH can allow the detection of an episomal nucleic acid sequence.

The systems and methods described herein can be useful in detecting or determining the presence, absence, identity, or quantity of a target nucleic acid sequence in a sample. In particular, the methods, compositions, and systems described herein can be used to efficiently detect, to identify, and to quantify a target nucleic acid sequence that is a short nucleic acid sequences. In some cases, a short nucleic acid sequence that can be detected or quantified using the disclosures of the present application may be from 15 nucleotides in length to about 12 kb in length. A short nucleic acid sequence can be less than 1 kb.

Methods for the detection, identification, and/or quantification of a short nucleic acid sequence of a sample can comprise contacting the short nucleic acid sequence with a probe comprising a detectable label and determining the presence, absence, or quantity of probes bound to the target nucleic acid sequence. Determination of the sequence position of the short nucleic acid sequence relative to other nucleotides or another short nucleic acid sequence (for instance, using a second probe capable of binding to a second target sequence of the nucleic acid) can be a step in the methods described herein. The methods described herein can also comprise determining the spatial position of the short nucleic acid sequence. For example, Nano-FISH can be used to measure the normalized inter-spot distance between a first short nucleic acid sequence encoding an enhancer or portion thereof and a second nucleic acid encoding a promoter of a gene or portion thereof which can be used to study changes in genome conformation that may be associated with gene function.

The methods described herein can comprise comparing the presence, absence, spatial position, sequence position, or quantity of a short nucleic acid sequence of a sample to a reference value. A non-limiting example of quantifying detection of a short nucleic acid sequence in a cell can comprise quantifying the number of copies of a nucleic acid sequence that has been incorporated into a modified cell (for example, a cell modified by the introduction of a nucleic acid sequence into the cell by genetic editing), which can be used as quality control for modified cells produced by cell engineering strategies.

The degree of precision and accuracy in nucleic acid sequence detection, identification, and quantification made possible by the methods, compositions, and systems of the present disclosure can enable the detection of viral nucleic acid sequences, which commonly range from about 1 kb in length to about 10 kb in length.

Also described herein are methods, compositions, and systems useful in characterizing and/or quantifying the presence, absence, position, or identity of a target nucleic acid sequence in a cell or sample derived therefrom relative to a reference nucleic acid sequence in the same cell or sample or relative to a control cell or sample. For example, improvements to the efficiency of detection and to a detection threshold, as described herein, can allow for the detection and characterization of short nucleic acid sequences (for instance, non-repeating nucleic acid sequence insertions) during analysis or validation of cell samples or cell lines.

Additionally, described herein, are methods, compositions, and systems for correlating protein expression with target nucleic acid sequence detection. For example, a target nucleic acid sequence can be associated with the expression of a target protein. Using Nano-FISH, the presence, absence, or quantity of the target nucleic acid sequence can be detected, and a detectable label may be used to detect a target protein expression, which therefore can allow for the correlation between the presence, absence, or quantity of the target nucleic acid sequence and the expression of the target protein.

The Nano-FISH methods as described herein can be used as a diagnostic for the detection, identification, and/or quantification of a short nucleic acid sequence of a sample. For example, Nano-FISH can be used as a diagnostic for HIV by detecting HIV nucleic acid sequences in a sample. The Nano-FISH methods as described herein can be used with therapeutics by detecting identifying and/or quantifying a short nucleic acid sequence of a sample. For example, Nano-FISH can be used with therapeutics in which a short nucleic acid sequence is integrated into a cell's DNA (e.g., chimeric antigen receptor T cell therapeutics) to determine detect, identify, and/or quantify the short nucleic acid sequence integration. This can be important for any type of viral-mediated (e.g., lentiviral-mediated) transgene integration because these integrations can be heterogeneous (i.e., some cells do not get infected, others are infected multiple times), and integrations occur randomly in the genome (i.e. inactive sequences, or active genes). In contrast to Nano-FISH, existing methods to measure transgene integration and expression suffer from limitations including lacking single-cell resolution (qPCR), providing data about protein products without DNA information (flow cell sorting), or being laborious (single-cell cloning).

Additionally, Nano-FISH is a significantly improved and distinct tool from conventional FISH for numerous reasons related to control over design of the probe set, which enable the detection of short nucleic acid sequences at high throughput and at a high signal-to-noise ratio.

In some embodiments, Nano-FISH probe sets of the present disclosure can be comprised of one or more short oligonucleotide Nano-FISH probes designed against a target, allowing for complete control over probe size. For example, using the Nano-FISH methods described herein, one or more oligonucleotide Nano-FISH probes of exact size can be designed against a transfer plasmid backbone. The oligonucleotide Nano-FISH probes of the present disclosure can be from 30 to 60 nucleotides in length. In certain embodiments, the oligonucleotide Nano-FISH probes of the present disclosure can be 40 nucleotides in length. In contrast, conventional FISH techniques require the use of fosmids (varying in size from 40-50 kilobases), BACs (varying in size from varying in size from 100-250 kilobases), or plasmids (varying in size from 5-10 kilobases), which are conventionally nick translated to incorporate hapten or fluorescently labeled-dUTP (or other nucleotide). The result of nick translating fosmids, BACs, and/or plasmids to obtain conventional FISH probes is the generation of a highly heterogeneous pool of probes of varying sizes. Conventional FISH probes average around 500 nucleotides in length but exhibit a size distribution from 100 bases to anywhere around 1.5 kilobases, which is up to 50 times larger than an oligonucleotide Nano-FISH probe. Alternatively, conventional probes can be generated by means of PCR with the incorporation of labeled nucleotides during the reaction. Thus, in contrast to the oligonucleotide Nano-FISH probes of this disclosure, there is poor control over the resulting probe size of nick translated conventional FISH probes made from fosmids, BACs, or plasmids.

In some embodiments, the Nano-FISH probes of the present disclosure are precisely controlled to introduce an exact number of fluorescent dye molecules per probe. For example, in some embodiments, each olignucleotide Nano-FISH probe of the present disclosure can have exactly a detectable agent at the 3′ end. The detectable agent can be any dye molecule, such as a Quasar Dye (e.g., Q570 and Q670). Oligonucleotide Nano-FISH probes of the present disclosure may be synthesized from the 3′ to 5′ end, and the fluorophore may be included on the first nucleotide at the 3′end. In some embodiments, an oligonucleotide Nano-FISH probe of the present disclosure can have 2 fluorescent dye molecules. For example, a Nano-FISH oligonucleotide probe of the present disclosure with a size of 55 to 60 nucleotides can have 2 fluorescence dye molecules. In this case, the second dye molecule may be placed on an internal nucleotide or at the 5′ end. Additionally, since the oligonucleotide Nano-FISH probes of the present disclosure directly incorporate a fluorophore at the 3′end of each probe, the present disclosure provides a probe set that can be directly labeled and, thus, offers direct labeling and detection of a target nucleotide sequence without any need for signal amplification.

In contrast, because conventional FISH probes can be nick translated to incorporate hapten-dUTPs or other labeled nucleotides for subsequent secondary detection by a fluorescent antibody/reagent, there is no control over the exact number of fluorescent dye molecules that are incorporated in a given probe. Thus, the resulting conventional FISH probes are a heterogeneous mixture with various degrees of fluorescent dye labels. Moreover, while some conventional FISH probes can directly incorporate a fluorescent dye, most conventional FISH probes contain Digoxigenin or biotin-labeled nucleotides, which are subsequently reacted to an antibody-fluorophore conjugate or a streptavidin-fluorophore conjugate. Thus, conventional FISH probes are indirectly labeled with a fluorophore. In contrast, the oligonucleotide Nano-FISH probes of the present disclosure are directly labeled with a fluorophore.

In some embodiments, the Nano-FISH probes of the present disclosure are designed to precisely target a desired strand of a target (e.g., the Watson strand, the Crick strand, or both strands). Moreover, the oligonucleotide Nano-FISH probes of the present disclosure can be designed to overlap by at least 5 base pairs. For example a first oligonucleotide Nano-FISH probe can be designed to target the Watson strand of a target sequence and a second oligonucleotide Nano-FISH probe can be designed to target an adjacent region on the Crick strand of a target sequence. The first and second probe can overlap by at least 5 nucleotides, can be directly adjacent to each other, or can be spaced apart by at least several nucleotides. In some embodiments, the first and second probe can overlap by 5-20 nucleotides. Overlapping probes on the plus and minus strands can allow for the design and hybridization of larger probe sets to target smaller nucleic acid sequences.

Finally, the oligonucleotide Nano-FISH probes of the present disclosure are designed and selected according to certain criteria in order to precisely target and detect an exogenous sequence (e.g., a viral nucleic acid sequence), while minimizing off-target binding that would increase the background noise during imaging. For example, a target can be selected and the hg38 coordinates can be determined. Next, a tiling density can be selected from all on one strand, a fixed 2 base pair spacing between adjacent oligonucleotide Nano-FISH probes, or a spacing of 30 base pairs on each DNA strands with a 5 base pair overlap between the top and bottom strands at each end. In some embodiments, oligonucleotide Nano-FISH probes of the present disclosure are tiled across a target to avoid steric hindrance between molecules. Next, oligonucleotide Nano-FISH probe sequences are tiled across regions of interest, such as the human genome or the human genome with an artificial extra chromosome representing the target (e.g., the CAR transfer plasmid). In some embodiments, a program can be used to tile oligonucleotide Nano-FISH probes across the region of interest. As an example, a 40 base pair probe pool can be generated by tiling 40 base pair oligonucleotide probes at a predetermined spacing between oligonucleotides across a target sequence. The tiled 40 base pair probe pool can be designed to provide a minimum spacing of 2 base pairs between each consecutive oligonucleotide Nano-FISH probe. Each oligonucleotide Nano-FISH probe in the resulting probe pool can be compared to a 16-mer database of genomic sequences to identify partial matches of probes to genomic sequences that can result in off-target background staining which would negatively affect the signal-to-noise ratio. An oligonucleotide Nano-FISH probe that comprises a total of 24 matches or less to the 16-mer database may be considered to be unique in the human genome and, thus, can be selected to move forward. A probe with more than 300 matches to the 16-mer database of genomic sequences can be discarded from consideration as it generates too many non-target hits. The number of matches of an oligonucleotide Nano-FISH probe can have to the 16-mer database of genomic sequences may depend on the size of the probe. For example, a 30 base pair long oligonucleotide Nano-FISH probe that exhibits a total of 14 matches or less to the 16-mer database may be considered to be unique in the human genome and, thus, may be selected to move forward. A 50 base pair long oligonucleotide Nano-FISH probe that exhibits a total of 34 matches or less to the 16-mer database may be considered to be unique in the human genome and, thus, may be selected to move forward. A 60 base pair long oligonucleotide Nano-FISH probe that exhibits a total of 44 matches or less to the 16-mer database may be considered to be unique in the human genome and, thus, may be selected to move forward. Thus, an oligonucleotide Nano-FISH probe of the present disclosure between 30 to 60 base pairs in length may exhibit 14 to 44 matches or less to the 16-mer database and be considered unique in the human genome. Oligonucleotide Nano-FISH probes of the present disclosure have less than 300 matches to the 16-mer database of genomic sequences. Pools of at least 30 oligonucleotide Nano-FISH probes that satisfied al design criteria can be selected to carry forward. Additional selection criteria that can be applied when selecting the oligonucleotide Nano-FISH probes of the present disclosure include percent GC content. For example, oligonucleotide Nano-FISH probes can have a percent GC content above at least 25%. In some embodiments, oligonucleotide Nano-FISH probes of the present disclosure are selected for use if they have less than 5 hits, less than 4 hits, less than 3 hits, less than 2 hits, or less than 1 hit of at least a 50% contiguous homology elsewhere in the human genome (e.g., by a BLAT search of each oligo against the genome). A BLAT search of each oligo against the genome may result in larger stretches of homology. A probe that exhibits less than 50% (˜20 bases) homology may be considered to be unique and, thus, may be selected to move forward. When designing a probe set for enhanced resolution, the probe set can be designed to have a limited number of oligonucleotide Nano-FISH probes, such as 25-35 probes, that can be closely spaced. When designing a probe set for enhanced detection, the probe set can be designed include from 100-150 probes.

Additionally, oligonucleotide Nano-FISH probes of the present disclosure may be selected to not include a repetitive element. For example, a repetitive element may be short interspersed nuclear elements (SINE) including ALUs, long interspersed nuclear elements (LINE), long terminal repeat elements (LTR) including retroposons, DNA repeat elements, simple repeats (micro-satellites), low complexity repeats, satellite repeats, RNA repeats such as RNA, tRNA, rRNA, snRNA, scRNA, or srpRNA, or other repeats such as the class rolling circle (RC). Any one or more of the above design criteria may be used to select the oligonucleotide Nano-FISH probes that make up a probe set of the present disclosure. As described above, the process of comparing each oligonucleotide Nano-FISH probe against a 16-mer database of human genomic sequences may result in the selecting for probes that do not comprise repetitive elements.

In contrast to the designed and selected oligonucleotide Nano-FISH probes of the present disclosure, conventional FISH probes that are nick translated are not filtered for low homology to human genomic sequences. As a result, conventional FISH techniques incorporate a step of blocking the FISH probes with a blocking agent such as Cot-1 DNA, salmon sperm DNA, yeast tRNA, or any combination thereof which bind to any regions of the conventional FISH probes that are highly repetitive. The blocked conventional FISH probes are then incubated with cells. In contrast, the present oligonucleotide Nano-FISH probes can be directly incubated with cells for hybridization with a target sequence, without the need for a blocking agent. 10181 In some embodiments, a probe set is referred to herein as a “probe poor” or a “plurality of probes.” For example, an oligonucleotide Nano-FISH probe set can comprise from 20-200 oligonucleotide probes. In some embodiments, the probe set can comprise 20-200 oligonucleotide Nano-FISH probes.

Overall, the above described properties of the Nano-FISH probes of the present disclosure, can lead to increased precision in detecting a target sequence, especially detection of small target sequences that are less than 5 kilobases, and lower background signals stemming from off target probe-DNA interactions, as compared to conventional FISH probes. In other words, the Nano-FISH probes of the present disclosure can yield a better or higher signal-to-noise ratio than conventional FISH probes.

In some embodiments, 9 oligonucleotide-Nano-FISH probes of the present disclosure may be used visualize insertions of an exogenous nucleic acid sequence in the nucleus at a signal to noise ratio of about 1.2-1.5 to 1. In some embodiments, 15 oligonucleotide-Nano-FISH probes of the present disclosure may be used visualize insertions of an exogenous nucleic acid sequence in the nucleus at a signal to noise ratio of about 1.5:1. In some embodiments, 30 oligonucleotide-Nano-FISH probes of the present disclosure may be used visualize insertions of an exogenous nucleic acid sequence in the nucleus at a signal to noise ratio of about 4-8 to 1. In some embodiments, 60 oligonucleotide-Nano-FISH probes of the present disclosure may be used visualize insertions of an exogenous nucleic acid sequence in the nucleus at a signal to noise ratio of about 5-10:1. In some embodiments, 90 oligonucleotide Nano-FISH probes of the present disclosure may result in at least one detected allele (in a triploid cell background) in about 98% of cells. In some embodiments, 60 oligonucleotide Nano-FISH probes of the present disclosure may result in at least one detected allele (in a triploid cell background) in about 92% of cells. In some embodiments, 30 oligonucleotide Nano-FISH probes of the present disclosure may result in at least one detected allele (in a triploid cell background) in about 89% of cells. In some embodiments, 15 oligonucleotide Nano-FISH probes of the present disclosure may result in at least one detected allele (in a triploid cell background) in about 34% of cells.

In some embodiments, the target exogenous nucleic acid sequence does not need to be amplified prior to detection. Thus, the exogenous nucleic acid sequences of the present disclosure are non-amplified exogenous nucleic acid sequences. In some embodiments, the signal from the oligonucleotide Nano-FISH probes of the present disclosure does not need to be amplified prior to detection. Thus, the Nano-FISH methods of the present disclosure provide methods of non-signal amplified detection. In other words, the Nano-FISH methods of the present disclosure provide methods of direct, non-amplified signal detection.

The compositions and methods provided herein can also comprise a plurality of probe sets, wherein each probe set can contain any number of oligonucleotide Nano-FISH probes described above. Within a probe set, oligonucleotide Nano-FISH probes may an labeled with the same fluorophore. Each probe set in the plurality of probe sets may be labeled with different fluorophores. Each probe set in the plurality of probe sets may further comprise oligonucleotide Nano-FISH probes for the detection of unique target sequences (e.g., exogenous or viral nucleic acid sequences). Thus, a plurality of probe sets can be used to detect multiple target sequences simultaneously, with each target sequence being labeled with a unique fluorophore.

A. Types of Regulatory Elements

A regulatory element may be DNA, RNA, a polypeptide, or a combination thereof. A regulatory element may be DNA. A regulatory element may be RNA. A regulatory element may be a polypeptide. A regulatory element may be any combination of DNA, RNA, and/or polypeptide (e.g., protein-protein complexes, protein-DNA/RNA complexes, and the like).

A regulatory element may be DNA. A regulatory element may be a single-stranded DNA regulatory element, a double-stranded DNA regulatory element, or a combination thereof. The DNA regulatory element may be single-stranded. The DNA regulatory element may be double-stranded. The DNA regulatory element may encompass a DNA fragment. The DNA regulatory element may encompass a gene. The DNA regulatory element may encompass a chromosome. The DNA regulatory element may include endogenous DNA regulatory elements (e.g., endogenous genes). The DNA regulatory element may include artificial DNA regulatory elements (e.g., foreign genes introduced into a cell).

A regulatory element may be RNA. A regulatory element may be a single-stranded RNA regulatory element, a double-stranded RNA regulatory element, or a combination thereof. The RNA regulatory element may be single-stranded. The RNA regulatory element may be double-stranded. The RNA regulatory element may include endogenous RNA regulatory elements. The RNA regulatory element may include artificial RNA regulatory elements. The RNA regulatory element may include microRNA (miRNA), transfer RNA (tRNA), ribosomal RNA (rRNA), messenger RNA (mRNA), pre-mRNA, transfer-messenger RNA (tmRNA), heterogeneous nuclear RNA (hnRNA), short interfering RNA (siRNA), or short hairpin RNA (shRNA). The RNA regulatory element may be a RNA fragment. The RNA regulatory element may be an anti-sense RNA.

An RNA regulatory element may be an enhancer RNA (eRNA). An enhancer RNA may be a non-coding RNA molecule transcribed from an enhancer region of a DNA molecule, and may be from about 50 base-pairs (bp) in length to about 3 kilo base pairs in length. An enhancer RNA may be a 1D eRNA or an eRNA that may be unidirectionally transcribed. An enhancer RNA may also be a 2D eRNA or an eRNA that may be bidirectionally transcribed. An eRNA may be polyadenylated. Alternatively, an eRNA may be non-polyadenylated.

A regulatory element may be a DNaseI hypersensitive site (DHS). DHS may be a region of chromatin unoccupied by transcription factors and which is sensitive to cleavage by the DNase I enzyme. The presence of DHS regions within a chromatin may demarcate transcription factory occupancy at a nucleotide resolution. The presence of DHS regions may further correlate with activation of cis-regulatory elements, such as an enhancer, promoter, silencer, insulator, or locus control region DHS variation may be correlated to variation in gene expression in healthy or diseased cells (e.g., cancerous cells) and/or correlated to phenotypic traits.

A DHS pattern may encode memory of prior cell fate decisions and exposures. For example, upon differentiation, a DHS pattern of a progeny may encode transcription factor occupancy of its parent. Further, a DHS pattern of a cell may encode an environmentally-induced transcription factor occupancy from an earlier time point.

A DHS pattern may encode cellular maturity. An embryonic stem cell may encode a set of DHSs that may be transmitted combinatorially to a differentiated progeny, and this set of DHSs may be decreased with each cycle of differentiation. As such, the set of DHSs may be correlated with time, thereby allowing a DHS pattern to be correlated with cellular maturity.

A DHS pattern may also encode splicing patterns. Protein coding exons may be occupied by transcription factors, which may further be correlated with codon usage patterns and amino acid choice on evolutionary time scales and human fitness. A transcription factory occupancy may further modulate alternative splicing patterns, for example, by imposing sequence constraints at a splice junction. As such, a DHS pattern may encode transcription factor occupancy of one or more exons of interest and may provide additional information on alternative splicing patterns.

A DHS pattern may encode a cell type. For example, within each cell type, about 100,000 to about 250,000 DHSs may be detected. About 5% of the detected DHSs may be located within a transcription start site and the remaining DHSs may be detected at a distal site from the transcription start site. Each cell type may contain a distinct DHS pattern at the distal site and mapping the DHS pattern at the distal site may allow identification of a cell type. An overlap may further be present within two DHS patterns from two different cell types, for example, an overlap of a set of detected DHSs within the two DHS patterns. An overlap may be less than about 70 of the detected DHSs. The presence of an overlap may not affect the identification of a cell type.

A regulatory element may be a polypeptide. The polypeptide may be a protein or a polypeptide fragment. For example, a regulatory element may be a transcription factor, DNA-binding protein or fictional fragment, RNA-binding protein or functional fragment, protein involved in chemical modification (e.g., involved in histone modification), or gene product. A regulatory element may be a transcription factor. A regulatory element may be a DNA or RNA-binding protein or fictional fragment. A regulatory element may be a product of a gene transcript. A regulatory element may be a chromatin.

B. Methods of Detecting a Regulatory Element

Described herein is a method of detecting a regulatory element. The detection may encompass identification of the regulatory element, determining the presence or absence of the regulatory element, and/or determining the activity of the regulatory element A method of detecting a regulatory element may include contacting a cell sample with a detection agent, binding the detection agent to the regulatory element, and analyzing a detection profile from the detection agent to determine the presence, absence, or activity of the regulatory element.

The method may involve utilizing one or more intrinsic properties associated with a detection agent to aid in detection of the regulatory element. The intrinsic properties may encompass the size of the detection agent, the intensity of the signal, and the location of the detection agent. The size of the detection agent may include the length of the probe and/or the size of the detectable moiety (e.g., the size of a fluorescent dye molecule) may modulate the specificity of interaction with a regulatory element. The intensity of the signal from the detection agent may correlate to the sensitivity of detection. For example, a detection agent with a molar extinction coefficient of about 0.5-5×10⁶ M⁻¹cm⁻¹ may have a higher intensity signal relative to a detection agent with a molar extinction coefficient outside of the 0.5-5×10⁶ M⁻¹cm⁻¹ range and may have lower attenuation due to scattering and absorption. Further, a detection agent with a longer excited state lifetime and a large Stoke shift (measured by the distance between the excitation and emission peaks) may further improve the sensitivity of detection. The location of the detection agent may, for example, provide the activity state of a regulatory element. A combination of intrinsic properties of the detection agent may be used to detect a regulatory element of interest.

A detection agent may comprise a detectable moiety that is capable of generating a light, and a probe portion that is capable of hybridizing to a target site on a regulatory element. As described herein, a detection agent may include a DNA probe portion, an RNA probe portion, a polypeptide probe portion, or a combination thereof. Sometimes, a DNA or RNA probe portion may be between 10 and about 100 nucleotides in length. Sometimes, a DNA or RNA probe portion may be 10 to 100, or more nucleotides in length. A DNA or RNA probe portion may be a TALEN probe, ZFN probe, or a CRISPR probe. A DNA or RNA probe portion may be a padlock probe. A polypeptide probe may comprise a DNA-binding protein, a RNA-binding protein, a protein involved in the transcription/translation process, a protein that detects the transcription/translation process, a protein that may detect an open or relaxed portion of a chromatin, or a protein interacting partner of a product of a regulatory element (e.g., an antibody or binding fragment thereof).

A detection agent may comprise a DNA or RNA probe portion which may be between about 10 and about 100 nucleotides in length. A detection agent may comprise a DNA or RNA probe portion which may be about 10 to 100, or more nucleotides in length.

A set of detection agents may be used to detect a regulatory element. The set of detection agents may comprise 2 to 20, or more detection agents may be used for detection of a regulatory element. A detection agent may comprise a polypeptide probe selected from a DNA-binding protein, a RNA-binding protein, a protein involved in the transcription/translation process, a protein that detects the transcription/translation process, a protein that may detect an open or relaxed portion of a chromatin, or a protein interacting partner of a product of a regulatory element (e.g., an antibody or binding fragment thereof).

A detectable moiety that is capable of generating a light may be directly conjugated or bound to a probe portion. A detectable moiety may be indirectly conjugated or bound to a probe portion by a conjugating moiety. As described herein, a detectable moiety may be a small molecule (e.g., a dye) which may be directly conjugated or bound to a probe portion. A detectable moiety may be a fluorescently labeled protein or molecule which may be attached to a conjugating moiety (e.g., a hapten group, an azido group, an alkyne group) of a probe.

A profile or a detection profile or signature may include the signal intensity, signal location, or size of the signal of the detection agent. The profile or the detection profile may comprise about 100 image frames to 50,000 frames, or more frames. Analysis of the profile or the detection profile may determine the activity of the regulatory element. The degree of activation may also be determined from the analysis of the profile or detection profile. Analysis of the profile or the detection profile may further determine the optical isolation and localization of the detection agents, which may correlate to the localization of the regulatory element.

In additional cases, a detection agent may comprise a polypeptide probe selected from a DNA-binding protein, a RNA-binding protein, a protein involved in the transcription/translation process or detects the transcription/translation process, a protein that may detect an open or relaxed portion of a chromatin, or a protein interacting partner of a product of a regulatory element (e.g., an antibody or binding fragment thereof).

Sometimes, a detectable moiety that is capable of generating a light is directly conjugated or bound to a probe portion. Other times, a detectable moiety is indirectly conjugated or bound to a probe portion by a conjugating moiety. As described elsewhere herein, a detectable moiety may be a small molecule (e.g., a dye) which may be directly conjugated or bound to a probe portion. Alternatively, a detectable moiety may be a fluorescently labeled protein or molecule which may be attached to a conjugating moiety (e.g., a hapten group, an azido group, an alkyne group) of a probe.

In some instances, a profile or a detection profile or signature may include the signal intensity, signal location, or size of the signal of the detection agent. Sometimes, the profile or the detection profile may comprise about 100 frames to 50,000 frames or more images. Analysis of the profile or the detection profile may determine the activity of the regulatory element. In some cases, the degree of activation may also be determined from the analysis of the profile or detection profile. In additional cases, analysis of the profile or the detection profile may further determine the optical isolation and localization of the detection agents, which may correlate to the localization of the regulatory element.

I. Detection of DNA and/or RNA Regulatory Elements

A regulatory element may be DNA. Described herein is a method of detecting a DNA regulatory element, which may include contacting a cell sample with a detection agent, binding the detection agent to the DNA regulatory element, and analyzing a profile from the detection agent to determine the presence, absence, or activity of the DNA regulatory element.

A regulatory element may be RNA. Described herein is a method of detecting a RNA regulatory element, which may include contacting a cell sample with a detection agent, binding the detection agent to the RNA regulatory element, and analyzing a profile from the detection agent to determine the presence, absence, or activity of the RNA regulatory element.

A regulatory element may be an enhancer RNA (eRNA). The presence of an eRNA may correlate to an activated regulatory element. For example, the production of an eRNA may correlate to the transcription of a target gene. As such, the detection of an eRNA element may indicate that a target gene downstream of the eRNA element may be activated.

Provided herein is a method of detecting an eRNA regulatory element, which may include contacting a cell sample with a detection agent, binding the detection agent to the eRNA regulatory element, and analyzing a profile from the detection agent to determine the presence, absence, or activity of the eRNA regulatory element Described herein is an in situ method of detecting an activated regulatory DNA site, which may include incubating a sample with a set of detection agents (e.g., fluorescently-labeled probes), hybridizing the set of detection agents to at least one enhancer RNA (eRNA), and analyzing a profile (e.g., a fluorescent profile) from the set of detection agents to determine the presence of an eRNA, in which the presence of eRNA correlates to an activated regulatory DNA site.

II. Detection of a DNaseI Hypersensitive Site, Generation of a DNaseI Hypersensitive Site Map, and Determination of a Cell Type Based on a DNaseI Hypersensitive Site Profile

A regulatory element may be a DNaseI hypersensitive site (DHS). A DNaseI hypersensitive site may be an inactivated DNaseI hypersensitive site. A DNaseI hypersensitive site may be an activated DNaseI hypersensitive site. Described herein is a method of detecting a DHS, which may include contacting a cell sample with a detection agent, binding the detection agent to the DHS, and analyzing a profile from the detection agent to determine the presence, absence, or activity of the DHS.

The DHS may be an active DHS and may further contain a single stranded DNA region. The single stranded DNA region may be detected by S1 nuclease. A method of detecting a DHS may further be extended to detect the presence of a single stranded DNA region within a DHS. Such a method, for example, may comprise contacting a cell sample with a detection agent, binding the detection agent to a single stranded region of a DHS, and analyzing a profile from the detection agent to determine the presence or absence of the single stranded region within a DHS.

Also described herein is a method of determining the activity level of a regulatory element, which may include incubating a cell sample with a set of detection agents (e.g., fluorescently labeled probes), in which each detection agent hybridizes to a DHS, measuring a signature (e.g., a fluorescent signature) from the set of detection agents, and based on the signature, determining a DHS profile, and comparing the DHS profile with a control, in which a correlation with the control indicates the activity level of the regulatory element in the cell sample. The signature (e.g., the fluorescent signature) may further correlate to a signal intensity (or a peak height). A set of signal intensities may be compiled into a DHS profile and compared with a control to generate a second DHS profile which comprises a set of relative signal intensities (or relative peak heights). The set of relative signal intensities may correlate to the activity level of a regulatory element.

Also described herein is a method of generating a DHS map, which may provide information on cell-to-cell variation in gene expression, memory of early developmental fate decisions which establish lineage hierarchies, quantitation of embryonic stem cell DHS sites which decreases with cell passage, and presence of oncogenic elements.

The location of a set of DHS sites may be correlated to a cell type. For example, the location of about 1 to 60, or more DHS may be used to determine a cell type. The cell may be a normal cell or a cancerous cell. DHS variation may be used to determine the presence of cancerous cells in a sample. A method of determining a cell type (e.g., a cancerous cell) may include incubating a cell sample with a set of detection agents (e.g., fluorescently labeled probes), in which each detection agent hybridizes to a DHS, measuring a signature (e.g., a fluorescent signature) from the set of detection agents, and based on the signature, determining a DHS profile, and comparing the DHS profile with a control, in which a correlation with the control indicates the cell type of the sample.

A DHS site may be visualized through a terminal deoxynucleotidyl transferase (TdT) dUTP Nick-End labeling (TUNEL) assay. A TUNEL assay may utilize a terminal deoxynucleotidyl transferase (TdT) which may catalyze the addition of a dUTP at the site of a nick or strand break. A fluorescent moiety may further be conjugated to dUWP. A TUNEL assay may be utilized for visualization of a plurality of DHSs present in a cell.

The sequence of a DHS site may be detected in situ, by utilizing an in situ sequencing methodology. For example, the two ends of a padlock probe may be hybridized to a target regulatory element sequence and the two ends may be further ligated together by a ligase (e.g., T4 ligase) when bound to the target sequence. An amplification (e.g., a rolling circle amplification or RCA) may be performed utilizing a polymerase (e.g., 29 polymerase), which may result in a single stranded DNA comprising at least about 1 to at least about 10, or more tandem copies of the target sequence. The amplified product at least about be sequenced by ligation in situ using partition sequencing compatible primers and labeled probes (e.g., fluorescently labeled probes). For example, each target sequence within the amplified product may bind to a primer and probe set resulting in a bright spot detectable by, e.g., an immunofluorescence microscopy. The labeled probe (e.g., the fluorescent label on the probe) may identify the nucleotide at the ligation site, thereby allowing the color detected to define the nucleotide at the respective ligation position. Sometimes, at least 1 to at least 20, or more rounds of ligation and detection may occur for detection of a DHS site.

A control as used herein may refer to a DHS profile generated from a regulatory element whose activity level is known. A control may also refer to a DHS profile generated from an inactivated regulatory element. A control may further refer to a DHS profile generated from an activated or inactivated regulatory element from a specific cell type. For example, the cell type may be an epithelial cell, connective tissue cell, muscle cell, or nerve cell type. The cell may be a cell derived from heart, lung, kidney, stomach, intestines, liver, pancreas, brain, esophagus, and the like. The cell type may be a hormone-secreting cell, such as a pituitary cell, a gut and respiratory tract cell, thyroid gland cell, adrenal gland cell, Leydig cell of testes, Theca interna cell of ovarian follicle, Juxtaglomerular cell, Macula densa cell, Peripolar cell, or Mesangial cell type. The cell may be a blood cell or a blood progenitor cell. The cell may be an immune system cell, e.g., monocytes, dendritic cell, neutrophile granulocyte, eosinophil granulocyte, basophil granulocyte, hybridoma cell, mast cell, helper T cell, suppressor T cell, cytotoxic T cell, Natural Killer T cell, B cell, or natural killer cell.

III. Detection and Mapping of a Chromatin

A regulatory element may also be a chromatin. Provided herein is a method of detecting a chromatin, which may include contacting a cell sample with a detection agent, binding the detection agent to the chromatin, and analyzing a profile from the detection agent to determine the activity state of the chromatin. The activity level of a chromatin may be determined based on the presence or activity level of a nucleic acid of interest or the presence or absence of a chromatin associated protein. The activity level of a chromatin may be determined based on DHS locations. The one or more DHS locations on a chromatin may be used to map chromatin activity state. For example, one or more DHSs may be localized in a region and the surrounding chromatin may be decompacted and readily visualized relative to an inactive chromatin state when a DHS is not present. The one or more DHSs within a localized region may further form a localized DHS set and a plurality of localized DHS sets may further provide a global map or pattern of chromatin activity (e.g., an activity pattern).

Also included herein is a method of generating a chromatin map based on the pattern of DNaseI hypersensitive sites, RNA regulatory elements (e.g., eRNA), chromatin associated proteins or gene products, or a combination thereof. The method of generating a chromatin map may be based on the pattern of DNaseI hypersensitive sites. The method may comprise generating a 3-dimensional map from a detection profile (or a 2-dimensional detection profile). A chromatin map may provide information on the compaction of chromatin, the spatial structure, spacing of regulatory elements, and localization of the regulatory elements to globally map chromatin structure and accessibility.

A chromatin map for a cell type may also be generated, in which each cell type comprises a different chromatin pattern. Each cell type may be associated with at least one unique marker. The at least one unique marker (or fiduciary marker) may be a genomic sequence. The at least one unique marker (or fiduciary marker) may be DHS. A cell type may comprise about 5, about 10, about 15, about 20, about 25, about 30, about 35, about 40, about 45, about 50, about 60, or more unique markers (or fiduciary markers). The cell type may be an epithelia cell, a connective tissue cell, a muscle cell, a nerve cell, a hormone-secreting cell, a blood cell, an immune system cell, or a stem cell type. The cell type may be a cancerous cell type.

A chromatin profile (e.g., based on DHSs) in the presence of an exogenous agent or condition may also be generated. The method may comprise incubating a cell sample with a set of fluorescently labeled probes specific to target sites (e.g., target DHSs) on a chromatin in the presence of an exogenous agent or condition; measuring a fluorescent signature of the set of fluorescently labeled probes; based on the fluorescent signature, generating a fluorescent profile of the chromatin; and comparing the fluorescent profile with a second fluorescent profile of a chromatin obtained from an equivalent sample incubated with an equivalent set of fluorescently labeled probes in the absence of the exogenous agent or condition, wherein a difference between the two sets of fluorescent profiles indicates a change in the chromatin density (e.g., changes in the presences or activation of DHSs) induced by the exogenous agent or condition. The exogenous agent or condition may comprise a small molecule or a drug. The exogenous agent may be a small molecule, such as a steroid. The exogenous agent or condition may comprise an environmental factor, such as a change in pH, temperature, nutrient, or a combination thereof.

C. Methods of Determining the Localization of a Regulatory Element

Also described herein is a method for determining the localization of a regulatory element. The localization of a regulatory element may provide an activity state of the regulatory element. The localization of a regulatory element may also provide an interaction state with at least one additional regulatory element. For example, the localization of a first regulatory element with respect to a second regulatory element may provide spatial coordinate and distance information between the two regulatory elements, and v further provide information regarding whether the two regulatory elements may interact with each other. The activity state of a regulatory element may include, for example, a transcription or translation initiation event, a translocation event, or an interaction event with one or more additional regulatory elements. The regulatory element may comprise DNA, RNA, polypeptides, or a combination thereof. The regulatory element may be DNA. The regulatory element may be RNA. The regulatory element may be an enhancer RNA (eRNA). The regulatory element may be a DNaseI hypersensitive site (DHS). The DHS may be an inactive DHS or an active DHS. The regulatory element may be a polypeptide. The regulatory element may be chromatin.

The localization of a regulatory element may include contacting a regulatory element with a first set of detection agents, photobleaching the first set of detection agents for a first time point at a first wavelength to generate a second set of detection agents capable of generating a light at a second wavelength, detecting at least one burst generated by the second set of detection agents to generate a detection profile of the second set of detection agents, and analyzing the detection profile to determine the localization of the regulatory element.

A detection agent may comprise a detectable moiety that is capable of generating a fight, and a probe portion that is capable of hybridizing to a target site on a regulatory element. Each detection agent within the first set of detection agents may have the same or a different detectable moiety. Each detection agent within the first set of detection agents may have the same detectable moiety. A detectable moiety may comprise a small molecule (e.g., a fluorescent dye). A detectable moiety may comprise a fluorescently labeled polypeptide, a fluorescently labeled nucleic acid probe, and/or a fluorescently labeled polypeptide complex.

Upon photobleaching a second set of detection agents may be generated from the first set of detection agents, in which the second set may include detection agents that are capable of generating a burst of light detectable at a second wavelength. For example, bleaching of the set of detection agents may lead to about 50%, about 60%, about 70%, about 80%, about 90%, or more detection agents within the set to enter into an “OFF-state.” An “OFF-state” may be a dark state in which the detectable moiety crosses from the singlet excited or ON state to the triplet state or OFF-state in which detection of light (e.g., fluorescence) may be low (e.g., less than 10%, less than 5%, less than 1%, or less than 0.5% of the light may be detected). The remainder of the detection agents that have not entered into the OFF-state may generate bursts of lights, or to cycle between a singlet excited state (or ON-state) and a singlet ground state. As such, bleaching of the set of detection agents may generate about 40%, about 30%, about 20%, about 10%, about 5%, or less detection agents within the set that may generate bursts of lights. The bursts of lights may be detected stochastically, at a single burst level in which each burst of light correlates to a single detection agent.

A single wavelength may be used for photobleaching a set of detection agents. At least two wavelengths may be used for photobleaching a set of detection agents. A wavelength at 491 nm may be used. A wavelength at 405 m may be used in combination with the wavelength at 491 nm. The two wavelengths may be applied simultaneously to photobleach a set of detection agents. Alternatively, the two wavelengths may be applied sequentially to photobleach a set of detection agents.

The time for photobleaching a set of detection agents may be from about 10 seconds to about 4 hours, or more. The concentration of the detection agents may be from about 5 nM to about 1 μM. The burst of lights from the set of detection agents may generate a detection profile. The detection profile may comprise about 100 image frames to about 50,000 frames, or more. The detection profile may also include the signal intensity, signal location, or size of the signal. Analysis of the detection profile may determine the optical isolation and localization of the detection agents, which may correlate to the localization of the regulatory element.

The detection profile may comprise a chromatic aberration correction. The detection profile may comprise less than 5%, chromatic aberration. The detection profile may comprise 0% chromatic aberration.

More than one regulatory element may be detected at the same time. At least 2 to 20, or more regulatory elements may be detected at the same time. Each of the regulatory elements may be detected by a set of detection agents. The detectable moiety between the different set of detection agents may be the same. For example, two different sets of detection agents may be used to detect two different regulatory elements and the detectable moieties from the two sets of detection agents may be the same. As such, at least 2 to at least 20, or more regulatory elements may be detected at the same time at the same wavelength. Sometimes, the detectable moiety between the different set of detection agents may also be different. For example, two different sets of detection agents may be used to detect two different regulatory elements and the detectable moiety from one set of detection agents may be detected at a different wavelength from the detectable moiety of the second set of detection agents. As such, at least 2 to 20, or more regulatory elements may be detected at the same time in which each of the regulatory elements may be detected at a different wavelength. The regulatory element may comprise DNA, RNA, polypeptides, or a combination thereof.

D. Methods of Measuring the Activity of a Regulatory Element

Also described herein is a method of measuring the activity of a target regulatory element. The method may include detection of a regulatory element and one or more products of the regulatory element. One or more products of the regulatory element may also include intermediate products or elements. The method may comprise contacting a cell sample with a first set and a second set of detection agents, in which the first set of detection agents interact with a target regulatory element within the cell and the second set of detection agents interact with at least one product of the target regulatory element, and analyzing a detection profile from the first set and the second set of detection agents, in which the presence or the absence of the at least one product indicates the activity of the target regulatory element.

As discussed herein, a detection agent may comprise a detectable moiety that is capable of generating a light, and a probe portion that is capable of hybridizing to a target site on a regulatory element. Each detection agent within the first set of detection agents may have the same or a different detectable moiety. Each detection agent within the first set of detection agents may have the same detectable moiety. A detectable moiety may comprise a small molecule (e.g., a fluorescent dye). A detectable moiety may comprise a fluorescently labeled polypeptide, a fluorescently labeled nucleic acid probe, and/or a fluorescently labeled polypeptide complex.

The method may also allow photobleaching of the first set and the second set of detection agents, thereby generating a subset of detection agents capable of generating a burst of light. A detection profile may be generated from the detection of a set of light bursts, in which the presence or the absence of the at least one product may indicate the activity of the target regulatory element.

The regulatory element may comprise DNA, RNA, polypeptides, or a combination thereof. The regulatory element may be DNA. The regulatory element may be RNA. The regulatory element may be an enhancer RNA (eRNA). The presence of an eRNA may correlate with target gene transcription that is downstream of eRNA. The regulatory element may be a DNaseI hypersensitive site (DHS). The DHS may be an activated DHS. The pattern of the DHS on a chromatin may correlate to the activity of the chromatin. The regulatory element may be a polypeptide, e.g., a transcription factor, a DNA or RNA-binding protein or binding fragment thereof or a polypeptide that is involved in chemical modification. The regulatory element may be chromatin.

E. Target Nucleic Acid Sequence

A target nucleic acid sequence may be a nucleic acid sequence of interest or may encode a DNA, RNA, or protein of interest or a portion thereof. A DNA, RNA, or protein of interest may be a DNA, RNA, or protein produced by a cell or contained within a cell. A target nucleic acid sequence may be incorporated into a structure of a cell. A target nucleic acid sequence may also be associated with a cell. For example, a target nucleic acid sequence may be in contact with the exterior of a cell. A target nucleic acid sequence may be unassociated with a structure of a cell. For example, a target nucleic acid sequence may be a circulating nucleic acid sequence. A target nucleic acid sequence or a portion thereof may be artificially constructed or modified. A target nucleic acid sequence may be a natural biological product. A target nucleic acid sequence may be a short nucleic acid sequence. A target nucleic acid sequence may be a nucleic acid sequence that is from a source that is exogenous to a cell. A target nucleic acid sequence may be an endogenous nucleic acid sequence. A target nucleic acid sequence may be a nucleic acid sequence that comprises a combination of an endogenous nucleic acid sequence and a nucleic acid sequence from a source that is exogenous to a cell. A target nucleic acid sequence may be a chromosomal nucleic acid sequence or fragment thereof. A target nucleic acid sequence may be an episomal nucleic sequence or fragment thereof. A target nucleic acid sequence may be a sequence resulting from somatic rearrangement or somatic hypermutation, such as a nucleic acid sequence from a T cell receptor, B cell receptor, or fragment thereof.

A nucleic acid of a cell or sample, which may comprise the target nucleic acid sequence, may comprise a deoxyribonucleic acid (DNA) or a ribonucleic acid (RNA), or a combination thereof. A nucleic acid may be a chromosome, an oligonucleotide, a plasmid, an artificial chromosome, or a fragment or portion thereof. A nucleic acid may comprise genomic DNA, episomal DNA, complementary DNA, mitochondrial DNA, recombinant DNA, cell-free DNA (cfDNA), messenger RNA (mRNA), pre-mRNA, microRNA (miRNA), transfer RNA (tRNA), transfer messenger RNA (tmRNA), ribosomal RNA (rRNA), heterogeneous nuclear RNA (hnRNA), short interfering RNA (siRNA), anti-sense RNA, or short hairpin RNA (shRNA). A nucleic acid may be singe-stranded, double-stranded, or a combination thereof.

A target nucleic acid sequence may comprise a naturally occurring nucleic acid sequence, an artificially constructed nucleic acid sequence (such as an artificially synthesized nucleic acid sequence), or a modified nucleic acid sequence (such as a naturally occurring nucleic acid sequence that has been altered or modified through a natural or artificial process).

A naturally occurring nucleic acid sequence may comprise a nucleic acid sequence present in a cellular sample. A naturally occurring nucleic acid sequence may comprise a nucleic acid sequence present in an unfixed cell. A naturally occurring nucleic acid sequence may comprise a nucleic acid sequence derived from a cellular sample. A nucleic acid sequence may also be derived from a virus (such as a viral nucleic acid sequence from a lentivirus or adenovirus).

A naturally occurring nucleic acid sequence may comprise a nucleic acid sequence present in an acellular sample. A naturally occurring nucleic acid sequence may comprise a nucleic acid sequence derived from an acellular sample. For example, a nucleic acid sequence may be a cell-free DNA sequence present in a bodily fluid (such as a sample of cerebrospinal fluid). A nucleic acid may comprise a target nucleic acid sequence that is not endogenous to the source (exogenous) from which it was taken or in which it is analyzed. A nucleic acid may be an artificially synthesized oligonucleotide.

A nucleic acid sequence may comprise one or more modifications. A modification may be a post-translational modification of a nucleic acid sequence or an epigenetic modification of nucleic acid sequence (e.g., modification to the methylation of a nucleic acid sequence). A modification may be a genetic modification. A genetic modification to a nucleic acid sequence may be an insertion, a deletion, or a substitution of a nucleic acid sequence. A nucleic acid sequence modification may comprise an insertion may comprise transformation, transduction, or transfection of a sample. For example, a nucleic acid sequence modification comprising an insertion may result from infection or transduction of a cell with a virus and subsequent incorporation of a viral nucleic acid sequence into a nucleic acid sequence of the cells, such as the cell's genomic DNA. The integrated viral nucleic acid sequence (viral integrant) or fragment thereof may be the target nucleic acid sequence. Modification of a nucleic acid sequence may be an artificial modification, resulting from, for instance, genetic engineering or intentional nucleic acid sequence modification during nucleic acid fabrication. A nucleic acid sequence may be the result of somatic rearrangement.

A modification to a nucleic acid sequence comprising an insertion, deletion or substitution may comprise a difference between the nucleic acid sequence and a reference sequence. A reference sequence may be a nucleic acid sequence in a database, an artificial nucleic acid, a viral nucleic acid sequence, a nucleic acid sequence of the same cell, a nucleic acid sequence of a cell from the tissue, a nucleic acid sequence from a different tissue of the same subject, or a nucleic acid sequence from a subject of a different species.

A modification to a nucleic acid sequence may comprise a difference in 1 nucleotide (a single nucleotide polymorphism, SNP), from 1 to 1,000 nucleotides. Modification to a nucleic acid sequence comprising a difference in a plurality of nucleotides may comprise differences in two or more adjacent nucleotides or nucleotide sequences relative to a reference nucleic acid sequence. Modifications to a nucleic acid sequence comprising a difference in a plurality of nucleotides may also comprise differences in two or more non-adjacent nucleotides or nucleotide sequences (such as two or more modifications to the nucleic acid sequence that are separated by at least one nucleotide) relative to a reference nucleic acid sequence.

A target sequence may be assayed in situ or it may be isolated and/or purified from a cellular or acellular sample. For example, a target sequence comprising a nucleic acid may comprise a portion (a region) of genomic DNA located in situ in the nucleus of a fixed (intact) cell. A target sequence may comprise a nucleic acid sequence that is isolated from a sample (such as an aliquot of cerebrospinal fluid).

F. Detection Agents

Detection agents may be utilized to detect nucleic acid sequence of interest. A detection agent may comprise a probe portion. The probe portion may include a probe, or a combination of probes. The probe portion may comprise a nucleic acid molecule, a polypeptide, or a combination thereof. The detection agents may further comprise a detectable moiety. The detectable moiety may comprise a fluorophore. A fluorophore may be a molecule that may absorb light at a first wavelength and transmit or emit light at a second wavelength. The fluorophore may be a small molecule (such as a dye) or a fluorescent polypeptide. The detectable moiety may be a fluorescent small molecule (such as a dye). The detectable moiety may not contain a fluorescent polypeptide. The detection agent may further comprise a conjugating moiety. The conjugating moiety may allow attachment of the detection agent to a nucleic acid sequence of interest. The detection agent may comprise a probe that is synthesized with direct dye incorporation at the 3′ end or 5′ end.

G. Probes

A detection agent may comprise a probe portion. A probe portion may comprise a probe or a combination of probes. A probe may be a nucleic acid probe, a polypeptide probe, or a combination thereof. A probe portion may be an unconjugated probe that does not contain a detectable moiety. A probe portion may be a conjugated probe which comprises a single probe with a detectable moiety, or two or more probes in which at least one probe may be an unconjugated probe bound to at least a second probe which comprises a detectable moiety.

A probe may be a nucleic acid probe. The nucleic acid probe may be a DNA probe, a RNA probe, or a combination thereof. The nucleic acid probe may be a DNA probe. The nucleic acid probe may be a RNA probe. The nucleic acid probe may be a double stranded nucleic acid probe, a single stranded nucleic acid probe, or may contain single-stranded and/or double stranded portions. The nucleic acid probe may further comprise overhangs on one or both termini, may further comprises blunt ends on one or both termini, or may further form a hairpin.

The nucleic acid probe may be at least 10 to about 100 nucleotides in length. TABLE 3 lists exemplary nucleotide sequences according to the present disclosure.

TABLE 3
Exemplary Probe Nucleotide Sequences

| | | % GC |
|---|---|---|
| SEQ ID NO | Sequence | Content |
| SEQ ID NO: 1 | TTTCCCTTGCTCTTCATGATTTTAACAACATGATGGATTT | 33 |
| SEQ ID NO: 2 | CCCTGCCCCCCATTAACTCACATCCTGAATTTTATGTTTA | 43 |
| SEQ ID NO: 3 | GCACTTCATCATCGTCTTTGAAGTCCCCTTCTTGTCCTCC | 50 |
| SEQ ID NO: 4 | TATGATGAACACCATGCACCACATGCAGGTTCTGGTGAAG | 48 |
| SEQ ID NO: 5 | GATACAAAAGAATATTGGTATGTATGTTGCACAGACTCAT | 33 |
| SEQ ID NO: 6 | CCTATTTCCCCCACACAGCCTTCCCACATTGGCCAACCCT | 58 |
| SEQ ID NO: 7 | TACAAAGGGCTTCTCTGGCCAGAGAGAGCCGGTGTCTGCT | 58 |
| SEQ ID NO: 8 | TGGGGGGGTTAATGGAGTTATGGACTGGGATGGGCAGCCT | 58 |
| SEQ ID NO: 9 | ACCTACCTAGGGAACTCTTTCTCCCTGGCACTAGGCTAGT | 53 |
| SEQ ID NO: 10 | ACTGACTGAGCTGACCTCCAGTACAGGGCCTGAGGCCACT | 60 |
| SEQ ID NO: 11 | CTGGGAGCTAAATAGAAGCAAATATCCCCAGGCCTGGGTG | 53 |
| SEQ ID NO: 12 | ATGCGTCAAGCAACTACACTCCCACAGTAAACTGGGAACC | 50 |
| SEQ ID NO: 13 | CAGCTCCTTGGCAGCCTAGGCTCTAGCTCAACATCTGCTT | 55 |
| SEQ ID NO: 14 | TGCTGGAGTCGCACCAACCTGGCTCTGCCTATCTCCAGCA | 60 |
| SEQ ID NO: 15 | CTCTGTAGGCTGCACAACGTGGAACAGATGAAAGGAACCA | 50 |
| SEQ ID NO: 16 | TGGGGTAAATTATAATCATGAAATTCCGTCAAGCTTGAAT | 33 |
| SEQ ID NO: 17 | AACATATTTAATATGGCATATTCAAATGACAGAAAGTACG | 28 |
| SEQ ID NO: 18 | CTTTATTCTTGCTAATGTTGACTCCTTAGCAAAGATAATT | 30 |
| SEQ ID NO: 19 | TGATCTTTGCTAAACTCTTCAGGAATAAATGAACATTTCC | 33 |
| SEQ ID NO: 20 | TTTTCAAGCAGTTAAGAAGCAAGAATTAATGACTCGAATA | 30 |
| SEQ ID NO: 21 | ATGAGAGTGTTGACTGATGAAGGGCTCCTATACGCGGGTT | 50 |
| SEQ ID NO: 22 | TCTTTCCCATCTGTTTCCCGGCCCCTACCAGAAATAAGTG | 50 |
| SEQ ID NO: 23 | ATGAACCTCCCTCGCTCCAAGACCAGAGCTCCTAGGAAGT | 55 |
| SEQ ID NO: 24 | TCTTTATTTTATTGGCCACAATTGAACATAGGTATAATTT | 25 |
| SEQ ID NO: 25 | CAGAAGCAAGCCCTGATCAAGGAAACCATTCACACTTGAT | 45 |
| SEQ ID NO: 26 | GTGGCTTTTGCTCAAAGTGAGGACGTTATCAGCTCTGCCC | 53 |
| SEQ ID NO: 27 | CTTTAAACAAAAACTAAAGGCGTAAGGAAAGATAACTACT | 30 |
| SEQ ID NO: 28 | CAGTTGCCACACTTTTTTTCACTGCTAAAGTTCGTAATGA | 38 |
| SEQ ID NO: 29 | GGCAATCAGAAGTATTTTGGTTGCTTCTAGGTCAGAATGA | 40 |
| SEQ ID NO: 30 | GGCAGCAAACTTGTTTAGGTATGATTCATCATTGTCTGCT | 40 |
| SEQ ID NO: 31 | CTACAAAACAATGAGTCTGATTACGACCCACAGAAATGAA | 38 |
| SEQ ID NO: 32 | CCTCCCACAGACCCAAACATGCTGCTGCAAATGTCTCACT | 53 |
| SEQ ID NO: 33 | GGACAAGCACACACATCGCTGGGAAGATCTGCAAGCCTCC | 58 |
| SEQ ID NO: 34 | TAAACCTGGATAACAAGAACACTGTTTCCACTGCGCTAGT | 43 |
| SEQ ID NO: 35 | TCATCACGATGACAATGGACAAGCCATATCCCTAACAGGG | 48 |
| SEQ ID NO: 36 | TTTCCATGACACCAGGACCGTAAAGCACCTTTTACACCGT | 48 |
| SEQ ID NO: 37 | AATTGGGATGTGCAAAACCTCTTAACTTGTAGCACCAAGT | 40 |
| SEQ ID NO: 38 | TCTTGTGTTATTCGCCTGCATTGAAATCCCATCCCAATCC | 45 |
| SEQ ID NO: 39 | TGAGTGATCTCTTTGCTGATCATAAACATATTCCTCCATC | 38 |
| SEQ ID NO: 40 | TGCATTCATTACTAAATACACAGGGCATAGCACATAGTAA | 35 |
| SEQ ID NO: 41 | CTTCAATGTTGCCAGGAAAATCCTTGCAGGAATCACACCC | 48 |
| SEQ ID NO: 42 | ATTTTTTTCTAAAGCTTTAGGAAATACACACGTTTCCCCT | 33 |
| SEQ ID NO: 43 | AGAGTAATCTTCAACAATCCTTGGTCTAAACACACACAAG | 38 |
| SEQ ID NO: 44 | CCCAGGGACCCACGCCAAGCTCACCGCACCTTCCACCAAA | 65 |
| SEQ ID NO: 45 | AGCTCCTGTACTAGCTGGTGGGGTGTGGAGCACACAGCCC | 63 |
| SEQ ID NO: 46 | TCACACAGGGAAAGTGAGGCTTGGTGGTTGATTTGAGCAA | 48 |
| SEQ ID NO: 47 | CCTTCCAACAGCCGTGTGAGACAAGAGGTCTTATCCTCTT | 50 |
| SEQ ID NO: 48 | ACAAGGGTCACTGAGCACATGCCATGTGTTGGGCACAGTG | 55 |
| SEQ ID NO: 49 | GTCTCCTAAGTCTCATTCTTTTCTTAGGATTCTTCAGATC | 38 |
| SEQ ID NO: 50 | TCCGCCTAAGTAAAACATAAAATTACTTAAGCTGCGTAAA | 33 |
| SEQ ID NO: 51 | CATTTTGACCTGATTATCTTTGTCTATAAGTCTTAAGCCA | 33 |
| SEQ ID NO: 52 | CCGGTTCCTCCACCCTCACTGCCCCAACAACTGAAAGAAG | 58 |
| SEQ ID NO: 53 | ACAGTGTGTTGAAAGAATCCATAACTCTTTCTTTCCAGCC | 40 |
| SEQ ID NO: 54 | GAAGTTTCATCTTTATCAAAATCTCCATTCCCAGGCGGAC | 43 |
| SEQ ID NO: 55 | AAGTCCATTTTTTTAAGCTTTGCGCTTCAGCTCCAGAACA | 40 |
| SEQ ID NO: 56 | TCTTCGTTATGAATACAAATAGGAAAACAATCAGACCCAA | 33 |
| SEQ ID NO: 57 | TCCTCGGGGCATTCTAGAACCGTAGCAGACCTGCTTACAT | 53 |
| SEQ ID NO: 58 | TCCTTATGTGGGAAAATAAAGAGGATAGACAGATTTGATT | 33 |
| SEQ ID NO: 59 | AGCTGCGAGTCCCTAACAGACTTCCAGGACAGCTGAAAAA | 50 |
| SEQ ID NO: 60 | AGGACAAGGGAGAGACGCCCACCCGCCTCTGTCAGGGATA | 63 |
| SEQ ID NO: 61 | AATCCATGAGGGTGACATACACATCCTTACTGTTCCCACA | 45 |
| SEQ ID NO: 62 | ACTTCCTTCCCTGAGATGCCCATCCTTTGATTCTGGGATT | 48 |
| SEQ ID NO: 63 | GCTCCCGGATAAATTAATTACCGTGACCCTGAGCTGCTTC | 50 |
| SEQ ID NO: 64 | TAGACTAAGAGAATCTAATTTGTGGCAAAGATCTTGAGTG | 35 |
| SEQ ID NO: 65 | TGAAGGATGACTAAGAGCTTCCCTATAAACCCCATACTGG | 45 |
| SEQ ID NO: 66 | AGCCAGGACTATAGAGTTTCAGAAAAGGGAGAAAATTCTA | 38 |
| SEQ ID NO: 67 | TGCTGCTAATTTAAGTTTCTGGCAAGTCAAAATAAATCTC | 33 |
| SEQ ID NO: 68 | CGAAAACCATCAATTAACTAGAATGATCAGGAAATTGCGT | 35 |
| SEQ ID NO: 69 | TTTATTTAGTCCCCAGGGTGTATGAAGTGCTCTTCCAGGC | 48 |
| SEQ ID NO: 70 | GGTCCTTCTTGGTACCGATATTGCCATATTGGCTGGACAT | 48 |
| SEQ ID NO: 71 | TGGCTTGGTAGGATGCACTCACATGGGCTGTAGTAATACT | 48 |
| SEQ ID NO: 72 | TATCACCAGCATAACTTGTGGTTCTTCAGCCAGTAATTTC | 40 |
| SEQ ID NO: 73 | GAACAACTGGGTATCTACAGGCAAAGAAATGAACCTTGAC | 43 |
| SEQ ID NO: 74 | TAGGTACTGTTGTGTCCCTATATATTTGACTTGGTAATAA | 33 |
| SEQ ID NO: 75 | TATGTGAACATCGGTGAATATCATAATTTATTATGCAAAC | 28 |
| SEQ ID NO: 76 | AGCTGAACACTCTTTGTGGTCCTCTTGAAGCCTAGAATTA | 43 |
| SEQ ID NO: 77 | CCCCACCTCACTGCCCCCCAGTTCTGACTCACGGTGTCCC | 68 |
| SEQ ID NO: 78 | ACTCCCATCACCTGGCCAGCTTGGCTGTCCCCTGACCCAC | 65 |
| SEQ ID NO: 79 | GGCTGCCCAGCTGCCCAGCAGCAAAACTGCATAGGAACTC | 60 |
| SEQ ID NO: 80 | GCCCAGGACGCCAAGTGTCACCACCCTCTCCCCAGGCAGG | 70 |
| SEQ ID NO: 81 | CACAAGGTCAGCTCCACCCGTGGGTCAGTGTGCCCCAGAT | 63 |
| SEQ ID NO: 82 | GGAGACAAAACGGGCACCCAGCCCAGTCATGCCCGTGCCT | 65 |
| SEQ ID NO: 83 | CTGAAATCAGTCAGCAGTTTCGGTGAGTCTGCAGCTGACA | 50 |
| SEQ ID NO: 84 | CGCCACATTTGGGGCTGGGAGAGATGTCACAGGGGCTGAC | 63 |
| SEQ ID NO: 85 | CACATGTTCTCTGCATAGGTTTTTAAGCAGCCAGCAGCTG | 48 |
| SEQ ID NO: 86 | TTTAAAATGAAAACCCACACTTCCAAAATAGCACTTGAGT | 33 |
| SEQ ID NO: 87 | AACATGTTTGTGTAATTAAGCATTTTAAAATCATAACCAT | 23 |
| SEQ ID NO: 88 | TGCTTATCTGTGCTTTTTATGTTCCACCCCCCCACCACCA | 50 |
| SEQ ID NO: 89 | ATTAATAATAATTCTGTGTTTATGGGGATTGCAGATACAT | 28 |
| SEQ ID NO: 90 | CCAGCTTTGTGTCTTCATGACCCAACTGGAGTAAGAATGG | 48 |
| SEQ ID NO: 91 | AAAGACCTCATTTGCAGCATGGTTAGCAGTGTCAAACATT | 40 |
| SEQ ID NO: 92 | TCTCGTAGCACTGGCTGCAGCCGGCCTGTGTGTGCCCACC | 68 |
| SEQ ID NO: 93 | GCCTTCATCCTGAACGGCTGACCAGCGGAAACAAAAGATC | 53 |
| SEQ ID NO: 94 | ATGGCCAGATAACAGTGTTTAGACATGTCTTTGATGTTTT | 35 |
| SEQ ID NO: 95 | CCCTGACTGTGTAAGGGGTCTCTCTCCATGGGGAATAGAG | 55 |
| SEQ ID NO: 96 | CTGAGCTTAGCTTCTACTGTGCTGTTAATTTCAGGCAAGA | 43 |
| SEQ ID NO: 97 | AGATCAATAATATTTGCATTAGCTACTTACATCAGTCTCT | 30 |
| SEQ ID NO: 98 | TAATTGCAGAAAACTTATAAAGCATGGAAGAATACAAAAC | 28 |
| SEQ ID NO: 99 | AAACAAATTCCTCTACCTGGACATGACTGTTGTTAGCATT | 38 |
| SEQ ID NO: 100 | GGGAGATTCTTCATATCCTTTTAATGTAGATATGCACATT | 33 |
| SEQ ID NO: 101 | ACAAAAAAGGCTATCATATTGTACATATAACTTTGCTGTA | 28 |
| SEQ ID NO: 102 | TCTGCTAGGAACCTGTACCCATGTCATTACTGTAAGCATT | 43 |
| SEQ ID NO: 103 | ACTACTCAAATTTTAGTATCTGCAGATATCAGATATCCTT | 30 |
| SEQ ID NO: 104 | TGAAATGGTATTGTTGCCCTTTCTGATTAGTAAAGTATAC | 33 |
| SEQ ID NO: 105 | TTATAATCTAGCAAGGTTAGAGATCATGGATCACTTTCAG | 35 |
| SEQ ID NO: 106 | ACAGCTTGCCTCCGATAAGCCAGAATTCCAGAGCTTCTGG | 53 |
| SEQ ID NO: 107 | TCAATCAACCTGATAGCTTAGGGGATAAACTAATTTGAAG | 35 |
| SEQ ID NO: 108 | GATCATGAAGGATGAAAGAATTTCACCAATATTATAATAA | 25 |
| SEQ ID NO: 109 | TTTAGCCATCTGTATCAATGAGCAGATATAAGCTTTACAC | 35 |
| SEQ ID NO: 110 | AGGGGTAGATTATTTATGCTGCCCATTTTTAGACCATAAA | 35 |
| SEQ ID NO: 111 | CACTACCATTTCACAATTCGCACTTTCTTTCTTTGTCCTT | 38 |
| SEQ ID NO: 112 | GCTCCATCAAATCATAAAGGACCCACTTCAAATGCCATCA | 43 |
| SEQ ID NO: 113 | TCCTACTTTCAGGAACTTCTTTCTCCAAACGTCTTCTGCC | 45 |
| SEQ ID NO: 114 | AATTCTATTTTTTCTTCAACGTACTTTAGGCTTGTAATGT | 28 |
| SEQ ID NO: 115 | TAAGATGCAAATAGTAAGCCTGAGCCCTTCTGTCTAACTT | 40 |
| SEQ ID NO: 116 | CTGTGTTTCAGAATAAAATACCAACTCTACTACTCTCATC | 35 |
| SEQ ID NO: 117 | GAAACCATGTTTATCTCAGGTTTACAAATCTCCACTTGTC | 38 |
| SEQ ID NO: 118 | CTTTGGAAAAGTAATCAGGTTTAGAGGAGCTCATGAGAGC | 43 |
| SEQ ID NO: 119 | GCTGAATCCCCAACTCCCAATTGGCTCCATTTGTGGGGGA | 55 |
| SEQ ID NO: 120 | GGTGTTATGAACTTAACGCTTGTGTCTCCAGAAAATTCAC | 40 |
| SEQ ID NO: 121 | AGTTAATGCACGTTAATAAGCAAGAGTTTAGTTTAATGTG | 30 |
| SEQ ID NO: 122 | TAATTGAGAAGGCAGATTCACTGGAGTTCTTATATAATTG | 33 |
| SEQ ID NO: 123 | CACGGTCAGATGAAAATATAGTGTGAAGAATTTGTATAAC | 33 |
| SEQ ID NO: 124 | CACAAGTCAGCATCAGCGTGTCATGTCTCAGCAGCAGAAC | 53 |
| SEQ ID NO: 125 | GGAGGTGGGGACTTAGGTGAAGGAAATGAGCCAGCAGAAG | 55 |
| SEQ ID NO: 126 | GTCACAGCATTTCAAGGAGGAGACCTCATTGTAAGCTTCT | 45 |
| SEQ ID NO: 127 | AAAGAGGTGAAATTAATCCCATACCCTTAAGTCTACAGAC | 38 |
| SEQ ID NO: 128 | CTTTACTAAGGAACTTTTCATTTTAAGTGTTGACGCATGC | 35 |
| SEQ ID NO: 129 | CAGGTTTTTCTTTCCACGGTAACTACAATGAAGTGATCCT | 40 |
| SEQ ID NO: 130 | GCTCTACAGGGAGGTTGAGGTGTTAGAGATCAGAGCAGGA | 53 |
| SEQ ID NO: 131 | TACTATTTCCAACGGCATCTGGCTTTTCTCAGCCCTTGTG | 48 |
| SEQ ID NO: 132 | AAGGTTTAGGCAGGGATAGCCATTCTATTTTATTAGGGGC | 43 |
| SEQ ID NO: 133 | AGGGGCTCAACGAAGAAAAAGTGTTCCAAGCTTTAGGAAG | 45 |
| SEQ ID NO: 134 | GGGCTGAACCCCCTTCCCTGGATTGCAGCACAGCAGCGAG | 65 |
| SEQ ID NO: 135 | CTGACGTCATAATCTACCAAGGTCATGGATCGAGTTCAGA | 45 |
| SEQ ID NO: 136 | GAAGGTAGAGCTCTCCTCCAATAAGCCAGATTTCCAGAGT | 48 |
| SEQ ID NO: 137 | CACCAATATTATTATAATTCCTATCAACCTGATAGGTTAG | 30 |
| SEQ ID NO: 138 | AGATATAAGCCTTACACAGGATTATGAAGTCTGAAAGGAT | 35 |
| SEQ ID NO: 139 | ACATGTATCTTTCTGGTCTTTTAGCCGCCTAACACTTTGA | 40 |
| SEQ ID NO: 140 | CAAAGAACAAGTGCAATATGTGCAGCTTTGTTGCGCAGGT | 45 |
| SEQ ID NO: 141 | TATTATTATGTGAGTAACTGGAAGATACTGATAAGTTGAC | 30 |
| SEQ ID NO: 142 | TAAAAATCTTTCTCACCCATCCTTAGATTGAGAGAAGTCA | 35 |
| SEQ ID NO: 143 | TTGGGTTCACCTCAGTCTCTATAATCTGTACCAGCATACC | 45 |
| SEQ ID NO: 144 | CACACCCATCTCACAGATCCCCTATCTTAAAGAGACCCTA | 48 |
| SEQ ID NO: 145 | ATGGAACCCAACCAGACTCTCAGATATGGCCAAAGATCTA | 45 |
| SEQ ID NO: 146 | GACACCAGTCTCTGACACATTCTTAAAGGTCAGGCTCTAC | 48 |
| SEQ ID NO: 147 | AGAGATTCAAAAGATTCACTTGTTTAGGCCTTAGCGGGCT | 43 |
| SEQ ID NO: 148 | TCCTTAGTCTGAGGAGGAGCAATTAAGATTCACTTGTTTA | 38 |
| SEQ ID NO: 149 | TAAATGGGGAAGTTGTTTGAAAACAGGAGGGATCCTAGAT | 40 |
| SEQ ID NO: 150 | GGGTTTATACATGACTTTTAGAACACTGCCTTGGTTTTTG | 38 |
| SEQ ID NO: 151 | AACTCTTAAAAGATATTGCCTCAAAAGCATAAGAGGAAAT | 30 |
| SEQ ID NO: 152 | AAATCGAGGAATAAGACAGTTATGGATAAGGAGAAATCAA | 33 |
| SEQ ID NO: 153 | TCAGTTAGGATTTAATCAATGTCAGAAGCAATGATATAGG | 33 |
| SEQ ID NO: 154 | CTTGAAAACACTTGAAATTGCTTGTGTAAAGAAACAGTTT | 30 |
| SEQ ID NO: 155 | ATAATCTTCAGAGGAAAGTTTTATTCTCTGACTTATTTAA | 25 |
| SEQ ID NO: 156 | AGATTCCTTCTGTCATTTTGCCTCTGTTCGAATACTTTCT | 38 |
| SEQ ID NO: 157 | ATTTCAGCTTCTAAACTTTATTTGGCAATGCCTTCCCATG | 38 |
| SEQ ID NO: 158 | GCAGGAGTTTGTTTTCTTCTGCTTCAGAGCTTTGAATTTA | 38 |
| SEQ ID NO: 159 | ACATATCAACGGCACTGGTTCTTTATCTAACTCTCTGGCA | 43 |
| SEQ ID NO: 160 | TTATGCTTCCCTGAAACAATACCACCTGCTATTCTCCACT | 43 |
| SEQ ID NO: 161 | TTCTCACTCCCTACCACTGAGGACAAGTTTATGTCCTTAG | 45 |
| SEQ ID NO: 162 | TTAGAGATTATGTCATTACCAGAGTTAAAATTCTATAATG | 25 |
| SEQ ID NO: 163 | GGTCATTCTTAGAATAGTAATCCAGCCAATAGTACAGGTT | 38 |
| SEQ ID NO: 164 | CAGGCAATAAGGGCTTTTTAAGCAAAACAGTTGTGATAAA | 35 |
| SEQ ID NO: 165 | ATGATGGGCACTGAAGGTTAAAACTTGAGTCTGTCAACTT | 40 |
| SEQ ID NO: 166 | AACTCATAAATATCCCATTTTCCGCTGAAATATAGCTTTA | 30 |
| SEQ ID NO: 167 | CCTGGTTTCTTTGACCTTTTGGGACCTTGAGTAAGTAAAG | 43 |
| SEQ ID NO: 168 | CTTCATTTATTTTCATGATTAAAATTCTAAGAAATTCTTG | 20 |
| SEQ ID NO: 169 | TTTTTAATTAAATTGCATTGCCTAATGTATTTATGAACTA | 20 |
| SEQ ID NO: 170 | CATAGAAATAAAACAATACTCTGAAGTAGTTCAGAATGTG | 30 |
| SEQ ID NO: 171 | CAATTTATATAAAGAGTTAATTCAAATGAGACTATTTTAA | 18 |
| SEQ ID NO: 172 | AGGGCTTTGAATCTTATGTCTAGAAATTTTGAAAAACCTC | 33 |
| SEQ ID NO: 173 | TATATGCTAAGATTCCACCTCTAGTGCTAGAACTGAGAAG | 40 |
| SEQ ID NO: 174 | TGACTTGGTGATCTTTTTTAAATTCTGAAACAACAGCAAC | 33 |
| SEQ ID NO: 175 | AGCTAAGGACTTTTTCTTGCCTATGCATGCTATCTTCAGT | 40 |
| SEQ ID NO: 176 | TGATTATTTAGTATTGAAACTATAACATAGTATGTTTCCT | 23 |
| SEQ ID NO: 177 | AAAAAATGTGTATTTCTCTGGAGAAGGTTAAAACTGAGGA | 33 |
| SEQ ID NO: 178 | CAAGTGAGCAAGGCTTAAATGGAAGAAGCAATGATCTCGT | 43 |
| SEQ ID NO: 179 | CCACCTTCATTAACGAGATCATCCATCATGAGGAAATATG | 40 |
| SEQ ID NO: 180 | ACCAGGCCCCCTCTGTTTTGTGTCACTAAGGGTGAGGATG | 55 |
| SEQ ID NO: 181 | ATGATTTTTCCCTCCCCCGGGCTTCTTTTAGCCATCAATA | 45 |
| SEQ ID NO: 182 | TAGCCCCACAGGAGTTTGTTCTGAAAGTAAACTTCCACAA | 43 |
| SEQ ID NO: 183 | AAGCTTATTGAGGCTAAGGCATCTGTGAAGGAAAGAAACA | 40 |
| SEQ ID NO: 184 | CTCTAAACCACTATGCTGCTAGAGCCTCTTTTCTGTACTC | 45 |
| SEQ ID NO: 185 | CTCATTCAGACACTAGTGTCACCAGTCTCCTCATATACCT | 45 |
| SEQ ID NO: 186 | TATTTTCTTCTTCTTGCTGGTTTAGTCATGTTTTCTGGGA | 35 |
| SEQ ID NO: 187 | GGCAAACCCATTATTTTTTTCTTTAGACTTGGGATGGTGA | 38 |
| SEQ ID NO: 188 | TGGGCAGCGTCAGAAACTGTGTGTGGATATAGATAAGAGC | 48 |
| SEQ ID NO: 189 | GACTATGCTGAGCTGTGATGAGGGAGGGGCCTAGCTAAAG | 55 |
| SEQ ID NO: 190 | TGAGAGTCAGAATGCTCCTGCTATTGCCTTCTCAGTCCCC | 53 |
| SEQ ID NO: 191 | TTGGTTTCTACACAAGTAGATACATAGAAAAGGCTATAGG | 35 |
| SEQ ID NO: 192 | TGTTTGAGAGTCCTGCATGATTAGTTGCTCAGAAATGCCC | 45 |
| SEQ ID NO: 193 | TTACAAATATGTGATTATCATCAAAACGTGAGGGCTAAAG | 33 |
| SEQ ID NO: 194 | CAGATAACTTGCAAGTCCTAGGATACCAGGAAAATAAATT | 35 |
| SEQ ID NO: 195 | AGCATTATGTCTGTCTGTCATTGTTTTTCATCCTCTTGTA | 35 |
| SEQ ID NO: 196 | TTCACAGTTACCCACACAGGTGAACCCTTTTAGCTCTCCT | 48 |
| SEQ ID NO: 197 | GAATGTTTCTTTCCTCTCAGGATCAGAGTTGCCTACATCT | 43 |
| SEQ ID NO: 198 | AATGCACCAAGACTGGCCTGAGATGTATCCTTAAGATGAG | 45 |
| SEQ ID NO: 199 | TCCCAGTAGCACCCCAAGTCAGATCTGACCCCGTATGTGA | 55 |
| SEQ ID NO: 200 | GTGTCCTCTAACAGCACAGGCCTTTTGCCACCTAGCTGTC | 55 |
| SEQ ID NO: 201 | GGCAAACAAGGTTTGTTTTCTTTTCCTGTTTTCATGCCTT | 38 |
| SEQ ID NO: 202 | TTCCATATCCTTGTTTCATATTAATACATGTGTATAGATC | 28 |
| SEQ ID NO: 203 | AAATCTATACACATGTATTAATAAAGCCTGATTCTGCCGC | 35 |
| SEQ ID NO: 204 | AGGTATAGAGGCCACCTGCAAGATAAATATTTGATTCACA | 38 |
| SEQ ID NO: 205 | CTAATCATTCTATGGCAATTGATAACAACAAATATATATA | 23 |
| SEQ ID NO: 206 | ATAATATATTCTAGAATATGTCACATTCTGTCTCAGGCAT | 30 |
| SEQ ID NO: 207 | TTTCTTTATGATGCCGTTTGAGGTGGAGTTTTAGTCAGGT | 40 |
| SEQ ID NO: 208 | AGCTTCTCCTTTTTTTTGCCATCTGCCCTGTAAGCATCCT | 45 |
| SEQ ID NO: 209 | GGGACCCAGATAGGAGTCATCACTCTAGGCTGAGAACATC | 53 |
| SEQ ID NO: 210 | CACACACCCTAAGCCTCAGCATGACTCATCATGACTCAGC | 53 |
| SEQ ID NO: 211 | CTGTGCTTGAGCCAGAAGGTTTGCTTAGAAGGTTACACAG | 48 |
| SEQ ID NO: 212 | AACTGCTCATGCTTGGACTATGGGAGGTCACTAATGGAGA | 48 |
| SEQ ID NO: 213 | CAGAAATGTAACAGGAACTAAGGAAAAACTGAAGCTTATT | 33 |
| SEQ ID NO: 214 | CAGAGATGAGGATGCTGGAAGGGATAGAGGGAGCTGAGCT | 55 |
| SEQ ID NO: 215 | AAAAGTATAGTAATCATTCAGCAAATGGTTTTGAAGCACC | 33 |
| SEQ ID NO: 216 | GTATCTTATTCCCCACAAGAGTCCAAGTAAAAAATAACAG | 35 |
| SEQ ID NO: 217 | GAAAAGAATGTTTCTCTCACTGTGGATTATTTTAGAGAGT | 33 |
| SEQ ID NO: 218 | AATGGTCAAGATTTTTTTAAAAATTAAGAAAACATAAGTT | 18 |
| SEQ ID NO: 219 | CTTGAGAAATGAAAATTTATTTTTTTGTTGGAGGATACCC | 30 |
| SEQ ID NO: 220 | TCTATCTCCCATCAGGGCAAGCTGTAAGGAACTGGCTAAG | 50 |
| SEQ ID NO: 221 | AGTGAGACAGAGTGACTTAGTCTTAGAGGCCCCACTGGTA | 50 |
| SEQ ID NO: 222 | GATGAGAAGGCACCTTCATCACTCATCACAGTCAGCTCTG | 50 |
| SEQ ID NO: 223 | TCTCCTCTCTCCTTTCTCATCAGAAATTTCATAAGTCTAC | 38 |
| SEQ ID NO: 224 | GTCAGGCAGATCACATAAGAAAAGAGGATGCCAGTTAAGG | 45 |
| SEQ ID NO: 225 | GTTGCTGTTAGACAATTTCATCTGTGCCCTGCTTAGGAGC | 48 |
| SEQ ID NO: 226 | TCTTTAATGAAAGCTAAGCTTTCATTAAAAAAAGTCTAAC | 25 |
| SEQ ID NO: 227 | TGCATTCGACTTTGACTGCAGCAGCTGGTTAGAAGGTTCT | 48 |
| SEQ ID NO: 228 | GAGGAGGGTCCCAGCCCATTGCTAAATTAACATCAGGCTC | 53 |
| SEQ ID NO: 229 | ACTGGCAGTATATCTCTAACAGTGGTTGATGCTATCTTCT | 40 |
| SEQ ID NO: 230 | CTTGCCTGCTACATTGAGACCACTGACCCATACATAGGAA | 48 |
| SEQ ID NO: 231 | ATAGCTCTGTCCTGAACTGTTAGGCCACTGGTCCAGAGAG | 53 |
| SEQ ID NO: 232 | CATCTCCTTTGATCCTCATAATAACCCTATGAGATAGACA | 38 |
| SEQ ID NO: 233 | TATTACTCTTACTTTATAGATGATGATCCTGAAAACATAG | 28 |
| SEQ ID NO: 234 | CAAGGCACTTGCCCCTAGCTGGGGGTATAGGGGAGCAGTC | 63 |
| SEQ ID NO: 235 | GTAGTAGTAGAATGAAAAATGCTGCTATGCTGTGCCTCCC | 45 |
| SEQ ID NO: 236 | CTTTCCCATGTCTGCCCTCTACTCATGGTCTATCTCTCCT | 50 |
| SEQ ID NO: 237 | CCTGGGAGTCATGGACTCCACCCAGCACCACCAACCTGAC | 63 |
| SEQ ID NO: 238 | CCACCTATCTGAGCCTGCCAGCCTATAACCCATCTGGGCC | 60 |
| SEQ ID NO: 239 | TAGCTGGTGGCCAGCCCTGACCCCACCCCACCCTCCCTGG | 73 |
| SEQ ID NO: 240 | TCTGATAGACACATCTGGCACACCAGCTCGCAAAGTCACC | 53 |
| SEQ ID NO: 241 | GGGTCTTGTGTTTGCTGAGTCAAAATTCCTTGAAATCCAA | 40 |
| SEQ ID NO: 242 | TTAGAGACTCCTGCTCCCAAATTTACAGTCATAGACTTCT | 40 |
| SEQ ID NO: 243 | GGCTGTCTCCTTTATCCACAGAATGATTCCTTTGCTTCAT | 43 |
| SEQ ID NO: 244 | CCATCCATCTGATCCTCCTCATCAGTGCAGCACAGGGCCC | 60 |
| SEQ ID NO: 245 | GCAGTAGCTGCAGAGTCTCACATAGGTCTGGCACTGCCTC | 58 |
| SEQ ID NO: 246 | ATGTCCGACCTTAGGCAAATGCTTGACTCTTCTGAGCTCA | 48 |
| SEQ ID NO: 247 | TGTCATGGCAAAATAAAGATAATAATAGTGTTTTTTTATG | 23 |
| SEQ ID NO: 248 | TAGCGTGAGGATGGAAAACAATAGCAAAATTGATTAGACT | 35 |
| SEQ ID NO: 249 | AAGGTCTCAACAAATAGTAGTAGATTTTATCGTCCATTAA | 30 |
| SEQ ID NO: 250 | TCCCTCTCCTCTCTTACTCATCCCATCACGTATGCCTCTT | 50 |
| SEQ ID NO: 251 | TTCCCTTACCTATAATAAGAGTTATTCCTCTTATTATATT | 25 |
| SEQ ID NO: 252 | TTATAGTGATTCTGGATATTAAAGTGGGAATGAGGGGCAG | 40 |
| SEQ ID NO: 253 | CTAACGAAGAAGATGTTTCTCAAAGAAGCCATTCTCCCCA | 43 |
| SEQ ID NO: 254 | GATCATCTCAGCAGGGTTCAGGAAGATAAAGGAGGATCAA | 45 |
| SEQ ID NO: 255 | TGTTGAGGTGGGAGGACCGCTTGAGCCTGGGAAGTGCAAG | 60 |
| SEQ ID NO: 256 | AGTGAGCCGAGATTTTGCCACTACACTCCCATTTGGGTGA | 50 |
| SEQ ID NO: 257 | GTGAGACCCTTTCTCAAAAACAAACTAATTAAAAAACCCT | 33 |
| SEQ ID NO: 258 | TTTACAGATGAAGAAACTGAGTCATACAACTACTAAGAGA | 33 |
| SEQ ID NO: 259 | GAGTCACTAATCACTCAGGTGGTCTGGCTCCAGCATCTGT | 53 |
| SEQ ID NO: 260 | TTAATCTCTGCTCTATACTGCCCAAGACTTTTATAAAGTC | 35 |
| SEQ ID NO: 261 | GTTGAGTCACTGAAATGAGTTATTGGGATGGCTGTGTGGG | 48 |
| SEQ ID NO: 262 | GTGCTAAGTTCTTTCCTAAAGGTATGTGAGAATACAAAGG | 38 |
| SEQ ID NO: 263 | AAGCATCCTCCTTTTTACACACGTGAACTAGTGCATGCAA | 43 |
| SEQ ID NO: 264 | GACACTCAGTGGGCCTGGGTGAAGGTGAGAATTTTATTGC | 50 |
| SEQ ID NO: 265 | TGAGAGCCTCTGGGGACATCTTGCCAGTCAATGAGTCTCA | 53 |
| SEQ ID NO: 266 | CAATTTCCTTCTCAGTCTTGGAGTAACAGAAGCTCATGCA | 43 |
| SEQ ID NO: 267 | ATAAACGGAAATTTTGTATTGAAATGAGAGCCATTGGAAA | 30 |
| SEQ ID NO: 268 | TTACTCCAGACTCCTACTTATAAAAAGAGAAACTGAGGCT | 38 |
| SEQ ID NO: 269 | GAAGGGTGGGGACTTTCTCAGTATGACATGGAAATGATCA | 45 |
| SEQ ID NO: 270 | TGGATTCAAAGCTCCTGACTTTCTGTCTAGTGTATGTGCA | 43 |
| SEQ ID NO: 271 | GCCCCTTTTCCTCTAACTGAAAGAAGGAAAAAAAAATGGA | 38 |
| SEQ ID NO: 272 | AAAATATTCTACATAGTTTCCATGTCACAGCCAGGGCTGG | 43 |
| SEQ ID NO: 273 | TCTCCTGTTATTTCTTTTAAAATAAATATATCATTTAAAT | 15 |
| SEQ ID NO: 274 | AAATAAGCAAACCCTGCTCGGGAATGGGAGGGAGAGTCTC | 53 |
| SEQ ID NO: 275 | GTCCACCCCTTCTCGGCCCTGGCTCTGCAGATAGTGCTAT | 60 |
| SEQ ID NO: 276 | GCCCTGACAGAGCCCTGCCCATTGCTGGGCCTTGGAGTGA | 65 |
| SEQ ID NO: 277 | GCCTAGTAGAGAGGCAGGGCAAGCCATCTCATAGCTGCTG | 58 |
| SEQ ID NO: 278 | GGAGAGAGAAAAGGGCTCATTGTCTATAAACTCAGGTCAT | 43 |
| SEQ ID NO: 279 | ATTCTTATTCTCACACTAAGAAAAAGAATGAGATGTCTAC | 30 |
| SEQ ID NO: 280 | ACCCTGCGTCCCCTCTTGTGTACTGGGGTCCCCAAGAGCT | 63 |
| SEQ ID NO: 281 | AAAAGTGATGGCAAAGTCATTGCGCTAGATGCCATCCCAT | 45 |
| SEQ ID NO: 282 | TATAAACCTGCATTTGTCTCCACACACCAGTCATGGACAA | 43 |
| SEQ ID NO: 283 | CCTCCTCCCAGGTCCACGTGCTTGTCTTTGTATAATACTC | 50 |
| SEQ ID NO: 284 | AATTTCGGAAAATGTATTCTTTCAATCTTGTTCTGTTATT | 25 |
| SEQ ID NO: 285 | TTTCAATGGCTTAGTAGAAAAAGTACATACTTGTTTTCCC | 33 |
| SEQ ID NO: 286 | ATTGACAATAGACAATTTCACATCAATGTCTATATGGGTC | 33 |
| SEQ ID NO: 287 | TGTTTGCTGTGTTTGCAAAAACTCACAATAACTTTATATT | 28 |
| SEQ ID NO: 288 | CTACTCTAAGAAAGTTACAACATGGTGAATACAAGAGAAA | 33 |
| SEQ ID NO: 289 | TTACAAGTCCAGAAAATAAAAGTTATCATCTTGAGGCCTC | 35 |
| SEQ ID NO: 290 | TTCTAGGAATAATATCAATATTACAAAATTAATCTAACAA | 18 |
| SEQ ID NO: 291 | GAACAGCAATGAGATAATGTGTACAAAGTACCCAGACCTA | 40 |
| SEQ ID NO: 292 | GTAGAGCATCAAGGAAGCGCATTGCGGAGCAGTTTTTTGT | 48 |
| SEQ ID NO: 293 | TTGTTTTTGTATTCTGTTTCGTGAGGCAAGGTTTCACTCT | 38 |
| SEQ ID NO: 294 | TCCAGGCTGGAGTGCAGTGGCAAGATCATGTCTCACTGCA | 55 |
| SEQ ID NO: 295 | TGACCTCCTGAGCTCAAGGGATCCTCCCATTTCGGCCTCC | 60 |
| SEQ ID NO: 296 | TAGCTGGGACTACAGGTGTACATCACATGCCTGGCTAATT | 48 |
| SEQ ID NO: 297 | TTTTTTTTTTAAGTAGAGACGAGGTCTTGCTATGTTGTCC | 35 |
| SEQ ID NO: 298 | TAATATCAAACTCTTGAGCTCAAGCAGTCCTCCCACTTCT | 43 |
| SEQ ID NO: 299 | TGGAGGTATCCAGTATGAAATTTAGATAATACCTGCCTTC | 38 |
| SEQ ID NO: 300 | GTTGAAATTAGAACTTAATGATATAATGCATCAATGAACT | 25 |
| SEQ ID NO: 301 | ATAGTTCCTAGCACAAAGTAAGAATCCTTTCAATGTGTGT | 35 |
| SEQ ID NO: 302 | GTGTATGTATTTATCTGTTATTAATAGGAATCTTATGGGC | 30 |
| SEQ ID NO: 303 | TCTCACTTAATCCTTATTAATAACTATGAAGCAGGTATTT | 28 |
| SEQ ID NO: 304 | GAGTTTTCCAAGTGAGTTAAGTATAGCTTGTAATACTTAA | 30 |
| SEQ ID NO: 305 | ATATCCACAGGTTACATAGCTAGTATATAACTGAGAAATA | 30 |
| SEQ ID NO: 306 | TATTTATATTATAAAACATTCTAACAATACAGATGTATAT | 15 |
| SEQ ID NO: 307 | TAAAAAACTGAAAGGGCTCATGCAACCCTACCTTCTCAAT | 40 |
| SEQ ID NO: 308 | CTTCTTCACTTAGAAAAAACCAGCCTTAGCTGTCTGCTAT | 40 |
| SEQ ID NO: 309 | CCTTTCAAAATATACTTCTGAGAAATGAGAGAGAGAAATG | 33 |
| SEQ ID NO: 310 | GGGTAGAAGGAAGGAAGATAGGGTAAGAGACAGGGAAGGA | 50 |
| SEQ ID NO: 311 | TGGGGAAAGAAATTAAATTATTCTTTTCTCTGTCTCTTGA | 30 |
| SEQ ID NO: 312 | GCTCTTTCCATTACATTGAATCAAAGGTAATGTTGCCATT | 35 |
| SEQ ID NO: 313 | GACTCTTGAAATAAAGAAAGACCGATGTATGAAATAATTT | 28 |
| SEQ ID NO: 314 | AGTCTATGGCATTTTCAAAATGCAAGGTGATGTCTTACTA | 35 |
| SEQ ID NO: 315 | GCCTTTGCTTTATTATTAGAAATGGGGAAGTGAGTATAGA | 35 |
| SEQ ID NO: 316 | TTATCAGGAGATATATTAGGAAAAAGGGAAACTGGAGAAA | 33 |
| SEQ ID NO: 317 | GAGGAGTATCCAGATGTCCTGTCCCTGTAAGGTGGGGGCA | 58 |
| SEQ ID NO: 318 | CCTTCAATCAAAAGGGCTCCTTAACAACTTCCTTGCTTGG | 45 |
| SEQ ID NO: 319 | CCACCATCTTGGACCATTAGCTCCACAGGTATCTTCTTCC | 50 |
| SEQ ID NO: 320 | AGTGGTCATAACAGCAGCTTCAGCTACCTCTCTAAAGAGT | 45 |
| SEQ ID NO: 321 | CCAGATATAGGTCAGGAAATATAATCCACTAATAAAAAGA | 30 |
| SEQ ID NO: 322 | CATTTTGACTGTAGTTGTTTGTTTTTTGTCATTGTGACTA | 30 |
| SEQ ID NO: 323 | TAACATTCTCACTCTTTCATCAGTAATCACTCAGGTTATT | 33 |
| SEQ ID NO: 324 | GACCAACAGACTGTGGGAAAAATCAGAGAAGGAGGCATCC | 50 |
| SEQ ID NO: 325 | GCTTACTAGCCTAAACTGAAATTGCTATAGCAGAGTGAAC | 40 |
| SEQ ID NO: 326 | AGGTTTACAGATATTTTCCACAAAGAGTAAAAGGATTGAA | 30 |
| SEQ ID NO: 327 | TCTCCAGATCAATGCATAGGAAATAATAATGGACCATAAA | 33 |
| SEQ ID NO: 328 | ATATTATGACGAACAACATTAGGATAAGTCCATATCAATT | 28 |
| SEQ ID NO: 329 | ATCCAGTCATAAGCACAGACTACGTGAAGCACGTCCAAGT | 48 |
| SEQ ID NO: 330 | GCAGGAGAAATGAGAGGAGCAAGAAAGAGGAGCCATTTGA | 48 |
| SEQ ID NO: 331 | GAATAGCAGAAAAAGGAAAGGCAAGTCATATTAACAAATG | 33 |
| SEQ ID NO: 332 | TCATGCCAACAGTACAGATAACTCTGCTAATAAAGGTAGA | 38 |
| SEQ ID NO: 333 | TAATACAGGTAGTAGCAGATATCTACATAGTAGTTAAAGG | 33 |
| SEQ ID NO: 334 | GGCCATCAGTACAGAAGATTCCATAAAGGAGAACCTAAAG | 43 |
| SEQ ID NO: 335 | AGAATAATTTGTCAGAAGCTTAAAAGCTGAACTCTGAGGC | 38 |
| SEQ ID NO: 336 | AACTACAATATCCTTTTGACTGTGGAAAGGGTGGTGAAAG | 40 |
| SEQ ID NO: 337 | GTTCAAGGACATTTGAGCCAACATAGAGAGGAACATTGGC | 45 |
| SEQ ID NO: 338 | TGAGGGATATCTGTCCTGATGTTGTCCAGGATGGTGATGA | 48 |
| SEQ ID NO: 339 | CATATAAATAACGTAGAGAAAACAGGAGGGGATAGAGATC | 38 |
| SEQ ID NO: 340 | CAAAGAGGCATCAAAGATAGGGATGTTTGTAAGGATGAAA | 38 |
| SEQ ID NO: 341 | CTGTTCTTCTCTGAGTAGCCAAGCTCAGCTTGGTTCAAGC | 50 |
| SEQ ID NO: 342 | CATACTGTGGATCTGTAGCAAATTCCCCCTGAAAACCCAG | 48 |
| SEQ ID NO: 343 | TCTGACCCTCACATTCAAGTTCTGAGGAAGGGCCACTGCC | 55 |
| SEQ ID NO: 344 | GCCTTGAGATACCTGGTCCTTATTCCTTGGACTTTGGCAA | 48 |
| SEQ ID NO: 345 | ATAGGGCTTGTTTTAGGGAGAAACCTGTTCTCCAAACTCT | 43 |
| SEQ ID NO: 346 | CTGGTGTCCATACTCTGAATGGGAAGAATGATGGGATTAC | 45 |
| SEQ ID NO: 347 | AGCAGGAGAGGATCAACCCCATACTCTGAATCTAAGAGAA | 45 |
| SEQ ID NO: 348 | TCAGATCCCTGGATGCAAGCCAGGTCTGGAACCATAGGCA | 55 |
| SEQ ID NO: 349 | CTCCTCCCTACCACCTTTAGCCATAAGGAAACATGGAATG | 48 |
| SEQ ID NO: 350 | GACACAAACCTGGGCCTTTCAATGCTATAACCTTTCTTGA | 43 |
| SEQ ID NO: 351 | CTACCTGACTTCTGAGTCAGGATTTATAAGCCTTGTTACT | 40 |
| SEQ ID NO: 352 | TGAACCAACAAGCATCGAAGCAATAATGAGACTGCCCGCA | 48 |
| SEQ ID NO: 353 | GAAAAGCAATAATCCATTTTTCATGGTATCTCATATGATA | 28 |
| SEQ ID NO: 354 | TAACACTTATCTCTCTGAACTTTGGGCTTTTAATATAGGA | 33 |
| SEQ ID NO: 355 | TTTTCTGACTGTCTAATCTTTCTGATCTATCCTGGATGGC | 40 |
| SEQ ID NO: 356 | ATCTTCATCGAATTTGGGTGTTTCTTTCTAAAAGTCCTTT | 33 |
| SEQ ID NO: 357 | GAAATTACAAATGCTAAAGCAAACCCAAACAGGCAGGAAT | 38 |
| SEQ ID NO: 358 | ATTAGGCATCTTACAGTTTTTAGAATCCTGCATAGAACTT | 33 |
| SEQ ID NO: 359 | TACAATATTTGACTCTTCAGGTTAAACATATGTCATAAAT | 25 |
| SEQ ID NO: 360 | AACATTCAGTGAAGTGAAGGGCCTACTTTACTTAACAAGA | 38 |
| SEQ ID NO: 361 | TCTTTTCCTATCAGTGGTTTACAAGCCTTGTTTATATTTT | 30 |
| SEQ ID NO: 362 | TATTTTTGTTCTGAGAATATAGATTTAGATACATAATGGA | 23 |
| SEQ ID NO: 363 | CAAAATCTAACACAAAATCTAGTAGAATCATTTGCTTACA | 28 |
| SEQ ID NO: 364 | AGAATTTATGACTTGTGATATCCAAGTCATTCCTGGATAA | 33 |
| SEQ ID NO: 365 | TTACACTAGAAAATAGCCACAGGCTTCCTGCAAGGCAGCC | 50 |
| SEQ ID NO: 366 | AGTTTGAACACTTGTTATGGTCTATTCTCTCATTCTTTAC | 33 |
| SEQ ID NO: 367 | ACTTCGTGAGAGATGAGGCAGAGGTACACTACGAAAGCAA | 48 |
| SEQ ID NO: 368 | TCTTGAGAATGAGCCTCAGCCCTGGCTCAAACTCACCTGC | 55 |
| SEQ ID NO: 369 | AATAGGATGTCTGTGCTCCAAGTTGCCAGAGAGAGAGATT | 45 |
| SEQ ID NO: 370 | ATTAAAGATCCCTCCTGCTTAATTAACATTCACAAGTAAC | 33 |
| SEQ ID NO: 371 | ACTTAAAGTAGCGATACCCTTTCACCCTGTCCTAATCACA | 43 |
| SEQ ID NO: 372 | TCTCAGGTGTTAACTTTATAGTGAGGACTTTCCTGCCATA | 40 |
| SEQ ID NO: 373 | ATAGTTTCATATAAATGGGTTCCTCATCATCTATGGGTAC | 35 |
| SEQ ID NO: 374 | GGTATTTACATTTGCCATTCCCTATGCCCTAAATATTTAA | 33 |
| SEQ ID NO: 375 | TATTGATATTCCTTGAAAATTCTAAGCATCTTACATCTTT | 25 |
| SEQ ID NO: 376 | CTTTTATTCTCCCCTTCACCGAATCTCATCCTACATTGGC | 45 |
| SEQ ID NO: 377 | TAGTGTCCCAAATTTTATAATTTAGGACTTCTATGATCTC | 30 |
| SEQ ID NO: 378 | ATATGGTCACCTCTTTGTTCAAAGTCTTCTGATAGTTTCC | 38 |
| SEQ ID NO: 379 | ACAATCTTCCTGCTTCTACCACTGCCCCACTACAATTTCT | 45 |
| SEQ ID NO: 380 | AGTCACTGTCACCACCACCTAAATTATAGCTGTTGACTCA | 43 |
| SEQ ID NO: 381 | CTGACCCCTTGCCTTCACCTCCAATGCTACCACTCTGGTC | 58 |
| SEQ ID NO: 382 | AGAAAATCCTGTTGGTTTTTCGTGAAAGGATGTTTTCAGA | 35 |
| SEQ ID NO: 383 | ACATATACTCACAGCCAGAAATTAGCATGCACTAGAGTGT | 40 |
| SEQ ID NO: 384 | ACCCAAAGACTCACTTTGCCTAGCTTCAAAATCCTTACTC | 43 |
| SEQ ID NO: 385 | TGAGGTAGAGACTGTGATGAACAAACACCTTGACAAAATT | 38 |
| SEQ ID NO: 386 | TCCATATCCACCCACCCAGCTTTCCAATTTTAAAGCCAAT | 43 |
| SEQ ID NO: 387 | AAGGTATGATGTGTAGACAAGCTCCAGAGATGGTTTCTCA | 43 |
| SEQ ID NO: 388 | CTCTGGTCAGCATCCAAGAAATACTTGATGTCACTTTGGC | 45 |
| SEQ ID NO: 389 | AACTGTGAACTTCCTTCAGCTAGAGGGGCCTGGCTCAGAA | 53 |
| SEQ ID NO: 390 | TGATTGTTCTCTGACTTATCTACCATTTTCCCTCCTTAAA | 35 |
| SEQ ID NO: 391 | AAACAAAACCCATCAAATTCCCTGACCGAACAGAATTCTG | 40 |
| SEQ ID NO: 392 | CAGAGGTCACAGCCTAAACATCAAATTCCTTGAGGTGCGG | 50 |
| SEQ ID NO: 393 | GAAGGCAGGTGTGGCTCTGCAGTGTGATTGGGTACTTGCA | 55 |
| SEQ ID NO: 394 | CATGGAGGAAAAACTCATCAGGGATGGAGGCACGCCTCTA | 53 |
| SEQ ID NO: 395 | AGCTTGTTAAATTGAATTCTATCCTTCTTATTCAATTCTA | 25 |
| SEQ ID NO: 396 | CATAGTTGTCAGCACAATGCCTAGGCTATAGGAAGTACTC | 45 |
| SEQ ID NO: 397 | GCAGATATAGCTTGATGGCCCCATGCTTGGTTTAACATCC | 48 |
| SEQ ID NO: 398 | CTAAATAACTAGAATACTCTTTATTTTTTCGTATCATGAA | 23 |
| SEQ ID NO: 399 | AGTGTTTAAAGGGTGATATCAGACTAAACTTGAAATATGT | 30 |
| SEQ ID NO: 400 | GGATGGGTCTAGAAAGACTAGCATTGTTTTAGGTTGAGTG | 43 |
| SEQ ID NO: 401 | TGCTGCCAACATTAACAGTCAAGAAATACCTCCGAATAAC | 40 |
| SEQ ID NO: 402 | TATTGTGAGAGGTCTGAATAGTGTTGTAAAATAAGCTGAA | 33 |
| SEQ ID NO: 403 | TTACAACATGATGGCTTGTTGTCTAAATATCTCCTAGGGA | 38 |
| SEQ ID NO: 404 | CTAAGTAGAAGGGTACTTTCACAGGAACAGAGAGCAAAAG | 43 |
| SEQ ID NO: 405 | GTCTTGTATTGCCCAGTGACATGCACACTGGTCAAAAGTA | 45 |
| SEQ ID NO: 406 | CCCTATGTCTTCCCTGATGGGCTAGAGTTCCTCTTTCTCA | 50 |
| SEQ ID NO: 407 | AAAGTTTCCCCAAATTTTACCAATGCAAGCCATTTCTCCA | 38 |
| SEQ ID NO: 408 | AACTGCAGATTCTCTGCATCTCCCTTTGCCGGGTCTGACA | 53 |
| SEQ ID NO: 409 | TAGTGCTGTGGTGCTGTGATAGGTACACAAGAAATGAGAA | 43 |
| SEQ ID NO: 410 | TAACTAGCGTCAAGAACTGAGGGCCCTAAACTATGCTAGG | 48 |
| SEQ ID NO: 411 | CATTGGCTCCGTCTTCATCCTGCAGTGACCTCAGTGCCTC | 58 |
| SEQ ID NO: 412 | TGTTTATGTGTTATAGTGTTCATTTACTCTTCTGGTCTAA | 30 |
| SEQ ID NO: 413 | CCTTTGACCCCTTGGTCAAGCTGCAACTTTGGTTAAAGGG | 50 |
| SEQ ID NO: 414 | TTCTCTTGGGTTACAGAGATTGTCATATGACAAATTATAA | 30 |
| SEQ ID NO: 415 | TGGAAGTTGTGGTCCAAGCCACAGTTGCAGACCATACTTC | 50 |
| SEQ ID NO: 416 | CTGCCCTGTGGCCCTTGCTTCTTACTTTTACTTCTTGTCG | 50 |
| SEQ ID NO: 417 | AACTCAGATATTGTGGATGCGAGAAATTAGAAGTAGATAT | 33 |
| SEQ ID NO: 418 | TACAGAACCACCAAGTAGTAAGGCTAGGATGTAGACCCAG | 48 |
| SEQ ID NO: 419 | TGAGCTCTCCTACTGTCTACATTACATGAGCTCTTATTAA | 38 |
| SEQ ID NO: 420 | AAGCTAATAAGTAGACAATTAGTAATTAGAAGTCAGATGG | 30 |
| SEQ ID NO: 421 | AGCCCAATGTACTTGTAGTGTAGATCAACTTATTGAAAGC | 38 |
| SEQ ID NO: 422 | CCAATACTCAGAAGTAGATTATTACCTCATTTATTGATGA | 30 |
| SEQ ID NO: 423 | GCTAGAATCAAATTTAAGTTTATCATATGAGGCCGGGCAC | 40 |
| SEQ ID NO: 424 | TAATACTAATGATAAGTAACACCTCTTGAGTACTTAGTAT | 28 |
| SEQ ID NO: 425 | ATGGTAATTCTGTGAGATATGTATTATTGAACATACTATA | 25 |
| SEQ ID NO: 426 | TGAAAGAGAAGTGGGAATTAATACTTACTGAAATCTTTCT | 30 |
| SEQ ID NO: 427 | GAGAGACACGAGGAAATAGTGTAGATTTAGGCTGGAGGTA | 45 |
| SEQ ID NO: 428 | GTTGAGAGGGAAACAAGATGGTGAAGGGACTAGAAACCAC | 48 |
| SEQ ID NO: 429 | CAAGGTTCTGAACATGAGAAATTTTTAGGAATCTGCACAG | 38 |
| SEQ ID NO: 430 | TGCCATCTAAAAAAATCTGACTTCACTGGAAACATGGAAG | 38 |
| SEQ ID NO: 431 | GGGATCCTCTCTTAAGTGTTTCCTGCTGGAATCTCCTCAC | 50 |
| SEQ ID NO: 432 | GTTTCCTTCATGTGACAGGGAGCCTCCTGCCCCGAACTTC | 58 |
| SEQ ID NO: 433 | TTGGATAAGAGTAGGGAAGAACCTAGAGCCTACGCTGAGC | 50 |
| SEQ ID NO: 434 | ATCTGGGGCTTTGTGAAGACTGGCTTAAAATCAGAAGCCC | 48 |
| SEQ ID NO: 435 | ACCGCAATGCTTCCTGCCCATTCAGGGCTCCAGCATGTAG | 58 |
| SEQ ID NO: 436 | TATGGGGAAGCAGGGTATGAAAGAGCTCTGAATGAAATGG | 45 |
| SEQ ID NO: 437 | GGTTGCATGAATCAGATTATCAACAGAAATGTTGAGACAA | 35 |
| SEQ ID NO: 438 | AATGCAGGCCTAGGCATGACTGAAGGCTCTCTCATAATTC | 48 |
| SEQ ID NO: 439 | TAACGTTTTCTTGTCTGCTACCCCATCATATGCACAACAA | 40 |
| SEQ ID NO: 440 | TTAATTCCCAAACTCATATAGCTCTGAGAAAGTCTATGCT | 35 |
| SEQ ID NO: 441 | CCCTATAGGGGATTTCTACCCTGAGCAAAAGGCTGGTCTT | 50 |
| SEQ ID NO: 442 | TCCTCACCATATAGAAAGCTTTTAACCCATCATTGAATAA | 33 |
| SEQ ID NO: 443 | TAAGCTGTCTAGCAAAAGCAAGGGCTTGGAAAATCTGTGA | 43 |
| SEQ ID NO: 444 | AGGATTAGAAGATTCTTCTGTGTGTAAGAATTTCATAAAC | 30 |
| SEQ ID NO: 445 | ATTATCTTCTGGAATAGGGAATCAAGTTATATTATGTAAC | 28 |
| SEQ ID NO: 446 | CTCTCTGGTTGACTGTTAGAGTTCTGGCACTTGTCACTAT | 45 |
| SEQ ID NO: 447 | TCTTCAGTTAGATGGTTAACTTTGTGAAGTTGAAAACTGT | 33 |
| SEQ ID NO: 448 | CTACACCATGTGGAGAAGGGGTGGTGGTTTTGATTGCTGC | 53 |
| SEQ ID NO: 449 | ACTTTCCTAACCTGAGCCTAACATCCCTGACATCAGGAAA | 45 |
| SEQ ID NO: 450 | TACACTTTATTCGTCTGTGTCCTGCTCTGGGATGATAGTC | 45 |
| SEQ ID NO: 451 | TACTCTTTGCATTCCACTGTTTTTCCTAAGTGACTAAAAA | 33 |
| SEQ ID NO: 452 | AAAGGCCTCCCAGGCCAAGTTATCCATTCAGAAAGCATTT | 45 |
| SEQ ID NO: 453 | TATTGACATGTACTTCTTGGCAGTCTGTATGCTGGATGCT | 43 |
| SEQ ID NO: 454 | TTTGGTCCTAATTATGTCTTTGCTCACTATCCAATAAATA | 30 |
| SEQ ID NO: 455 | GTTAAAAAAACTACCTCTCAACTTGCTCAAGCATACACTC | 38 |
| SEQ ID NO: 456 | TAATTAGTGCTTTGCATAATTAATCATATTTAATACTCTT | 20 |
| SEQ ID NO: 457 | ACTAGTGTTCTGTACTTTATGCCCATTCATCTTTAACTGT | 35 |
| SEQ ID NO: 458 | GTATTTTTTGTTTAACTGCAATCATTCTTGCTGCAGGTGA | 35 |
| SEQ ID NO: 459 | GCAGTGACTTATAAATGCTAACTACTCTAGAAATGTTTGC | 35 |
| SEQ ID NO: 460 | TTATAAGCATGATTACAGGAGTTTTAACAGGCTCATAAGA | 33 |
| SEQ ID NO: 461 | AGTATCCCTCAAGTAGTGTCAGGAATTAGTCATTTAAATA | 33 |
| SEQ ID NO: 462 | AGTCACCCATTTGGTATATTAAAGATGTGTTGTCTACTGT | 35 |
| SEQ ID NO: 463 | TGGTCATAAAACATTGAATTCTAATCTCCCTCTCAACCCT | 38 |
| SEQ ID NO: 464 | ACAGTTGAAAAGACCTAAGCTTGTGCCTGATTTAAGCCTT | 40 |
| SEQ ID NO: 465 | CAACTACAGGGCCTTGAACTGCACACTTTCAGTCCGGTCC | 55 |
| SEQ ID NO: 466 | GTGGTTCTTTGAAGAGACTTCCACCTGGGAACAGTTAAAC | 45 |
| SEQ ID NO: 467 | TGGAGGAAATATTTATCCCCAGGTAGTTCCCTTTTTGCAC | 43 |
| SEQ ID NO: 468 | GCCTGGTGCTTTTGGTAGGGGAGCTTGCACTTTCCCCCTT | 58 |
| SEQ ID NO: 469 | TCTCATTTCTTTGAGAACTTCAGGGAAAATAGACAAGGAC | 38 |
| SEQ ID NO: 470 | CAAACTTTTCAAGCCTTCTCTAATCTTAAAGGTAAACAAG | 33 |
| SEQ ID NO: 471 | TCAACAAAGGAGAAAAGTTTGTTGGCCTCCAAAGGCACAG | 45 |
| SEQ ID NO: 472 | GATGCAACAGACCTTGGAAGCATACAGGAGAGCTGAACTT | 48 |
| SEQ ID NO: 473 | CATCTGAGATCCCAGCTTCTAAGACCTTCAATTCTCACTC | 45 |
| SEQ ID NO: 474 | TATCTTAACAGTGAGTGAACAGGAAATCTCCTCTTTTCCC | 40 |
| SEQ ID NO: 475 | AACTCATGCTTTGTAGATGACTAGATCAAAAAATTTCAGC | 33 |
| SEQ ID NO: 476 | TCAAAGGAAGTCAAAAGATGTGAAAAACAATTTCTGACCC | 35 |
| SEQ ID NO: 477 | TGCCTTCACTTAAGTAATCAATTCCTAGGTTATATTCTGA | 33 |
| SEQ ID NO: 478 | CCCTACCTTGTTCAAAATGTTCCTGTCCAGACCAAAGTAC | 45 |
| SEQ ID NO: 479 | GCACTTACAAATTATACTACGCTCTATACTTTTTGTTTAA | 28 |
| SEQ ID NO: 480 | CTTTAGTTTCATTTCAAACAATCCATACACACACAGCCCT | 38 |
| SEQ ID NO: 481 | TAGGGACCACAGGGTTAAGGGGGCAGTAGAATTATACTCC | 50 |
| SEQ ID NO: 482 | CTCACAATTAAGCTAAGCAGCTAAGAGTCTTGCAGGGTAG | 45 |
| SEQ ID NO: 483 | GTTGAAAGACAGAGAGGATGGGGTGCTATGCCCCAAATCA | 50 |
| SEQ ID NO: 484 | GCTTGTCTAATTTTATATATCACCCTACTGAACATGACCC | 38 |
| SEQ ID NO: 485 | AATATTGTACACGTACACCAAAGCATCATGTTGTACCCCA | 40 |
| SEQ ID NO: 486 | TGTGAAGTGGTGGATTTGTTAATTAGCCTTATTTAACCAT | 33 |
| SEQ ID NO: 487 | TGACACATATGACATTTTAACTATGTTCCAGATTTTTGAA | 28 |
| SEQ ID NO: 488 | GCAAGGAATCATTCAATGTTTTCTAAATCTATTACTGCAT | 30 |
| SEQ ID NO: 489 | CATTTTCATAGGTTTTCCTCGATTGATCATTATTCATGAT | 30 |
| SEQ ID NO: 490 | AAAGTGATCAAGATATTTTTAGTTCAGGCTCCAAAATTTT | 28 |
| SEQ ID NO: 491 | CTTTACAGGCCGAGAAAAATGAATCTGAATTCCTGACCTC | 43 |
| SEQ ID NO: 492 | TCCACTCAAGGCCTACATTCTGCTATAATGCAATTTCAAG | 40 |
| SEQ ID NO: 493 | AACTGCTTAAAATTAATGGCACAAGTCATGTTTTTGATGT | 30 |
| SEQ ID NO: 494 | CTGACTGTGACGTAGCAATAAAGAAACCCACGTTTCATAT | 40 |
| SEQ ID NO: 495 | CTGGCCCACTGCTTGGAGGAGAGCACTCAGGACCATGAAC | 60 |
| SEQ ID NO: 496 | TTCTGAAATGATAAAGTCAATCACAGGAAGGCACCTGGAC | 43 |
| SEQ ID NO: 497 | ATCATTCTCTTTCCCTTCCTCTATGTGGCAGAAAGTAAAA | 38 |
| SEQ ID NO: 498 | GGAGATAATAATGTGTTACTCCCTAAGGCAGAGTGCCCTT | 45 |
| SEQ ID NO: 499 | CAATTAACTTGGCCATGTGACTGGTTGTGACTAAAATAAT | 35 |
| SEQ ID NO: 500 | CACTAAATCAATATACTTCTCAACAATTTCCAACAGCCCT | 35 |
| SEQ ID NO: 501 | CTAGGCTCCTGAGTTTGCTGGGGATGCGAAGAACCCTTAT | 53 |
| SEQ ID NO: 502 | CCGAGGACCCCGCACTCGGAGCCGCCAGCCGGCCCCACCG | 83 |
| SEQ ID NO: 503 | TTGGAAGCACAGGGTGTGGGATAATGCTAATTACTAGTGA | 43 |
| SEQ ID NO: 504 | GTTCAGTATGCCTTTGATTTTACAATAATATTCCTGTTAT | 28 |
| SEQ ID NO: 505 | AGATTCCATGAAGTATTACAGCATTTGGTAGTCTTTTTGC | 35 |
| SEQ ID NO: 506 | TATTTGCTCTGAAATAAGACATAATTTGGGGTGAGAAAGC | 35 |
| SEQ ID NO: 507 | ACTCATGATATTTGGCTCTAGAATACATGCTCTGAATCAT | 35 |
| SEQ ID NO: 508 | TCCAAGATGAAGTGGCTACTAACTGACAGAGGGCATAATT | 43 |
| SEQ ID NO: 509 | TATTCACAGTAACTCTGTGCCTCAAGTACTATTGTAATAC | 35 |
| SEQ ID NO: 510 | ACATCCTCAATCTACACACTAGGATAGTATAAAAGTAATA | 30 |
| SEQ ID NO: 511 | GTCTACCCATATGTGACCTTCATGTCTTTGCTCTAAGCCC | 48 |
| SEQ ID NO: 512 | CGTGTAATCCTTGACAATGTCATCTCATCTATTTATTCCC | 38 |
| SEQ ID NO: 513 | TCTGAAAGAGACTAACCTTCCCTCGCTTTGCAGAGAAAGA | 45 |
| SEQ ID NO: 514 | ATGCATGGATTCTCTTGAAAAAATGTTTCTGCCATGATGT | 35 |
| SEQ ID NO: 515 | TAGTTGAAGACCTACTGTGTTCAGGGCCGTGAGCCAGGGC | 58 |
| SEQ ID NO: 516 | CAACGTGGAGAGCTGTCCTGGCACCATTTCTTCCTGCTGT | 55 |
| SEQ ID NO: 517 | ATCCTCAAAGGAGCCTGGCTTGGGCTAACAAGGAAGAACT | 50 |
| SEQ ID NO: 518 | TGCCTGGGACCCTGCCCCAAGCAAAGTAATAATCTGAATG | 50 |
| SEQ ID NO: 519 | CTGGTGTGTCCAGTGTGATCCCTGCACCCATGCCCGGAGC | 65 |
| SEQ ID NO: 520 | CTGCCCCCTGCAGCAGGGAAGGGGCTCTGGAAGGGTCTGA | 68 |
| SEQ ID NO: 521 | TAGCTGCTGCCCCACTATGCACCATCGCTTATCTGTTCTT | 50 |
| SEQ ID NO: 522 | GAAACCCGAAAAATGTCCTGGTCCTCTTCTTAAGTCTGGG | 48 |
| SEQ ID NO: 523 | GCTGAGAACATGACTCTGCTTGGCGTTCCATTTAATTGAC | 45 |
| SEQ ID NO: 524 | GAGAGGGTGTGCATTTGAAGTATAGATTTGTTAAACATAG | 35 |
| SEQ ID NO: 525 | CATCAGGCAAAAATACTTCGATGGGACTGTGTTCTTTCAG | 43 |
| SEQ ID NO: 526 | TCTAAAGTGATGTAATGTTGCCACGGAAATTCTAATCCCT | 38 |
| SEQ ID NO: 527 | CGTGCAGAACCAGCTCTGTCTTCCCAGACACTGTCGCTTT | 55 |
| SEQ ID NO: 528 | ACCCCTGAGCACCTCAGTGTCCGTGACTGTGGAGCGGAGG | 65 |
| SEQ ID NO: 529 | CTGCCTGGGACACGTACGGCTGCCCAGTGATCCTGAGCGC | 68 |
| SEQ ID NO: 530 | CACAGCCGGATGGTGTGGGAGCTGGCACTGCCGGGGCTCC | 73 |
| SEQ ID NO: 531 | CGTCTTGGCAGAGGCTCCCTGTCATCAAGGACCTGAGGTT | 58 |
| SEQ ID NO: 532 | GACCCCACAAAGATGAGCGGGTCCCCTTCCCAATTTTCGG | 58 |
| SEQ ID NO: 533 | TCAGGAAGCCGGTGCTCAGCAAACTTATCTGAAGCTCTTG | 50 |
| SEQ ID NO: 534 | GAGGCTGCAGAGGAACATCGTTTGGTCAAATGTGAAATGT | 45 |
| SEQ ID NO: 535 | CTAGCTTCTAGAAAGTGCTGCCAATTTGGGGACCAAGGGA | 50 |
| SEQ ID NO: 536 | GGAAACACTTCTTTTTCCCTTGACAAAGGACATCCTCTGC | 45 |
| SEQ ID NO: 537 | GCATGTGCATAAACACTCGTGTGTGTGTCCTTTTATCCCA | 45 |
| SEQ ID NO: 538 | CCAAATCTCTATACATGTCCATAGAGAGAGGCAGACGTAT | 43 |
| SEQ ID NO: 539 | GGGTTGAAGACAAGGGGCTCAGAGCTTGCTTTTTATACAC | 48 |
| SEQ ID NO: 540 | AGATTCATCTTCATGGCAGGACTTCAGGCAAGAGAGGCCC | 53 |
| SEQ ID NO: 541 | CTCACCCCTTAGCAGGACCCTGACGGAACTGGGTACAGGC | 63 |
| SEQ ID NO: 542 | GGTTGGGAGACAATGGGTGGCCCCTCGGTGTGGTGTCCTC | 65 |
| SEQ ID NO: 543 | AGAGTCTAGAGGGCCCGTGGGGACGGGAGTCCTGGGAACC | 68 |
| SEQ ID NO: 544 | GCGGCATGTCCGGCTTCACCCTGCCCAGAATCACAGCCTC | 65 |
| SEQ ID NO: 545 | ATGGTTAAAAAATTCTCCTACTTAAGACTCCCAGACCCCT | 40 |
| SEQ ID NO: 546 | TGAGATTCCAGGGCTGGTTCCACAACGGCCGGCATCGGCC | 65 |
| SEQ ID NO: 547 | CTGAGTCACTAACAAAGCTCAGGCCTGACCACAGGACATT | 50 |
| SEQ ID NO: 548 | GGCTGGCCTACCTGCCACGGGGCCAGGGCTGGGTGCTTTC | 73 |
| SEQ ID NO: 549 | GGGCTCTGGACGCTGGAGGCCTGAGGCTGCACCCCAGGTT | 70 |
| SEQ ID NO: 550 | ACAGTGGCCACTCACCCACTGGGCCCACATCCCCACAGGC | 68 |
| SEQ ID NO: 551 | ACTCTGCCAGCCTTTGATGCCTCGCTGAGACAGAGGGTCT | 58 |
| SEQ ID NO: 552 | AGCCGGGGCTCTGGCCCCATCCAGGGGCTCCCCCAGCAGC | 78 |
| SEQ ID NO: 553 | CCTTGGAAGTCAGTCAGCAGGTCAGGACACAGTTCAGCCC | 58 |
| SEQ ID NO: 554 | TTACATGCAGTTGGTCTTCTCCTGTGAATGGGGAAACTGA | 45 |
| SEQ ID NO: 555 | CTGCATCACAGAACAGCTGCATTTCTAATGTCAGGCTTCT | 45 |
| SEQ ID NO: 556 | CAGCCTGGGAGGCTTGTCAACCTCCTTTGACAAGCACGCC | 60 |
| SEQ ID NO: 557 | AGAAACTGGGGCTCCAGGGCATGGAGGCTGCCTGTGGCCA | 65 |
| SEQ ID NO: 558 | TCCCGGCCTGGAGGAAGTCTTATTAGCCTCATTTCATGGA | 50 |
| SEQ ID NO: 559 | TCCTGCCAGCCCCCTCACGCTCACGAATTCAGTCCCAGGG | 65 |
| SEQ ID NO: 560 | AATTCTAAAGGTGAAGGGACGTCTACACCCCCAACAAAAC | 45 |
| SEQ ID NO: 561 | GGAAATATTAGTCCCCTCTGCCTGGGACAAGACCACCGAA | 53 |
| SEQ ID NO: 562 | AAACACACCTCTGAATGGAAAGCTGAGAAACAGTGATCTC | 43 |
| SEQ ID NO: 563 | ACTGCACCCCCTCCCTTCCCGTGCCGGCAATTTAACCGGG | 65 |
| SEQ ID NO: 564 | TGCCTTCCTACCTTGACCAGTCGGTCCTTGCGGGGGTCCC | 65 |
| SEQ ID NO: 565 | ATTTCCTTCATCTTGTCCTTCTAGCCTGGAGACTCTTCGG | 48 |
| SEQ ID NO: 566 | AATGCCCGAAAATTCCAGCAGCAGCCCAAGATGGTGGCCA | 55 |
| SEQ ID NO: 567 | CGTTGCAAATGCCCAAGGGGGTAACCCTAAAAGTTAAAGG | 48 |
| SEQ ID NO: 568 | ACACAACCCCTGTGCAAGTTTCATTCCGGCGCACAGGGGC | 60 |
| SEQ ID NO: 569 | TGCAAGAACTAATTTAGCATGCAAGGACGGGGAGGACCGG | 53 |
| SEQ ID NO: 570 | GCCACGAGGGCACCCACGGGCGGACAGACGGCCAAAGAAT | 68 |
| SEQ ID NO: 571 | ACCCCATATCCAAGCCGGCAGAATGGGCGCATTTCCAAGA | 55 |
| SEQ ID NO: 572 | GCCTGGGGAGACCACGAGAAGGGGTGACTGGGGCGCGGCG | 75 |
| SEQ ID NO: 573 | CTGCAGTAGGGGACAACTAGGAAGGCCGGCAGGCCACACG | 65 |
| SEQ ID NO: 574 | GAGTGGGTCCCCCGGGATTTAGGGGGTGAGGTGGAGGTGG | 68 |
| SEQ ID NO: 575 | TCCCCGCCAGGGAAGAGGGGTGCAGGGGGCCCCGTCCGCC | 80 |
| SEQ ID NO: 576 | TGAGGCGCCGCGCCTGCCCTGCGGCGGAGTTGCCCCTGTA | 75 |
| SEQ ID NO: 577 | AAACGCCGGGAGCAGCGAGGGGCAGAGCCCAAAAGCCATC | 65 |
| SEQ ID NO: 578 | TTGTTAAGCAAAGATCAAAGCCCGGCAGAGAATGGGAGCG | 50 |
| SEQ ID NO: 579 | CAACTTCAACAAAACTCCCCTGTAGTCCGTGTGACGTTAC | 48 |
| SEQ ID NO: 580 | CTGCTACTGCGCCGACAGCCCTCTGGAGGCTCCAGGACTT | 65 |
| SEQ ID NO: 581 | GCTCTTCTGCCCCTCGCCGGAGCGTGCGGACTCTGCTGCT | 70 |
| SEQ ID NO: 582 | TCCGCGCTCGGCTCTCGCTTCTGCTGCCCCGCGCTCCCTC | 75 |
| SEQ ID NO: 583 | TTTCCACTTCGCAGCACAGGAGCTGGTGTTCCATGGCTGG | 58 |
| SEQ ID NO: 584 | GGTCGTTGAGGAGGTTGGCATCGGGGTACGCGCGGCGGAT | 68 |
| SEQ ID NO: 585 | TGTCCTACTTCAAATGTGTGCAGAAGGAGGTCCTGCCGTC | 53 |
| SEQ ID NO: 586 | TCGGGCGGCTCTCTTAAGACTTCCCTGCAACTTGTTGCCC | 58 |
| SEQ ID NO: 587 | ACCCACGTTTCTTTGCTACTCACCCCCCTCCCTTCTCTCC | 58 |
| SEQ ID NO: 588 | CTAGAACTTTGAAGTTTGCCGTGGTGTTTCTAGGGATCCG | 48 |
| SEQ ID NO: 589 | AGAAGGGGGTCCGGGAGGGGTGCCTTCGGGAGAAGCCAGT | 68 |
| SEQ ID NO: 590 | CAGGGGCACCCCAATGGGCCCGAGGGTGCGGGCTGGCAGG | 78 |
| SEQ ID NO: 591 | GGGTGCGCTTTGTGTCCCCCGCCTGCGCCCCAGCCCGGCT | 78 |
| SEQ ID NO: 592 | GCCTCAGCGGCCGGGAGCCGCCAACTCCGGGGGGAGGGGG | 83 |
| SEQ ID NO: 593 | AAAGTGCAGTAATACCCTTGATCAGAGTTGATGACTTGAA | 38 |
| SEQ ID NO: 594 | GAGAGAAATAAAGTAGTTGCTCTATTTGTAAATTGAAAAG | 28 |
| SEQ ID NO: 595 | GGTAGCAGTGATTGCTGTATATTTGTGAAAAGGAGGCAAG | 43 |
| SEQ ID NO: 596 | TGCTGATAATGGAAGTGCAGTGGGTTAGCTTTGTTTCCAT | 43 |
| SEQ ID NO: 597 | CCGTTCTACCGTGACTAGTATGGAATTGTGGGAACCAGAA | 48 |
| SEQ ID NO: 598 | TTAACATCAGTGTCAACTGCAGTGTTGTTTCTGAGTAATA | 35 |
| SEQ ID NO: 599 | CATAACTCCATGCTCTCAAACCAATCACTCCTTCATTCAT | 40 |
| SEQ ID NO: 600 | TTCTCCTATGCTGCACCAGAAAGGGTTTTGTGGGTTATCA | 45 |
| SEQ ID NO: 601 | ATCGTTCAGCATCTTTAGGAAATATCCAGAGACTGCATTG | 40 |
| SEQ ID NO: 602 | TTTATTAAGAGCAAAAAAAGCCTGTTTCGTTAGCCAGTCA | 35 |
| SEQ ID NO: 603 | TTGTTCATATGCCTAACTTAATAAATTCTTCATACAGAAA | 25 |
| SEQ ID NO: 604 | ATAACTTTTAAACCCAAACACCTAGAGATTTCATTATGTA | 28 |
| SEQ ID NO: 605 | TTCTTACCATTAAGTCTTCCAAATGATAATTTATTATAAA | 20 |
| SEQ ID NO: 606 | TATGTAAGGACAACTTCATTATATGCTTGAAGAAATTGTT | 28 |
| SEQ ID NO: 607 | AATCTTAAAAGTGACACTAGTCACATTCCACACGGTTAAA | 35 |
| SEQ ID NO: 608 | ATTTTGAAAACTATTCCTTTATCTGGAATGAATGTAAACC | 28 |
| SEQ ID NO: 609 | TTGCATTAAGGGCACCAGAAACTTATAGAAAACCAAAAAG | 35 |
| SEQ ID NO: 610 | TAAAAGACAGTGAACTGAACAGTAATTAACATTACATCCA | 30 |
| SEQ ID NO: 611 | CAAAAAACTGTGTTTATCATATACCAAACATTTTCAAGTT | 25 |
| SEQ ID NO: 612 | TCTCAGGATATTTTGTTCTCTGACACAAATACACCAGTCA | 38 |
| SEQ ID NO: 613 | TAGCTTTACATCTCAGAATGAATCAATGTGGGGGCAGAAA | 40 |
| SEQ ID NO: 614 | AGACCTATATACCTATAGTGCCTAATAGACAATAAGCCAC | 38 |
| SEQ ID NO: 615 | TCTCTCCCCTGCCTAGACTAAGGTAAGTGGGTCTTACCTT | 50 |
| SEQ ID NO: 616 | CATCCTGCTTTTAAAACCCTTAGTGCTCAGCGGCTTGTCT | 48 |
| SEQ ID NO: 617 | AGCTTATAAACTTCAGAGTAATGTAGCACAAATGTCTGTC | 35 |
| SEQ ID NO: 618 | AACTTGAAATAAAACTTTAAACGTTGATTGATTCTTTCCC | 28 |
| SEQ ID NO: 619 | GACAGGCTTAGAGTCCATAACAAACAATCTTAGCTGGAAA | 40 |
| SEQ ID NO: 620 | TGCTCAACAACACTTGTGGAAGAGCAGGGCAAGCTATTTC | 48 |
| SEQ ID NO: 621 | TTACAACATCACTGTAGACATTACTTTTACCCACAGTGCC | 40 |
| SEQ ID NO: 622 | ATCCTAGTTGTATATACTTCTTGGATAAAGTATCTTCGTA | 30 |
| SEQ ID NO: 623 | ATTTTTGGGGAGTGCCATTCCTGCAGGTCTTGAAGACAGG | 50 |
| SEQ ID NO: 624 | CACACAGCCAATGAAACTGACAGAGCCAATGCAACCAAAA | 45 |
| SEQ ID NO: 625 | ACGACTTCAATCAAGAGAAACAGGCAGGTCAGAGTGTGAA | 45 |
| SEQ ID NO: 626 | CTGGTTATCAGGGTTCATAGCACATAGGTTTGACAACCAC | 45 |
| SEQ ID NO: 627 | TTTATTATTCAGCTGGGTAAGCCAAGTGACAGTCTTCCCC | 45 |
| SEQ ID NO: 628 | GTTTTATTCTAGGAATCAACTGCTTTCTAAAAATGTCTAA | 28 |
| SEQ ID NO: 629 | TTTACTGATGGTACTTATTCCCCCAATTATTGATTATTGA | 30 |
| SEQ ID NO: 630 | GCATTTAGGAATATTCAATATTGATACTAAGGTCATCTTT | 28 |
| SEQ ID NO: 631 | TACTCTGTAATGTAGTAATCTTTATGAAGAAATAAATTTG | 23 |
| SEQ ID NO: 632 | ATTTTGAAAAAATGTTTCACTGCATTTTACTATACAAGCT | 25 |
| SEQ ID NO: 633 | ACCACACATTCATCAAAAAATACCTCAAAGAAAATTCTGC | 33 |
| SEQ ID NO: 634 | GTTGTCACAATAAACTCAGTACTGAGTAAAATATCACAAA | 30 |
| SEQ ID NO: 635 | GAGTATATATTGTATTACTTACCTGATGCGCAAAGACCCA | 38 |
| SEQ ID NO: 636 | AAAATGACAGCAACATAGGTGCCACCTGAGGTCCACATCT | 48 |
| SEQ ID NO: 637 | TGGAGAGAGTGGGGTTAATCTGTTACTACACTTTGCTACT | 43 |
| SEQ ID NO: 638 | ATTTCCATCATTTTGTCTTTCAGTAAGCATGTACGAAGTA | 33 |
| SEQ ID NO: 639 | GAGATGAAGATGGTACATCAGTAGGGAGCCCCTCTACTGG | 53 |
| SEQ ID NO: 640 | TCTAATTCATCAAAGTATTCTGGGTTGATTCCAGGTACGT | 38 |
| SEQ ID NO: 641 | ACAAACTCGTTTTGTACAGAGAGGAAAATATTAAAACACC | 33 |
| SEQ ID NO: 642 | ATGTTAATTATAAACACTGTTATAAGTTTTACAAATGTAA | 18 |
| SEQ ID NO: 643 | TCCACTGGCAGAGAGAATATATGTTTCCATTACGGTCCCA | 45 |
| SEQ ID NO: 644 | TCAAAGGTTTTCTATCACGTTTTCTATTATTTACTCACAT | 28 |
| SEQ ID NO: 645 | AAAAACAAGAGTCACACAACCTATGCTCCACAATATCTGC | 40 |
| SEQ ID NO: 646 | ATAGGTTATTCTACAATCGACACCAACTATCAGCGGCTTT | 40 |
| SEQ ID NO: 647 | ATTGAATTAAATGATGGCTTGATTATCCAGGAATCAGCCA | 35 |
| SEQ ID NO: 648 | CTTACCATAACAGAGTAATCTCTAGCTTATTCCAAGGATA | 35 |
| SEQ ID NO: 649 | ACCTAAAATTTAACTAGAATCACTTTTCAATGAAGCTGCT | 30 |
| SEQ ID NO: 650 | TAAACTAAGAGCCTTTGATCTTGCCTTATTCTGATAAAAT | 30 |
| SEQ ID NO: 651 | AAATAATAATTCACAAGGAAATCCTTATTGTTTATTTAAA | 18 |
| SEQ ID NO: 652 | GTAATATGTAGGTTAAACAGAAATGTTGGTTGAATCATGT | 30 |
| SEQ ID NO: 653 | TGCAGACACTAATCAAACCAAACAGGGCCAATTAAAATTG | 38 |
| SEQ ID NO: 654 | TAAAGTGCAATGGGACAGAGCAACTTCATTTTCACAAACA | 38 |
| SEQ ID NO: 655 | TAATCTAATTGCCAGAAATGCTTGCCCATTGCAATGGGAG | 43 |
| SEQ ID NO: 656 | AGTTGACAATGACTGCTTAGTTTAGGGTTTTGAAGTAAAC | 35 |
| SEQ ID NO: 657 | CAGATGGCAGGTATTCTGTGAATTAACACTGATGCTTCTG | 43 |
| SEQ ID NO: 658 | AGTCAAGTTCAGAAATGATCTGTTATGACCCCATGAAACG | 40 |
| SEQ ID NO: 659 | GGGATGCTCTGATACATCATTCAGTAAAATGATAGAAAAA | 33 |
| SEQ ID NO: 660 | TAGCTGTATTGCTTGATAGCTTCATAGCTTGATAACCATT | 35 |
| SEQ ID NO: 661 | TTTTAGCAGGGAATTAACACAGGTATATAAATGAAGAAAA | 28 |
| SEQ ID NO: 662 | TTGATTGTTTATGAAGCTGAGATTGTTTACTGGTTTCGAG | 35 |
| SEQ ID NO: 663 | TCTGTGTTTTTATGTTTGGGAACATGAGGGAATCAGTTCT | 38 |
| SEQ ID NO: 664 | TTCTTAAGCTTTCATTTTTCCAGTGGTGAATGTAGAGAGA | 35 |
| SEQ ID NO: 665 | ACGGTAACTGAATAAACTTAAGAACTGAGGTAAAGTTTTC | 33 |
| SEQ ID NO: 666 | TCAATATGTAAAATTGATCAATTCAGACACCTTTATATGG | 28 |
| SEQ ID NO: 667 | TGTCTCTTTCATGCTGTAAATAGAGCATTGCATGAAAGAT | 35 |
| SEQ ID NO: 668 | TTCATAGCACAGTTTATAAACCTAAGAAAGCAAAGATGAA | 30 |
| SEQ ID NO: 669 | AACCAAGCAGGATTCTATGACTAAAAAAGTGTATTTGTAT | 30 |
| SEQ ID NO: 670 | AGATAGAGAATTTCAAAGAAACCATCTTTATCAGCTGCAC | 35 |
| SEQ ID NO: 671 | CCAAGAATGAAAAGATGCACTAATTCGACTGAAAGCCAAG | 40 |
| SEQ ID NO: 672 | TCATAGTTGAGACATATAACAACCATAAAGGTCCGCATAT | 35 |
| SEQ ID NO: 673 | AGGAAAGGGTGGAAAGGCAAGCAGCGGGGAGTGTTGGCTG | 60 |
| SEQ ID NO: 674 | CTATAAATTGACCTATCCTGTAAAAAAGGATGTCACAGCA | 35 |
| SEQ ID NO: 675 | ACAATTGACCTAAGACTGTAAATTGTAAATTGACTATAAA | 25 |
| SEQ ID NO: 676 | GCAAGACTGGGTATACTATTAATAGGAAAAAATGAACTTC | 33 |
| SEQ ID NO: 677 | ATTGCTTTGATATTGATTGAATCACAGAGAAAATCCTAAG | 30 |
| SEQ ID NO: 678 | TAGATTATGCTGGCAAATCTCAGTGATCAGAGAATTATAT | 33 |
| SEQ ID NO: 679 | ATTCAGAAATGGAATAGGAAGATATTTATGTGCCATCCTG | 35 |
| SEQ ID NO: 680 | GTTTGAATTATTATTCAAACAGTGTATGTTTGTTTGTACT | 25 |
| SEQ ID NO: 681 | AATGCAACAGAGACAGGTATTTATAGCATCTGTTTTCCAT | 35 |
| SEQ ID NO: 682 | TTTAATATCCAAATATGTATGGACACATACAATTGTACAT | 25 |
| SEQ ID NO: 683 | ACGTCTACCGTCATTTTCGTAATTATTCGGTTTCCCTGTC | 43 |
| SEQ ID NO: 684 | GGAGCGCTCCTGCGCGCCTTGTTCGTTAGGATTTATTTTT | 50 |
| SEQ ID NO: 685 | GGTGGCTCCCTAATGCCTGCTCGTTTCAGGTCTCAGCTCT | 58 |
| SEQ ID NO: 686 | CCTTAGTGTGTTGAGGACGCTGCAGAAGGTACAGAGGAGA | 53 |
| SEQ ID NO: 687 | GACCAGATGGTAGGACAGTCATTCTCCTCTGCGTCTCCGC | 58 |
| SEQ ID NO: 688 | CGTGAGGCATGGAGTTTTTGTCCTGCCCCTGCCTGGTTAG | 58 |
| SEQ ID NO: 689 | TTTAAGTCTCTGGCACCGTGCATAGCAGAATTGGTTGGGA | 48 |
| SEQ ID NO: 690 | TCTTTCTCCAAGTGCCTCTATGTTGGCACATCTCTGAAAT | 43 |
| SEQ ID NO: 691 | TGCGTCCCGGCCAGGTAAGCAGCTTCCCTCTCAGCTGCCT | 65 |
| SEQ ID NO: 692 | GGGTGTATGTAGCTGGCAGAAGTGGGACTTGGTCGCAACC | 58 |
| SEQ ID NO: 693 | CGTGGCGAGTGGGCGGTAGCTGCTCGTAGAGCGTGTGAAA | 63 |
| SEQ ID NO: 694 | GTTGGCCCTAAAAGTTATCATTCATGCTAGTTTGACCAAT | 38 |
| SEQ ID NO: 695 | AAGTGGGAGGAGCTGGGCAAGAAAGTCCACCCCTTTTTCT | 53 |
| SEQ ID NO: 696 | GCCGAGCCGAAGTCATCTGCCAATCAAAACAGCCACAGGG | 58 |
| SEQ ID NO: 697 | CGCGTACCTAATGGGAGACAGACAGGTGCCTTTAAAGCGG | 55 |
| SEQ ID NO: 698 | TGGGGAAAGCGGAGGAAGGCATGGAGTGTGGGCGTTAGGG | 63 |
| SEQ ID NO: 699 | GCATATTCTGCCTTGAAGTCATTGGTTGGTCCTGGAAGTG | 48 |
| SEQ ID NO: 700 | AATTGGTCTGGGGGAGGAGCTACGACAGTCCAGGGGCGGG | 65 |
| SEQ ID NO: 701 | GTGTCGTGCTGATTGGATGTATCCGCCCCCCTCTCTTAAA | 53 |
| SEQ ID NO: 702 | CAACACGCCAGCGCGAGGACCCGAACGTCAATCAAGAGAC | 60 |
| SEQ ID NO: 703 | GCGTTCGATTGGCCTCCCGCGCAGGCTGCTAGGATTGGCT | 65 |
| SEQ ID NO: 704 | CCCTGCCCCCTTTCGCGGATTGGGTGATCGCTCCAAGGCG | 68 |
| SEQ ID NO: 705 | CTGACCCTTGGAGGCTTTCTATTGGTTCCTGGCAGGGATG | 55 |
| SEQ ID NO: 706 | TCCCGAATATAGGCCAGTCATTGCTCCTGCTGAACGTCGC | 55 |
| SEQ ID NO: 707 | CCCCTCCTCTCTTCTCGTCTCTGGCGCCGACCCGCCCCCG | 75 |
| SEQ ID NO: 708 | GCTCAAGGGAGGCCGCGGCGTCTGCCGATGGCTCCGCGGA | 75 |
| SEQ ID NO: 709 | TGGGGGAGTGGGCCCGGGGTTGTTCTGACGACGGGGGTCG | 73 |
| SEQ ID NO: 710 | CCCGGGCGCTATCGCGATAGCGGCGCGAAGCGGAAGTGGG | 73 |
| SEQ ID NO: 711 | CGGGGGAGGCGAGCGCCCGCCGCCTTTTTCTCGCGCCCCG | 80 |
| SEQ ID NO: 712 | CACAGGAGCTGGCGCCGCCGCTGAGGAGCGTATCGCGACA | 70 |
| SEQ ID NO: 713 | GTTGCCGACTCGCGCTCTCGGCTTCTGCTCCGGGGCTTCT | 68 |
| SEQ ID NO: 714 | ACTCGGAGCTCGGATCCCAGTGTGGACCTGGACTCGAATC | 60 |
| SEQ ID NO: 715 | GGCTCCTCCTTGTTCCGAGCCCGAAGGCCCGCCCCTTCAC | 70 |
| SEQ ID NO: 716 | CTTTCCGGAGCCCGTCTGTTCCCCTTCGGGTCCAAAGCTT | 60 |
| SEQ ID NO: 717 | GACCCCGCCTCATTCCTCACGGCGAGCTCCAGACCCCGCC | 73 |
| SEQ ID NO: 718 | AGAACTCAAGCTCCCGATTGTGCCCGAAGGAACCCGAAGG | 58 |
| SEQ ID NO: 719 | ACTATTGCCGAAGTGAGCCGAAGTTTGTGGCCCCGCTTCC | 58 |
| SEQ ID NO: 720 | ACATGTGGCTCCGCCCACACTGGCCTCAGCTCTCCGTTCT | 63 |
| SEQ ID NO: 721 | ACAGTGACCCTAAGGACTCGACTACCTCCGAAGAAAGCCG | 55 |
| SEQ ID NO: 722 | CTTGTACCCAACTATCTACGAAGTAAACCGAAGCTTGTGG | 45 |
| SEQ ID NO: 723 | TATCTGGCGAACCTGTTGACTCCGCCTATCATCCTAGCGT | 53 |
| SEQ ID NO: 724 | GGCAAGTCGCTTTCGCCCCGCCCCCTTGTAAATACTCATG | 58 |
| SEQ ID NO: 725 | CTCCTCTACTTGGGAACTTGAGGATCGTCACCCTGGCCCG | 60 |
| SEQ ID NO: 726 | TTGGCTCCGCCCCACTGAGCGCACCTCCCTCTGCCGCTTC | 70 |
| SEQ ID NO: 727 | TCCTTGCTCCACCCCCTCATGCCGACACCCTCGTCAACTT | 60 |
| SEQ ID NO: 728 | TCCACCGATAGAACCAGCGAGTCACCTCATAAACAGTAAT | 45 |
| SEQ ID NO: 729 | CGCTCAGTCCGCCTCCTTGCCTCCCTTCAGAATGTCCCAC | 63 |
| SEQ ID NO: 730 | GCCGTCCACTCTCCGCTCGGGCGGGCTCACCCCAATTGGG | 73 |
| SEQ ID NO: 731 | CGACCGAACCCCACAGCCGAAAGCCCCGCCCCCTGGACAC | 73 |
| SEQ ID NO: 732 | CTCCGAGCGCCAGCGCACCCCAGTTGGGGAGTTCCCGCCC | 75 |
| SEQ ID NO: 733 | AGCCCCGCCTCCTCCCGGACGCAATAGGTTCGGCGTTCGG | 70 |
| SEQ ID NO: 734 | AGCAATTTGACGTTCGGGTGTTCTCGGCTCGGCCGAATCC | 58 |
| SEQ ID NO: 735 | TGCCCCCTCCCGAGCACAGGAAGTTCGGCGTTCGGGCGTC | 70 |
| SEQ ID NO: 736 | TTTCGGACCTCCTCGCTCTCAGACTCCCACAGTACAAAAC | 53 |
| SEQ ID NO: 737 | CGAGCCTTCGCTCCTCCTCTTTCCGAACGACTGTGATTCG | 58 |
| SEQ ID NO: 738 | GAGGCTAAGGCACCGCCGAGGCCACACCCTCTTCCGGACG | 70 |
| SEQ ID NO: 739 | GCGTCCCCCTTCGGGTGTTCCCGTCAGCGGTCAGAAGCTC | 68 |
| SEQ ID NO: 740 | CCTTACAAAGGTCCATTTTGGCACCACCCTCTTGCAAAGT | 48 |
| SEQ ID NO: 741 | GGAGCGTGAAAAACAAACCTCCGCAAGCGCGGCGACACGC | 63 |
| SEQ ID NO: 742 | ACCCGCTCTGTGCCCGCACTGCCGTACCTACCATTGCGCC | 68 |
| SEQ ID NO: 743 | GGTCCTCAGCATCTGCATATGTAGCCCCTCCCGCTGGTCA | 60 |
| SEQ ID NO: 744 | CCCAACCCCTACCCCCAATCCATCTTAGAGCTGATTCTCT | 53 |
| SEQ ID NO: 745 | ACTCCAGTGATTCTTCCTTATGCTAGGGACTCGAGGACCC | 53 |
| SEQ ID NO: 746 | GAGAATTGAGAAGTCAGTGTGGGAGGGGATGTCCCAGTAC | 53 |
| SEQ ID NO: 747 | TTTCTGGTTCGCGTTGGCTGCATTGTGGAGCTGAGGGATG | 55 |
| SEQ ID NO: 748 | TAGCTTCTTAATCTCCTTCTTTAGGTCAGCCTCATACTTT | 38 |
| SEQ ID NO: 749 | TTCTCCCTGGGACCCAGCAGTCCACTCTCCCAGTTCCCTC | 63 |
| SEQ ID NO: 750 | AAAGTCAGACCTCAGGACCCAGGAACTGGGGCCCACAGCT | 60 |
| SEQ ID NO: 751 | TCTTGATTTGGTCCCTCAGCCGCTGCAGATGGGAAAAGCA | 53 |
| SEQ ID NO: 752 | TAAGCTGCCTCTTGTCCTTGATCTCGTTGGACGCTACCCA | 53 |
| SEQ ID NO: 753 | GGCTCTGGGCTCCTACCGTCTCAATGAGCTTGCGGTTGTC | 60 |
| SEQ ID NO: 754 | TGAGGACCTCTGGGGTCTGGCCGCTCTGCCTCCGCCCCTT | 70 |
| SEQ ID NO: 755 | CTGCCTCTTCACTTCCCTTAGGTGCAGAAACCTTACTTCT | 48 |
| SEQ ID NO: 756 | CGACCTGAGCCTCGTGACCCTACTTTCTGAGCTCTGAGTC | 58 |
| SEQ ID NO: 757 | TCAAAGGTGGGAAAGGAGCTGACTAAGGGCCAGCAGACAC | 55 |
| SEQ ID NO: 758 | CCGTTCCATTTGCTGTAGAGAGTGCAGTTGGCAGGGGGGC | 60 |
| SEQ ID NO: 759 | GCTGTAAGCTTTGGTTTTGGTCTCTCGTTCCACAACTTTG | 45 |
| SEQ ID NO: 760 | CCAACTCACCGTGAGCCACTGGCCAACCTCTTCCTTCTCC | 60 |
| SEQ ID NO: 761 | CCAGGGCTCAGGATCCTCAGAGTTCACCTCCTCTTCTCTA | 55 |
| SEQ ID NO: 762 | GTCCACCTGCATGTTGAGCGTGTCGATGGTATTCTAGGGG | 55 |
| SEQ ID NO: 763 | GCGTGTCTGCACTGACAGTGACTCCACTTCACTCTCAAAC | 53 |
| SEQ ID NO: 764 | TGTCGGGTCTCCCTCACTCACATCCTTGTCGCCCTTCTTC | 58 |
| SEQ ID NO: 765 | CTGCTGGCCAGCCCATTCCCATGCCCATCCCCATCCCAAA | 63 |
| SEQ ID NO: 766 | GAATCCAGGCCCCAACTCCCAGGAGCATAAATGACTGGCC | 58 |
| SEQ ID NO: 767 | TCTCAAATCCCTAATCCCGGCTGTTGGCCCTGTCCGCCTG | 60 |
| SEQ ID NO: 768 | CCTGCCCCACGCGTGCAGCTGCTAAGCCCTCCCAATCCTG | 68 |
| SEQ ID NO: 769 | CCCAGACACCCAGGGGACCCTGAGATTCTGTCTGACCTCC | 63 |
| SEQ ID NO: 770 | CTTCCCCCAAGTCGCTCCTCTTCACAAAGGCCCCACGGTC | 63 |
| SEQ ID NO: 771 | CCTCTGGGTGCCAGGAGGCCTCTTGCCATGGGTGTCCTTC | 65 |
| SEQ ID NO: 772 | CTGCCTTGTCTCTACCCACTGTGCTCTCCCTAGGACCAGG | 60 |
| SEQ ID NO: 773 | GGCGAGGGGGAGGTCCTGCAGCTGCTCGCGTGGGCTGCCC | 78 |
| SEQ ID NO: 774 | TGCGCTCGATCTCATCCTTCAGTTCGTAGCCCACCTGGGG | 60 |
| SEQ ID NO: 775 | TCACCTGCTTCACAGGCGGCGGCTCCTGCCACTTGTCGAA | 63 |
| SEQ ID NO: 776 | CTCGCTTCTTCCGCTGTCCATCCAGGGGCGCAGGCAGCGG | 70 |
| SEQ ID NO: 777 | CCCATGCCTACCGGACCCCCAGGGCCCCTCACCTGCGGCC | 78 |
| SEQ ID NO: 778 | AGTCGGCTGGGAGGAGGACGCCGGCTTCTCCCCTCCATGA | 68 |
| SEQ ID NO: 779 | ATCTTGCGGTACCTGGGGACGGGTGGGTGGGCGGCGCCAG | 73 |
| SEQ ID NO: 780 | TTGGCCTGCTTCCGGATCTCCGTCAGCCCCAGCCGCTCCT | 68 |
| SEQ ID NO: 781 | GGAGGGCGCTCTGGGAGTCTGACCTCTCCGAAGCTCATAC | 63 |
| SEQ ID NO: 782 | AGGAGGCAGAGGGCGGTGGCGGCTGGCTGGCTGTGGGGTT | 73 |
| SEQ ID NO: 783 | AGACATGAGCCAGGGCCACAGGACGAGAGGAGGGGCGGTG | 68 |
| SEQ ID NO: 784 | CCAAGGGCCGCGAGGGTCGCTTTGGGGCTGAATGGATGGA | 65 |
| SEQ ID NO: 785 | GATGGGAAGCCGCGGGGGCTCTAAGCAGCGGAGACACAGG | 68 |
| SEQ ID NO: 786 | GGAGCCTCTGGGCAGGGAGGAACCGGCCAAGGAGCCCGGG | 75 |
| SEQ ID NO: 787 | GGCGGGGCCCAGGGACGGGGCGGCCGTGCAGCAGGGCACT | 83 |
| SEQ ID NO: 788 | CTGCAGGACCAAGGGGATGACGCTGGGATAACAGAGGAGA | 58 |
| SEQ ID NO: 789 | CAGAACAGGTTTAATAGGATGAGGTGGCCTCTGAGTTCGG | 50 |
| SEQ ID NO: 790 | CCATTCCTTCCTTACTCGTGTGGGTCGGGGGATGTCAGGA | 58 |
| SEQ ID NO: 791 | GGCCCGGTCCCAGCACTGCTCTGTGAGCTCAGAGTTGGGA | 65 |
| SEQ ID NO: 792 | TGGGGGCCCACACACGCGGGGGATGCCGGGGAGCCTGAGA | 75 |
| SEQ ID NO: 793 | CACGGGCACCTGCTCCGGTACCCACTCGGCCCGGCTGAGG | 75 |
| SEQ ID NO: 794 | CTCCACCAGCCGGAAGCCCAGCGGTCACCAGCCGGCCGGT | 75 |
| SEQ ID NO: 795 | AGGCGTCCTCCTCGATCTAGGGGGAAGAGGAGGCGCCCTG | 68 |
| SEQ ID NO: 796 | ACTTGCCCAGGTGGCCCAGGCTGAATCCCAGGTCCTCCTG | 65 |
| SEQ ID NO: 797 | TGGCCTCGTTTACCTGTGTCTGCCGCACACGCCCACTGCC | 65 |
| SEQ ID NO: 798 | GTCTGGCCCATACCTGCAGCGTCTTGGAGATCCTGGCCTT | 60 |
| SEQ ID NO: 799 | GCTCCCCCCACCTTGTGTCCCTCGGTCCCCAGCCCCACCT | 73 |
| SEQ ID NO: 800 | TGCAGGGTCCGCTGTGGGGAGGACAGGGAGGCTGCGATCT | 68 |
| SEQ ID NO: 801 | TCGCGGATGGTGGACTTCCCGCCATATACGACGCTCTGCT | 60 |
| SEQ ID NO: 802 | AGTGGGGTGAAGGCCACGCTGGAGGCCGTGCCCGAGGAGC | 73 |
| SEQ ID NO: 803 | CGGCTGCTGAGCCTAACCACCTCCTGGGCTTCTTTCCAGC | 63 |
| SEQ ID NO: 804 | GCTCATGGTATCCCTACCGCAGGCAATCTGTGGACAGCAC | 58 |
| SEQ ID NO: 805 | CTGAATGTCACCTGAAGGGTCACAGAAGCTACTCACAGGG | 53 |
| SEQ ID NO: 806 | TTAAGTGTTCTCAATATGAGATTAGCTGGAGCCGCCTAAT | 40 |
| SEQ ID NO: 807 | GAAGATCCATCTGTTGGAAGCCAGAGGACTAGTGGGAAAC | 50 |
| SEQ ID NO: 808 | CCCCCACAGGGATCTGACACACAACTTAGGTTGTCAGCCA | 55 |
| SEQ ID NO: 809 | GCCCAGCTTCCCAAGTCCTGCCTGGACACCGCCCCATGGA | 68 |
| SEQ ID NO: 810 | AATCACCTTCATGCTTAAAACACTCACACTGATTTCCAGC | 40 |
| SEQ ID NO: 811 | CCTCTTGGGGACCTGGGTGACCTTACTCACCCTCATGGCT | 60 |
| SEQ ID NO: 812 | GTTGCTGTGGACAGGCTTGGAGCCGTTTTTGGCTGGAGAC | 58 |
| SEQ ID NO: 813 | GGAGGGGTAGGTGGGCGGCACAGCTGGGGACTGAGGGTGC | 73 |
| SEQ ID NO: 814 | GCCAGGAGTGGTGCTCAAGGCAGAGGCAGCAGGCGGGGGG | 73 |
| SEQ ID NO: 815 | CAGGGCACTTGGGGGTGCTGCGGGGGCGGGGACCCCATTG | 75 |
| SEQ ID NO: 816 | GGTGCCCGAGTTGTGGCTGGGAGCTGGACTGGCCTTGGGG | 70 |
| SEQ ID NO: 817 | CTGCTTGCCAGCCCCTCCACCGGCACTGCTGTTACTACTG | 63 |
| SEQ ID NO: 818 | GCCCCCCACCCCGCTGCCTCCTCACTCACTGGTGGCGCCA | 75 |
| SEQ ID NO: 819 | CGGGCTGTCTGCCACAACTGAGCTGTAACCTGGGAACAAA | 55 |
| SEQ ID NO: 820 | GCTGGCATTGTTGCCCCCACTGCTGCTCAAAGCCACCTCT | 60 |
| SEQ ID NO: 821 | AGGTGGGTTGTGGGGGCCGGAAGGGGGGCCCAAGGCCTGG | 75 |
| SEQ ID NO: 822 | TCCCAACCCTGCCGATGGCCGAGACACTCACGAGGTGCTG | 65 |
| SEQ ID NO: 823 | GGGGGTGAGGCGCCTGCGCCTCTCTGTTTCAAAAGGCTGC | 65 |
| SEQ ID NO: 824 | ATTCCCAGCAGCAAGGGCGGGGGGTTCAGAACCCACCGAT | 63 |
| SEQ ID NO: 825 | GGGGGTGTAACACCCGAGGGAGATGGAGGATAGCGCTTGG | 63 |
| SEQ ID NO: 826 | CAAAGCAGGGAGGCTGATGTAGTTTCCTTGCTGGAAAGAA | 48 |
| SEQ ID NO: 827 | CTTCCACTTAGATGAGAACGTATTTTAGAATGTTCTGAAG | 35 |
| SEQ ID NO: 828 | TAACAGAAATGGGGAGGAAAGGGTATGGGGCTCTTGAGAA | 48 |
| SEQ ID NO: 829 | AAACAGTGACCCTCCGGTGGCAGTCAATTGGCCTCAGGCA | 58 |
| SEQ ID NO: 830 | GCAGAGGAATAAGGACTTCGGGACAATTCACTTTGAAAAG | 43 |
| SEQ ID NO: 831 | GACCCAGTGGAATGGTCTGAGCTAAGATTTGAAGGAGTGG | 50 |
| SEQ ID NO: 832 | TGCACACTGATCTTTCTTAGGGCATTCTTCGGGAAACAGG | 48 |
| SEQ ID NO: 833 | GGCTCAGGATGAACAGCAACAGGGGTTGGGATGATCACTG | 55 |
| SEQ ID NO: 834 | GATCATGGAGATGTGATCTAGGGAACAAAGCCAGAGAAGG | 48 |
| SEQ ID NO: 835 | AGGCATTCCCACGGTGTGAGGTCAGATTGGGCAGGGCCTA | 60 |
| SEQ ID NO: 836 | AGAGCCAGCACTTGCTGTTCCACACATACTAGATCAGTCT | 48 |
| SEQ ID NO: 837 | TGGACAACCCCCTCCCACACCCAGAGCTGTGGAAGGGGAG | 65 |
| SEQ ID NO: 838 | CACCTAGATGCTGACCAAGGCCCTCCCCATGCTGCTGGAG | 63 |
| SEQ ID NO: 839 | ATAAAGCCTTCATTCTCCAGGACCCCGCCCTTGCCCTGTT | 55 |
| SEQ ID NO: 840 | AGGTGGTGAGTTTGGGGCTGGGGGGCCTCCCTGAGGAGCC | 70 |
| SEQ ID NO: 841 | GAGAGAACCAGGTCCCACATGCTGACACAGGTGTCCACGG | 60 |
| SEQ ID NO: 842 | ATCCCCCCAATCTCACCAGTGCACCCCACAGACAAGGCGA | 60 |
| SEQ ID NO: 843 | AAGGGCTTCAGCATAAGAGTCAGAACCCGCCCCCCTTCCT | 58 |
| SEQ ID NO: 844 | TGTGGGCTGAAGGGACGAGGCTGGGGCACTGGGTGGGAGG | 70 |
| SEQ ID NO: 845 | TTGCAATGTGGAAGAGTCAGGGGCACATTGTCTGGGCTGA | 53 |
| SEQ ID NO: 846 | TAAGTGGGAGGGAGCGGGGACCTAGTGTGGGCATGAGGAC | 63 |
| SEQ ID NO: 847 | GGAGCAGGGATTTGGCTGGGCAATGGAGAGAAAGGTCTGA | 55 |
| SEQ ID NO: 848 | ACACAGAGATGCCCAGGAACTTGCTCTTTAGTAAAGCAGC | 48 |
| SEQ ID NO: 849 | TGGAGAGAGGTCCTTGAAAGGTTTTGAACCCCATAAAGAG | 45 |
| SEQ ID NO: 850 | TCAGGAGGCAGCCCAGTGATAGGGTCCAAGGAACCAGTGG | 60 |
| SEQ ID NO: 851 | ACAGTCTACTGACTTTTCCTATTCAGCTGTGAGCATTCAA | 40 |
| SEQ ID NO: 852 | CTGTCCCCTGGACCTTGACACCTGGCTCCCCAACCCTGTC | 65 |
| SEQ ID NO: 853 | AGGAAACCCAGATTCCACCAGACACTTCCTTCTTCCCCCC | 55 |
| SEQ ID NO: 854 | GGCTATCTGGCCTGAGACAACAAATGCTGCCTCCCACCCT | 58 |
| SEQ ID NO: 855 | GTCTGGCACTGGGACTTTCAGAACTCCTCCTTCCCTGACT | 55 |
| SEQ ID NO: 856 | TTGCCCCAGACCCGTCATTCAATGGCTAGCTTTTTCCATG | 50 |
| SEQ ID NO: 857 | AAAAACACGAGCACCCCCAACCACAACGGCCAGTTCTCTG | 55 |
| SEQ ID NO: 858 | TTAACCTTGGACATGGTAAACCATCCAAAACCTTCCTCTC | 43 |
| SEQ ID NO: 859 | AGCAACTAAACCTCTCCACTGGGCACTTATCCTTGGTTTC | 48 |
| SEQ ID NO: 860 | GAACCTCTTATTCTCTTAGAACCCACAGCTGCCACCACAG | 50 |
| SEQ ID NO: 861 | TCCCTTCTCCCAGTGTAAGACCCCAAATCACTCCAAATGA | 48 |
| SEQ ID NO: 862 | CAACCCCCAACCCGATGCCTGCTTCAGATGTTTCCCATGT | 55 |
| SEQ ID NO: 863 | CATAAACCTGGCTCCTAAAGGCTAAATATTTTGTTGGAGA | 38 |
| SEQ ID NO: 864 | CTGCTGACCTGCCCTCCCAGGTCAGAATCATCCTCATGCA | 58 |
| SEQ ID NO: 865 | TGTTCTCCAGACCTGTGCACTCTATCTGTGCAACAGAGAT | 48 |
| SEQ ID NO: 866 | CGTGCAGCAAACAATGTGGAATTCCAATAACCCCCCACTC | 50 |
| SEQ ID NO: 867 | AAATATGAGTCTCCCAAAGTTCCCTAGCATTTCAAAATCC | 38 |
| SEQ ID NO: 868 | CATCATAAAAAGATCTTGTGGTCCACAGATCCTCTAGCCC | 45 |
| SEQ ID NO: 869 | CTCCCAACCCAGAATCCAGCTCCACAGATACATTGCTACT | 50 |
| SEQ ID NO: 870 | CACTCTGAGACCAGAAACTAGAACTTTTATTCCTCATGCT | 40 |
| SEQ ID NO: 871 | CACCAGCACTCAGGAGATTGTGAGACTCCCTGATCCCTGC | 58 |
| SEQ ID NO: 872 | TGCCTAGATCCTTTGCACTCCAAGACCCAGTGTGCCCTAA | 53 |
| SEQ ID NO: 873 | GGGGGTGGGTACGATCCCCGATTCTTCATACAAAGCCTCA | 55 |
| SEQ ID NO: 874 | GGACAAAGGCAGAGGAGACACGCCCAGGATGAAACAGAAA | 53 |
| SEQ ID NO: 875 | TGGATGCACCAGGCCCTGTAGCTCATGGAGACTTCATCTA | 53 |
| SEQ ID NO: 876 | GGGAGAGCTAGCACTTGCTGTTCTGCAATTACTAGATCAC | 48 |
| SEQ ID NO: 877 | GGCTGGACAACCCCCTCCCACACCCAGAGCTGTGGAAGGG | 68 |
| SEQ ID NO: 878 | TGGCACCCAGAGGCTGACCAAGGCCCTCCCCATGCTGCTG | 68 |
| SEQ ID NO: 879 | CCTATAAAACCTTCATTCCCCAGGACTCCGCCCCTGCCCT | 58 |
| SEQ ID NO: 880 | TGCAGGTGGTAAGCTTGGGGCTGGGGAGCCTCCCCCAGGA | 68 |
| SEQ ID NO: 881 | AGGAAGACAACCGGGACCCACATGGTGACACAGCTCTCCG | 60 |
| SEQ ID NO: 882 | CAACCATGGCCCCTCTCACCAATCCACGTCACGGACAGGG | 63 |
| SEQ ID NO: 883 | TCAGCTTGACAGTCAGGGCTGGCTCCCTCTCCTGCATCCC | 63 |
| SEQ ID NO: 884 | TCCCTGTCTGGGCTGGGGTGCTGGGTTGGGGGGGAAAGAG | 68 |
| SEQ ID NO: 885 | TGTGGGAGTGAGGACTGTTGCAATATGGAGGGGCTGGGGG | 60 |
| SEQ ID NO: 886 | GGGAGAAAGTTCTGGGGTAAGTGGGAGGGAGCGGGGACCT | 63 |
| SEQ ID NO: 887 | TTGTGGGGCTCAAAACCTCCAAGGACCTCTCTCAATGCCA | 53 |
| SEQ ID NO: 888 | TGCCCAACCCTATCCCAGAGACCTTGATGCTTGGCCTCCC | 60 |
| SEQ ID NO: 889 | TCTTGCCCTAGGATACCCAGATGCCAACCAGACACCTCCT | 55 |
| SEQ ID NO: 890 | TTCCTAGCCAGGCTATCTGGCCTGAGACAACAAATGGGTC | 53 |
| SEQ ID NO: 891 | TCTTAGCCCCAGACTCTTCATTCAGTGGCCCACATTTTCC | 50 |
| SEQ ID NO: 892 | AGGAAAAACATGAGCATCCCCAGCCACAACTGCCAGCTCT | 53 |
| SEQ ID NO: 893 | CCCCTTCAGAGTTACTGACAAACAGGTGGGCACTGAGACT | 53 |
| SEQ ID NO: 894 | TGGAAAGTTAGCTTATTTGTTTGCAAGTCAGTAAAATGTC | 33 |
| SEQ ID NO: 895 | GACTCAGGAGTCTCATGGACTCTGCCAGCATTCACAAAAC | 50 |
| SEQ ID NO: 896 | ATGCTGTCTGCTAAGCTGTGAGCAGTAAAAGCCTTTGCCT | 48 |
| SEQ ID NO: 897 | GATTTGGGGGGGGCAAGGTGTACTAATGTGAACATGAACC | 50 |
| SEQ ID NO: 898 | GTGTGCACAGCATCCACCTAGACTGCTCTGGTCACCCTAC | 58 |
| SEQ ID NO: 899 | AGGATTCCTAATCTCAGGTTTCTCACCAGTGGCACAAACC | 48 |
| SEQ ID NO: 900 | CAAAGGCTGAGCAGGTTTGCAAGTTGTCCCAGTATAAGAT | 45 |
| SEQ ID NO: 901 | GTCAAGGACAATCGATACAATATGTTCCTCCAGAGTAGGT | 43 |
| SEQ ID NO: 902 | GCAAGATGATATCTCTCTCAGATCCAGGCTTGCTTACTGT | 45 |
| SEQ ID NO: 903 | TCTGTGTGTCTTCTGAGCAAAGACAGCAACACCTTTTTTT | 40 |
| SEQ ID NO: 904 | AACGTTGAGACTGTCCTGCAGACAAGGGTGGAAGGCTCTG | 55 |
| SEQ ID NO: 905 | CATAAATAAGCAGGATGTGACAGAAGAAGTATTTAATGGT | 33 |
| SEQ ID NO: 906 | GCTGCCAGACACAGTCGATCGGGACCTAGAACCTTGGTTA | 55 |
| SEQ ID NO: 907 | GGGATCCTGAGCGCTGCCTTATTCTGGGTTTGGCAGTGGA | 58 |
| SEQ ID NO: 908 | TCACTCAAACCCAGAAGTTCTGATCCCCAGCCATGCCCCT | 55 |
| SEQ ID NO: 909 | AGCCTCTTCCTCCTTTGAAATTCAAGAGGGTGGACCCACT | 50 |
| SEQ ID NO: 910 | GGAGCTGGGACCTTACCAGTCTCCTCCCTCATTGACCTAA | 55 |
| SEQ ID NO: 911 | GAGGATATGAGATTCTTAGGCCATTCCCACATCAGTACCT | 45 |
| SEQ ID NO: 912 | TACCCAGAACTCTACCCCTCAGGATTCCAGCACCTTCTTC | 53 |
| SEQ ID NO: 913 | GCCTCTGCCCTTCAGGGGCCAAAGAGCCTTAAGCCACAAA | 58 |
| SEQ ID NO: 914 | ATCCCATTACTATCACCCCAAACCCTGGACCTAATGGTTC | 48 |
| SEQ ID NO: 915 | AATGGGCAACCCTCGATCCTCAGACTCTTGAGGAATCAAG | 50 |
| SEQ ID NO: 916 | GATACCCTCAAGTGGAGTAAGGATTAGGTGGCAAGATGGA | 48 |
| SEQ ID NO: 917 | GTGCTTGCCCAGGGGCACCTTCATGGAGCTAGAAGGGCTG | 63 |
| SEQ ID NO: 918 | GATGACACCCAAGGCCTCTGGGGCATCTTTCATGCTCAGA | 55 |
| SEQ ID NO: 919 | TGCTGGCCACACCCTCAGAGTGTGGATGCTGGATGATGAG | 58 |
| SEQ ID NO: 920 | GAGGCACGCTGCAGGGATAGTCACAGCAACATGACGTCAT | 55 |
| SEQ ID NO: 921 | AGAGGAGGATGTCGGCAGCTCTACGGTTGGCAGGTGGCTG | 63 |
| SEQ ID NO: 922 | GACACTAGGCCTCAGCCTGGCACCATGCAGGCCACTCCCA | 65 |
| SEQ ID NO: 923 | ACTTTTGAGTCCTGGATCCCTATGATTCCAGGCTCCCTGT | 50 |
| SEQ ID NO: 924 | CCTTGAGATTTCATGGATGGTGACATATGGCCATTCTCTA | 43 |
| SEQ ID NO: 925 | AAAACCCATAAGTTCAGGTCCCTGTGCCCTCCACCCAGAA | 53 |
| SEQ ID NO: 926 | TCGTATCTGGGAGACTCACTTGGGAGAGCAATAGACTTGG | 50 |
| SEQ ID NO: 927 | TACAAGATGTGGTGGAGATAAGGCTGATGCTGGCACAGTG | 50 |
| SEQ ID NO: 928 | GTACACACCATGGTGTTCATCAGGGCCCTGGGTAGTCCCT | 58 |
| SEQ ID NO: 929 | GCTGTGACCTCACAGGAGTCCGTGCCTCCACCCCCTACTC | 65 |
| SEQ ID NO: 930 | TTGGCTGACCTGATTGCTGTGTCCTGTGTCAGCTGCTGCT | 55 |
| SEQ ID NO: 931 | ATGTACCATTTGCCCCTGGATGTTCTGCACTATAGGGTAA | 45 |
| SEQ ID NO: 932 | TACTTTTACCCATGCATTTAAAGTTCTAGGTGATATGGCC | 38 |
| SEQ ID NO: 933 | AAACATGGGTATCACTTCTGGGCTGAAAGCCTTCTCTTCT | 45 |
| SEQ ID NO: 934 | GGTGTTTAAATCTTGTGGGGTGGCTCCTTCTGATAATGCT | 45 |
| SEQ ID NO: 935 | CATTTGCATGGCTGCTTGATGTCCCCCCACTGTGTTTAGC | 53 |
| SEQ ID NO: 936 | CATCTGGCCTGGTGCAATAGGCCCTGCATGCACTGGATGC | 60 |
| SEQ ID NO: 937 | GGTACTAGTAGTTCCTGCTATGTCACTTCCCCTTGGTTCT | 48 |
| SEQ ID NO: 938 | GATAGGTGGATTATTTGTCATCCATCCTATTTGTTCCTGA | 38 |
| SEQ ID NO: 939 | GTCCAGAATGCTGGTAGGGCTATACATTCTTACTATTTTA | 38 |
| SEQ ID NO: 940 | GTCTACATAGTCTCTAAAGGGTTCCTTTGGTCCTTGTCTT | 43 |
| SEQ ID NO: 941 | CTCCTGTGAAGCTTGCTCGGCTCTTAGAGTTTTATAGAAC | 45 |
| SEQ ID NO: 942 | CGCATTTTGGACCAACAAGGTTTCTGTCATCCAATTTTTT | 38 |
| SEQ ID NO: 943 | TCCTACTCCCTGACATGCTGTCATCATTTCTTCTAGTGTA | 43 |
| SEQ ID NO: 944 | GCTCATTGCTTCAGCCAAAACTCTTGCCTTATGGCCGGGT | 53 |
| SEQ ID NO: 945 | ATTGCCTCTCTGCATCATTATGGTAGCTGAATTTGTTACT | 38 |
| SEQ ID NO: 946 | GCCACAATTGAAACACTTAACAATCTTTCTTTGGTTCCTA | 35 |
| SEQ ID NO: 947 | TTTCCTAGGGGCCCTGCAATTTCTGGCTGTGTGCCCTTCT | 55 |
| SEQ ID NO: 948 | CCCAGACCTGAAGCTCTCTTCTGGTGGGGCTGTTGGCTCT | 60 |
| SEQ ID NO: 949 | GTCTATCGGCTCCTGCTTCTGAGGGGGAGTTGTTGTCTCT | 55 |
| SEQ ID NO: 950 | GCCAAAGAGTGACCTGAGGGAAGTTAAAGGATACAGTTCC | 48 |
| SEQ ID NO: 951 | CCTTTAGTTGCCCCCCTATCTTTATTGTGACGAGGGGTCG | 53 |
| SEQ ID NO: 952 | CTTCTAATACTGTATCATCTGCTCCTGTATCTAATAGAGC | 38 |
| SEQ ID NO: 953 | GTATCTGATCATACTGTCTTACTTTGATAAAACCTCCAAT | 33 |
| SEQ ID NO: 954 | CTAATACTGTACCTATAGCTTTATGTCCACAGATTTCTAT | 33 |
| SEQ ID NO: 955 | TCAACAGATTTCTTCCAATTATGTTGACAGGTGTAGGTCC | 40 |
| SEQ ID NO: 956 | TTGGGCCATCCATTCCTGGCTTTAATTTTACTGGTACAGT | 43 |
| SEQ ID NO: 957 | CAAATACTGGAGTATTGTATGGATTTTCAGGCCCAATTTT | 35 |
| SEQ ID NO: 958 | CTTCCCAGAAGTCTTGAGTTCTCTTATTAAGTTCTCTGAA | 38 |
| SEQ ID NO: 959 | CTGAAAAATATGCATCACCCACATCCAGTACTGTTACTGA | 40 |
| SEQ ID NO: 960 | TGGTAAATGCAGTATACTTCCTGAAGTCTTCATCTAAGGG | 40 |
| SEQ ID NO: 961 | ACTGATATCTAATCCCTGGTGTCTCATTGTTTATACTAGG | 38 |
| SEQ ID NO: 962 | ATATTGCTGGTGATCCTTTCCATCCCTGTGGAAGCACATT | 45 |
| SEQ ID NO: 963 | GTTTTCTAAAAGGCTCTAAGATTTTTGTCATGCTACTTTG | 33 |
| SEQ ID NO: 964 | ACAAATCATCCATGTATTGATAGATAACTATGTCTGGATT | 30 |
| SEQ ID NO: 965 | TTTTTGTTCTATGCTGCCCTATTTCTAAGTCAGATCCTAC | 38 |
| SEQ ID NO: 966 | TGGTAAGTCCCCACCTCAACAGATGTTGTCTCAGCTCCTC | 53 |
| SEQ ID NO: 967 | TAGGCTGTACTGTCCATTTATCAGGATGGAGTTCATAACC | 43 |
| SEQ ID NO: 968 | GTATGTCATTGACAGTCCAGCTGTCTTTTTCTGGCAGCAC | 48 |
| SEQ ID NO: 969 | GGTAAATCTGACTTGCCCAATTCAATTTCCCCACTAACTT | 40 |
| SEQ ID NO: 970 | TTCCTCTAAGGAGTTTACATAATTGCCTTACTTTAATCCC | 35 |
| SEQ ID NO: 971 | CTGCTTCTTCTGTTAGTGGTATTACTTCTGTTAGTGCTTT | 38 |
| SEQ ID NO: 972 | CTGCTATTAAGTCTTTTGATGGGTCATAATACACTCCATG | 38 |
| SEQ ID NO: 973 | AAATTTGATATGTCCATTGGCCTTGCCCCTGCTTCTGTAT | 43 |
| SEQ ID NO: 974 | CTGTTAATTGTTTTACATCATTAGTGTGGGCACCCCTCAT | 40 |
| SEQ ID NO: 975 | ATGTTTCCTTTTGTATGGGCAGTTTAAATTTAGGAGTCTT | 33 |
| SEQ ID NO: 976 | GAATCCAGGTGGCTTGCCAATACTCTGTCCACCATGTTTC | 50 |
| SEQ ID NO: 977 | ATAATTTCACTAAGGGAGGGGTATTAACAAACTCCCACTC | 40 |
| SEQ ID NO: 978 | AGGTTTCTGCTCCTACTATGGGTTCTTTCTCTAACTGGTA | 43 |
| SEQ ID NO: 979 | TTCCTAATTTAGTCTCCCTGTTAGCTGCCCCATCTACATA | 43 |
| SEQ ID NO: 980 | TTGCTTGTAACTCAGTCTTCTGATTTGTTGTGTCAGTTAG | 38 |
| SEQ ID NO: 981 | CTATGTTTACTTCTAATCCCGAATCCTGCAAAGCTAGATA | 38 |
| SEQ ID NO: 982 | GTTGTGCTTGAATGATTCCTAATGCATATTGTGAGTCTGT | 38 |
| SEQ ID NO: 983 | GCTCTATTATTTGATTGACTAACTCTGATTCACTTTGATC | 33 |
| SEQ ID NO: 984 | TCCAATTACTGTGATATTTCTCATGTTCATCTTGGGCCTT | 38 |
| SEQ ID NO: 985 | TTGCTACTACAGGTGGCAGGTTAAAATCACTAGCCATTGC | 45 |
| SEQ ID NO: 986 | CTCCTTTTAGCTGACATTTATCACAGCTGGCTACTATTTC | 40 |
| SEQ ID NO: 987 | CTACCAGGATAACTTTTCCTTCTAAATGTGTACAATCTAG | 35 |
| SEQ ID NO: 988 | GAATAACTTCTGCTTCTATATATCCACTGGCTACATGAAC | 38 |
| SEQ ID NO: 989 | ACCAACAGGCGGCCCTAACCGTAGCACCGGTGAAATTGCT | 58 |
| SEQ ID NO: 990 | GGGGATTGTAGGGAATTCCAAATTCCTGCTTGATTCCCGC | 50 |
| SEQ ID NO: 991 | TCTTAAGATGTTCAGCCTGATCTCTTACCTGTCCTATAAT | 38 |
| SEQ ID NO: 992 | CTACTATTCTTTCCCCTGCACTGTACCCCCCAATCCCCCC | 58 |
| SEQ ID NO: 993 | TCCAGAGGAGCTTTGCTGGTCCTTTCCAAAGTGGATTTCT | 48 |
| SEQ ID NO: 994 | TTATGTCACTATTATCTTGTATTACTACTGCCCCTTCACC | 38 |
| SEQ ID NO: 995 | CCTGTCTACTTGCCACACAATCATCACCTGCCATCTGTTT | 48 |
| SEQ ID NO: 996 | CATATGGTGTTTTACTAAACTTTTCCATGTTCTAATCCTC | 33 |
| SEQ ID NO: 997 | GTGATGTCTATAAAACCATCCCCTAGCTTTCCCTGAAACA | 43 |
| SEQ ID NO: 998 | GATGTGTACTTCTGAACTTATTCTTGGATGAGGGCTTTCA | 40 |
| SEQ ID NO: 999 | ACCCCAATATGTTGTTATTACCAATCTAGCATCCCCTAGT | 40 |
| SEQ ID NO: 1000 | GTCAAAGTAATACAGATGAATTAGTTGGTCTGCTAGTTCA | 35 |
| SEQ ID NO: 1001 | GTGTCCTAATAAGGCCTTTCTTATAGCAGAGTCTGAAAAA | 38 |
| SEQ ID NO: 1002 | CTTGTTATGTCCTGCTTGATATTCACACCTAGGGCTAACT | 43 |
| SEQ ID NO: 1003 | TGTTATTAATGCTGCTAGTGCCAAGTATTGTAGAGATCCT | 38 |
| SEQ ID NO: 1004 | CAGTTTCGTAACACTAGGCAAAGGTGGCTTTATCTTTTTT | 38 |
| SEQ ID NO: 1005 | GTGGCCCTTGGTCTTCTGGGGCTTGTTCCATCTATCCTCT | 55 |
| SEQ ID NO: 1006 | CCTCTAAAAGCTCTAGTGTCCATTCATTGTGTGGCTCCCT | 48 |
| SEQ ID NO: 1007 | GCCAAATCCTAGGAAAATGTCTAACAGCTTCATTCTTAAG | 38 |
| SEQ ID NO: 1008 | TATCCCCATAAGTTTCATAGATATGTTGCCCTAAGCCATG | 40 |
| SEQ ID NO: 1009 | GTTGTTGCAGAATTCTTATTATGGCTTCCACTCCTGCCCA | 45 |
| SEQ ID NO: 1010 | TCTGCTATGTCGACACCCAATTCTGAAAATGGATAAACAG | 40 |
| SEQ ID NO: 1011 | ACTGGCTCCATTTCTTGCTCTCCTCTGTCGAGTAACGCCT | 53 |
| SEQ ID NO: 1012 | GGCTGACTTCCTGGATGCTTCCAGGGCTCTAGTCTAGGAT | 55 |
| SEQ ID NO: 1013 | GAGATGCCTAAGGCTTTTGTTATGAAACAAACTTGGCAAT | 38 |
| SEQ ID NO: 1014 | TGATGAGCTCTTCGTCGCTGTCTCCGCTTCTTCCTGCCAT | 55 |
| SEQ ID NO: 1015 | ACTTACTGCTTTGATAGAGAAGCTTGATGAGTCTGACTGT | 40 |
| SEQ ID NO: 1016 | GCTACTATTGCTACTATTGGTATAGGTTGCATTACATGTA | 35 |
| SEQ ID NO: 1017 | CTGTCTTCTGCTCTTTCTATTAGTCTATCAATTAACCTGT | 35 |
| SEQ ID NO: 1018 | TCATCAACATCCCAAGGAGCATGGTGCCCCATCTCCACCC | 58 |
| SEQ ID NO: 1019 | CATAATAGACTGTGACCCACAATTTTTCTGTAGCACTACA | 38 |
| SEQ ID NO: 1020 | CACAAAATAGAGTGGTGGTTGCTTCCTTCCACACAGGTAC | 48 |
| SEQ ID NO: 1021 | AAACATTATGTACCTCTGTATCATATGCTTTAGCATCTGA | 33 |
| SEQ ID NO: 1022 | CTTGTGGGTTGGGGTCTGTGGGTACACAGGCATGTGTGGC | 60 |
| SEQ ID NO: 1023 | AACTGATTATATCCTCATGCATCTGTTCTACCATGTCATT | 35 |
| SEQ ID NO: 1024 | GTGGGGTTAATTTTACACATGGCTTTAGGCTTTGATCCCA | 43 |
| SEQ ID NO: 1025 | TAGTATCATTCTTCAAATCAGTGCACTTTAAACTAACACA | 30 |
| SEQ ID NO: 1026 | CTCCTTTCTCCATTATCATTCTCCCGCTACTACTATTGGT | 43 |
| SEQ ID NO: 1027 | TTGTCAACTTATAGCTGGTAGTATCATTATCTATTGGTAT | 30 |
| SEQ ID NO: 1028 | ATACCTTTGGACAGGCCTGTGTAATGACTGAGGTGTTACA | 45 |
| SEQ ID NO: 1029 | TTCCATGTGTACATTGTACTGTGCTGACATTTGTACATGG | 40 |
| SEQ ID NO: 1030 | GACTGCCATTTAACAGCAGTTGAGTTGATACTACTGGCCT | 45 |
| SEQ ID NO: 1031 | CCGTGAAATTGACAGATCTAATTACTACCTCTTCTTCTGC | 40 |
| SEQ ID NO: 1032 | CTACAGATGTGTTCAGCTGTACTATTATGGTTTTAGCATT | 35 |
| SEQ ID NO: 1033 | CTATTGTAACAAATGCTCTCCCTGGTCCTCTCTGGATACG | 48 |
| SEQ ID NO: 1034 | TACTAATGTTACAATGTGCTTGTCTCATATTTCCTATTTT | 28 |
| SEQ ID NO: 1035 | ATTTGCTAGCTATCTGTTTTAAAGTGTTATTCCATTTTGC | 30 |
| SEQ ID NO: 1036 | TAAAACTGTGCGTTACAATTTCTGGGTCCCCTCCTGAGGA | 48 |
| SEQ ID NO: 1037 | ACAGTTGTGTTGAATTACAGTAGAAAAATTCCCCTCCACA | 38 |
| SEQ ID NO: 1038 | ACCCTTCAGTACTCCAAGTACTATTAAACCAAGTACTATT | 35 |
| SEQ ID NO: 1039 | TGCATGGGAGGGTGATTGTGTCACTTCCTTCAGTGTTATT | 45 |
| SEQ ID NO: 1040 | ATGAACATCTAATTTGTCCACTGATGGGAGGGGCATACAT | 43 |
| SEQ ID NO: 1041 | TATTACCACCATCTCTTGTTAATAGCAGCCCTGTAATATT | 35 |
| SEQ ID NO: 1042 | TATCTCCTCCTCCAGGTCTGAAGATCTCGGACTCATTGTT | 48 |
| SEQ ID NO: 1043 | GTGGTAGCTGAAGAGGCACAGGCTCCGCAGATCGTCCCAG | 63 |
| SEQ ID NO: 1044 | TTCCACAATCCTCGTTACAATCAAGAGTAAGTCTCTCAAG | 40 |
| SEQ ID NO: 1045 | CCACCAATATTTGAGGGCTTCCCACCCCCTGCGTCCCAGA | 60 |
| SEQ ID NO: 1046 | AGCACTATTCTTTAGTTCCTGACTCCAATACTGTAGGAGA | 40 |
| SEQ ID NO: 1047 | CCCCTCAGCTACTGCTATGGCTGTGGCATTGAGCAAGCTA | 55 |
| SEQ ID NO: 1048 | AGCTCTACAAGCTCCTTGTACTACTTCTATAACCCTATCT | 40 |
| SEQ ID NO: 1049 | ACACTACTTTTTGACCACTTGCCACCCATCTTATAGCAAA | 40 |
| SEQ ID NO: 1050 | TCAGCTCGTCTCATTCTTTCCCTTACAGTAGGCCATCCAA | 48 |
| SEQ ID NO: 1051 | TCCAGGTCTCGAGATGCTGCTCCCACCCTATCTGCTGCTG | 60 |
| SEQ ID NO: 1052 | TTGGTAGCTGCTGTATTGCTACTTGTGATTGCTCCATGTT | 43 |
| SEQ ID NO: 1053 | GTCATTGGTCTTAAAGGTACCTGAGGTGTGACTGGAAAAC | 45 |
| SEQ ID NO: 1054 | TCTTGTCTTCTTTGGGAGTGAATTAGCCCTTCCAGTCCCC | 50 |
| SEQ ID NO: 1055 | GGGAAGTAGCCTTGTGTGTGGTAGATCCACAGATCAAGGA | 50 |
| SEQ ID NO: 1056 | GGATATCTGACCCCTGGCCCTGGTGTGTAGTTCTGCTAAT | 53 |
| SEQ ID NO: 1057 | GGCTCAACTGGTACTAGCTTGTAGCACCATCCAAAGGTCA | 50 |
| SEQ ID NO: 1058 | AAGCTGGTGTTCTCTCCTTTATTGGCCTCTTCTATCTTAT | 40 |
| SEQ ID NO: 1059 | CTCTCCGGGTCATCCATCCCATGCAGGCTCACAGGGTGTA | 60 |
| SEQ ID NO: 1060 | TGAAATGCTAGGCGGCTGTCAAACCTCCACTCTAACACTT | 48 |
| SEQ ID NO: 1061 | CAGTTCTTGAAGTACTCCGGATGCAGCTCTCGGGCCACGT | 58 |

A nucleic acid probe may be a non-labeled probe, or a probe that does not contain a detectable moiety. A non-labeled probe may further interact with a labeled probe (e.g., a labeled nucleic acid probe). A non-labeled probe may hybridize with a labeled nucleic acid probe. A non-labeled probe may also interact with a labeled polypeptide probe. The labeled polypeptide probe may be a protein that recognizes a sequence within the non-labeled probe. A labeled probe may include a nucleic acid portion and a polypeptide tag portion and the polypeptide tag portion may further interact with a molecule comprising a detectable moiety. For example, a non-labeled probe may be a nucleic acid probe comprising a streptavidin which may interact with a biotinylated molecule comprising a detectable moiety.

A nucleic acid probe may comprise about 95%, about 96%, about 97%, about 98%, about 99%, or about 100% sequence specificity or sequence complementarity to a target site of a regulatory element. A nucleic acid probe may comprise about 95%, about 96%, about 97%, about 98%, about 99%, or about 100% sequence specificity or sequence complementarity to a target nucleic acid sequence. A nucleic acid probe may comprise about 95%, about 96%, about 97%, about 98%, about 99%, or about 100% sequence specificity or sequence complementarity to a target viral nucleic acid sequence The hybridization may be a high stringent hybridization condition.

A nucleic acid probe may hybridize with a genomic sequence that is present in low or single copy numbers (e.g., genomic sequences that are not repetitive elements). As used herein, repetitive element refers to a DNA sequence that is present in many identical or similar copies in the genome. Repetitive elements are not intended to refer to a DNA sequence that is present on each copy of the same chromosome (e.g., a DNA sequence that is present only once, but is found on both copies of chromosome 11, would not be considered a repetitive element, and would be considered a sequence that is present in the genome as one copy). The genome may consist of three broad sequence components: single copy or at least very low copy number DNA (approximately 60% of the human genome); moderately repetitive elements (approximately 30% of the human genome); and highly repetitive elements (approximately 10% of the human genome). For a review, see Human Molecular Genetics, Chapter 7 (1999), John Wiley & Sons, Inc.

A nucleic acid probe may have reduced off-target interaction. For example, “off-target” or “off-target interaction” may refer to an instance in which a nucleic acid probe against a given target hybridizes or interact with another target site (e.g., a different DNA sequence, RNA sequence, or a cellular protein or other moiety).

A nucleic acid probe may further be cross-linked to a target site of a regulatory element. For example, the nucleic acid probe may be cross-linked by a photo-crosslinking means such as UV or by a chemical cross-linking means such as by formaldehyde, or through a reactive group within the nucleic acid probe. Reactive group may include sulfhydryl-reactive linkers such as bismaleimidohexane (BMH), and the like.

A nucleic acid probe may include natural or unnatural nucleotide analogues or bases or a combination thereof. The unnatural nucleotide analogues or bases may comprise modifications at one or more of ribose moiety, phosphate moiety, nucleoside moiety, or a combination thereof. The unnatural nucleotide analogues or bases may comprise 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, locked nucleic acid (LNA), ethylene nucleic acid (ENA), peptide nucleic acid (PNA), 1′, 5′-anhydrohexitol nucleic acids (HNA), morpholino, methylphosphonate nucleotides, thiophosphonate nucleotides, or 2′-fluoro N3-P5′-phosphoramidites. The nucleic acid probes may further comprise one or more abasic sites. The abasic site may further be functionalized with a detectable moiety.

A nucleic acid probe may be a locked nucleic acid probe (such as a labeled locked nucleic acid probe), a labeled or unlabeled peptide nucleic acid (PNA) probe, a labeled or unlabeled oligonucleotide, an oligopaint, an ECHO probe, a molecular beacon probe, a padlock (or molecular inversion probe), a labeled or unlabeled toe-hold probe, a labeled TALE probe, a labeled ZFN probe, or a labeled CRISPR probe.

A nucleic acid probe may be a labeled or unlabeled locked nucleic acid probe or a labeled or unlabeled peptide nucleic acid probe. Locked nucleic acid probes and peptide nucleic acid probes are known to those of skill in the art and are described in Briones et al., Anal Bioanal Chem (2012) 402:3071-3089.

A nucleic acid probe may be a padlock (or molecular inversion probe). A padlock probe may be hybridized to a target regulatory element sequence in which the two ends may correspond to the target sequence. A padlock probe may be ligated together by a ligase (such as T4 ligase) when bound to the target sequence. An amplification (such as a rolling circle amplification or RCA) may be performed utilizing for example 29 polymerase, which may result in a single stranded DNA comprising multiple tandem copies of the target sequence.

A nucleic acid probe may be an oligopaint as described in U.S. Publication No. 2010/0304994; and in Beliveau, et al., “Versatile design and synthesis platform for visualizing genomes with oligopaint FISH probes,” PNAS 109(52): 21301-21306 (2012). Oligopaint may refer to detectably labeled polynucleotides that have sequences complementary to an oligonucleotide sequence (such as a portion of a DNA sequence, like a particular chromosome or sub-chromosomal region of a particular chromosome). Oligopaints may be generated from synthetic probes and arrays that are, optionally, computationally patterned (rather than using natural DNA sequences and/or chromosomes as a template).

A nucleic acid probe can be a labeled or unlabeled toe-hold probe. Toe-hold probes are known to those of skill in the art as described in Zhang et al., Optimizing the Specificity of Nucleic Acid Hybridization, Nature Chemistry 4: 208-214 (2012).

A nucleic acid probe may be a molecular beacon. Molecular beacons may be hairpin shaped molecules with an internally quenched fluorophore whose fluorescence is restored when they bind to a target nucleic acid sequence. Molecular beacons are known to those of skill in the art as described in Guo et al., Anal. Bioanal. Chem. (2012) 4023115-3125.

A nucleic acid probe may be an ECHO probe. ECHO probes may be sequence-specific, hybridization-sensitive, quencher-free fluorescent probes for RNA detection, which may be designed using the concept of fluorescence quenching caused by intramolecular excitonic interaction of fluorescent dyes. ECHO probes are known to those of skill in the art as described in Kubota et al., PLoS ONE, Vol. 5, Issue 9, e13003 (2010); or Okamoto, Chem. Soc. Rev., 2011, 40, 5815-5828, Wang et al., RNA (2012), 18:166-175.

A probe may be a clustered regularly interspaced palindromic repeat (CRISPR) probe. The CRISPR system may use a Cas9 protein to recognize DNA sequences, in which the target specificity may be solely determined by a small guide (sg) RNA and a protospacer adjacent motif (PAM). Upon binding to target DNA, the Cas9-sgRNA complex may generate a DNA double-stranded break. For imaging applications, a Cas9 protein may be replaced with an endonuclease-deactivated Cas9 (dCas9) protein. For example, imaging a cell, such as by fluorescence in situ hybridization (FISH), may be achieved by synthesizing a dCas9 within the cell, synthesizing RNA within the cell to bind genomic DNA and to complex with the dCas9 forming a dCas9/RNA complex, labeling the dCas9/RNA complex, and imaging the labeled dCas9/RNA complex within the live cell bound to genomic DNA. The endonuclease-deactivated Cas9 may be synthesized in vivo by using an integrated construct, a transiently transfected construct, by injection into the cell of a syncytia of nuclei or via electroporation into cells and/or nuclei.

A probe may comprise an endonuclease-deactivated Cas9 (dCas9) protein as described in Chen et al., “Dynamic imaging of genomic loci in living human cells by an optimized CRISPR/Cas system,” Cell 155(7): 1479-1491 (2013); or Ma et al., “Multicolor CRISPR labeling of chromosomal loci in human cells,” PNAS 112(10): 3002-3007 (2015). The dCas9 protein may be further labeled with a detectable moiety.

The RNA of the Cas9/RNA complex may be synthesized in vivo by using an integrated construct, a transiently transfected construct, by injection into the cell of a syncytia of nuclei or via electroporation into cells and/or nuclei. The Cas9/RNA complex may be labeled by making a fusion protein that includes Cas9 and a reporter, by injection of RNA that has been attached to a reporter into the cell or by a syncytia of nuclei including RNA that has been attached to a reporter, by electroporation into cells or nuclei or by indirect labeling of the RNA by hybridization with a labeled secondary oligonucleotide. The label may be a conditional reporter, based on the binding of Cas9/RNA to the target nucleic acid. The label may be quenched and may then be activated upon the Cas9/RNA complex binding to the target nucleic acid. A probe may be a transcription activator-like effector nuclease (TALEN) probe or a zinc-finger nuclease (ZFN) probe.

A probe disclosed herein may be a polypeptide probe. A polypeptide probe may include a protein or a binding fragment thereof that interacts with a target site (such as a nucleic acid target site or a protein target) of interest. A polypeptide probe may comprise a DNA-binding protein, a RNA-binding protein, a protein involved in the transcription/translation process or detects the transcription/translation process, a protein that may detect an open or relaxed portion of a chromatin, or a protein interacting partner of a product of a regulatory element.

A polypeptide probe may be a DNA-binding protein. The DNA-binding protein may be a transcription factor that modulates the transcription process, polymerases, or histones. A DNA-binding protein may comprise a zinc finger domain, a helix-turn-helix domain, a leucine zipper domain (such as a basic leucine zipper domain), a high mobility group box (HMG-box) domain, and the like. The DNA-binding protein may interact with a nucleic acid region in a sequence specific manner. The DNA-binding protein may interact with a nucleic acid region in a sequence non-specific manner. The DNA-binding protein may interact with single-stranded DNA. The DNA-binding protein may interact with double-stranded DNA. The DNA-binding protein probe may further comprise a detectable moiety.

A polypeptide probe may be a RNA-binding protein. The RNA-binding protein may participate in forming ribonucleoprotein complexes. The RNA-binding protein may modulate post-transcription such as in splicing, polyadenylation, mRNA stabilization, mRNA localization, or in translation. A RNA-binding protein may comprise a RNA recognition motif (RRM), dsRNA binding domain, zinc finger domain, K-Homology domain (KH domain), and the like. The RNA-binding protein may interact with single-stranded RNA. The RNA-binding protein may interact with double-stranded RNA. The RNA-binding protein probe may further comprise a detectable moiety.

A polypeptide probe may be a protein that may detect an open or relaxed portion of a chromatin. The polypeptide probe may be a modified enzyme that lacks cleavage activity. The modified enzyme may be an enzyme that recognizes DNA or RNA (double-stranded or single-stranded). Examples of modified enzymes may be obtained from oxidoreductases, transferases, hydrolases, lyases, isomerases, or ligases. A modified enzyme may be an endonuclease (such as a deactivated restriction endonuclease such as the TALEN or CRISPR probes described herein).

A polypeptide probe may be an antibody or binding fragment thereof. The antibody or binding fragment thereof may be a protein interacting partner of a product of a regulatory element. The antibody or binding fragment thereof may comprise a humanized antibody or binding fragment thereof, murine antibody or binding fragment thereof, chimeric antibody or binding fragment thereof, monoclonal antibody or binding fragment thereof, monovalent Fab′, divalent Fab2, F(ab)′3 fragments, single-chain variable fragment (scFv), bis-scFv, (scFv)2, diabody, minibody, nanobody, triabody, tetrabody, disufide stabilized Fv protein (dsFv), single-domain antibody (sdAb), Ig NAR, camelid antibody or binding fragment thereof or a chemically modified derivative thereof. The antibody or binding fragment thereof may further comprise a detectable moiety.

Multiple probes may be used together in a probe set to detect a nucleic acid sequence using Nano-FISH. A probe set can also be referred to herein as a “probe pool.” The probe set may be designed for the detection of the target nucleic acid sequence. For example, the probe set may be optimized for probes based on GC content, 16mer base matches (for determining binding specificity of the probe), and their predicted melting temperature when hybridized. The 16mer base matches may have a total of 24 matches to the 16mer database. In some embodiments, probe sets with greater than 100 16-mer database matches may be discarded.

Exemplary probe nucleotide sequences are shown in TABLE 3 for probe sets for different target sequences. Some exemplary probe sequences may be target sequences located in the GREB1 promoter of chromosome 2, ER iDHS1 of chromosome 2, ER iDHS2 of chromosome 2, HBG1up of chromosome 11, HBG2 up of chromosome 11, HS1 of chromosome 11, HS2 of chromosome 11, HS3 of chromosome 11, HS4 of chromosome 11, HS5 of chromosome 11, HS1 Lflank of chromosome 11, HS1 2flank of chromosome 11, HS2 3 flank of chromosome 11, HS3 4flank of chromosome 11, HS4 5 flank of chromosome 11, HS5 Rflank of chromosome 11, CCND1 SNP of chromosome 11, CCND1 CTL of chromosome 11, the CCND1 promoter of chromosome 11, Chromosome 18 dead1 of chromosome 18, Chromosome 18 dead2 of chromosome 18, Chromosome dead3 of chromosome 18, CNOT promoter of chromosome 19, CNOT inter1 of chromosome 19, CNOT inter2 of chromosome 19, CNOT inter3 of chromosome 19, TSEN promoter of chromosome 19, KLK2 promoter of chromosome 19, KLK3 promoter of chromosome 19, or KLK eRNA of chromosome 19. GREB1 is gene that may be induced by estrogen stimulation of MCF-7 breast cancer cells. ER iDHS1 and ER iDHS2 are DHS that may be induced by estrogen stimulation of MCF-7 breast cancer cells. HBG1up and HBG2up are hemoglobin genes expressed in K562 erythroleukemia cells. HS1, HS2, HS3, HS4, and HS5 are hypersensitive sits in the beta-globin locus control region, and HS1 Lflank, HS2 3flank, HS3 4flank, HS4 5flank, HS5 Rflank are sequences in the intervening regions between HS1-HS5. CCND SNP is an enhancer for the CCND1 gene, CCND1 CTL is a control region adjacent to the CCND1 SNP, and the CCND1 promoter is the promoter region of the CCND1 gene. Chromosome 18 dead1, Chromosome 18 dead 2, and Chromosome 18 dead3 are non-hypersensitive regions of chromosome 18. The CNOT promoter is the promoter (active region) of CNOT. The TSEN promoter is the promoter (active region) of TSEN. The KLK2 promoter is the promoter KLK2. The KLK3 promoter is the promoter of KLK3. KLK eRNA is an enhancer for the KLK2 gene and/or the KLK3 gene, and which may also enhance RNA. For example, a probe set comprising at least nine different Q570 labeled probes selected from the group consisting of SEQ ID NO: 1-SEQ ID NO: 39 may be used to detect the GREB1 promoter in chromosome 2. A Q570 labeled probe set comprising probes with SEQ ID NO: 7-SEQ ID NO: 35 may be used to detect the GREB1 promoter in chromosome 2. A probe set comprising at least nine different Q670 labeled probes selected from the group consisting of SEQ ID NO: 40-SEQ ID NO: 72 may be used to detect the ER iDHS 1 in chromosome 2. A probe set comprising at least nine different Q670 labeled probes selected from the group consisting of SEQ ID NO: 73-SEQ ID NO: 104 may be used to detect the ER iDHS 2 in chromosome 2. A probe set comprising at least nine different Q570 labeled probes selected from the group consisting of SEQ ID NO: 105-SEQ ID NO: 134 may be used to detect the HBG1up in chromosome 11. A probe set comprising at least nine different Q570 labeled probes selected from the group consisting of SEQ ID NO: 135-SEQ ID NO: 164 may be used to detect the HBG2up in chromosome 11. A probe set comprising at least nine different Q570/670 labeled probes selected from the group consisting of SEQ ID NO: 165-SEQ ID NO: 194 may be used to detect HS1 in chromosome 11. A probe set comprising at least nine different Q570/670 labeled probes selected from the group consisting of SEQ ID NO: 195-SEQ ID NO: 224 may be used to detect HS2 in chromosome 11. A probe set comprising at least nine different Q570/670 labeled probes selected from the group consisting of SEQ ID NO: 225-SEQ ID NO: 254 may be used to detect HS3 in chromosome 11. A probe set comprising at least nine different Q670 labeled probes selected from the group consisting of SEQ ID NO: 255-SEQ ID NO: 298 may be used to detect HS4 in chromosome 11. A probe set comprising at least nine different Q570/670 labeled probes selected from the group consisting of SEQ ID NO: 299-SEQ ID NO: 340 may be used to detect HS5 in chromosome 11. A probe set comprising at least nine different Q670 labeled probes selected from the group consisting of SEQ ID NO: 341-SEQ ID NO: 370 may be used to detect HS1 Lflank in chromosome 11. A probe set comprising at least nine different Q570 labeled probes selected from the group consisting of SEQ ID NO: 371-SEQ ID NO: 400 may be used to detect HS1 2flank in chromosome 11. A probe set comprising at least nine different Q670 labeled probes selected from the group consisting of SEQ ID NO: 401-SEQ ID NO: 430 may be used to detect HS2 3flank in chromosome 11. A probe set comprising at least nine different Q570 labeled probes selected from the group consisting of SEQ ID NO: 431-SEQ ID NO: 460 may be used to detect HS3 4flank in chromosome 11. A probe set comprising at least nine different Q670 labeled probes selected from the group consisting of SEQ ID NO: 461-SEQ ID NO: 484 may be used to detect HS4 5flank in chromosome 11. A probe set comprising at least nine different Q570 labeled probes selected from the group consisting of SEQ ID NO: 485-SEQ ID NO: 514 nay be used to detect HS5 Rflank in chromosome 11. A probe set comprising at least nine different Q570 labeled probes selected from the group consisting of SEQ ID NO: 515-SEQ ID NO: 544 may be used to detect CCND1 SNP in chromosome 11. A probe set comprising at least nine different Q670 labeled probes selected from the group consisting of SEQ ID NO: 545, SEQ ID NO: 539-SEQ ID NO: 544, or SEQ ID NO: 546-SEQ ID NO: 564 may be used to detect CCND1 CTL in chromosome 11. A probe set comprising at least nine different Q670 labeled probes selected from the group consisting of SEQ ID NO: 559-SEQ ID NO: 592 may be used to detect the CCND1 promoter in chromosome 11. A probe set comprising at least nine different Q670 labeled probes selected from the group consisting of SEQ ID NO: 593-SEQ ID NO: 622 may be used to detect Chromosome 18 dead1 in chromosome 18. A probe set comprising at least nine different Q670 labeled probes selected from the group consisting of SEQ ID NO:623-SEQ ID NO: 652 may be used to detect Chromosome 18 dead2 in chromosome 18. A probe set comprising at least nine different Q670 labeled probes selected from the group consisting of SEQ ID NO: 653-SEQ ID NO: 682 may be used to detect Chromosome 18 dead3 in chromosome 18. A probe set comprising at least nine different Q670 labeled probes selected from the group consisting of SEQ ID NO: 683-SEQ ID NO: 712 may be used to detect the CNOT3 promoter in chromosome 19. A probe set comprising at least nine different Q670 labeled probes selected from the group consisting of SEQ ID NO: 713-SEQ ID NO: 742 may be used to detect the TSEN34 promoter in chromosome 19. A probe set comprising at least nine different Q670 labeled probes selected from the group consisting of SEQ ID NO: 743-SEQ ID NO: 772 may be used to detect CNOT3 inter1 in chromosome 19. A probe set comprising at least nine different Q670 labeled probes selected from the group consisting of SEQ ID NO: 773-SEQ ID NO: 802 may be used to detect CNOT3 iner2 in chromosome 19. A probe set comprising at least nine different Q670 labeled probes selected from the group consisting of SEQ ID NO: 803-SEQ ID NO: 832 may be used to detect CNOT3 inter3 in chromosome 19. A probe set comprising at least nine different Q570 labeled probes selected from the group consisting of SEQ ID NO: 833-SEQ ID NO: 862 may be used to detect the KLK2 promoter in chromosome 19. A probe set comprising at least nine different Q570 labeled probes selected from the group consisting of SEQ ID NO: 863-SEQ ID NO: 892 may be used to detect the KLK3 promoter in chromosome 19. A probe set comprising at least nine different Q670 labeled probes selected from the group consisting of SEQ ID NO: 893-SEQ ID NO: 929 may be used to detect KLK eRNA in chromosome 19. A probe set comprising at least at least nine different probes labeled with a detection agent selected from the group consisting of SEQ ID NO: 930-SEQ ID NO: 1061 may be used to detect an HIV nucleic acid sequence.

H. Detectable Moieties

A detecting agent may comprise a detectable moiety. A detectable moiety may be a small molecule (such as a dye) or a macromolecule. A macromolecule may include polypeptides (such as proteins and/or protein fragments), nucleic acids, carbohydrates, lipids, macrocycles, polyphenols, and/or endogenous macromolecule complexes. A detectable moiety may be a small molecule. A detectable moiety may be a macromolecule.

A detectable moiety may include a moiety that is detectable by a colorimetric method or a fluorescent method. For example, a colorimetric method may be an assay which utilizes reagents that undergo a measurable color change in the presence of an analyte (such as an enzyme, an antibody, a compound, a hormone). Exemplary colorimetric method may include enzyme-mediated detection method such as tyramide signal amplification (TSA) which utilizes horseradish peroxidase (HRP) to generate a signal when digested by tyramide substrate and 3,3′,5,5′-Tetramethylbenzidine (TMB) which generates a blue color upon oxidation to 3,3′5,5′-tetramethylbenzidine diamine in the presence of a peroxidase enzyme such as HRP. A detectable moiety described herein may include a moiety that is detectable by a colorimetric method.

A detectable moiety may also include a moiety that is detectable by a fluorescent method. Sometimes, the detectable moiety may be a fluorescent moiety. A fluorescent moiety may be a small molecule (such as a dye) or a fluorescently labeled macromolecule. A fluorescently labeled macromolecule may include a fluorescently labeled polypeptide (such as a labeled protein and/or a protein fragment), a fluorescently labeled nucleic acid molecule, a fluorescently labeled carbohydrate, a fluorescently labeled lipid, a fluorescently labeled macrocycle, a fluorescently labeled polyphenol, and/or a fluorescently labeled endogenous macromolecule complex (such as a primary antibody-secondary antibody complex).

A fluorescent small molecule may comprise rhodamine, rhodol, fluorescein, thiofluorescein, aminofluorescein, carboxyfluorescein, chlorofluorescein, methylfluorescein, sulfofluorescein, aminorhodol, carboxyrhodol, chororhodol, methylrhodol, sulforhodol; aminorhodamine, carboxyrhodamine, chlororhodamine, methylrhodamine, sulforhodamine, thiorhodamine, cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiamle derivatives, pyridyloxamole, nitrobenzoxadiazole, benzoxadiazole, pyren derivatives, cascade blue, oxazine derivatives, Nile red, Nile blue, cresyl violet, oxazine 170, acridine derivatives, proflavin, acridine orange, acridine yellow, arylmethine derivatives, auramine, crystal violet, malachite green, tetrapyrrole derivatives, porphin, phtalocyanine, bilirubin 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl)maleimide, stilbenes, pyrenes, 6-FAM (Fluorescein), 6-FAM (NHS Ester), 5(6)-FAM, 5-FAM, Fluorescein dT, 5-TAMRA-cadavarine, 2-aminoacridone, HEX, JOE (NHS Ester), MAX, TET, ROX, TAMRA, TARMA™ (NHS Ester), TEX 615, ATTO™ 488, ATTO™ 532, ATTO™ 550, ATTO™ 565, ATTO™ Rho101, ATTO™ 590, ATTO™ 633, ATTO™ 647N, TYE™ 563, TYE™ 665, or TYE™ 705.

A fluorescent moiety may comprise Cy3, Cy5, Cy5.5, Cy7, Q570, Alexa488, Alexa555, Alexa594, Alexa647, Alexa680, Alexa 750, Alexa 790, TexasRed, CF610, Propidium iodide, Quasar 570 (Q570), Quasar 670 (Q670), IRDye700, IRDye800, Indocyanine green, Pacific Blue dye, Pacific Green dye, or Pacific Orange dye.

A fluorescent moiety may comprise a quantum dot (QD). Quantum dots may be a nanoscale semiconducting photoluminescent material, for example, as described in Alivisatos A. P., “Semiconductor clusters, nanocrystals, and quantum dots,” Science 271(5251): 933-937 (1996).

Exemplary QDs may include, but are not limited to, CdS quantum dots, CdSe quantum dots, CdSe/CdS core/shell quantum dots, CdSe/ZnS core/shell quantum dots, CdTe quantum dots, PbS quantum dots, and/or PbSe quantum dots. As used herein, CdSe/ZnS may mean that a ZnS shell is coated on a CdSe core surface (a “core-shell” quantum dot). The shell materials of core-shell QDs may have a higher bandgap and passivate the core QDs surfaces, resulting in higher quantum yield and higher stability and wider applications than core QDs.

QDs may absorb a wide spectrum of light, and may be physically tuned with emission bandwidths in various wavelengths. See, e.g., Badolato, et al., Science 208:1158-61 (2005). For example, the emission bandwidth may be in the visible spectrum (from about 350 to about 750 un), the ultraviolet-visible spectrum (from about 100 nm to about 750 nm), or in the near-infrared spectrum (from about 750 nm to about 2500 nm). QDs that emit energy in the visible range may include, bit are not limited to, CdS, CdSe, CdTe, ZnSe, ZnTe, GaP, and GaAs. QDs that emit energy in the blue to near-ultraviolet range include, but are not limited to, ZnS and GaN. QDs that emit energy in the near-infrared range include, but are not limited to, InP, InAs, InSb, PbS, and PbSe.

The radius of a QD may be modulated to manipulate the emission bandwidth. For example, a radius of between about 5 and about 6 nm QD may emit wavelengths resulting in emission colors such as orange or red. A radius of between about 2 and about 3 nm may emit wavelengths resulting in emission colors such as blue or green.

A QD may further form a QD microstructure, which encompasses one or more layers of QD. For example, each quantum dot containing layer may comprise a single type of quantum dot of a specific emission color. For example, each layer may be made of any material suitable for use that (a) allows excitation light to reach the quantum dot and allows fluorescence generated from the quantum dot to pass through the layer(s) for detection and (b) may be combined with a quantum dot to form a layer. Examples of materials that may be used to form layers containing quantum dots include, but are not limited to, inorganic, organic, or polymeric material, each with or without biodegradable properties, and combinations thereof. The layers may comprise silica-based compounds or polymers. Exemplary silica-based layers may include, but are not limited to, those comprising tetramethoxy silane or tetraethylorthosilicate. Exemplary polymer layers may include, but are not limited to, those comprising polystyrene, poly (methyl methacrylate), polyhydroxyalkanoate, polylactide, or co-polymers thereof.

The quantum dot further may comprise a spacer layer which serves as a barrier to prevent interactions between different QD layers, and may be made of any material suitable for use that (a) allows excitation light to reach the quantum dots in the quantum dot containing layer(s) below it and allows fluorescence generated from those quantum dots to pass through it and (b) may segregate the quantum dots in one layer from those in other layers. Examples of materials that may be used to form spacer layers are the same as for the quantum dot containing layers.

The materials used for the quantum dot containing and spacer layers may be the same or different. The same material may be used in the quantum dot containing layers and the spacer layers.

The quantum dot containing layers and the spacer layers within a given QD molecule may be any thickness and may be varied. For example, thicker QD-containing layers may allow for the loading of increased QDs in the shell, resulting in greater fluorescence intensity for that layer than for a thinner layer containing the same concentration of QDs. Thus, varying layer thickness may facilitate preparing QD-containing layer of various intensities, thereby generating spectrally distinct QD bar codes. In various instances, the QD-containing layers may be between 5 nm and 500 nm. Those of skill in the art will understand that other methods for varying intensity also exist, for example, modifying concentrations of the same QD in one microstructure with a first unique barcode compared to a second QD microstructure with a different fluorescent barcode. The ability to vary the intensities for the same QD color allows for an increased number of distinct and distinguishable microstructures (e.g., spectrally distinct barcodes). The spacer layers may be greater than 10 nm, up to approximately 5 μm thick; the spacer layers may be greater than 10 nm, up to approximately 500 nm thick; the space layers may be greater than 10 nm, up to approximately 100 nm thick.

The quantum dot-containing and spacer layers may be arranged in any order. Examples include, but are not limited to, alternating QD-containing layers and spacer layers, or quantum dot containing layers separated by more than one spacer layer. Tus, a “spacer layer” may comprise a single layer, or may comprise two or more such spacer layers.

The QD microstructure may comprise any number of quantum dot containing layers suitable for use with the microstructure. For example, a microstructure described herein may comprise 2 or more quantum dot-containing layers and an appropriate number of spacer layers based on the number of quantum dot-containing layers. Further, the number of quantum dot containing layers in a given microstructure may range from 1 to “m,” where “m” is the number of quantum dots that may be used.

A defined intensity level may refer to a known amount of quantum dots in each quantum dot containing layer, resulting in a known amount of fluorescent intensity generated from the QD containing layer upon appropriate stimulation. Since each QD containing layer has a defined intensity level, each microstructure may possess a defined ratio of fluorescence intensities generated from the various QD-containing layers upon stimulation. This defined ratio is referred to herein as a barcode. Thus, each type of microstructure with the same QD layers possesses a similar barcode that may be distinguished from microstructures with different QD layers.

Tus, each quantum dot containing layer may comprise a single type of quantum dot of a specific emission color and the layer is produced to possess a defined intensity level, based on the concentration of the QD in the layer. By varying the intensity levels of QDs (“n”) in different microstructures and using a variety of different quantum dots (“m”), the number of different unique barcodes (and thus the number of different unique microstructure populations that may be produced) is approximated by the equation, (n^{m}−1) unique codes. This may provide the ability to generate a large number of different populations of microstructures each with its own unique barcode.

A set of QD-labeled probes may further generate a spectrally distinct barcode. For example, each probe with the set of QD-labeled probes may comprise a QD with a distinct excitation wavelength and the combination of the set may generate a distinct barcode. A set of spectrally distinct QD-labeled probes may be utilized to detect a regulatory element. As such, when detecting two or more regulatory elements, each regulatory element may be spectrally barcoded.

A quantum dot provided herein may include QDot525, QDot 545, QDot 565, QDot 585, QDot 605, or QDot 655. A probe described herein may comprise a quantum dot. A quantum dot may comprise a quantum dot as described in Han et al., “Quantum-dot-tagged microbeads for multiplexed optical coding of biomolecules,” Nat. Biotechnol. 19:631-635 (2001); Gao X., “QD barcodes for biosensing and detection,” Conf Proc IEEE Eng Med Biol Soc 2009: 6372-6373 (2009); and Zrazhevskiy, et al., “Multicolor multicycle molecular profiling with quantum dots for single-cell analysis,” Nat Protoc 8:1852-1869 (2013).

A QD may further comprise a functional group or attachment moiety. One example of such a QD that has a functional group or attachment moiety is a QD with a carboxylic acid terminated surface, such as those commercially available though, for example, Quantum Dot, Inc., Hayward, Calif.

I. Conjugating Moiety

The probe may include a conjugating moiety. The conjugation moiety may be attached at the 5′ terminus, the 3′ terminus, or at an internal site. The conjugating moiety may be a nucleotide analog (such as bromodeoxyuridine). The conjugating moiety may be a conjugating functional group. The conjugating functional group may be an azido group or an alkyne group. The probe may further be derivatized through a chemical reaction such as click chemistry. The click chemistry may be a copper(I)-catalyzed [3+2]-Huisgen 1,3-dipolar cyclo-addition of alkynes and azides leading to 1,2,3-triazoles. The click chemistry may be a copper free variant of the above reaction.

The conjugating moiety may comprise a hapten group. A hapten group may include digoxigenin, 2,4-dinitrophenyl, biotin, avidin, or are selected from azoles, nitroaryl compounds, benzofuazans, triterpenes, ureas, thioureas, rotenones, oxazoles, thiazoles, coumarins, cyclolignans, heterobiaryl compounds, azoaryl compounds or benzodiazepines. A hapten group may include biotin.

The probe comprising the conjugating moiety may further be linked to a second probe (such as a nucleic acid probe or a polypeptide probe), a fluorescent moiety (such as a dye such as a quantum dot), a target nucleic acid, or a conjugating partner such as a polymer (such as PEG), a macromolecule (such as a carbohydrate, a lipid, a polypeptide), and the like.

J. Detection of a Target Nucleic Acid Sequence

The method may comprise an operation of providing one or more probes capable of binding to a target nucleic acid sequence, as described herein. The method may comprise an operation of binding the one or more probes to the target nucleic acid sequence, as described herein. The method may comprise an operation of detecting a signal associated with binding of the one or more probes to the target nucleic acid sequence, as described herein.

The target nucleic acid sequence may be detected in an intact cell. The target nucleic acid sequence may be detected in a fixed cell. The target nucleic sequence may be detected in a lysate or chromatin spread.

A probe may be used to detect a nucleic acid sequence in a sample. For example, a probe comprising a probe sequence capable of binding a nucleic acid sequence (such as a target nucleic acid sequence) and a detectable label (such as a detectable agent) may be used to detect the nucleic acid sequence. A method for detecting a nucleic acid sequence may comprise contacting a nucleic acid sequence with a probe comprising a probe sequence configured to bind at least a portion of the nucleic acid sequence and detecting the probe (such as detecting the detectable label of the probe). The detection of a nucleic acid sequence may comprise binding the probe to the nucleic acid sequence. For example, the detection of a nucleic acid sequence may comprise binding the probe sequence, such as the sequence of an oligonucleotide probe, to a target nucleic acid sequence. In some cases, the detection of a nucleic acid sequence may comprise hybridizing the probe sequence (such as the nucleic acid binding region) of a nucleic acid probe to a target nucleic acid sequence. The nucleic acid sequence may be a virus nucleic acid sequence. The nucleic acid sequence may be an agricultural viral nucleic acid sequence. The nucleic acid sequence may be a lentivirus nucleic acid sequence, an adenovirus nucleic acid sequence, an adeno-associated virus nucleic acid sequence, or a retrovirus nucleic acid sequence.

A nucleic acid sequence may be contacted with a plurality of probes. A nucleic acid sequence may be contacted with a number of probes ranging from about 1 to about 108 probes, from about 2 to about to about 50 million probes. The probes of the plurality of probes may be the same. A plurality of probes may have sequences such that the probes are tiled across the nucleic acid sequence. Each probe can bind to a target nucleic acid sequence along the nucleic acid sequence. The probes of a plurality may be different. A first probe of the plurality of probes may be different than a second probe of the plurality of probes. The plurality of probes may bind to the nucleic acid sequence with from 0 to 10 nucleotides separating each probe.

A nucleic acid sequence may be washed after it has been contacted with a probe. Washing a nucleic acid sequence after it has been contacted with a probe may reduce background signal for detection of the detectable label of the probe.

A nucleic acid sequence (such as a target nucleic acid sequence) can be contacted by a plurality of probes. A nucleic acid sequence can be contacted with a plurality of types of probes. That is, a method of detection of a nucleic acid sequence (such as a target nucleic acid sequence) may comprise contacting the target nucleic acid sequence with a plurality of sets of probes (such as a plurality of types of probes). A first probe set (such as a first type of probe) may be different from a second probe set (such a second type of probe) in that the first probe type comprises a first probe sequence which is different than the probe sequence of the second probe type. The probe sequence of a first type of probe may be the same as the probe sequence of a second type of probe. A first probe set may comprise a first detectable label and a first probe sequence and a second probe set may comprise a second detectable label and a second probe sequence, wherein the first and second probe sequences are the same and the first and second detectable labels are different. The first and second probe sequences may be different and the first and second detectable labels of a first and second probe set may be the same. The first and second probe sequences of a first and second probe set may be different and the first and second detectable labels of a first and second probe set may be different. A method of detecting a nucleic acid sequence may comprise contacting a nucleic acid sequence with 1 to 20 types of probes.

A first probe sequence may be configured to specifically recognize (such as to bind to or to hybridize with) a first nucleic acid sequence (such as a first target nucleic acid sequence). A second probe sequence may be configured to specifically recognize (such as to bind to or to hybridize with) a second nucleic acid sequence (such as a second target nucleic acid sequence).

A detectable label may be detected with a detector. A detector may detect the signal intensity of the detectable label. A detector may spatially distinguish between two detectable labels. A detector may also distinguish between a first and second detectable label based on the spectral pattern produced by the first and second detectable labels, wherein the first and second detectable label do not produce an identical spectral intensity pattern. For example, a detector may distinguish between a first and second detectable signal, wherein the wavelength of the signal produced by the first detectable label is not the same as the wavelength of the signal produced by the second detectable label. A detector may resolve (such as by spatially distinguishing or spectrally distinguishing) a first and second detectable label that are less than 1 kb apart to less than 100 kb apart on a chromosome. The detectable label of the probe may be detected optically. For example, a detectable label of a probe may be detected by light microscopy, fluorescence microscopy, or chromatography. Detection of the detectable label of a probe may comprise stimulating the probe or a portion thereof (such as the detectable label) with a source of radiation (such as a light source, such as a laser). Detection of the detectable label of a probe may also comprise an enzymatic reaction.

Detection of the target nucleic acid sequence may be within a period of not more than 12 hours to not more than 48 hours.

Determining the presence of a genetic modification in a cell using the Nano-FISH method described herein may be useful is assessing the phenotype of the cell resulting from the genetic modification. A method for assessing a phenotype of an intact genetically modified cell may comprise: a) providing the intact genetically modified cell comprising a target nucleic acid sequence less than 2.5 kilobases in length; b) contacting the intact genetically modified cell with a first plurality of probes, wherein each probe comprises a first detectable label and a probe sequence that binds to a portion of the target nucleic acid sequence; c) detecting a presence of the first detectable label in the intact cell, wherein the presence of the first detectable label indicates the presence of the target nucleic acid sequence; d) determining a phenotype of the intact genetically modified cell; and e) correlating the phenotype of the intact genetically modified cell with the presence of the target nucleic acid sequence. The method may further comprise determining a number or location of genetic modifications in the intact genetically modified cell. The method may further comprise f) selecting a first intact genetically modified cell comprising a phenotype of interest; g) determining a set of conditions used for a genetic modification of the first intact genetically modified cell; and h) preparing a second genetically modified cell using the set of conditions for genetic modification. The intact genetically modified cell may be a eukaryotic cell that was genetically modified. The intact genetically modified cell may be a bacteria cell that was genetically modified. The intact genetically modified cell may be a mammalian cell that was genetically modified. The intact genetically modified cell may be any cell as described herein that was genetically modified. The phenotype may be a product expressed as a result of the genetic modification of the cell. The phenotype may be an increased level or decreased level of the product expressed as a result of the genetic modification of the cell. The phenotype may be an increased quality of the product expressed as a result of the genetic modification of the cell. The expressed product may be protein, such as an enzyme. The expressed product may be a transgene protein, RNA, or a secondary product of the genetic modification. For example, if an enzyme is produced as a result of the genetic modification of the cell, a secondary product of the genetic modification is a product of the enzyme.

Determining the number of target nucleic acid sequences in a cell may be useful in determining the phenotype of the cell. Cells with a specific number of target nucleic acid sequences may be tested for increased cellular activity, decreased cellular activity, or toxicity. Increased cellular activity may be increased expression of a protein or a cellular product. Decreased cellular activity may be decreased expression of a protein or a cellular product. Toxicity may be a result of cellular activity that may be too high or too low, resulting in cell death. For example, the contacting a sample of virally transduced cells with a probe configured to bind to a particular target viral nucleic acid sequence and then determining the number of viral integrants may be an expedient means of determining whether virus has successfully integrated in the cells of the sample in way in which a desired therapeutic effect may result if given to a patient as a therapy.

Determining the presence, absence, identity, spatial position or sequence position of a target nucleic acid sequence in a sample may be useful in determining a condition of a patient. For example, the contacting a sample of cells with a probe configured to bind to a particular target nucleic acid sequence and then determining the number of target nucleic acid sequences in the cell may be an expedient means of determining the number of target nucleic acid sequences may be affecting the cell phenotype or function. For example, contacting a patient sample with a probe configured to bind to a particular nucleic acid sequence may be an expedient means of determining whether the patient has the nucleic acid sequence. As another example, contacting a sample of virally transduced cells with a probe configured to bind to a particular target viral nucleic acid sequence may be an expedient means of determining whether virus has successfully integrated in the cells of the sample. Similarly, contacting a patient sample with a plurality of types of probes, each configured to bind to a different nucleic acid sequence, may be an expedient means of screening patients for various genetic or acquired conditions, such as inherited mutations.

K. Quantification of a Target Nucleic Acid Sequence in a Cell

A method of detecting or determining the presence of a nucleic acid sequence may comprise determining the number of probes associated with the nucleic acid sequence. A method of detecting or determining the presence of a nucleic acid sequence may comprise determining the number of probes hybridized to the nucleic acid sequence.

It may also be possible to determine the quantity of target nucleic acid sequences in this manner. If a viral nucleic acid sequence comprises the target nucleic acid sequence, the number of viral nucleic acid sequences may be quantified using the methods described herein. Quantification of the number of viral nucleic acid sequences in a sample (such as a cell comprising viral integrations) may be useful in determining the multiplicity of infection. This quantification may also be useful for methods of enriching heterogeneous populations of transduced cells to a more homogenous cell population or to a cell population comprising a greater percentage of cells comprising a specific number or a specific range of viral integrations. Quantification of target nucleic acid sequences in a sample using the methods, compositions, and systems described herein may be useful in determining the number of repeated sequences in a nucleic acid of a sample.

In some embodiments, this method can be used for quantifying populations of cells transduced to express chimeric antigen receptors (CARs) in order to determine the average number of viral insertions per cell or the distribution of viral insertions per cell within the cell populations.

For example, a Nano-FISH probe or a Nano-FISH probe set of this disclosure can be used to verify the number of viral insertions in T cells that have been engineered to express CARs, such as BCMA, CD19, CD22, WT1, L1CAM, MUC16, ROR1, or LeY. Thus, the Nano-FISH probe or Nano-FISH probe sets of the present disclosure can be used as a quality control step to verify that engineered CAR T cells have truly been transduced with a vector encoding for a given CAR, prior to administering the CAR T cells to a subject in need thereof.

In some embodiments, this method can be used for quantifying populations of CD34+ hematopoietic stem cells (HSCs) transduced to express a gene of interest for the purpose of gene therapy, in order to determine the average number of viral insertions per cell or the distribution of viral insertions per cell within the cell populations.

For example, a Nano-FISH probe or a Nano-FISH probe set of this disclosure can be used to verify the number of viral insertions in CD34+ cells that have been engineered with any vector, such as a lentivirus vector or an adeno-associated virus vector to express any gene of interest. Thus, the Nano-FISH probe or Nano-FISH probe sets of the present disclosure can be used as a quality control step to verify that engineered CD34+ cells have truly been transduced with a vector encoding for a given gene, prior to administering the engineered CD34+ cells to a subject in need thereof. For example, in some embodiments a CD34+ cell from a human donor is transduced with the lentivirus vector encoding for any gene. A subset of the engineered CD34+ cells can be subject to viral Nano-FISH validation wherein, the CD34+ cells are hybridized to a Nano-FISH probe or Nano-FISH probe set of the present disclosure and imaged to detect and quantify spots in the cell nuclei corresponding to viral insertions. The engineered CD34+ cells can, thus, be verified for successful transduction of any gene. Furthermore, the engineered CD34+ cells can, thus, be characterized for the average number of insertions per cell and/or the distribution of viral insertions per cell. Viral Nano-FISH can provide these valuable metrics characterizing the heterogeneity and quality of the engineered CD34+ cells prior to administration to a subject in need thereof. The above described methods can be used to validate CD34+ cells engineered to in any of the following gene therapies: thalassemia, sickle cell disease, muscular dystrophy, or an immune disorder.

L. Enrichment and Optimintion for the Number of Target Nucleic Acid Sequences in a Cell

The quantification of a target nucleic acid sequence, such as a viral nucleic acid sequence, may allow for the precise tuning of per-cell viral integrant number among a pool of cells transduced with a virus, such as a retrovirus.

Viral transduction of cells may be heterogeneous, producing cells with no viral integrant, a single copy of a viral integrant, or two or more copies of a viral integrant. Using Nano-FISH, a pool of cells with a consistent number of viral integrants may be produced, wherein cells comprising an undesirable number of viral integrants (e.g., too many or no viral integrants) may be reduced or eliminated. Viral integrants may be detected using the methods as described herein for Nano-FISH, also referred to herein as “viral Nano-FISH.” This may use microscopic imaging of fixed cells, and thus the imaged cells may not themselves be collected for subsequent use. However, pairing the Nano-FISH with a statistical approach may allow for (i) inferring the distribution of viral integrants in subpools of cells expanding in culture, and (ii) combining subpools to create a refined pool of cells with uniform viral integrants number. The pool of cells with the uniform number of viral integrants may be a therapeutic used to treat a disease.

In some embodiments, this method may be used for enriching populations of cells transduced to express chimeric antigen receptors (CARs) in order to deliver a cell population with a uniform number of CAR integrations to a patient as a cancer therapy.

The enrichment process may comprise the following steps: a) quantify the number of viral integrants in a sample from a source pool of cells; b) subdivide the remaining cells of the source pool into K subpools, each with approximately N cells (the value of N may be chosen to ensure a high likelihood of subpools having zero or a greatly reduced fraction of cells with more than one viral integrant; c) allow each subpool to undergo multiple cell divisions to create cell clones with identical numbers of viral integrants per cell; d) perform Nano-FISH on a representative sample from each subpool to assess the number of viral integrants in each cell; e) based on the assessment of step d) estimate the distribution of viral integrants for each subpool and eliminate the subpools with the unfavorable distribution of viral integrants; and f) combine the remaining subpools to create a single enriched pool comprising cells with a more homogenous number of viral integrants.

In some instances, the number of cell divisions and fraction of cells drawn for Nano-FISH analysis may be selected to ensure a high likelihood of detecting the presence of a multiple integration event given the random set of cells drawn. In some instances, any subpool may be eliminated if the proportion of cells with more than one viral integrants exceeds a specified threshold (which may be 0). Subpools may also be eliminated if the proportion of cells with no viral integrant is above a specified threshold. This secondary selection criterion may increase the relative abundance of the single viral integrant phenotype.

The above method for enrichment may allow numerous parameters to be specified in order to achieve a given goal. These parameters may include the number of cells per subpool, the number of subpools, the number of cell divisions (i.e., time in culture), and fraction of cells withdrawn for Nano-FISH. In addition, the optimal protocol may depend on the underlying rate of multiple viral insertions and the probability of detecting a spot with Nano-FISH. Finally, the approach may depend on the tolerance for allowing cells with multiple or no viral integrants into the enriched pool.

In some cases, subpools may be enriched so that no cells comprise multiple integrants. To achieve this, for example, a statistical model may be used. For example, the probability of a given pool of N cells containing zero cells with multiple insertions is given by (1−p)^{N}. If there are K subpools, then the total number of cells contained in subpools without any multiple insertions may be M=KN(1−p)^{N}. Therefore, K=M/[N(1−p)^{N}] subpools may be needed to achieve a total of M progenitor cells without multiple integrations. The optimal value of N may be 1/p.

In addition to the parameters N and K, the target number of cell division cycles D and fraction of cells F to be withdrawn for Nano-FISH may need to be determined. For this determination, all cells may undergo the same number of cell divisions, resulting in 2 copies of each. Thus, the probability of withdrawing k of the cells with 2 integrants in a fraction F of all cells in the subpool may be given by P(k|N,D,F) a hypergeometric probability distribution with 2^{D} positive items in N2^{D} total items with FN2^{D} drawn from the total. In some cases, the likelihood of a Nano-FISH spot being detected may be S, then the overall probability of detection may be given by

ρₖ₌₁²^{^{D}}p(k|N,D,F)(1−(1−S²)^{k})

Determining the presence, absence, identity, spatial position or sequence position of a target nucleic acid sequence in a sample may be useful in determining a condition of a patient. For example, contacting a patient sample with a probe configured to bind to a particular nucleic acid sequence may be an expedient means of determining whether the patient has the nucleic acid sequence. Similarly, contacting a patient sample with a plurality of types of probes, each configured to bind to a different nucleic acid sequence, may be an expedient means of screening patients for various genetic or acquired conditions, such as inherited mutations.

M. Determination of the Spatial Position of a Target Nucleic Acid Sequence

The method may comprise an operation of providing one or more probes capable of binding to a target nucleic acid sequence, as described herein. The method may comprise an operation of binding the one or more probes to the target nucleic acid sequence, as described herein. The method may comprise an operation of imaging a signal associated with binding of the one or more probes to the target nucleic acid sequence, as described herein.

A method of detecting or determining the presence of a nucleic acid sequence may comprise determining the spatial position of a nucleic acid sequence (such as a target nucleic acid sequence). Determining the spatial position of a nucleic acid sequence may comprise contacting a nucleic acid sequence with a probe, which may comprise a detectable label and a probe sequence configured to bind to the nucleic acid sequence, and detecting the detectable label of the probe.

The spatial position of the nucleic acid sequence may be determined relative to features of the sample (such as features of a cell), structures of the sample (such structures or organelles of the cell), or other nucleic acids by using the same or a different imaging modality to detect the reference features, structures, or nucleic acids. For instance, the spatial position of a nucleic acid sequence in a cell relative to the nucleus of a cell by using a plurality of antibodies with a detectable label to counter-label structures of the cell, such as the cell membrane. A cell line expressing a detectable label (such as a fusion protein with a structural protein expressed by the cell) may be used to determine spatial position of a nucleic acid sequence in a cell. If the target nucleic acid sequence comprises a viral nucleic acid sequence, the spatial location of the viral nucleic acid sequence may be determined by the methods as described herein.

Data collected from detection of all or a portion of the detectable labels in a sample may be used to form one or more two-dimensional images or a three-dimensional rendering or to make calculations determining or estimating the spatial position of the target nucleic acid sequence.

A first probe comprising a first detectable label and a first probe sequence configured to bind to a nucleic acid sequence (such as a target nucleic acid sequence) may be used as a reference position for a second probe comprising a second detectable label and a second probe sequence configured to bind to a second nucleic acid sequence (such as a second target nucleic acid sequence). For example, a first probe specific to a first target nucleic acid sequence of a nucleic acid with a known or anchored position on the nucleic acid may be used as a reference to determine the spatial position of a second target nucleic acid sequence bound by a second probe prior to or during imaging.

N. Detection of the Sequence Position of a Target Nucleic Acid Sequence

The method may comprise an operation of providing a first set of one or more probes capable of binding to one or more reference nucleic acid sequences with known positions in the genome, as described herein. The method may comprise an operation of binding the first set of one or more probes to the one or more reference nucleic acid sequences, as described herein. The method may comprise an operation of providing a second set of one or more probes capable of binding to a target nucleic acid sequence, as described herein. The method may comprise an operation of binding the second set of one or more probes to the target nucleic acid sequence, as described herein. The method may comprise an operation of detecting a signal associated with binding of the first set of one or more probes to the one or more reference nucleic acid sequences and of the second set of one or more probes to the target nucleic acid sequence, as described herein. The method may comprise an operation of comparing the signals associated with binding of the first set of one or more probes to the reference nucleic acid sequences to the signal associated with binding of the second set of one or more probes to the target nucleic acid sequence.

A method of detecting or determining the presence of a nucleic acid sequence may comprise determining the sequence position of a nucleic acid sequence (such as a target nucleic acid sequence). For example, a probe with a probe sequence configured to recognize a first target sequence with a known position in the sequence of a nucleic acid may be used as reference for calculations or estimations of the sequence position of a second target nucleic acid sequence on the nucleic acid. For example, a first probe having a probe sequence configured to recognize a first target sequence with a first known position in the sequence of a nucleic acid and a second probe having a probe sequence configured to recognize a second target nucleic acid sequence with a second known position in the sequence of the nucleic acid may be used as reference points for a third probe configured to recognize a third target nucleic acid sequence with an unknown position in the nucleic acid. The relative sequence position of the third target nucleic acid sequence may be determined or estimated by comparing it to the positions of the first and second target nucleic acid sequences, as indicated by the signals from the first and second probes.

O. Detection of Target Nucleic Acid Sequences in a Sample Relative to a Control

The method may comprise an operation of providing a one or more probes capable of binding to a target nucleic acid sequence in a reference sample and a target nucleic acid sequence in a sample under test, as described herein. The method may comprise an operation of binding the one or more probes to the target nucleic acid sequence in the reference sample and the target nucleic acid sequence in the sample under test, as described herein. The method may comprise an operation of detecting a signal associated with binding of the set of one or more probes to the target nucleic acid sequence in the reference sample and the target nucleic acid sequence in the sample being tested, as described herein. The method may comprise an operation of comparing the signal associated with binding of the one or more probes to the target nucleic acid sequence in the reference sample to the signal associated with binding of the one or more probes to the target nucleic acid sequence in the sample under test, as described herein.

P. Correlation of the Detection of a Target Nucleic Acid Sequence in a Sample with a Target Protein Expression

The detection of a target nucleic acid sequence in a cell may be correlated with a target protein expression in the same cell. The method may comprise providing a one or more probes capable of binding to a target nucleic acid sequence in a sample and a target nucleic acid sequence in a sample being tested, as described herein, and further comprise providing one or more detectable labels to detect the target protein expression. The presence, absence, or quantity of the detected target nucleic acid sequence may be correlated to the presence, absence, or quantity of the target protein expression. This information may be used to further investigate the relationship between the target nucleic acid sequence and the target protein, and/or how different treatments may perturb this correlation.

A viral nucleic acid sequence may be introduced into a cell by a viral vector, such as a virus particle, which may be called a virus or a virion. A virus particle may also be introduced to a cell by a bacteriophage. A virus particle may introduce a viral nucleic acid sequence into a cell through a series of steps that may include attachment (such as binding) of the virus particle to the cell membrane of the cell, internalization (such as penetration) of the viral particle into the cell (such as via formation of a vesicle around the virus particle), breakdown of the vesicle containing the virus particle (such as through uncoating, which may comprise breakdown of the portions of the virus such as a the viral coat), expression of the viral nucleic acid sequence or a portion thereof processing and/or maturation of the viral nucleic acid sequence's expression product, incorporation of the viral nucleic acid sequence or its expression product into a DNA sequence of the host cell, and/or or replication of the viral nucleic acid sequence or a portion thereof. A viral nucleic acid sequence may be targeted to the nucleus of the cell after internalization.

Introduction of a viral nucleic acid sequence into a cell by a virus particle may lead to permanent integration of the viral nucleic acid sequence into a DNA sequence of the cell. For example, a viral nucleic acid sequence introduced into a cell by a retrovirus, such as a lentivirus or adeno-associated virus, may be integrated directly into the DNA sequence of a cell. Introduction of a viral nucleic acid sequence into a cell by a virus particle may not lead to integration into a DNA sequence of the cell.

A viral particle may be a double-stranded DNA (dsDNA) virus, a single-stranded DNA (ssDNA) virus, a double-stranded RNA (dsRNA) virus, a sense single-stranded RNA (+ssRNA) virus, an antisense single-stranded RNA (−ssRNA). Some viral particles may introduce a reverse transcriptase, integrase, and/or protease (such as a reverse transcriptase encoded by a pol gene sequence, which may be a portion of the viral nucleic acid sequence) into the infected cell. Examples of virus particles that introduce reverse transcriptase into an infected cell include single-stranded reverse transcriptase RNA (ssRNA-RT) viruses and double-stranded DNA reverse transcriptase (dsDNA-RT) viruses. Examples of ssRNA-RT viruses include metaviridae, pseudoviridae, and retroviridae. Examples of dsDNA-RT viruses include hepadnaviridae (e.g., Hepatitis B virus) and caulimoviridae. Additional examples of viruses include lentiviruses, adenoviruses, adeno-associated viruses, and retroviruses.

A viral nucleic acid sequence may be introduced into a cell by a non-viral vector, such as a plasmid. A plasmid may be a DNA polynucleotide encoding one or more genes. A plasmid may comprise a viral nucleic acid sequence. A viral nucleic acid sequence of a plasmid may encode a non-coding RNA (such as a transfer RNA, a ribosomal RNA, a microRNA, an siRNA, a snRNA, a shRNA, an exRNA, a piwi RNA, a snoRNA, a scaRNA, or a long non-coding RNA) or a coding RNA (such as a messenger RNA). A coding RNA may be modified (such as by splicing poly-adenylation, or addition of a 5′ cap) or translated into a polypeptide sequence (such as a protein) after being transcribed from a DNA nucleic acid sequence of a plasmid.

Samples for Analysis of Protein (e.g., p53BP1) Accumulation in Response to a Cellular Perturbation and Nano-FISH Analysis

A sample described herein may be a fresh sample or a fixed sample. The sample may be a fresh sample. The sample may be a fixed sample. The sample may be a live sample. The sample may be subjected to a denaturing condition. The sample may be cryopreserved.

The sample may be a cell sample. The cell sample may be obtained from the cells or tissue of an animal. The animal cell may comprise a cell from an invertebrate, fish, amphibian, reptile, or mammal. The mammalian cell may be obtained from a primate, ape, equine, bovine, porcine, canine, feline, or rodent. The mammal may be a primate, ape, dog, cat, rabbit, ferret, or the like. The rodent may be a mouse, rat, hamster, gerbil, hamster, chinchilla, or guinea pig. The bird cell may be from a canary, parakeet, or parrot. The reptile cell may be from a turtle, lizard, or snake. The fish cell may be from a tropical fish. For example, the fish cell may be from a zebrafish (such as Danio rerio). The amphibian cell may be from a frog. An invertebrate cell may be from an insect, arthropod, marine invertebrate, or worm. The worm cell may be from a nematode (such as Caenorhabditis elegans). The arthropod cell may be from a tarantula or hermit crab.

The cell sample may be obtained from a mammalian cell. For example, the mammalian cell may be an epithelial cell, connective tissue cell, hormone secreting cell, a nerve cell, a skeletal muscle cell, a blood cell, an immune system cell, or a stem cell. A cell may be a fresh cell, live cell fixed cell, intact cell, or cell lysate. Cell samples can be any primary cell, such as a hematopoetic stem cell (HSCs) or naïve or stimulated T cells (e.g., CD4+ T cells).

Cell samples may be cells derived from a cell line, such as an immortalized cell line. Exemplary cell lines include, but are not limited to, 293A cell line, 293FT cell line, 293F cell line, 293 H cell line, HEK 293 cell line, CHO DG44 cell line, CHO-S cell line, CHO-K1 cell line, Expi293F™ cell line, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cell line, FreeStyle™ CHO-S cell line, GripTite™ 293 MSR cell line, GS-CHO cell line, HepaRG™ cell line, T-REx™ Jurkat cell line, Per.C6 cell line, T-REx™-293 cell line, T-REx™-CHO cell line, T-REx™-HeLa cell line, NC-HIMT cell line, PC12 cell line, A549 cells, and K562 cells.

The cell sample may be obtained from cells of a primate. The primate may be a human, or a non-human primate. The cell sample may be obtained from a human. For example, the cell sample may comprise cells obtained from blood, urine, stool, saliva, lymph fluid, cerebrospinal fluid, synovial fluid, cystic fluid, ascites, pleural effusion, amniotic fluid, chorionic villus sample, vaginal fluid, interstitial fluid, buccal swab sample, sputum, bronchial lavage, Pap smear sample, or ocular fluid. The cell sample may comprise cells obtained from a blood sample, an aspirate sample, or a smear sample.

The cell sample may be a circulating tumor cell sample. A circulating tumor cell sample may comprise lymphoma cells, fetal cells, apoptotic cells, epithelia cells, endothelial cells, stem cells, progenitor cells, mesenchymal cells, osteoblast cells, osteocytes, hematopoietic stem cells (HSC) (e.g., a CD34+ HSC), foam cells, adipose cells, transcervical cells, circulating cardiocytes, circulating fibrocytes, circulating cancer stem cells, circulating myocytes, circulating cells from a kidney, circulating cells from a gastrointestinal tract, circulating cells from a king, circulating cells from reproductive organs, circulating cells from a central nervous system, circulating hepatic cells, circulating cells from a spleen, circulating cells from a thymus, circulating cells from a thyroid, circulating cells from an endocrine gland, circulating cells from a parathyroid, circulating cells from a pituitary, circulating cells from an adrenal gland, circulating cells from islets of Langerhans, circulating cells from a pancreas, circulating cells from a hypothalamus, circulating cells from prostate tissues, circulating cells from breast tissues, circulating cells from circulating retinal cells, circulating ophthalmic cells, circulating auditory cells, circulating epidermal cells, circulating cells from the urinary tract, or combinations thereof.

The cell can be a T cell. For example, in some embodiments, the T cell can be an engineered T cell transduced to express a chimeric antigen receptor (CAR) or engineered T cell receptor (TCR). The CAR, or TCR T cell can be engineered to bind to BCMA, CD19, CD22, WT1, L1CAM, MUC16, ROR1, or LeY.

A cell sample may be a peripheral blood mononuclear cell sample.

A cell sample may comprise cancerous cells. The cancerous cells may form a cancer which may be a solid tumor or a hematologic malignancy. The cancerous cell sample may comprise cells obtained from a solid tumor. The solid tumor may include a sarcoma or a carcinoma. Exemplary sarcoma cell sample may include, but are not limited to, cell sample obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Exemplary carcinoma cell samples may include, but are not limited to, cell samples obtained from an anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

The cancerous cell sample may comprise cells obtained from a hematologic malignancy. Hematologic malignancy may comprise a leukemia, a lymphoma, a myeloma, a non-Hodgkin's lymphoma, or a Hodgkin's lymphoma. The hematologic malignancy may be a T-cell based hematologic malignancy. The hematologic malignancy may be a B-cell based hematologic malignancy. Exemplary B-cell based hematologic malignancy may include, but are not limited to, chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy may include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

A cell sample described herein may comprise a tumor cell line sample. Exemplary tumor cell line sample may include, but are not limited to, cell samples from tumor cell lines such as 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

A cell sample may comprise cells obtained from a biopsy sample, necropsy sample, or autopsy sample.

The cell samples (such as a biopsy sample) may be obtained from an individual by any suitable means of obtaining the sample using we-known and routine clinical methods. Procedures for obtaining tissue samples from an individual are well known. For example, procedures for drawing and processing tissue sample such as from a needle aspiration biopsy are well-known and may be employed to obtain a sample for use in the methods provided. Typically, for collection of such a tissue sample, a thin hollow needle is inserted into a mass such as a tumor mass for sampling of cells that, after being stained, will be examined under a microscope.

A cell may be a live cen. A cell may be a eukaryotic cell. A cell may be a yeast cell. A cell may be a plant cen. A cell may be obtained from an agricultural plan.

### High-Throughput Assay for Analysis of Protein Markers of Cellular Perturbation and Nano-FISH

In some embodiments, the present disclosure provides methods of high-throughput assaying of target nucleic acid cells in multi-well format. For example, the present disclosure provides methods for depositing cells in at least 24 wells, hybridizing oligonucleotide Nano-FISH probes with cells after denaturation, covering cells in each well with a glass coverslip, and imaging the cells with the microscopy techniques disclosed herein. As an example, PLL-coated 24-well glass-bottom plates can be used to hold 24 samples, wherein each sample contains a cell population. The cell population in each well can be the same or the cell population in each well can be different. Thus, at least 24 unique samples can be processed at the same time. Cells can be deposited into the 24-well plate, treated with fixative solution (e.g., 4$ formaldehyde in 1×PBS or 3 parts methanol and 1 part glacial acetic acid), washed, and hybridized to oligonucleotide Nano-FISH probes. The 24-well plate can then be washed and cells can be mounted with glass coverslips containing an anti-fade solution (e.g., Prolong Gold) prior to imaging. In some embodiments, up to 1 to 10 plates can be simultaneously processed.

Optical Detection of Surrogate Protein Markers (e.g., p53BP1) and/or Nucleic Acid Sequences

Described herein is a method of detecting a protein, such a surrogate protein marker (e.g., p53BP1) of a cellular response induced by a cellular perturbation (genome editing and methods of detecting a nucleic acid sequence. The detection may encompass identification of the nucleic acid sequence, determining the presence or absence of the nucleic acid sequence, and/or determining the activity of the nucleic acid sequence. A method of detecting a nucleic acid sequence may include contacting a cell sample with a detection agent, binding the detection agent to the nucleic acid sequence, and analyzing a detection profile from the detection agent to determine the presence, absence, or activity of the nucleic acid sequence.

The method may involve utilizing one or more intrinsic properties associated with a detection agent to aid in detection of the nucleic acid sequence. The intrinsic properties may encompass the size of the detection agent, the intensity of the signal, and the location of the detection agent. The size of the detection agent may include the length of the probe and/or the size of the detectable moiety (such as the size of a fluorescent dye molecule) may modulate the specificity of interaction with a regulatory element. The intensity of the signal from the detection agent may correlate to the sensitivity of detection. For example, a detection agent with a molar extinction coefficient of about 0.5-5×10⁶ M⁻¹cm⁻¹ may have a higher intensity signal relative to a detection agent with a molar extinction coefficient outside of the 0.5-5×10⁶ M⁻¹cm⁻¹ range and may have lower attenuation due to scattering and absorption. Further, a detection agent with a longer excited state lifetime and a large Stoke shift (measured by the distance between the excitation and emission peaks) may further improve the sensitivity of detection. The location of the detection agent may, for example, provide the activity state of a nucleic acid sequence. A combination of intrinsic properties of the detection agent may be used to detect a regulatory element of interes.

A detection agent may comprise a detectable moiety that is capable of generating a light, and a probe portion that is capable of hybridizing to a target site on a nucleic acid sequence. As described herein, a detection agent may include a DNA probe portion, an RNA probe portion, a polypeptide probe portion, or a combination thereof. A DNA or RNA probe portion may be between about 10 and about 100 nucleotides in length. A DNA or RNA probe portion may be a TALEN probe, ZFN probe, or a CRISPR probe. A DNA or RNA probe portion may be a padlock probe. A polypeptide probe may comprise a DNA-binding protein, a RNA-binding protein, a protein involved in the transcription/translation process or detects the transcription/translation process, a protein that may detect an open or relaxed portion of a chromatin, or a protein interacting partner of a product of a regulatory element (such as an antibody or binding fragment thereof). In some instances, a detection agent may comprise a DNA or RNA probe portion which may be between about 10 and about 100 nucleotides in length.

A set of detection agents may be used to detect a nucleic acid sequence. The set of detection agents may comprise about 2 to about 20, or more detection agents may be used for detection of a nucleic acid sequence. A detection agent may comprise a polypeptide probe selected from a DNA-binding protein, a RNA-binding protein, a protein involved in the transcription/translation process or detects the transcription/translation process, a protein that may detect an open or relaxed portion of a chromatin, or a protein interacting partner of a product of a regulatory element (such as an antibody or binding fragment thereof).

A detectable moiety that is capable of generating a light may be directly conjugated or bound to a probe portion. A detectable moiety may indirectly conjugated or bound to a probe portion by a conjugating moiety. As described herein, a detectable moiety may be a small molecule (such as a dye) which may be directly conjugated or bound to a probe portion. A detectable moiety may be a fluorescently labeled protein or molecule which may be attached to a conjugating moiety (such as a hapten group, an azido group, an alkyne group) of a probe.

A profile or a detection profile or signature may include the signal intensity, signal location, and/or size of the signal of the detection agent. The profile or the detection profile may comprise about 100 image frames to about 50,000 frames, or more image frames. Analysis of the profile or the detection profile may determine the activity of the regulatory element. The degree of activation may also be determined from the analysis of the profile or detection profile. Analysis of the profile or the detection profile may further determine the optical isolation and localization of the detection agents, which may correlate to the localization of the nucleic acid sequence.

The method may comprise an operation of providing one or more probes capable of binding to a target nucleic acid sequence, as described herein. The method may comprise an operation of binding the one or more probes to the target nucleic acid sequence, as described herein. The method may comprise an operation of photobleaching the one or more probes at one or more wavelengths, as described herein. The method may comprise an operation of detecting a profile of optical emissions associated with the photobleaching, as described herein. The method may comprise an operation of analyzing the detection profile to determine the localization of the target nucleic acid sequence, as described herein.

The localization of a nucleic acid sequence may include contacting a nucleic acid sequence with a first set of detection agents, photobleaching the first set of detection agents for a first time point at a first wavelength to generate a second set of detection agents capable of generating a light at a second wavelength, detecting at least one burst generated by the second set of detection agents to generate a detection profile of the second set of detection agents, and analyzing the detection profile to determine the localization of the nucleic acid sequence.

A detection agent may comprise a detectable moiety that is capable of generating a light, and a probe portion that is capable of hybridizing to a target site on a nucleic acid sequence. Each detection agent within the first set of detection agents may have the same or a different detectable moiety. Each detection agent within the first set of detection agents may have the same detectable moiety. A detectable moiety may comprise a small molecule (such as a fluorescent dye). A detectable moiety may comprise a fluorescently labeled polypeptide, a fluorescently labeled nucleic acid probe, and/or a fluorescently labeled polypeptide complex.

Upon photobleaching, a second set of detection agents may be generated from the first set of detection agents, in which the second set may include detection agents that are capable of generating a burst of light detectable at a second wavelength. For example, bleaching of the set of detection agents may lead to about 50%, or more detection agents within the set to enter into an “OFF-state”. An “OFF-state” may be a dark state in which the detectable moiety crosses from the singlet excited electronic or ON state to the triplet electronic state or OFF-state in which detection of light (such as fluorescence) may be low (for instance, less than 10%, less than 5%, less than 1%, or less than 0.5% of light may be detected). The remainder of the detection agents that have not entered into the OFF-state may generate bursts of lights, or to cycle between a singlet excited electronic state (or ON-state) and a singlet ground electronic state. As such, bleaching of the set of detection agents may generate about 40% or less detection agents within the set that may generate bursts of lights. The bursts of lights may be detected stochastically, at a single burst level in which each burst of light correlates to a single detection agent.

A single wavelength may be used for photobleaching a set of detection agents. At least two wavelengths may be used for photobleaching a set of detection agents. A wavelength at 491 nm may be used. A wavelength at 405 nm may be used in combination with the wavelength at 491 nm. The two wavelengths may be applied simultaneously to photobleach a set of detection agents. The two wavelengths may be applied sequentially to photobleach a set of detection agents. The time for photobleaching a set of detection agents may be from about 10 seconds to about 4 hours, or more. The concentration of the detection agents may be from about 5 nM to about 1 μM.

The burst of lights from the set of detection agents may generate a detection profile. The detection profile may comprise about 100 image frames to about 50,000 frames, or more image frames. The detection profile may also include the signal intensity, signal location, or size of the signal. Analysis of the detection profile may determine the optical isolation and localization of the detection agents, which may correlate to the localization of the nucleic acid sequence.

The detection profile may comprise a chromatic aberration correction. The detection profile may comprise less than 5% or 0% chromatic aberration.

More than one nucleic acid sequence may be detected at the same time. Sometimes, at least 2 to at least 20 or more nucleic acid sequence may be detected at the same time. Each of the nucleic acid sequences may be detected by a set of detection agents. The detectable moiety between the different set of detection agents may be the same. For example, two different sets of detection agents may be used to detect two different nucleic acid sequences and the detectable moieties from the two sets of detection agents may be the same. As such, at least 2 to at least 20 or more nucleic acid sequences may be detected at the same time at the same wavelength. The detectable moiety between the different set of detection agents may also be different. For example, two different sets of detection agents may be used to detect two different nucleic acid sequences and the detectable moiety from one set of detection agents may be detected at a different wavelength from the detectable moiety of the second set of detection agents. As such, at least 2 to at least 20, or more nucleic acid sequences may be detected at the same time in which each of the nucleic acid sequences may be detected at a different wavelength. The nucleic acid sequence may comprise DNA, RNA, polypeptides, or a combination thereof.

The activity of a target nucleic acid sequence may be measuring utilizing the methods described herein. The methods may include detection of a nucleic acid sequence and one or more products of the nucleic acid sequence. One or more products of the nucleic acid sequence may also include intermediate products or elements. The method may comprise contacting a cell sample with a first set and a second set of detection agents, in which the first set of detection agents interact with a target nucleic acid sequence within the cell and the second set of detection agents interact with at least one product of the target nucleic acid sequence, and analyze a detection profile from the first set and the second set of detection agents, in which the presence or the absence of the at least one product indicates the activity of the target nucleic acid sequence.

As described herein, a detection agent may comprise a detectable moiety that is capable of generating a light, and a probe portion that is capable of hybridizing to a target site on a nucleic acid sequence. Each detection agent within the first set of detection agents may have the same or a different detectable moiety. Each detection agent within the first set of detection agents may have the same detectable moiety. A detectable moiety may comprise a small molecule (such as a fluorescent dye). A detectable moiety may comprise a fluorescently labeled polypeptide, a fluorescently labeled nucleic acid probe, and/or a fluorescently labeled polypeptide complex.

The method may also allow photobleaching of the first set and the second set of detection agents, whereby generating a subset of detection agents capable of generating a burst of light. A detection profile may be generated from the detection of a set of light bursts, in which the presence or the absence of the at least one product may indicate the activity of the target nucleic acid sequence.

The nucleic acid sequence may comprise DNA, RNA, polypeptides, or a combination thereof. The nucleic acid sequence may be DNA. The nucleic acid sequence may be RNA. The nucleic acid sequence may be an enhancer RNA (eRNA). The presence of an eRNA may correlate with target gene transcription that is downstream of eRNA. The nucleic acid sequence may be a DNaseI hypersensitive site (DHS). The DHS may be an activated DHS. The pattern of the DHS on a chromatin may correlate to the activity of the chromatin. The nucleic acid sequence may be a polypeptide, such as a transcription factor, a DNA or RNA-binding protein or binding fragment thereof or a polypeptide that is involved in chemical modification. The nucleic acid sequence may be chromatin.

Image Analysis of Protein Markers (e.g., p53BP1) of Cellular Perturbation and Nano-FISH

The below disclosed imaging and image analysis techniques can be used to analyze protein markers (e.g., p53BP1) of cellular perturbation and/or Nano-FISH.

A. Epifluorescence Imaging

One or more far-field or near-field fluorescence techniques may be utilized for the detection, localization, activity determination, and mapping of one or more protein agglomerations or nucleic acid sequences described herein. A microscopy method may be an air or an oil immersion microscopy method used in a conventional microscope, a holographic or tomographic imaging microscope, or an imaging flow cytometer instrument. In such a method, imaging flow cytometers such as the ImageStream (EMD Millipore), conventional microscopes or commercial high-content imagers (such as the Operetta (Perkin Elmer), IN Cell (GE), etc.) deploying wide-field and/or confocal imaging modes may achieve subcellular resolution to detect signals of interest. For example, DAPI (4′,6-diamidino-2-phenylindole) stain may be used to identify cell nuclei and another stain may be used to identify cells containing a nuclease protein.

B. Super-Resolution Imaging

A microscopy method may utilize a super-resolution microscopy, which allows images to be taken with a higher resolution than the diffraction limit. A super-resolution microscopy method may utilize a deterministic super-resolution microscopy method, which utilizes a fluorophore's nonlinear response to excitation to enhance resolution. Exemplary deterministic super-resolution methods may include stimulated emission depletion (STED), ground state depletion (GSD), reversible saturable optical linear fluorescence transitions (RESOLFT), and/or saturated structured illumination microscopy (SSIM). A super-resolution microscopy method may also include a stochastic super-resolution microscopy method, which utilizes a complex temporal behavior of a fluorophore, to enhance resolution. Exemplary stochastic super-resolution method may include super-resolution optical fluctuation imaging (SOFI), all single-molecular localization method (SMLM) such as spectral precision determination microscopy (SPDM), SPDMphymod, photo-activated localization microscopy (PALM), fluorescence photo-activated localization microscopy (FPALM), selective plane illumination microscopy (SPIM), stochastic optical reconstruction microscopy (STORM), and dSTORM.

A microscopy method may be a single-molecular localization method (SMLM). A microscopy method may be a spectral precision determination microscopy (SPDM) method. A SPDM method may rely on stochastic burst or blinking of fluorophores and subsequent temporal integration of signals to achieve lateral resolution at, for example, between about 10 nm and about 100 nm.

A microscopy method may be a spatially modulated illumination (SMI) method. A SMI method may utilize phased lasers and interference patterns to illuminate specimens and increase resolution by measuring the signal in fringes of the resulting Moire patterns.

A microscopy method may be a synthetic aperture optics (SAO) method. A SAO method may utilize a low magnification, low numerical aperture (NA) lens to achieve large field of view (FOV) and depth of field, without sacrificing spatial resolution. For example, an SAO method may comprise illuminating the detection agent-labeled target (such as a target protein agglomeration or nucleic acid sequence) with a predetermined number (N) of selective excitation patterns, where the number (N) of selective excitation patterns is determined based upon the detection agent's physical characteristics corresponding to spatial frequency content (such as the size, shape, and/or spacing of the detection agents on the imaging target) from the illuminated target, optically imaging the illuminated target at a resolution insufficient to resolve the objects on the target, and processing optical images of the illuminated target using information on the selective excitation patterns to obtain a final image of the illuminated target at a resolution sufficient to resolve the objects on the target. The number (N) of selective excitation patterns may correspond to the number of k-space sampling points in a k-space sampling space in a frequency domain, with the extent of the k-space sampling space being substantially proportional to an inverse of a minimum distance (Δx) between the objects that is to be resolved by SAO, and with the inverse of the k-space sampling interval between the k-space sampling points being less than a width (w) of a detected area captured by a pixel of a system for said optical imaging. The number (N) may include a function of various parameters of the imaging system (such as a magnification of the objective lens, numerical aperture of the objective lens, wavelength of the light emitted from the imaging target, and/or effective pixel size of the pixel sensitive area of the image detector, etc.).

A SAO method may analyze a set of detection agent profiles from at least 100, at least 200, at least 250, at least 500, at least 1000, or more cells imaged simultaneously within one field of view utilizing an imaging instrument. The one field of view may be a single wide field of view (FOV) allowing image capture of at least 50, at least 100, at least 200, at least 250, at least 500, at least 1000, or more cells. The single wide field of view may be about 0.70 mm by about 0.70 mm field of view. The SAO imaging instrument may enable a resolution of about 0.25 μm with a 20×/0.45NA lens. The SAO imaging instrument may enable a depth of field of about 2.72 μm with a 20×/0.45NA lens. The imaging instrument may enable a working distance of about 7 mm with a 20×/0.45NA lens. The imaging instrument may enable a z-stack of 1 with a 20×/0.45NA lens. The SAO method may further integrate and interpolate 3-dimensional images from 2-dimensional images. The SAO method may enable the image acquisition of cell images at high spatial resolution and FOV. For example, for a given cell type, the SAO method may provide a FOV that is at least about 1.5×, at least about 2×, at least about 3×, at least about 4×, at least about 5×, at least about 6×, at least about 7×, at least about 8×, at least about 9×, at least about 10×, at least about 15×, at least about 20×, or more as compared to a FOV provided by a method of microscope imaging using a 40× or 60× objective. For example, the SAO method may provide a FOV corresponding to a 20× microscope lens with a spatial resolution corresponding to a 100× microscope lens.

The SAO imaging instrument may be, for example, an SAO instrument as described in U.S. Patent Publication No. 2011/0228073 (Lee et al.). The SAO imaging instrument may be, for example, a StellarVision™ imaging platform supplied by Optical Biosystems, Inc. (Santa Clara, Calif.).

### Analysis of Fluorescence Images

Fluorescence images may be processed by a method for analysis of, e.g., cell nuclei, target protein agglomerations (e.g., p53BP1), diffused localization of target proteins, and/or FISH signals. The method may comprise obtaining a fluorescence image of one or more probes bound to one or more target proteins or nucleic acid sequences, as described herein. The method may comprise deconvolving the image one or more times, as described herein. The method may comprise generating a region of interest (ROI) from the deconvolved image, as described herein. The method may comprise analyzing the ROI to determine the locations of all target proteins or nucleic acid sequences, as described herein.

Images obtained using the systems and methods described herein may be subjected to an image analysis method. The images may be obtained using the epifluorescence imaging systems and methods described herein. The image may be obtained using the super-resolution imaging systems and methods described herein.

The image analysis method may allow a quantitative morphometric analysis to be conducted on regions of interest (ROIs) within the images. The image analysis method may be implemented using Matlab, Octave, Python, Java, Perl, Visual Studio, C, or ImageJ. The image analysis method may be adapted from methods for processing fluorescence microscopy images of cells for segmentation of cell nuclei, protein agglomerations, Nano-FISH signals, and/or nuclease localization. The image analysis method may be fully automated and/or tunable by the user. The image analysis method may be configurable to identify p53BP1 foci regardless of the shapes of the foci. The image analysis method may be configurable to process two-dimensional and/or three-dimensional images. The image analysis method may allow high throughput of estimation of cell count and boundaries in cell populations, which may be obtained with a speed-up of at least about 2 times, at least about 5 times, at least about 10 times, at least about 15 times, at least about 20 times, at least about 25 times, at least about 30 times, at least about 35 times, at least about 40 times, at least about 45 times, at least about 50 times, at least about 100 times, or more, as compared to manual identification and counting of cell populations.

The image analysis method may comprise a deconvolution of the image. The deconvolution process may improve the contrast and resolution of cell images for further analysis. The image analysis method may comprise an iterative deconvolution of the image. The image analysis method may comprise 1, 2, 3, 4, 5, 6, 7, 8, 9, or 10 iterations of deconvolving the image. The image analysis method may comprise more than 1, more than 2, more than 3, more than 4, more than 5, more than 6, more than 7, more than 8, more than 9, or more than 10 iterations of deconvolving the image. The deconvolution procedure may remove or reduce out-of-focus blur or other sources of noise in the epifluorescence images or super-resolution images, thereby enhancing the signal-to-noise ratio (SNR) within ROIs.

The image analysis method may further comprise an identification of the ROIs (e.g., candidate cells). The ROIs may be identified using an automated detection method. The ROIs may be identified by processing the raw or deconvolved or reconstructed or pre-processed images by applying a segmentation algorithm. This may allow the rapid delineation of ROIs within the epifluorescence or super-resolution images, thereby allowing scalability of processing images. The segmentation of ROIs may comprise planarization of three-dimensional images (e.g., generated by z-stacking to obtain three-dimensional cell volumes) by utilizing a maximum intensity projection image to generate a two-dimensional ROI mask. For rapid segmentation, the two-dimensional ROI mask may act as a template for an initial three-dimensional mask. For instance, the initial three-dimensional mask may be generated by projecting the two-dimensional ROI mask into a third spatial dimension. The projection may be a weighted projection. The initial three-dimensional mask may be further refined to obtain a refined three-dimensional ROI mask. Refinement of the initial three-dimensional mask may be achieved utilizing adaptive thresholding and/or region growing methods. Refinement of the initial three-dimensional mask may be achieved by iteratively applying adaptive thresholding and/or region growing methods. The iterative procedure may result in a final three-dimensional ROI mask. The final three-dimensional ROI mask may comprise information regarding the locations of all fluorescently-labeled proteins or FISH-labeled nucleic acid sequences within each cell in a sample.

The segmentation may detect ROIs using two-dimensional or three-dimensional computer vision methods such as edge detection and morphology. The ROIs may include cell nuclei, protein (e.g., p53BP1) foci, FISH foci, nuclease localization, or a combination thereof within each cell in a cell population within a field of view (FOV).

The image analysis method may further comprise feature extraction/computation from the segmented ROIs (e.g., detected candidate cells). Such sets of features may be selected to enable high performance (e.g., accuracy, throughput, sensitivity, specificity, etc.) of identifying/counting ROIs. Morphological features/parameters may be extracted from the segmented ROIs, such as count, spatial location, size (area/volume), shape (circularity/sphericity, eccentricity, irregularity (concavity/convexity)), diameter, perimeter/surface area, etc. In addition, other image parameters may also be extracted from the segmented ROIs, such as quantitative measures of image texture that may be pixel-based or region-based over a tunable length scale (e.g., nuclear diameter, nuclear area, nuclear volume, perimeter, surface area, DNA content, DNA texture measures).

In the case of ROIs that include protein foci, extracted features may include number of protein marker foci, size of protein marker foci, shape of protein marker foci, amount of protein marker per cell, spatial location and localization pattern of protein marker foci. In the case of ROIs that include nuclease localization, number of nuclease per cell, amount of nuclease per cell, nuclease localization or texture, number of cell engineering tool foci, size of cell engineering tool foci, shape of cell engineering tool foci, amount of cell engineering tool foci per cell, spatial location and localization pattern of cell engineering tool foci. In addition, in the case of ROIs that include Nano-FISH foci, additional features may be extracted, such as number, size, shape, amount, spatial location and localization pattern of Nano-FISH foci.

After the image analysis method has analyzed the cell nuclei, target protein agglomerations (e.g., p53BP1), diffused localization of target proteins, and/or FISH signals, further informatics and analysis may be performed based on the image analysis results. For example, specificity analysis may be performed by analyzing locations of co-localization between Nano-FISH-labeled genomic loci and p53BP1. Cell images with high co-localization and similar counts between Nano-FISH-labeled genomic loci and p53BP1 may indicate samples with high potency and specificity of nuclease activity (e.g., with minimal off-target effects), while cell images without co-localization between immunoNanoFISH and p53BP1 may indicate samples with issues such as decreased potency of nuclease activity, decreased specificity of nuclease activity (e.g., with some off-target effects), or that an editing event was not detected by the assay.

The image analysis method may analyze acquired image data comprising a cell population to generate an output of estimating a count and/or boundaries (e.g., segmented ROIs) of the cell population. For example, the image analysis method may apply a prediction algorithm (e.g., a predictive analytics algorithm) to the acquired data to generate output of estimating a count and/or boundaries (e.g., segmented ROIs) of the cell population. The prediction algorithm may comprise an artificial intelligence based predictor, such as a machine learning based predictor, configured to process the acquired image data comprising a cell population to generate the output of estimating a count and/or boundaries (e.g., segmented ROIs) of the cell population. The machine learning predictor may be trained using datasets from one or more sets of images of known cell populations as inputs and known counts and/or boundaries (e.g., segmented ROIs) of the cell populations as outputs to the machine learning predictor.

The machine learning predictor may comprise one or more machine learning algorithms. Examples of machine learning algorithms may include a support vector machine (SVM), a naïve Bayes classification, a random forest, a neural network, deep learning, or other supervised learning algorithm or unsupervised learning algorithm for classification and regression. The machine learning predictor may be trained using one or more training datasets corresponding to image data comprising cell populations.

Training datasets may be generated from, for example, one or more sets of image data having common characteristics (features) and outcomes (labels). Training datasets may comprise a set of features and labels corresponding to the features. Features may comprise characteristics such as, for example, certain ranges or categories of cell measurements, such as morphological features/parameters (count, size, diameter, area, volume, perimeter length, circularity, irregularity, eccentricity, etc.), other image parameters (contrast, correlation, entropy, energy, and homogeneity/uniformity, etc.), nuclear size (diameter, area, or volume), perimeter or surface area, shape (e.g., circularity, irregularity, eccentricity, etc.), DNA content, DNA texture measures, characteristics of p53BP1 foci (e.g., number, size, shape, etc.), amount of p53BP1 protein per cell, spatial location and localization pattern of p53BP1 foci, amount of nuclease per cell, nuclease localization or texture, and characteristics of FISH signals (number, size, shape, amount, spatial location and localization pattern). Labels may comprise outcomes such as, for example, estimated or actual counts and boundaries of cells in a cell population or nuclease specificity or its activity.

Training sets (e.g., training datasets) may be selected by random sampling of a set of data corresponding to one or more sets of image data. Alternatively, training sets (e.g., training datasets) may be selected by proportionate sampling of a set of data corresponding to one or more sets of image data. The machine learning predictor may be trained until certain predetermined conditions for accuracy or performance are satisfied, such as having minimum desired values corresponding to cell identification accuracy measures. For example, the cell identification accuracy measure may correspond to estimated or actual counts and boundaries (e.g., segmented ROIs) of cells in a cell population. Examples of cell identification accuracy measures may include sensitivity, specificity, positive predictive value (PPV), negative predictive value (NPV), accuracy, and area under the curve (AUC) of a Receiver Operating Characteristic (ROC) curve corresponding to the accuracy of generating estimated or actual counts and boundaries (e.g., segmented ROIs) of cells in a cell population.

For example, such a predetermined condition may be that the sensitivity of identifying a cell of interest comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the specificity of identifying a cell of interest comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the positive predictive value (PPV) of identifying a cell of interest comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the negative predictive value (NPV) of identifying a cell of interest comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the area under the curve (AUC) of a Receiver Operating Characteristic (ROC) curve of identifying a cell of interest comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

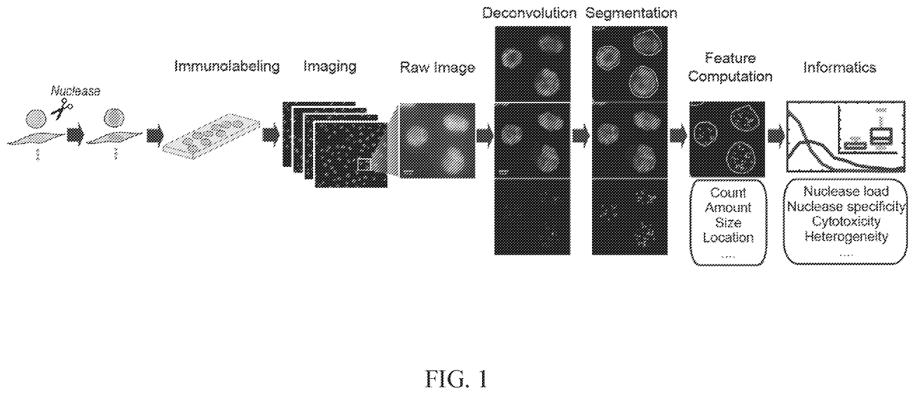

In some embodiments, image analysis can also be carried out as shown in FIG. 1, which illustrates an assay workflow for cellular imaging of phospho-53BP1 (p53BP1) foci.

The image analysis method may be implemented in an automated manner, such as using the digital processing devices described herein.

In certain aspects, % nuclease specificity for a nuclease can be computed from the per-cell p53bp1 foci count data. The data distributions for the nuclease-treated and the corresponding untreated reference (background) cell samples are computed. Given the detection efficiency of the p53bp1 assay (PD) at the target site and the proliferating cell fraction (Fp), a theoretical on-target distribution is calculated for the on-target activity of the nuclease. Subsequently, the distribution of the nuclease-treated sample is normalized by the distribution of the control sample and the theoretical on-target distribution using a process of non-negative least squares deconvolution. Lastly, the specificity is calculated as follows from the distribution of the background-normalized cell population: Given the ploidy (P_{T}) of the editing target, nuclease specificity is the % fraction of background-normalized cells containing p53BP1 foci from 0 to P_{T}. For simplicity in modeling, F_{P} and P_{D} are set to 0 and 1.

Baseline level or threshold level above which a DNA binding domain of a gene editing tool (e.g., a nuclease) is deemed to be non-specific can be calculated empirically by carrying out the imaging assays described herein. Such baseline or threshold level may be application-specific and can be determined by the requirements of an application as a set threshold on the magnitude of change in protein load in response to treatment (relative to background protein load in reference untreated cells) beyond which cell engineering tool is deemed non-specific, or as a relative ranking of cell engineering tools in a screening application when one or several best performing tools are picked.

In one case, protein indicative of cellular response is stained and imaged in fixed cells, total protein load is calculated by measuring intensity of protein staining within a cell. Change in total protein load is used as a measure of cell response to treatment.

In another case, protein indicative of cellular response is stained and imaged in fixed cells, and protein accumulation at distinct locations within the cell is detected and enumerated. Change in the number of protein foci is used as a measure of cell response to treatment. In some instances, this change can be expressed as a specificity score.

In yet another case, protein indicative of cellular response is stained with immunofluorescence and target DNA loci are stained with nanoFISH and imaged in fixed cells. Protein accumulation at distinct locations and co-localization with nanoFISH spots within the cell are detected and enumerated. Change in the number of protein foci not co-localized with target nanoFISH spots is used as a measure of off-target cell response to treatment.

A. Digital Processing Device

The systems, apparatus, and methods described herein may include a digital processing device, or use of the same. The digital processing device may include one or more hardware central processing units (CPU) that carry out the device's functions. The digital processing device may further comprise an operating system configured to perform executable instructions. In some instances, the digital processing device is optionally connected to a computer network, is optionally connected to the Internet such that it accesses the World Wide Web, or is optionally connected to a cloud computing infrastructure. In other instances, the digital processing device is optionally connected to an intranet. In other instances, the digital processing device is optionally connected to a data storage device.

In accordance with the description herein, suitable digital processing devices may include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers may include those with booklet, slate, and convertible configurations, known to those of skill in the art.

The digital processing device may include an operating system configured to perform executable instructions. The operating system may be, for example, software, including programs and data, which may manage the device's hardware and provides services for execution of applications. Those of skill in the art will recognize that suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some cases, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® U®, and Ouya®.

In some instances, the device may include a storage and/or memory device. The storage and/or memory device may be one or more physical apparatuses used to store data or programs on a temporary or permanent basis. In some instances, the device is volatile memory and requires power to maintain stored information. In other instances, the device is non-volatile memory and retains stored information when the digital processing device is not powered. In still other instances, the non-volatile memory comprises flash memory. The non-volatile memory may comprise dynamic random-access memory (DRAM). The non-volatile memory may comprise ferroelectric random access memory (FRAM). The non-volatile memory may comprise phase-change random access memory (PRAM). The device may be a storage device including, by way of non-limiting examples, CD-ROMs, DVDs, flash memory devices, magnetic disk drives, magnetic tapes drives, optical disk drives, and cloud computing based storage. The storage and/or memory device may also be a combination of devices such as those disclosed herein.

The digital processing device may include a display to send visual information to a user. The display may be a cathode ray tube (CRT). The display may be a liquid crystal display (LCD). Alternatively, the display may be a thin film transistor liquid crystal display (TFT-LCD). The display may further be an organic light emitting diode (OLED) display. In various cases, on OLED display is a passive-matrix OLED (PMOLED) or active-matrix OLED (AMOLED) display. The display may be a plasma display. The display may be a video projector. The display may be a combination of devices such as those disclosed herein.

The digital processing device may also include an input device to receive information from a user. For example, the input device may be a keyboard. The input device may be a pointing device including, by way of non-limiting examples, a mouse, trackball, track pad, joystick, game controller, or stylus. The input device may be a touch screen or a multi-touch screen. The input device may be a microphone to capture voice or other sound input. The input device may be a video camera or other sensor to capture motion or visual input. Alternatively, the input device may be a Kinect™, Leap Motion™, or the like. In further aspects, the input device may be a combination of devices such as those disclosed herein.

B. Non-Transitory Computer Readable Storage Medium

In some instances, the systems, apparatus, and methods disclosed herein may include one or more non-transitory computer readable storage media encoded with a program including instructions executable by the operating system of an optionally networked digital processing device. In further instances, a computer readable storage medium is a tangible component of a digital processing device. In still further instances, a computer readable storage medium is optionally removable from a digital processing device. A computer readable storage medium may include, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

C. Computer Program

The systems, apparatus, and methods disclosed herein may include at least one computer program, or use of the same. A computer program includes a sequence of instructions, executable in the digital processing device's CPU, written to perform a specified task. In some embodiments, computer readable instructions are implemented as program modules, such as functions, objects, Application Programming Interfaces (APIs), data structures, and the like, that perform particular tasks or implement particular abstract data types. In light of the disclosure provided herein, those of skill in the art will recognize that a computer program, in certain embodiments, is written in various versions of various languages.

The functionality of the computer readable instructions may be combined or distributed as desired in various environments. A computer program may comprise one sequence of instructions. A computer program may comprise a plurality of sequences of instructions. In some instances, a computer program is provided from one location. In other instances, a computer program is provided from a plurality of locations. In additional cases, a computer program includes one or more software modules. Sometimes, a computer program may include, in part or in whole, one or more web applications, one or more mobile applications, one or more standalone applications, one or more web browser plug-ins, extensions, add-ins, or add-ons, or combinations thereof.

D. Web Application

A computer program may include a web application. In light of the disclosure provided herein, those of skill in the art will recognize that a web application, in various aspects, utilizes one or more software frameworks and one or more database systems. In some cases, a web application is created upon a software framework such as Microsoft® .NET or Ruby on Rails (RoR). In some cases, a web application utilizes one or more database systems including, by way of non-limiting examples, relational, non-relational, object oriented, associative, and XML database systems. Sometimes, suitable relational database systems may include, by way of non-limiting examples, Microsoft® SQL Server, mySQL™ and Oracle®. Those of skill in the art will also recognize that a web application, in various instances, is written in one or more versions of one or more languages. A web application may be written in one or more markup languages, presentation definition languages, client-side scripting languages, server-side coding languages, database query languages, or combinations thereof. A web application may be written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CS S). A web application may be written to some extent in a client-side scripting language such as Asynchronous Javascript and XML (AJAX), Flash® Actionscript, Javascript, or Silverlight®. A web application may be written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. Sometimes, a web application may be written to some extent in a database query language such as Structured Query Language (SQL). Other times, a web application may integrate enterprise server products such as IBM® Lotus Domino®. In some instances, a web application includes a media player element. In various further instances, a media player element utilizes one or more of many suitable multimedia technologies including, by way of non-limiting examples, Adobe® Flash®, HTML 5, Apple® QuickTime®, Microsoft® Silverlight®, Java™, and Unity®.

E. Mobile Application

A computer program may include a mobile application provided to a mobile digital processing device. In some cases, the mobile application is provided to a mobile digital processing device at the time it is manufactured. In other cases, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

F. Standalone Application

A computer program may include a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. A computer program may include one or more executable complied applications.

### Web Browser Plug-in

The computer program may include a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) may be software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

A. Software Modules

The systems and methods disclosed herein may include software, server, and/or database modules, or use of the same. In view of the disclosure provided herein, software modules may be created by techniques known to those of skill in the art using machines, software, and languages known to the art. The software modules disclosed herein may be implemented in a multitude of ways. A software module may comprise a file, a section of code, a programming object, a programming structure, or combinations thereof. A software module may comprise a plurality of files, a plurality of sections of code, a plurality of programming objects, a plurality of programming structures, or combinations thereof. In various aspects, the one or more software modules comprise, by way of non-limiting examples, a web application, a mobile application, and a standalone application. In some instances, software modules are in one computer program or application. In other instances, software modules are in more than one computer program or application. In some cases, software modules are hosted on one machine. In other cases, software modules are hosted on more than one machine. Sometimes, software modules may be hosted on cloud computing platforms. Other times, software modules may be hosted on one or more machines in one location. In additional cases, software modules are hosted on one or more machines in more than one location.

B. Databases

The methods, apparatus, and systems disclosed herein may include one or more databases, or use of the same. In view of the disclosure provided herein, those of skill in the art will recognize that many databases are suitable for storage and retrieval of analytical information described elsewhere herein. In various aspects described herein, suitable databases may include, by way of non-limiting examples, relational databases, non-relational databases, object oriented databases, object databases, entity-relationship model databases, associative databases, and XML databases. A database may be Internet-based. A database may be web-based. A database may be cloud computing-based. Alternatively, a database may be based on one or more local computer storage devices.

C. Services

Methods and systems described herein may further be performed as a service. For example, a service provider may obtain a sample that a customer wishes to analyze. The service provider may then encode the sample to be analyzed by any of the methods described herein, performs the analysis and provides a report to the customer. The customer may also perform the analysis and provides the results to the service provider for decoding. In some instances, the service provider then provides the decoded results to the customer. In other instances, the customer may receive encoded analysis of the samples from the provider and decodes the results by interacting with softwares installed locally (at the customer's location) or remotely (e.g. on a server reachable through a network). Sometimes, the softwares may generate a report and transmit the report to the costumer. Exemplary customers include clinical laboratories, hospitals, industrial manufacturers and the like. Sometimes, a customer or party may be any suitable customer or party with a need or desire to use the methods provided herein.

D. Server

The methods provided herein may be processed on a server or a computer server). The server may include a central processing unit (CPU, also “processor”) which may be a single core processor, a multi core processor, or plurality of processors for parallel processing. A processor used as part of a control assembly may be a microprocessor. The server may also include memory (e.g. random access memory, read-only memory, flash memory); electronic storage unit (e.g. hard disk); communications interface (e.g. network adaptor) for communicating with one or more other systems; and peripheral devices which includes cache, other memory, data storage, and/or electronic display adaptors. The memory, storage unit, interface, and peripheral devices may be in communication with the processor through a communications bus (solid lines), such as a motherboard. The storage unit may be a data storage unit for storing data. The server may be operatively coupled to a computer network (“network”) with the aid of the communications interface. A processor with the aid of additional hardware may also be operatively coupled to a network. The network may be the Internet, an intranet and/or an extranet, an intranet and/or extranet that is in communication with the Internet, a telecommunication or data network. The network with the aid of the server, may implement a peer-to-peer network, which may enable devices coupled to the server to behave as a client or a server. The server may be capable of transmitting and receiving computer-readable instructions (e.g., device/system operation protocols or parameters) or data (e.g., sensor measurements, raw data obtained from detecting metabolites, analysis of raw data obtained from detecting metabolites, interpretation of raw data obtained from detecting metabolites, etc.) via electronic signals transported through the network. Moreover, a network may be used, for example, to transmit or receive data across an international border. The server may be in communication with one or more output devices such as a display or printer, and/or with one or more input devices such as, for example, a keyboard, mouse, or joystick. The display may be a touch screen display, in which case it functions as both a display device and an input device. Different and/or additional input devices may be present such an enunciator, a speaker, or a microphone. The server may use any one of a variety of operating systems, such as for example, any one of several versions of Windows®, or of MacOS®, or of Unix®, or of Linux®.

The storage unit may store files or data associated with the operation of a device, systems or methods described herein. The server may communicate with one or more remote computer systems through the network. The one or more remote computer systems may include, for example, personal computers, laptops, tablets, telephones, Smart phones, or personal digital assistants. A control assembly may include a single server. In other situations, the system may include multiple servers in communication with one another through an intranet, extranet and/or the Internet. The server may be adapted to store device operation parameters, protocols, methods described herein, and other information of potential relevance. Such information may be stored on the storage unit or the server and such data is transmitted through a network.

### Kits

A composition described herein may be supplied in the form of a kit. A composition may be materials and software for image analysis of a protein marker (e.g., p53BP1) of a cellular response induced by a cellular perturbation. Materials can include a detectable agent that binds to the protein (e.g., a primary antibody fluorophore conjugate or a primary antibody against the protein and a secondary antibody-fluorophore conjugate). Materials can further include a detectable agent that binds to a cell engineering tool (e.g., genome editing complex, gene regulator) to be tested (e.g., a primary antibody fluorophore conjugate or a primary antibody against the protein and a secondary antibody-fluorophore conjugate). A composition can be an oligonucleotide Nano-FISH probe set designed for a target nucleic acid sequence. The kits of the present disclosure may further comprise instructions regarding the method of using the detectable agents to detect protein (e.g., p53BP1) load, cell engineering tool, or probe set to detect the target nucleic acid sequence.

The components of the kit may be in dry or liquid form. If they are in dry foam, the kit may include a solution to solubilize the dried material. The kit may also include transfer factor in liquid or dry form. In some embodiments, if the transfer factor is in dry form, the kit includes a solution to solubilize the transfer factor. The kit may also include containers for mixing and preparing the components. The kits as described herein also may include a means for containing compositions of the present disclosure in close confinement for commercial sale and distribution.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts may be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

### EXAMPLES

These examples are provided for illustrative purposes only and not to limit the scope of the claims provided herein.

### Example 1

### Assay Workflow for Cellular Imaging of p53BP1 Foci

This example illustrates an assay workflow for cellular imaging of phospho-53BP1 (p53BP1) foci. FIG. 1 shows a brief summary of the assay workflow including the steps of nuclease transfection in cells, immunolabeling, imaging processing raw images by deconvolution, enhancement, or reconstruction and segmentation, feature computation (e.g., count, amount, size, location), and informatics and analysis (determining nuclease load and/or specificity, cytotoxicity, and/or heterogeneity) from the extracted/computed features.

A nuclease (e.g., TALENs or Cas9) was delivered to cells by electroporation. Cells were incubated for a period of time, such as 24 hours, necessary for nuclease activity and cell response to nuclease-induced DNA double-stranded breaks.

The cells were sampled for evaluation of nuclease specificity. Cells were fixed onto glass slides, coverslips, or glass-bottom well-plates, stained with fluorescent labeled antibodies against p53BP1 and the nuclease protein, and imaged with a fluorescence microscope (e.g., Nikon). For microscopy on a Nikon, raw fluorescence microscopy images were deconvolved (e.g., by processing the raw images with a deconvolution algorithm), regions of interest such as cell nuclei, p53BP1 foci, and nuclease localization were algorithmically delineated (e.g., by processing the deconvolved images with a segmentation algorithm), and morphological features/parameters (such as count, size, diameter, area, volume, perimeter length, circularity, irregularity, eccentricity, etc.) and other image parameters (such as contrast, correlation, entropy, energy, and homogeneity/uniformity) were computed for each cell (e.g., by applying one or more feature extraction algorithms to the segmented images). The measured per-cell feature information was statistically analyzed to produce quantitative specificity metrics for the tested nuclease(s). FIG. 17 shows an assay workflow for microscopy on a Stellar-Vision microscope. Images are captured on the Stellar-Vision microscope, images were reconstructed, images were segmented for regions of interest such as cell nucleic, p53BP1 foci, and nuclease localization, features were computed (such as count, size, diameter, area, volume, perimeter length, circularity, irregularity, eccentricity, etc.). The measured per-cell feature information was statistically analyzed to produce quantitative specificity metrics for the tested nuclease(s).

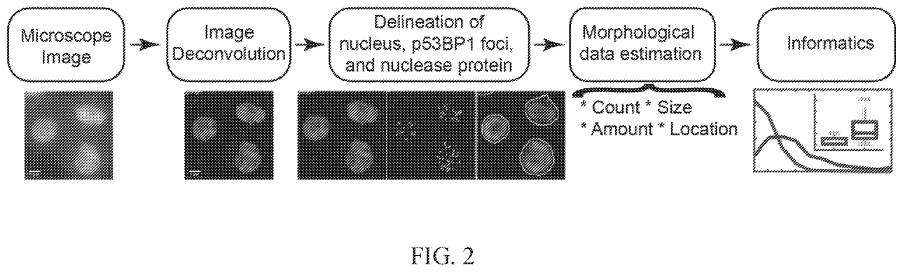

FIG. 2 shows further details on image analysis including the steps of obtaining a fluorescence microscopy image, image deconvolution, delineation/segmentation of cell nuclei, p53BP1 foci, and nuclease protein, morphological data estimation, and informatics/analysis as described in FIG. 1. Acquired cell images were first deconvolved to minimize the effect of out-of-focus blurring caused by the widefield imaging optics. Subsequently, automated 2D/3D computer vision methods were used to delineate regions of interest (ROIs) such as the nucleus, p53BP1 foci, and nuclease protein localization within every cell in the field of view (FOV). The derived ROI masks were used to estimate per-cell morphological parameters (or features) such as count, size, amount, location, and heterogeneity as needed. The estimated morphological parameters and other image parameters of the cells were analyzed using informatics methods to obtain statistical inferences on the activity and specificity of the delivered nuclease relative to control cell samples.

### Example 2

### Transfection of Cells with Nucleases

This example illustrates transfection of cells with nucleases. For all transfections a BTX ECM830 device with a 2 mm gap cuvette was used. TALEN mRNAs were prepared using a mMessageMachine T7 Ultra Kit (#AM1345, Ambion). For each transfection, 0.2×10⁶ cells were washed twice with PBS and centrifuged. Cell pellets were resuspended in 100 μl BTexpress solution (BTX Harvard Apparatus, Cat #45-0805) and 2 μg mRNA per TALEN Monomer was added. Cell/mRNA mixtures were transferred to a transfection cuvette and electroporated with one pulse of 250V for 5 msec. Following electroporation, cells were transferred to pre-warmed media. K562 cells or A549 cells were transferred to 2 mL of pre-warmed IMDM/10% FBS/1% PS (for K562 cells) or 2 mL of pre-warmed F-12K/10% FBS/1% PS (for A549 cells) and CD34 cells were transferred to 600 μl xvivo/CC110/IL6. Cells were incubated at 30° C. for 24 hours prior to imaging. Genotyping was performed 24 and 48 hours post-transfection.

### Example 3

### T Cell Stimulation, and Transfection Methods

This example illustrates T cell stimulation and transfection methods. Human CD4⁺ T lymphocytes were isolated from peripheral blood mononuclear cells (PBMCs) of non-mobilized healthy donors by negative selection. Human CD4+ T lymphocyte culture medium was prepared with X-VIVO 15 (Lonza, Basel, Switzerland) supplemented with 10% FBS, 2 mM L-glutamine, 1% penicillin/streptomycin, and 20 ng/ml IL2 (PeproTech, Rocky Hill, N.J., USA). Cell washing media was prepared with 10% FBS in PBS. Cells were cultured by pre-warming the culture media and washing media to 37° C. Cell tubes were filled with 30 ml washing media and cells were counted. Cells were centrifuged at 400×g for 8 minutes at room temperature, resuspended in complete culture media to a concentration of 1-2×10⁶ cells/mL, and placed in 37° C., 5% CO2 humidified incubator for further experimentation.

T cells were activated with Anti-CD3/CD28-Dynabeads (Life Technologies, Cat #11132D). Dynabeads washing buffer was prepared containing PBS with 0.1% BSA and 2 mM EDTA, pH 7.4. Anti-CD3/CD28-Dynabeads were resuspended and transferred to a tube. An equal volume of Dynabeads washing buffer was added, the tube was placed on a magnet for 1 min, and the supernatant was discarded. Washed Dynabeads were resuspended in culture media. Washed Dynabeads were added to the CD4+ T cell culture suspension at a bead to cell ratio of 1:1 and the cells were mixed with a pipette. Plates were incubated at 37° C., 5% CO2 humidified incubator for 24 hours to activate T cells. Activated T cells were mixed and placed on the magnet for 5 min and supernatants containing cells were collected. This step was repeated 2-3 times to obtain activated T cells (without Dynabeads) for further experimentation. For transfection of T cells, after transfection cell maintain medium was prepared containing X-VIVO 15 (Lonza, Basel, Switzerland) supplemented with 10% FBS, 2 mM L-glutamine, 1% penicillin/streptomycin, 20 ng/ml IL2 (PeproTech, Rocky Hill, N.J., USA), and 20 ng/ml IL7 (PeproTech, Rocky Hill, N.J., USA).

Electroporation settings included a choose mode of LV, set voltage of 250 V, set pulse length of 5 ms, 1 set number of pulses, a BTX Disposable Cuvette (2 mm gap) electrode type and a desired field strength of 3000 V/cm. Cell culture plates were prepared with after transfection cell maintain medium by filling appropriate number of wells with desired 800 μl. Plates were pre-incubated/equilibrated in a humidified 37° C., 5% CO₂ incubator. 1-2 μs of TALEN mRNA was aliquoted in a separate tube. BTXpress high performance electroporation solution (BTX, Holliston, Mass., USA) was brought to room temperature. Activated CD4+ T cells were collected and counted to determine cell density. Total cells needed (0.2-0.5×10⁶ cells per sample) were centrifuged at 300×g for 8 minutes at room temperature and washed twice with PBS. For transfection, CD4⁺ T cells were resuspended in BTXpress high performance electroporation solution (Harvard Apparatus, Holliston, Mass., USA), to a final density of 2-5×10⁶ cells/mL. 100 ul of cells was mixed with aliquoted mRNA. Cell-mRNA mixture was added to a well of MOS Multi-Well Electroporation Plate, sealed, and placed into the HT Electroporation System. T cells were electroporated in a BTX ECM830 Square Wave electroporator using a single pulse of 250 V for 5 ms. Electroporated CD4+ T cells were placed in an Axygen Deep 96-well plate or 12/24 well Falcon Polystyrene Microplates with pre-warmed cell maintain medium. Cells were “cold shocked” in a humidified 30° C., 5% CO₂ incubator for 16-24 hour, then incubated in a humidified 37° C., 5% CO₂ incubator until analysis. Gene expression or down regulation was detectable as early as 4-8 hours post electroporation. For imaging cells were collected 24 hours after transfection. For genomic DNA isolation, cells were incubated for around 48-72 hours. For RNA collection, cells were incubated up to 4-5 days.

### Example 4

### p53BP1 Immunofluorescence Imaging

This example illustrates p53BP1 immunofluorescence analysis using the compositions and methods of the present disclosure.

### Coverslip Format

Cell preparation. Cells were prepared for immunofluorescence staining and image analysis on a coverslip and in 24 well plates. For preparation of cells on coverslips, cells were seeded onto a poly-1-lysine coated #1.5 glass coverslip (12 mm round or 18 mm square). First, coverslips were placed into a well of a 6-well tissue culture plate. Cells were pre-washed with PBS, resuspended to ^{˜}2,000,000 cells/mL in PBS, and 50-100 uL cells were spotted onto the center of each coverslip. Cells were allowed to settle for 10-15 minutes at room temperature. Next cells were fixed in 2 mL/well of fresh fixative (4% formaldehyde in 1×PBS) and incubated for 10 minutes at room temperature. Cells were washed twice with 3 mL/well 1×PBS over 5 minutes, permeabilized in 2 mL/well with 0.5% Triton X-100, 1×PBS for 15 minutes at room temperature. Cells were washed three times for 5 minutes per wash with 3 mL/well of 1×PBS. Cells were stored at 4° C. in 1×PBS prior to staining.

Staining. Blocking buffer was prepared to contain 2% BSA (from 10% BSA/PBS), 0.05% Tween-20, and 1×PBS. Cells were blocked with 1.5 mL/well blocking buffer (in a 6-well plate) for 30 minutes at room temperature. Primary antibody incubation was carried out as follows. Primary antibodies were diluted in blocking buffer at the following ratios: 1:500 for anti-p53BP1 (tagging for p53BP1, which accumulates at the site of double strand breaks) and 1:2000 for anti-FLAG (tagging for FLAG label on a nuclease). A humidified chamber was prepared and a sheet of Parafilm was placed inside with 100 μL spots of the primary antibody solution. Coverslips were removed from the 6-well plate, inverted onto the primary antibody spots inside the humidified chamber, and incubated for 2 hours at room temperature. Coverslips were returned into the original 6-well plate with blocking buffer and cells were washed with 2 mL/well with 1×PBS three times for 5 minutes per wash. Samples were protected from light for subsequent steps performed with the secondary antibody labeled with a fluorophore. Secondary antibody incubation was carried out as follows. The secondary antibodies (donkey-anti-rabbit-Cy3 and donkey-anti-mouse-AF647) were diluted in a blocking buffer at 1:500. A new sheet of Parafilm was placed inside the humidified chamber with 100 μl spots of the secondary antibody solution. Coverslips were removed from the 6-well plate and inverted onto secondary antibody spots. Coverslips were incubated for 1.5 hours at room temperature. Coverslips were returned into the original 6-well plate and washed three times with 3 mL/well with 1×PBS for 5 minutes per wash. Finally, cells were stained with DAPI for visualization of the nucleus. Cells were incubated at 1.5 mL/well of 1×PBS with 100 ng/mL of DAPI for 10 minutes at room temperature. Cells were washed once with 1×PBS.

Mounting. 10 μl of Prolong Gold was dropped onto a clean microscope slide (up to 2 coverslips per slide), coverslips were removed from the 6-well plate using tweezers and inverted onto Prolong Gold, and Prolong Gold was allowed to cure for 24 hours at room temperature. After 24 hours, the edges of coverslips were further sealed with nail polish and coverslips were cleaned with water and wiped dry prior to imaging.

### 24 Well Format

Plate Coating with PLL. 0.5 mL/well of poly-L-lysine solution (0.1%, SigmaAldrich, cat. no. P8920) was added to 24-well glass-bottom plates (#1.5H), Cellvis, cat. no. P24-1.5H-N and incubated for 1-2 hours at room temperature. PLL was aspirated, the plate was rinsed with 0.5 mL/well of ddH₂O three times, water was removed from wells, and plates were dried overnight at room temperature.

Cell Preparation. Cells were seeded onto PLL coated glass bottom 24 well plates as follows. Cells were pre-washed with PBS and resuspended to ^{˜}2,000,000 cells/mL in PBS. 20-50 μL of cells were spotted onto the center of each well and allowed to settle for 10-15 minutes at room temperature. Cells were fixed in 0.5 mL/well of fresh fixative (4% formaldehyde in 1×PBS) as follow. 500 μL was added to each well, plates were shaked to dislodge poorly attached cells, and incubated for 10 minutes at room temperature. Cells were washed twice with 0.5 mL/well for 5 minutes each with 1×PBS, permeabilized in 0.5 mL/well 0.5% Triton X-100, 1×PBS for 15 minutes at room temperature, washed with 0.5 mL/well 1×PBS three times for 5 minutes each, and stored at 4° C. in 1×PBS prior to staining.

Staining. A blocking buffer containing 2% BSA (from 10% BSA/PBS), 0.05% Tween-20, 1×PBS. Cells were blocked with 0.4 mL/well blocking buffer for 30 minutes at room temperature. Primary antibody incubation was carried out as follows. Primary antibodies were diluted in blocking buffer (1:500 for anti-p53BP1, 1:2000 for anti-FLAG), blocking buffer was removed from cells and 300 uL/well of the primary antibody solution was added to cells. Cells were incubated for 2 hours at room temperature and washed three times with 0.5 mL/well 1×PBS for 5 minutes each. Samples were protected from light for subsequent steps performed with the secondary antibody labeled with a fluorophore. Secondary antibody incubation was carried out as follows. Secondary antibody diluted in blocking buffer at a ratio of 1:500 was added at 300 uL/well. Cells were incubated for 1.5 hours at room temperature, washed three times with 0.5 mL/well of 1×PBS for 5 minutes per wash. Cells were stained with DAPI for visualization of the nucleus by incubating cells in 0.3 mL/well of 1×PBS+100 ng/mL DAPI for 10 minutes at room temperature. Cells were washed once with 1×PBS.

Mounting. 10 uL drop of Prolong Gold was placed on 12 mm round glass coverslips, PBS was aspirated from wells, coverslips with Prolong Gold were inverted onto cells in a well, and Prolong Gold was allowed to cure for 24 hours at room temperature.

### 96 Well Format

Cell Preparation. Cells were seeded onto coated glass bottom 96 well plates (e.g., PLL-coated plates, CC² Nunc Micro-well plates) as follows. Cells were pre-washed with PBS and resuspended to ˜2,000,000 cells/mL in PBS. 10 μL of cells were spotted onto the center of each well and allowed to settle for 10-15 minutes at room temperature. Cells were fixed in 0.1 mL/well of fresh fixative (4% formaldehyde in 1×PBS) as follow. 100 μL was added to each well, plates were shaked to dislodge poorly attached cells, and incubated for 10 minutes at room temperature. Cells were washed twice with 0.1 mL/well for 5 minutes each with lx PBS, permeabilized in 0.1 mL/well 0.5% Triton X-100, 1×PBS for 15 minutes at room temperature, washed with 0.1 mL/well 1×PBS three times for 5 minutes each, and stored at 4° C. in 1×PBS prior to staining.

Staining. A blocking buffer containing 2% BSA (from 10% BSA/PBS), 0.05% Tween-20, 1×PBS. Cells were blocked with 75 uL/well blocking buffer for 30 minutes at room temperature. Primary antibody incubation was carried out as follows. Primary antibodies were diluted in blocking buffer (1:500 for anti-p53BP1, 1:2000 for anti-FLAG), blocking buffer was removed from cells and 75 uL/well of the primary antibody solution was added to cells. Cells were incubated for 2 hours at room temperature and washed three times with 0.1 mL/well 1×PBS for 5 minutes each. Samples were protected from light for subsequent steps performed with the secondary antibody labeled with a fluorophore. Secondary antibody incubation was carried out as follows. Secondary antibody diluted in blocking buffer at a ratio of 1:500 was added at 75 uL/well. Cells were incubated for 1.5 hours at room temperature, washed three times with 0.1 mL/well of 1×PBS for 5 minutes per wash. Cells were stained with DAPI for visualization of the nucleus by incubating cells in 0.1 mL/well of 1×PBS+100 ng/mL DAPI for 10 minutes at room temperature. Cells were washed once with 1×PBS.

Mounting. No mounting was applied for 96 well format. Plate was filled with 0.1 mL/well of 1×PBS and stored at 4° C. prior to imaging. Imaging was performed at room temperature with wells filled with 1×PBS.

### Example 5

### Dose Response Assessment of Nucleases in Multiple Cell Types Using p53BP1 Analysis

This example illustrates dose response assessment of nucleases in multiple cell types using p53BP1 analysis. Several TALENs (GA6, GA7, AAVS1) were tested for editing efficiency (quantification of the number of target sites with indels over the total number of target sites) and dose dependent generation of double stranded breaks, as determined by imaging for and counting p53BP1 foci. TALENs were transfected in cells as described in EXAMPLE 2 and p53BP1 was stained for and imaged as described in EXAMPLE 4 and EXAMPLE 1.

TABLE 4 below shows the nuclease designs including the left TALEN arm (bold), the right TALEN arm (italics), and the target sequence (underlined).

TABLE 4
TALEN Nuclease Constructs

| Nuclease | Sequence |
|---|---|
| GA6 | T GTGTAACAATGCCT gtggctctctgatgac |
| | AGTGCATGGCTGCAATGTGTG A |
| | (SEQ ID NO: 1063) |
| GA7 | T GCTCAGCCCAGCTCAGCCT gcagccctgtgggaa |
| | ATGGTAGAGAATGAGAGGGGG A |
| | (SEQ ID NO: 1064) |
| AAVS1 | T CCCCTCCACCCCACAGT gtccctagtggcccc |
| | AGGATTGGTGACAGAA A |
| | (SEQ ID NO: 1065) |

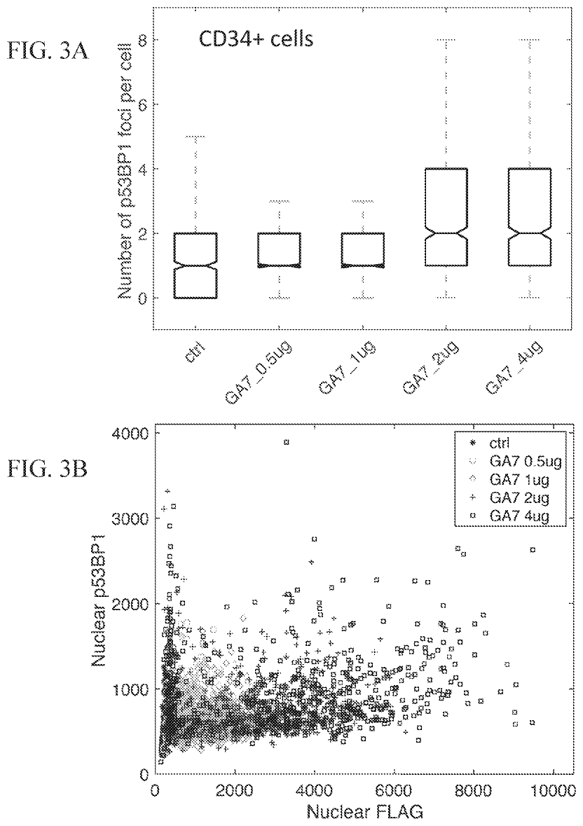

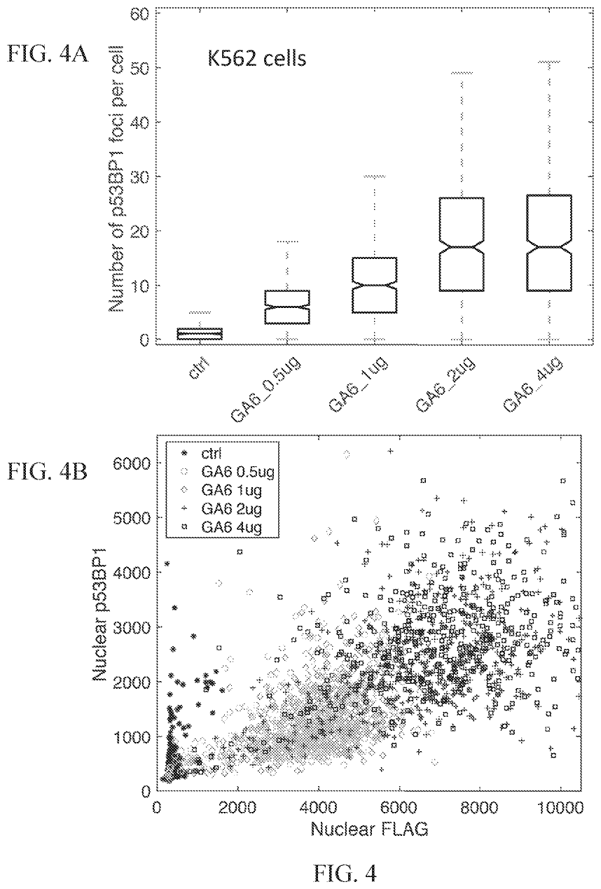

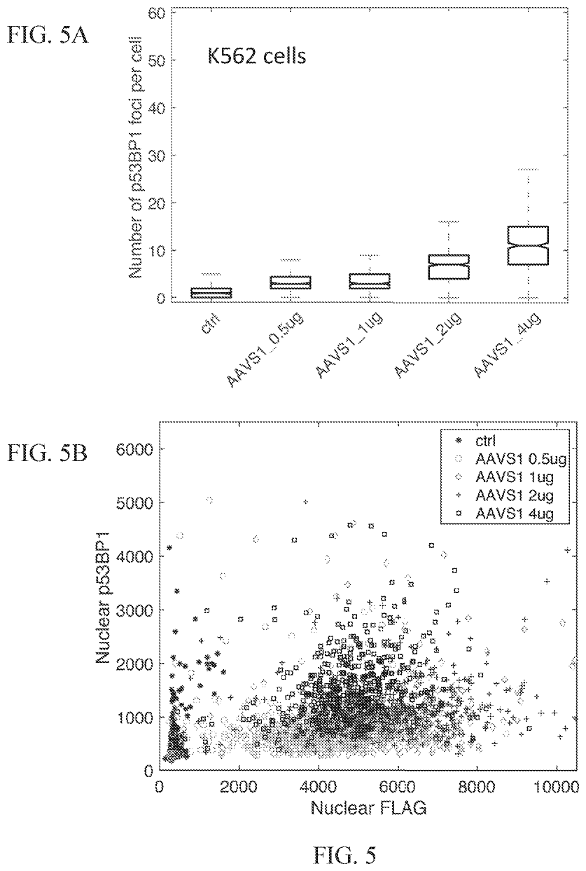

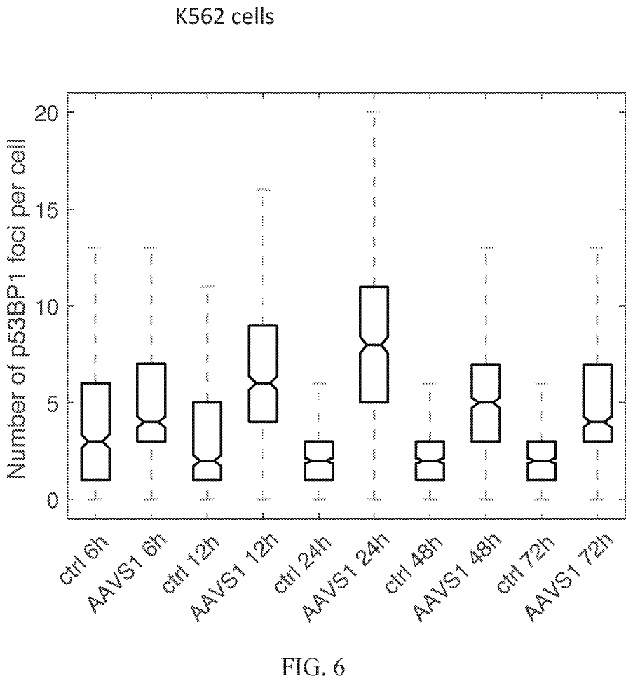

FIG. 3, FIG. 4, and FIG. 5 illustrate dose response assessments of GA7 TALENs in primary CD34+ hematopoietic stem cells, GA6 TALENs in immortalized K562 cells, and AAVS1 TALENs in immortalized K562 cells. FIG. 3A shows the number of p53BP1 foci per cell for CD34+ primary cells treated with a blank transfection control, 0.5 μg GA7 per TALEN monomer, 1 μg GA7 per TALEN monomer, 2 μg GA7 per TALEN monomer, and 4 μg GA7 per TALEN monomer. FIG. 3B shows the total p53BP1 content (fluorescence intensity) per nucleus normalized by the nuclear size versus total FLAG tag content per nucleus normalized by the nuclear size indicative of a nuclease for CD34+ primary cells treated with a blank transfection control, 0.5 μg GA7 per TALEN monomer, 1 μg GA7 per TALEN monomer, 2 μg GA7 per TALEN monomer, and 4 μg GA7 per TALEN monomer.

FIG. 4A shows the number of p53BP1 foci per cell for immortalized K562 cells treated with a blank transfection control, 0.5 μg GA6 per TALEN monomer, 1 μg GA6 per TALEN monomer, 2 μg GA6 per TALEN monomer, and 4 μg GA6 per TALEN monomer. FIG. 4B shows the total p53BP1 content (fluorescence intensity) per nucleus normalized by the nuclear size versus total FLAG tag content per nucleus normalized by the nuclear size indicative of a nuclease for immortalized K562 cells treated with a blank transfection control, 0.5 μg GA6 per TALEN monomer, 1 μg GA6 per TALEN monomer, 2 μg GA6 per TALEN monomer, and 4 μg GA6 per TALEN monomer.

FIG. 5A shows the number of p53BP1 foci per cell for immortalized K562 cells treated with a blank transfection control, 0.5 μg AASV1 per TALEN monomer, 1 μg AASV1 per TALEN monomer, 2 μg AAS per TALEN monomer, and 4 μg AAS per TALEN monomer. FIG. 5B shows the total p53BP1 content (fluorescence intensity) per nucleus normalized by the nuclear size versus total FLAG tag content per nucleus normalized by the nuclear size indicative of a nuclease for immortalized K562 cells treated with a blank transfection control, 0.5 μg AAS per TALEN monomer, 1 μg GA6, 2 μg AAS per TALEN monomer, and 4 μg AASV1 per TALEN monomer.

The corresponding editing efficiency of GA7 TALENs, GA6 TALENs, and AASV1 TALENS are shown below in TABLE 5.

TABLE 5
Gene Editing Efficiency

| Dose (μg) | GA7 | GA6 | AASV1 |
|---|---|---|---|
| 0.5 | 50% | 85% | 82% |
| 1 | 51% | 87% | 88% |
| 2 | 70% | 91% | 93% |
| 4 | 57% | 95% | 82% |

Nuclease specificity was assessed for each of GA7, GA6, and AASV1-targeting TALENs by evaluating the impact of nuclease dose on off-target cutting activity. TALENs that exhibited a high number of p53BP1 foci, indicative of double stranded breaks, in a dose-dependent manner indicate a nuclease with low specificity. For example, as shown in FIG. 3 CD34+ primary progenitor cells treated with a GA7 targeting TALEN exhibited only minimal increases in the DNA damage response, as indicated by the number of p53BP1 foci, as the delivered dose of the TALEN was increased. In contrast, the less specific GA6 (FIG. 4) and AASV1 (FIG. 5)-targeting TALENs resulted in increased off-target activity (increased number of p53BP1 foci) as the delivered dose of each of the TALENs was increased in K562 cells. The editing efficiency of each of the TALENs did not markedly change as dose was increased. Thus, examining off-target activity using the p53BP1-based image analysis disclosed herein, was used to optimize the nuclease dosage for low off-target activity while maintaining gene editing efficiency.

### Example 6

### Time Course Assessment of Nuclease Activity Using p53BP1 Analysis

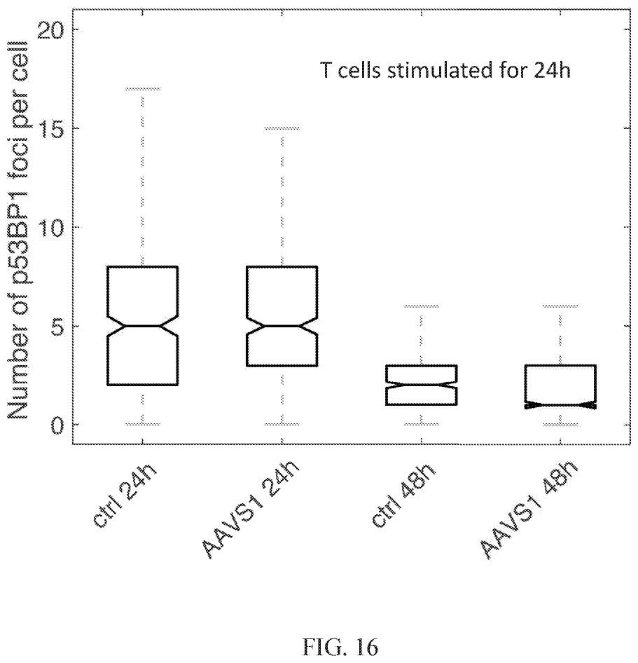

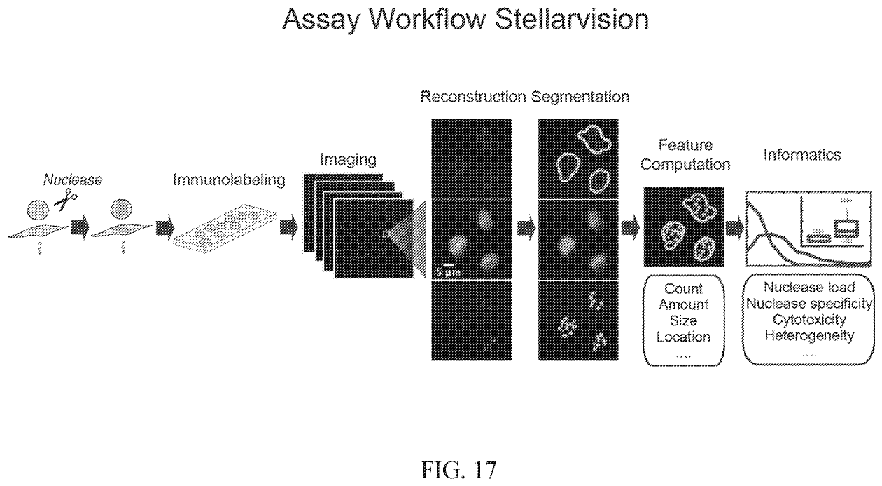

This example illustrates a time course assessment of nuclease activity using the p53BP1 analysis of the present disclosure. Nuclease specificity was used to study the cellular response to nuclease activity at various times after treatment of immortalized K562 cells. K562 cells were transfected with mRNA encoding TALENs targeting the AAVS1 DNA locus. Cells were transfected as described in EXAMPLE 2 and p53BP1 was stained for and imaged as described in EXAMPLE 4 and EXAMPLE 1. Cells were sampled and imaged at 6 hours, 12 hours, 24 hours, 48 hours, and 72 hours post-transfection. FIG. 6 shows a graph of the number of p53BP1 foci per K562 cells at 6 hours, 12 hours, 24 hours, 48 hours, and 72 hours as compared to a control at each time point. The editing efficiency was determined to be 91% at 48 hours tested. Peak activity was observed for the AAVS1-targeting TALENs at 24 hours, and persisted beyond the 72 hour post-transfection time point. Additionally, an initial increase in the DNA damage response triggered by electroporation was detected in control cells. In a separate experiment, AASV1-targeting TALENs transfected in CD4+ T cells ceased all activity by 48 hours post-transfection, as shown in FIG. 16. FIG. 16 shows a graph of the number of p53BP1 foci per CD4+ T cell at 24 hours and 48 hours post-transfection with AASV1-targeting TALENs as compared to blank transfection controls at each time point.

### Example 7

### Utility of p53BP1 Analysis for Pan-Cell Type Assessment of AAVS1-Targeting TALEN Specificity

This example illustrates the utility of p53BP1 analysis of the present disclosure for pan-cell type assessment of AAVS1-targeting TALEN specificity. To demonstrate that nuclease specificity as determined by p53BP1 analysis can be measured across several cell types, TALENs targeting AAVS1 region were transfected in adherent immortalized A549 cells, suspension immortalized K562 cells, and primary cell samples isolated from blood including CD34+ progenitor cells and CD4+ T cells. Non-T cells were transfected as described in EXAMPLE 2, T cells were transfected as described in EXAMPLE 3, and p53BP1 was stained for and imaged as described in EXAMPLE 4 and EXAMPLE 1. All cells were transfected with 2 mRNAs encoding the respective TALEN monomers (one targeting a top strand of the target DNA genomic locus and the second targeting a bottom strand of the target DNA genomic locus). Cells were sampled for evaluation of p53BP1 foci 24 hours post-transfection.

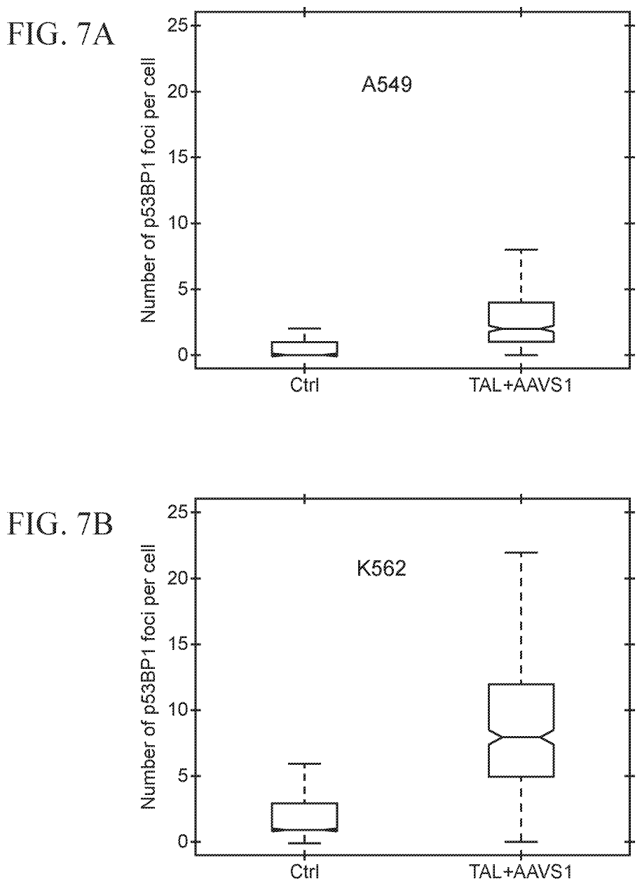

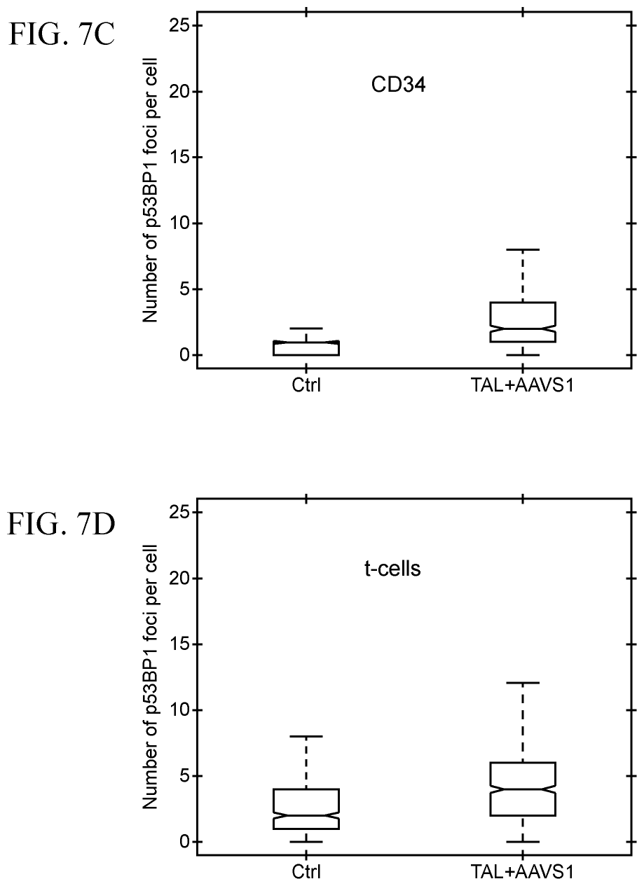

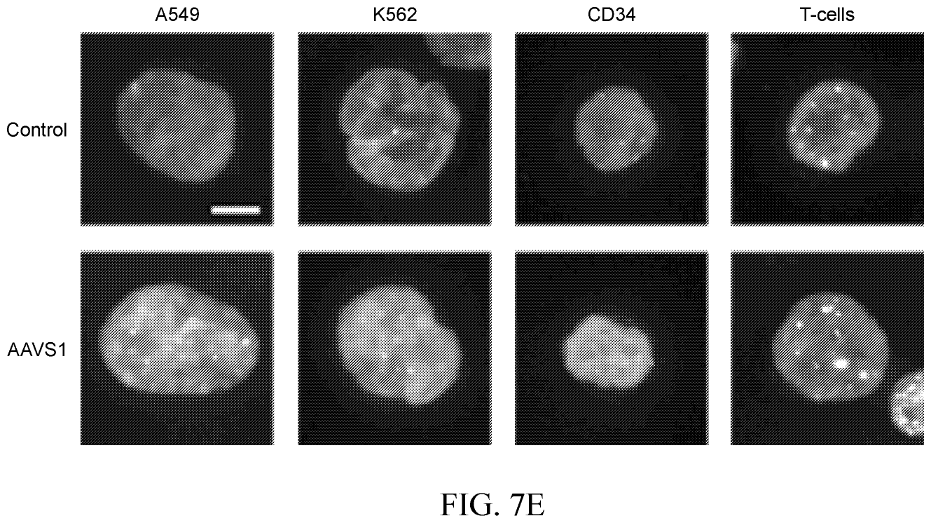

FIG. 7 shows the results of control transfection and AASV1-targeting TALEN transfection in various cell types. FIG. 7A shows the number of p53BP1 foci in adherent immortalized A549 cells transfected with a control and with an AASV1-targeting TALEN 24 hours post-transfection. FIG. 7B shows the number of p53BP1 foci in suspension immortalized K562 cells transfected with a control and with an AASV1-targeting TALEN 24 hours post-transfection. FIG. 7C shows the number of p53BP1 foci in primary CD34+ progenitor cells transfected with a control and with an AASV1-targeting TALEN 24 hours post-transfection. FIG. 7D shows the number of p53BP1 foci in primary CD4+ T cells transfected with a control and with an AASV1-targeting TALEN 24 hours post-transfection. FIG. 7E shows representative images of cells treated with AAVS1 TALENs versus untreated controls. Cells were stained for p53BP1 with an antibody and are visualized in green. TALENs were stained with a FLAG tag and are visualized in red. Nuclei were stained with DAPI and are visualized in grey. The scale bar indicates a size of 5 μm.

TABLE 6 below shows the gene editing efficiency of AAVS1-targeting TALENs in A549 cells, K562 cells, CD34+ cells, and CD4+ T cells.

TABLE 6
Gene Editing Efficiency of AAVS1-targeting TALENs in A549 cells,
K562 cells, CD34+ cells, and CD4+ T cells

| Cell Type | Gene Editing Efficiency |
|---|---|
| A549 | 54% |
| K562 | 94% |
| CD34+ progenitors | 74% |
| CD4+ T cells | 93% |

All cells exhibited an increase in the number of p53BP1 DNA repair foci upon treatment with TALENs in comparison to untreated controls. Moreover, p53BP1 image analysis revealed differences in the level of background DNA repair activity as well as the magnitude of response to nuclease treatment between different cell types.

### Example 8

### Utility of p53BP1 Analysis for Pan-Nuclease Type Assessment of Genome Editing Specificity

This example illustrates the utility of p53BP1 analysis for pan-nuclease type assessment of genome editing specificity. To demonstrate that nuclease specificity as determined by p53BP1 analysis can be measured across various types of nucleases, TALENs and Cas9 nucleases targeting the AAVS1 genomic locus were transfected in K562 cells. For Cas9 treatment, K562 cells were transfected with Cas9 protein along with AAVS1-targeting guide RNAs and incubated at 37° C. for 24 hours prior to sampling. For treatment with TALENs, K562 cells were transfected with 2 mRNAs encoding the respective TALEN monomers (one targeting a top strand of the target DNA genomic locus and the second targeting a bottom strand of the target DNA genomic locus) and incubated at 30° C. for 24 hours prior to sampling. Cells were transfected as described in EXAMPLE 2 and p53BP1 was stained for and imaged as described in EXAMPLE 4 and EXAMPLE 1.

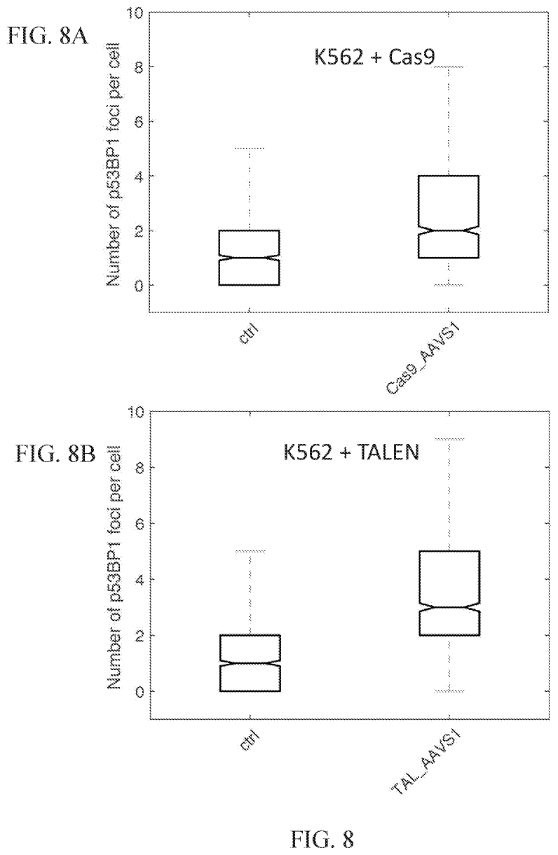

FIG. 8 illustrates assessment of nuclease specificity in K562 cells for TALENs and Cas9 nucleases targeting the AAVS1 genomic locus. FIG. 8A illustrates the number of p53BP1 foci per cell for K562 cells transfected with Cas9 protein along with AAVS1 guide RNAs as compared to a blank transfection control. FIG. 8B illustrates the number of p53BP1 foci per cell for K562 cells transfected with AAVS1-targeting TALENs as compared to a blank transfection control.

TABLE 7 below shows the editing efficiency of AAVS1-targeting Cas9 and AAVS1-targeting TALENs

TABLE 7
Editing Efficiency of AAVS1-Targeting Cas9 and TALENs

| Nuclease | Gene Editing Efficiency |
|---|---|
| AASV1-Targeting Cas9 | 86% |
| AASV1-Targeting TALEN | 95% |

Both Cas9 and TALENs produced measurable DNA damage responses as indicated by the increased number of p53BP1 foci relative to the untreated controls.

### Example 9

### Utility of p53BP1 Analysis for Assessing Nuclease Activity in Diverse Cell Types and Several Genomic Loci

This example illustrates the utility of p53BP1 analysis for assessing nuclease activity in diverse cell types targeting various genomic loci. To demonstrate that nuclease specificity as determined by p53BP1 analysis can be used to screen multiple nucleases in diverse cell types, the performance of TALENs targeting GA6, AAVS1, and GA7 in CD34+ progenitor cells and the performance of TALENs targeting TP150, AAVS1, and TP171 in stimulated CD4+ T cells was evaluated. Non-T cells were transfected as described in EXAMPLE 2, T cells were transfected as described in EXAMPLE 3, and p53BP1 was stained for and imaged as described in EXAMPLE 4 and EXAMPLE 1. The performance of GA6 and GA7-targeting TALENs with a homodimeric FokI nuclease domain was compared to TALENs with the obligate heterodimeric ELD/KKR FokI nuclease domains (GA6-EK and GA7-EK) in primary CD34+ progenitor cells.

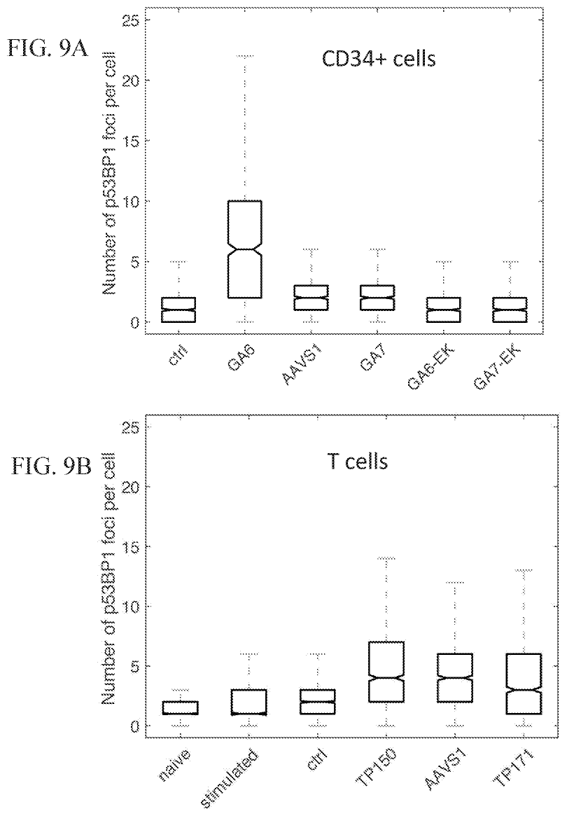

FIG. 9 shows the DNA damage response, as measured by p53BP1 foci quantification, in CD34+ cells and T cells with TALENs targeting various genomic loci. FIG. 9A shows the number of p53BP1 foci per cell in primary CD34+ progenitor cells after transfection with GA6-targeting TALENs, AAVS1-targeting TALENs, GA7-targeting TALENs, GA6-EK-targeting TALENs, and GA7-targeting TALENs. Controls include blank transfection controls. FIG. 9B shows the number of p53BP1 foci per cell in primary stimulated CD4+ T cells after transfection with TP150-targeting TALENs, AAVS1-targeting TALENs, and TP171-targeting TALENs Controls include non-electroporated naïve T cells, non-electroporated stimulated T cells, and untreated blank transfection control stimulated T cells.

TABLE 8 below shows the editing efficiency of several TALENs targeting different genomic loci after transfection of primary CD34+ progenitor cells.

TABLE 8
Editing Efficiency of TALENs in Primary CD34+ Progenitor Cells

| Nuclease | Gene Editing Efficiency |
|---|---|
| GA6-Targeting TALEN | 54% |
| AAVS1-Targeting TALEN | 26% |
| GA7-Targeting TALEN | 50% |
| GA6_EK-Targeting TALEN | 36% |
| GA7_EK-Targeting TALEN | 20% |

TABLE 9 below shows the editing efficiency of several TALENs targeting different genomic loci after transfection of CD4+ T cells.

TABLE 9
Editing Efficiency of TALENs in CD4+ T cells

| Nuclease | Gene Editing Efficiency |
|---|---|
| TP150-Targeting TALEN | 91% |
| AAVS1-Targeting TALEN | 90% |
| TP171-Targeting TALEN | 95% |

Determination of nuclease specificity by p53BP1 foci analysis showed a range of cell responses to different nucleases, from minimal activation of DNA repair with more specific GA7-EK TALEN activity to substantially higher levels of DNA repair with less specific GA6 TALEN activity.

### Example 10

### Use of p53BP1 Analysis for Improving Nuclease Design

This example illustrates the use of p53BP1 analysis for improving nuclease design. Specificity was assessed using the p53BP1 tools and methods of analysis of the present disclosure to evaluate different designs of nucleases targeting the same genomic locus. Non-T cells were transfected as described in EXAMPLE 2 and p53BP1 was stained for and imaged as described in EXAMPLE 4 and EXAMPLE 1.

K562 cells were transfected with GA6-targeting TALENs having homodimeric FokI nuclease domains (GA6) or GA6-targeting TALENs with the obligate heterodimeric ELD/KKR FokI nuclease domains (GA6_EK). ELD FokI has a sequence of QLVKSEEEKKSELRHKLKYVPHEYIELIEIARNSTQDRILEMKVMEFFMKVYGYRG KHLGGSRKPDGAIYTVGSPIDYGVIVDTKAYSGGYNLPIGQADEMERYVEENQTRD KHLNPNEWWKVYPSSVTEFKFLFVSGHFKGNYKAQLTRLNHITNCNGAVLSVEELLI GGEMIKAGTLTLEEVRRKFNNGEINFRS (SEQ ID NO: 1066) and KKR FokI has a sequence of

(SEQ ID NO: 1067)
QLVKSELEEKKSELRHKLKYVPHEYIELIEIARNSTQDRILEMKVMEFFM
KVYGYRGKHLGGSRKPDGAIYTVGSPIDYGVIVDTKAYSGGYNLPIGQAD
EMQRYVKENQTRNKHINPNEWWKVYPSSVTEFKFLFVSGHFKGNYKAQLT
RLNRKTNCNGAVLSVEELLIGGEMIKAGTLTLEEVRRKFNNGEINFRS.

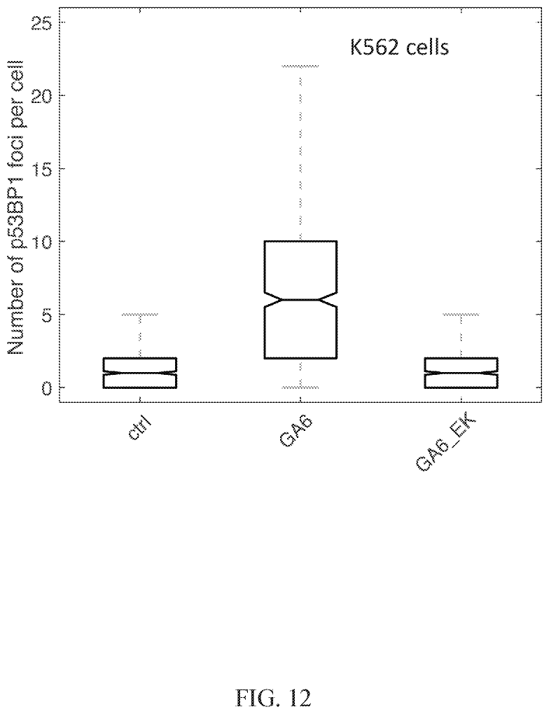

FIG. 12 shows the number of p53BP1foci per cell in K562 cells transfected with GA6 or GA6_EK TALENs.

TABLE 11 below shows the genome editing efficiency of GA6 and GA6_EK.

TABLE 11
Genome Editing Efficiency of GA6 and GA6_EK

| Nuclease | Gene Editing Efficiency |
|---|---|
| GA6-Targeting TALEN | 54% |
| GA6_EK-Targeting TALEN | 36% |

The results showed substantial off-target activity by GA6 (TALEN with homodimeric FokI), as evident from the large number of p53BP1 foci formed in response to transfection and also showed the high specificity of GA6_EK (TALEN with heterodimeric FokI).

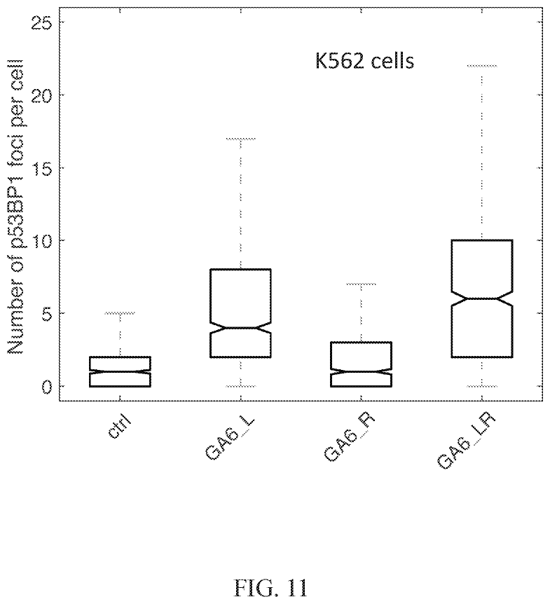

In another experiment, the p53BP1 tools and methods of analysis of the present disclosure were used to evaluate the contribution of individual components of a nuclease. For example, the specificity of individual monomers of GA6 TALEN (GA6_L (left TALEN) and GA6_R (right TALEN)) was measured in K562 cells and compared GA6 homodimers (GA6_LR (left and right TALENs)) and a blank transfection control. Cells were transfected with mRNA encoding either GA6_L, GA6_R, or both GA6_L+GA6_R (GA6_LR) and incubated at 30° C. for 24 hours prior to sampling. FIG. 11 shows the number of p53BP1 foci per cell in K562 cells transfected with GA6_L, GA6_R, GA6_LR versus untreated control cells. The genome editing efficiency of GA6_LR was 54%. The genome editing efficiencies of the individual monomers of the GA6 TALEN was 0% for GA6_L and GA6_R.

The results demonstrated substantial off-target DNA cutting by the GA6 homodimer, as evident from a large number of phospho-53BP1 foci forming in response to TALEN treatment. At the same time, it was evident that the GA6_L monomer alone contributed to the lack of specificity, being responsible for the majority of nuclease-induced DNA repair response while failing to produce DNA cleavage at the target site. Thus, it was possible to pinpoint the component responsible for the lack of nuclease specificity and guide design efforts in order to reduce off-target activity.

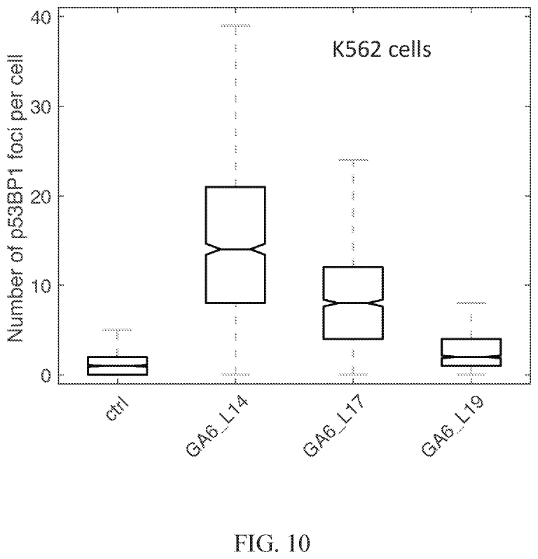

In another experiment, nuclease performance was optimized by varying the length of the DNA binding domain in a homodimeric FokI GA6-targeting TALEN As described above, the GA6_L monomer appeared responsible for the lack of specificity and high number of p53BP1 foci per cell, as shown in FIG. 11. To investigate if the specificity of the homodimeric FokI GA6-targeting TALEN could be improved, the DNA binding domain was extended from 14 repeat units (GA6_L14) to 17 repeat units (GA6_L17) and 19 repeat units (GA6_L19). FIG. 10 shows the number of p53BP1 foci per cell in K562 cells transfected with GA6_L14, GA6_L17, and GA6_L19.

TABLE 12 below shows the nuclease designs including the left TALEN arm (bold), the right TALEN arm (italics), and the target sequence (underlined).

TABLE 12
TALEN Nuclease Constructs

| Nuclease | Sequence |
|---|---|
| GA6_14 | T GTGTAACAATGCCT gtggctctctgatgac |
| | AGTGCATGGCTGCAATGTGTG A |
| | (SEQ ID NO: 1068) |
| GA6_17 | T GTGTAACAATGCCTGTG gctctctgatgac |
| | AGTGCATGGCTGCAATGTGTG A |
| | (SEQ ID NO: 1069) |
| GA6_19 | T GGAGTGTGTAACAATGCCT gtggctctctgatgac |
| | AGTGCATGGCTGCAATGTGTG A |
| | (SEQ ID NO: 1070) |

TABLE 13 below shows the genome editing efficiency of each GA6_L monomer with its corresponding GA6_R monomer.

TABLE 13
Genome Editing Efficiency

| Nuclease | Gene Editing Efficiency |
|---|---|
| GA6_L14 + GA6_R | 96% |
| GA6_L17 + GA6_R | 98% |
| GA6_L19 + GA6_R | 86% |

Assessment of p53BP1 foci showed that as the TALEN was tuned to have longer DNA binding domains, there was a dramatic reduction in off-target activity. At the same time, when combined with a match GA6_R monomer, GA6_L19 still exhibited unperturbed, high on-target editing efficiency.

### Example 11

### Multiplexed p53BP1, FLAG, and Nano-FISH Staining and Analysis Use of p53BP1 Analysis and Nano-FISH to Dissect On-Target Versus Off-Target Activity of Nucleases for Genome Editing

This example illustrates multiplexed p53BP1, FLAG, and Nano-FISH staining and analysis and the use of p53BP1 analysis and Nano-FISH to dissect on-target and off-target activity of nucleases for genome editing.

Multiplexed p53BP1, FLAG, and Nano-FISH Staining and Analysis

Nuclease specificity was assessed in a site-specific manner at the genomic locus of interest by imaging and analyzing nuclease (tagged with FLAG) induced double strand breaks (indicated by staining for p53BP1) at a particular genomic locus of interest, which is visualized by oligonucleotide Nano-FISH probe sets.

Cell Preparation. Cells were prepared for co-staining by seeding onto poly-1-lysine coated #1.5 glass coverslip (12 mm round or 18 mm square). Coverslips were placed into each well of a 6-well tissue culture plate, cells were prewashed with PBS and resuspended to 2,000,000 cells/mL in PBS. Cells were spotted (50-100 ul) onto the center of each coverslip and cells were allowed to settle for 10-15 minutes at room temperature. Cells were fixed in 2 mL/well with fresh fixative (4% formaldehyde in 1×PBS) and incubated for 10 minutes at room temperature. Cells were washed twice with 3 mL/well of 1×PBS, each over 5 minutes. Cells were permeabilized in 2 mL/well 0.5% Triton X-100, 1×PBS for 15 minutes at room temperature, cells were washed twice with 3 mL/well of 1×PBS for 5 minutes each, cells were incubated with 1.5 mL/well 0.1M HCl for 4 minutes at room temperature, and cells were washed twice with 3 mL/well of 2×SSC over 5 minutes. Cells were incubated in 1.5 mL/well of 2×SSC+25 ug/mL RNase A for 30 minutes at 37° C., washed twice with 3 mL/well of 2×SSC, for 5 minutes each. Finally, cells were pre-equilibrated with 1.5 mL/well of 50% Formamide, 2×SSC [pH 7] for at least 30 minutes at room temperature prior to denaturation.

Denaturation/Hybridization. Denaturation solution (70% formamide, 2×SSC) was added at 3 mL/well in a new 6-well plate and the well-plate was heated for at least 30 minutes on a hotplate set to 78° C. Denaturation was carried out as follows. Coverslips were transferred into the well plate with preheated denaturation solution and incubated for 4.5 minutes at 78° C., then immediately transferred onto hybridization solution. All subsequent steps were carried out so that samples were protected from light. Hybridization solution with oligonucleotide Nano-FISH probes was prepared as follows. A hybridization buffer containing 50% formamide, 10% dextran sulfate, 0.05% Tween-20, 2×SSC. Oligonucleotides Nano-FISH probes at a concentration of 10 uM were diluted in Hybridization buffer at a ratio of 1:40, such that the final concentration was 250 nM. Oligonucleotide Nano-FISH probes were synthesized to include the Quasar-670 dye, which was imaged in the Cy5 channel. A humidified chamber was set up by placing a sheet of Parafilm onto a wet paper towel inside a dark plastic container. On a sheet of Parafilm, Hybridization solution was spotted at a volume of 80 ul. Hybridization was carried out by removing coverslips from the denaturation solution, inverting onto Hybridization solution spots inside the humidified chamber, and incubating overnight at 37° C.

TABLE 10 below shows the oligonucleotide Nano-FISH probe set for AAVS1.

TABLE 10
AAVS1 Olignucleotide Nano-FISH Probe Set

| SEQ ID | |
|---|---|
| NO | Sequence |
| SEQ ID | TGCAAGAACCAAAACCCGTTCCTCCTGGCTCAGGCCGGAA |
| NO: 1071 | |
| SEQ ID | TCTGGCCCAGTCGACTCAGGGGCTGAATCGGGCATGACTC |
| NO: 1072 | |
| SEQ ID | TCGTGGCCTGGAGCCACCGCTCCCTCCAACACCGCAAAGT |
| NO: 1073 | |
| SEQ ID | CTGGGGTTCAGTGAGAGCACGTGATCTGCTCAGCCAGTCA |
| NO: 1074 | |
| SEQ ID | TTCGCTTTCCCTGGCTTACTTGCTGTTTTCCTCTCTCTGG |
| NO: 1075 | |
| SEQ ID | GCTGGGAGAGAAGACAGACCGGCCTCAGGCACGACCATCC |
| NO: 1076 | |
| SEQ ID | GCTCTGGCCATAGTGTGGCCCTGGCAGCCACTCACAGGCA |
| NO: 1077 | |
| SEQ ID | CCACATGATGCAGAATTCCCCGAGGTGCTGGCATCCAGAC |
| NO: 1078 | |
| SEQ ID | CTCTAAGGAGGGCGGGTCTTTTGCACCCCCTGCAGGACAC |
| NO: 1079 | |
| SEQ ID | GGGCTGCAGTGCGCAGGACCTGGATCACAGGCTGCACCCC |
| NO: 1080 | |
| SEQ ID | GTGACACCCTGTGACACCCGGCTCCACACAGGAGCCTCAG |
| NO: 1081 | |
| SEQ ID | CGGGGTGGGACTCTGCGGCCCCAAATCACAAGGCGACTGC |
| NO: 1082 | |
| SEQ ID | AAGACCACTGGGGCCACTGGAAAGACCCTCAGCCGTGCTG |
| NO: 1083 | |
| SEQ ID | ACATTGGTGGGGGATATTGGCTTGTAGGATCAGCCAGGAA |
| NO: 1084 | |
| SEQ ID | GAAATTGCTCATAACTTGCATCAGCTTCTCAGAGGGGGCC |
| NO: 1085 | |
| SEQ ID | TCCAGGGGGTCTGTGAACTTTCTGACGTTGTATTTTCCTG |
| NO: 1086 | |
| SEQ ID | GGATCCAGATCTGGGTGATTTAGGCTCCCTCTGTCTGGAT |
| NO: 1087 | |
| SEQ ID | ATTCTTTGTAGCCTCTCCCGCTCTGGTTCAGGGCCCAGCT |
| NO: 1088 | |
| SEQ ID | ACCAACCTTGATGCTACACTGTTGCCTGCGTTTCTCCTTG |
| NO: 1089 | |
| SEQ ID | CACCCACCGCACCAACCTTGATGCTACACTCTCACCCACT |
| NO: 1090 | |
| SEQ ID | GCTACACTCTCACCCACCGCACCAACCTTGATGCTACACT |
| NO: 1091 | |
| SEQ ID | CAACCTTGATGCTACACTCTCACCCACCGCACCAACCTTG |
| NO: 1092 | |
| SEQ ID | CCCACCGCACCAACCTTGATGCTACACTCTCACCCACCGC |
| NO: 1093 | |
| SEQ ID | CAACACGCTACCCCCTGTGTTGACCTTGATGCTACACTCT |
| NO: 1094 | |
| SEQ ID | CCTGCCACAAGGAAAACCTCCTGCAGAACCACAGTAGGGA |
| NO: 1095 | |
| SEQ ID | TGCAGGCATTGTACATCTTCGCCTGATGCACAGCAGGTAT |
| NO: 1096 | |
| SEQ ID | GATCTCTTCCCAGGTATAGACATAAACACATTTTTTCCTA |
| NO: 1097 | |
| SEQ ID | tcatcatcccccaacgaaaccctgcaaccgcttagccatc |
| NO: 1098 | |
| SEQ ID | acggggtcgggcatttatgaccacattggttgtagaacat |
| NO: 1099 | |
| SEQ ID | aattcacccaaagtgcacacttcagtgctttttagtctat |
| NO: 1100 | |
| SEQ ID | tttacagaaaagttgaagcaatagcatgtgactacccata |
| NO: 1101 | |
| SEQ ID | GAAATGGGGAGTGGGTCAAATCAGCCCTGGACCTGGATTC |
| NO: 1102 | |
| SEQ ID | CGTGACGGCGGAGATCTGAGGTTCGGGAGCCCCTCTTTGG |
| NO: 1103 | |
| SEQ ID | GGGGTCCACGAGAGCCATGCGGGAGGACTAGCTAGTGGGA |
| NO: 1104 | |
| SEQ ID | GCCGCTGGCCAGGCTGAAAGGATAGGATTCCGCGTGGGTT |
| NO: 1105 | |
| SEQ ID | ACCGGCAGCCTCCGAGACTTCTGACGCGGCTGTCCTGACG |
| NO: 1106 | |
| SEQ ID | GGACCGTGTGGAAGGAAAGGGAGACTGACGAGGAAATGAG |
| NO: 1107 | |
| SEQ ID | tggagtggaagggtgtgagcatggttcccggcagacTCCA |
| NO: 1108 | |
| SEQ ID | ctggtgccgcttcatggggtggttgtcagggtctggctgg |
| NO: 1109 | |
| SEQ ID | cgtccctgaagcttgcttccctgatttcctaaaacaggac |
| NO: 1110 | |
| SEQ ID | ggcttgcctcccagctctgcctgtgactggtgactccagg |
| NO: 1111 | |
| SEQ ID | ACACAGGATCCCTGGGTCCCCAGCATGTCTTCTAAagtcc |
| NO: 1112 | |
| SEQ ID | TTCTAGGGAAGGGGTGTTGCTTCTAGCAGGTGTGTGATGG |
| NO: 1113 | |
| SEQ ID | GGGTCCAGGAGCCCCTGAAACTGTGTCTGGCCAGGTTCAT |
| NO: 1114 | |
| SEQ ID | CCTGTCCTCTGAGACTCATCGTACCCCAGGAGCCTTCATA |
| NO: 1115 | |
| SEQ ID | GGGGGGAGTAGGGGCATGCAGGGGTTGCCAGGGACTGGTC |
| NO: 1116 | |
| SEQ ID | AACCCTGCCGCAGGTCTTTCTGGGAGGGGATGCGTTTACT |
| NO: 1117 | |
| SEQ ID | GTGGAGGGACTCACCCAGGAGTGCGTTAGGTAGGATTGCT |
| NO: 1118 | |
| SEQ ID | TGAGTAACTGAGGGGATTGGAATGCCGGGGCGGGGTGGGT |
| NO: 1119 | |
| SEQ ID | ATGAGAACTCAAACCCCTACCAACTGGGACTGTCAATCCC |
| NO: 1120 | |
| SEQ ID | ggcctgcctccaggattgcttggagCCCAGCACACGCACA |
| NO: 1121 | |
| SEQ ID | GCCTGGGCACCGAGGCTGACCCTGCTTCCTAGGATTGTCT |
| NO: 1122 | |
| SEQ ID | ACCTCCTCACCCGTGGTCTCCAGGCTGAGAGCTTTAGAGG |
| NO: 1123 | |
| SEQ ID | GAGTCGGACGCCATGGAGGGGCTGCTGAAGGCGGAGATCG |
| NO: 1124 | |
| SEQ ID | GCCGCCGTCAACAGTGACGGGGACCTGCCCCTGGACCTGG |
| NO: 1125 | |
| SEQ ID | GCCCCCACCCCCAGGTACCTCCTGAGCCACGGGGCCAACA |
| NO: 1126 | |
| SEQ ID | GGACCTGGTCGGGGTGGGGGCCTGGACCCTCAGCCCTGAC |
| NO: 1127 | |
| SEQ ID | GCTACCTAGATATCGCCAGGTGAGGCAAGGGAGGGCCGGG |
| NO: 1128 | |
| SEQ ID | ACAACGAGGGCTGGACGCCACTGCACGTGGCCGCCTCCTG |
| NO: 1129 | |
| SEQ ID | TGCGCTTCTTGGTGGAGCAGGGCGCCACTGTGAACCAGGC |
| NO: 1130 | |
| SEQ ID | TTTCCCACCCCCAGGCCTGCATTGATGAGAACCTGGAGGT |
| NO: 1131 | |
| SEQ ID | TTGCTGGGACACCGTGGCTGGGGTAGGTGCGGCTGACGGC |
| NO: 1132 | |
| SEQ ID | TGTCCCTGGATCTGTTTTCGTGGCTCCCTCTGGAGTCCCG |
| NO: 1133 | |
| SEQ ID | GCCAGAGGCTGTTGGGTCATTTTCCCCACTGTCCTAGCAC |
| NO: 1134 | |
| SEQ ID | GCCTGACCACTGGGCAACCAGGCGTATCTTAAACAGCCAG |
| NO: 1135 | |
| SEQ ID | GAGTCCTTTCGTGGTTTCCACTGAGCACTGAAGGCCTGGC |
| NO: 1136 | |
| SEQ ID | CCCCCTCCCTTCCCCGTTCACTTCCTGTTTGCAGATAGCC |
| NO: 1137 | |
| SEQ ID | TCTAACAGGTACCATGTGGGGTTCCCGCACCCAGATGAGA |
| NO: 1138 | |
| SEQ ID | CTGGAAGCGCCACCTGTGGGTGGTGACGGGGGTTTTGCCG |
| NO: 1139 | |
| SEQ ID | CTGCTGGGGTGGTTTCCGAGCTTGACCCTTGGAAGGACCT |
| NO: 1140 | |
| SEQ ID | CCTGCATAGCCCTGGGCCCACGGCTTCGTTCCTGCAGAGT |
| NO: 1141 | |
| SEQ ID | AGGCCCCTGAGTCTGTCCCAGCACAGGGTGGCCTTCCTCC |
| NO: 1142 | |
| SEQ ID | ACACAGGTGTGCAGCTGTCTCACCCCTCTGGGAGTCCCGC |
| NO: 1143 | |
| SEQ ID | GGGGCCTCAGTGAACTGGAGTGTGACAGCCTGGGGCCCAG |
| NO: 1144 | |
| SEQ ID | GGTGGCCCGTGTCAGCCCCTGGCTGCAGGGCCCCGTGCAG |
| NO: 1145 | |
| SEQ ID | TGTCCCCCCAAGTTTTGGACCCCTAAGGGAAGAATGAGAA |
| NO: 1146 | |
| SEQ ID | CCTGGGGCAAGTCCCTCCTCCGACCCCCTGGACTTCGGCT |
| NO: 1147 | |
| SEQ ID | AGCTCCAGTTCAGGTCCCGGAGCCCACCCAGTGTCCACAA |
| NO: 1148 | |
| SEQ ID | ATTTATCCCGTGGATCTAGGAGTTTAGCTTCACTCCTTCC |
| NO: 1149 | |
| SEQ ID | TCCAGATGGGCAGCTTTGGAGAGGTGAGGGACTTGGGGGG |
| NO: 1150 | |
| SEQ ID | ATGACCTCATGCTCTTGGCCCTCGTAGCTCCCTCCCGCCT |
| NO: 1151 | |
| SEQ ID | CGTTCCCAGGGCACGTGCGGCCCCTTCACAGCCCGAGTTT |
| NO: 1152 | |
| SEQ ID | CGCCATGACAACTGGGTGGAAATAAACGAGCCGAGTTCAT |
| NO: 1153 | |
| SEQ ID | GAAAGGGAAAGGCCCATTGCTCTCCTTGCCCCCCTCCCCT |
| NO: 1154 | |
| SEQ ID | TCAGGCATCTTTCACAGGGATGCCTGTACTGGGCAGGTCC |
| NO: 1155 | |
| SEQ ID | TTGggggctagagtaggaggggctggagccaggattctta |
| NO: 1156 | |
| SEQ ID | TGCCCCCATTCCTGCACCCCAATTGCCTTAGTGGCTAGGG |
| NO: 1157 | |
| SEQ ID | ACCCCACGTGGGTTTATCAACCACTTGGTGAGGCTGGTAC |
| NO: 1158 | |
| SEQ ID | AGCATCGCCCCCCTGCTGTGGCTGTTCCCAAGTTCTTAGG |
| NO: 1159 | |
| SEQ ID | GCTGTGTTTCTCGTCCTGCATCCTTCTCCAGGCAGGTCCC |
| NO: 1160 | |
| SEQ ID | ctctgggtGACTCTTGATTCCCGGCCAGTTTCTCCACCTG |
| NO: 1161 | |
| SEQ ID | gaaaccctcagtcctaggaaaacagggatggttggtcact |
| NO: 1162 | |
| SEQ ID | ccagcttatgctgtttgcccaggacagcctagttttagca |
| NO: 1163 | |
| SEQ ID | AGCAGGGGAGctgggtttgggtcaggtctgggtgtggggt |
| NO: 1164 | |
| SEQ ID | TTCAGAGAGGAGGGATTCCCTTCTCAGGTTACGTGGCCAA |
| NO: 1165 | |
| SEQ ID | CGGGGTATCCCAGGAGGCCTGGAGCATTGGGGTGGGCTGG |
| NO: 1166 | |
| SEQ ID | TCTCCTCCAACTGTGGGGTGACTGCTTGGCAAACTCACTC |
| NO: 1167 | |
| SEQ ID | GGCCACCCCAGCCCTGTCTACCAGGCTGCCTTTTGGGTGG |
| NO: 1168 | |
| SEQ ID | CCAGAGGCCCCAGGCCACCTACTTGGCCTGGACCCCACGA |
| NO: 1169 | |
| SEQ ID | cctgcatccccgttcccctgcatcccccttccccTGCATC |
| NO: 1170 | |
| SEQ ID | ACAGGGGTTCCTGGCTCTGCTCTTCAGACTGAGccccgtt |
| NO: 1171 | |
| SEQ ID | TCGTCCACCATCTCATGCCCCTGGCTCTCCTGCCCCTTCC |
| NO: 1172 | |
| SEQ ID | GCAAGCCCAGGAGAGGCGCTCAGGCTTCCCTGTCCCCCTT |
| NO: 1173 | |
| SEQ ID | TTCCCTAAGGCCCTGCTCTGGGCTTCTGGGTTTGAGTCCT |
| NO: 1174 | |
| SEQ ID | TGCTATCTGGGACATATTCCTCCGCCCAGAGCAGGGTCCC |
| NO: 1175 | |
| SEQ ID | GGTGCGTCCTAGGTGTTCACCAGGTCGTGGCCGCCTCTAC |
| NO: 1176 | |
| SEQ ID | gaggaGGGGGGTGTCCGTGTGGAAAACTCCCTTTGTGAGA |
| NO: 1177 | |
| SEQ ID | agataaggccagtagccagccccgtcctggcagggctgtg |
| NO: 1178 | |
| SEQ ID | ccccaatttatattgttcctccgtgcgtcagttttacctg |
| NO: 1179 | |
| SEQ ID | agttggtcctgagttctaactttggctcttcacctttcta |
| NO: 1180 | |
| SEQ ID | CTGGTGCGTTTCACTGATCCTGGTGCTGCAGCTTCCTTAC |
| NO: 1181 | |
| SEQ ID | CGCTACCCTCTCCCAGAACCTGAGCTGCTCTGACGCGGCC |
| NO: 1182 | |
| SEQ ID | GGGGGGGATGCGTGACCTGCCCGGTTCTCAGTGGCCACCC |
| NO: 1183 | |
| SEQ ID | TCCTTGCCAGAACCTCTAAGGTTTGCTTACGATGGAGCCA |
| NO: 1184 | |
| SEQ ID | CCTTATCTGGTGACACACCCCCATTTCCTGGAGCCATCTC |
| NO: 1185 | |

Post-hybridization washes. Coverslips were transferred from the humidified chamber into a new 6-well plate filled with 3 mL/well of 2×SSC and the plate was gently rocked to mix the remaining hybridization solution with SSC. SSC was aspirated and cells were washed with 3 mL/well of 2×SSC three times, each for 10 minutes, at room temperature. Cells were washed twice with 0.2×SSC, 0.2% Tween-20 with 2 mL/well of wash buffer on a digital hot plate set to 56° C. for 7 minutes. Cells were washed with 2 mL/well of 4×SSC, 0.2% Tween-20 for 5 minutes at room temperature and cells were subsequently washed twice with 2×SSC for 5 minutes per wash.

IF Staining for p53BP1 and FLAG. Blocking buffer was prepared containing 2% BSA (from 10% BSA/PBS), 0.05% Tween-20, 1×PBS. Cells were blocked with 1.5 mL/well of blocking buffer in a 6-well plate for 30 minutes at room temperature. Primary antibody incubation was carried out by first diluting the primary antibody in a blocking buffer at the following ratios: 1:500 for anti-p53BP1, 1:2000 for anti-FLAG. A humidified chamber was prepared and on a sheet of Parafilm inside the humidified chamber, 100 ul spots of primary antibody solution was placed. Coverslips were removed from the 6-well plate, inverted onto primary antibody spots, and incubated for 2 hours at room temperature. Coverslips were returned into the original 6-well plate with blocking buffer and cells were washed three times with 3 mL/well of 1×PBS for 5 minutes each. Secondary antibody incubation was carried out by first diluting secondary antibodies (donkey-anti-rabbit-AF488 and donkey-anti-mouse-AF594) in blocking buffer at a ratio of 1:500. On a new sheet of Parafilm inside the humidified chamber, secondary antibody solution was spotted at a volume of 100 ul. Coverslips were removed from the 6-well plate, inverted onto the secondary antibody spots, and incubated for 1.5 hours at room temperature. Coverslips were returned into the original 6-well plate and cells were washed three times with 3 mL/well of 1×PBS for 5 minutes each. Cells were stained with DAPI to visualize the nuclease by incubating cells in 1.5 mL/well of 1×PBS+100 ng/mL DAPI for 10 minutes at room temperature and cells were washed once with 1×PBS.

Mounting. Prolong Gold was placed at 10 ul drops onto pre-cleaned microscope slide. Coverslips were removed from the 6-well plate with tweezers, inverted onto Prolong Gold, and allowed to cure for 24 hours at room temperature. After 24 hours, coverslips were further sealed with nail polish, cleaned with water, and wiped dry prior to imaging.

Use of p53BP1 Analysis and Nano-FISH to Dissect On-Target Versus Off-Target Activity of Nucleases for Genome Editing

The combination of Nano-FISH imaging methods and p53BP1 imaging disclosed herein allows for in situ visualization of on-target versus off-target nuclease cutting activity. Fluorophore-conjugated oligonucleotide Nano-FISH probes were designed to hybridize to a target DNA genomic locus of interest. K562 cells were transfected with AAVS1-targeting TALENs for 24 hours as described in EXAMPLE 2. A fluorescently labeled Nano-FISH oligonucleotide probe was allowed to hybridize to the AAVS1 genomic locus in K562 cells and cells were additionally stained for p53BP1, as described above.

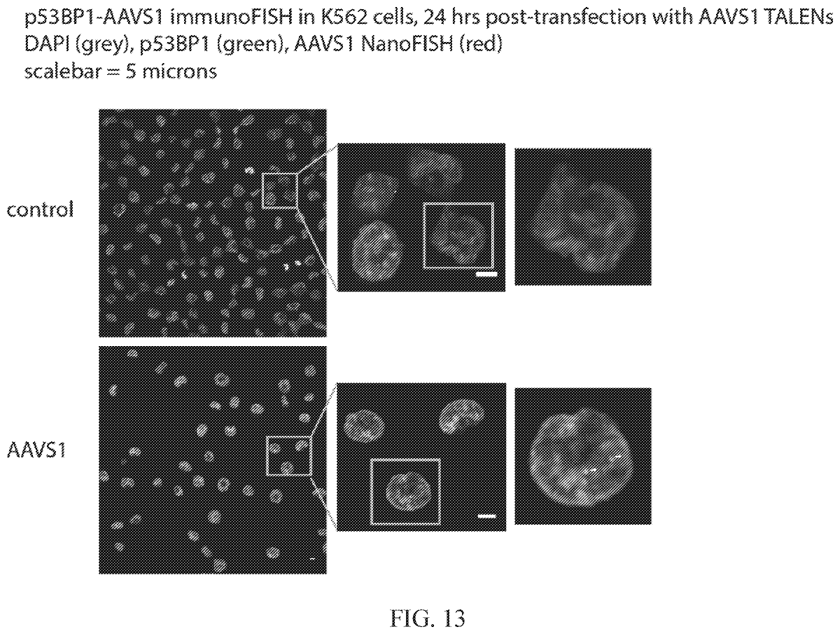

FIG. 13 shows fluorescence microscopy images of control cells and AAVS1-targeting TALEN treated cells. A DAPI stain (gray) was used to visualize nuclei, p53BP1 is shown in green and the AAVS1 oligonucleotide Nano-FISH probe was visualized in red. Imaging showed that in cells transfected with AAVS1-targeting TALEN, spots indicative of double stranded breaks (indicated by p53BP1 foci) co-localized with AAVS1 oligonucleotide Nano-FISH probe spots. These results showed that the AAVS1-targeting TALEN exhibited nuclease specificity, as confirmed by co-localization of DNA repair signals at the genomic locus of interest.

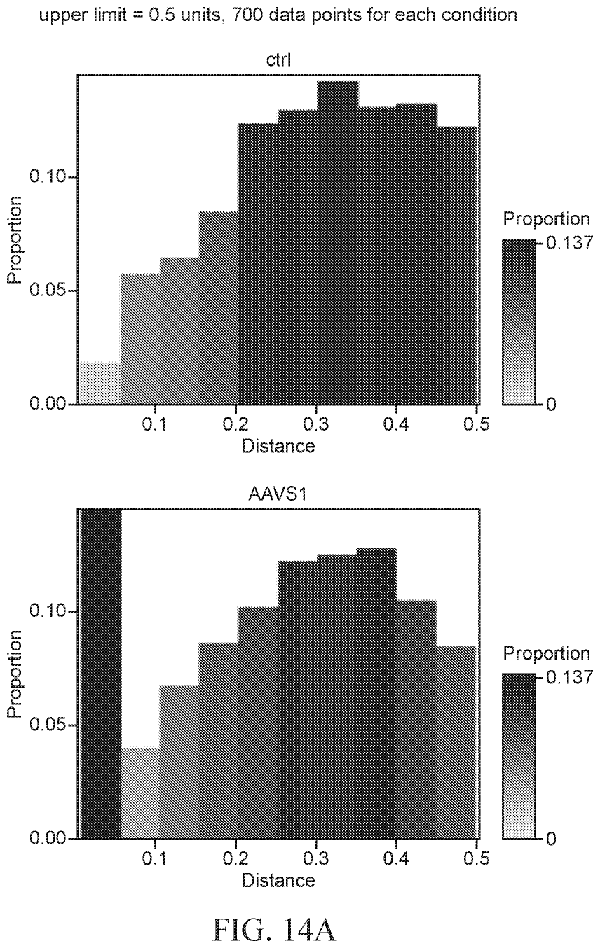

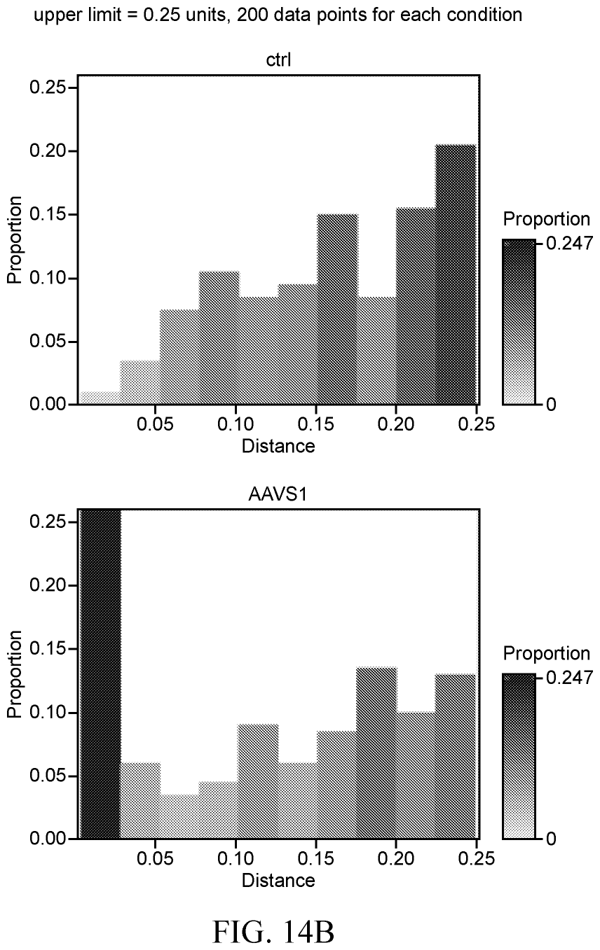

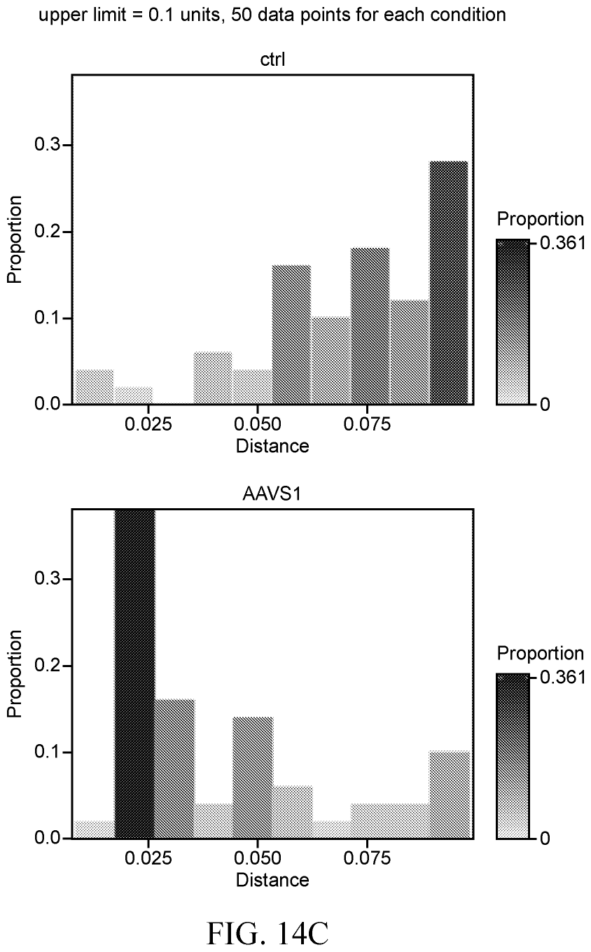

After imaging at high magnification on a fluorescence microscope, the pairwise distances between all AAVS1 Nano-FISH spots and p53BP1 foci were measured and quantified. FIG. 14 shows histograms of the proportion of pairwise distances between AAVS1 Nano-FISH spots and p53BP1 foci. FIG. 14A shows histograms of control and AAVS1 TALEN treated cells at pairwise distances of 0.1 to 0.5. FIG. 14B shows histograms of control and AAVS1 TALEN treated cells at pairwise distances of 0 to 0.025. FIG. 14C shows histograms of control and AAVS1 TALEN treated cells at pairwise distances of 0-0.08. Histograms showed a significantly higher co-location between AAVS1 loci and sites of DNA repair in TALEN-treated cells relative to untreated control cells. Thus, the combination of Nano-FISH and p53BP1 foci visualization enable the measurement of off-target activity (the number of p53BP1 foci not co-localized with their target genomic loci).

### Example 12

### Use of p53BP1 Analysis for Diverse Micro Imaging Platforms and Small Cell Samples

This example illustrates the use of p53BP1 analysis for diverse micro imaging platforms and small cell samples. Nuclease specificity has also been determined using the compositions and methods described herein in on several types of imaging platforms and in smaller sample sizes. Samples were imaged using a Nikon microscope or the Stellar-Vision microscope, as described in EXAMPLE 1.

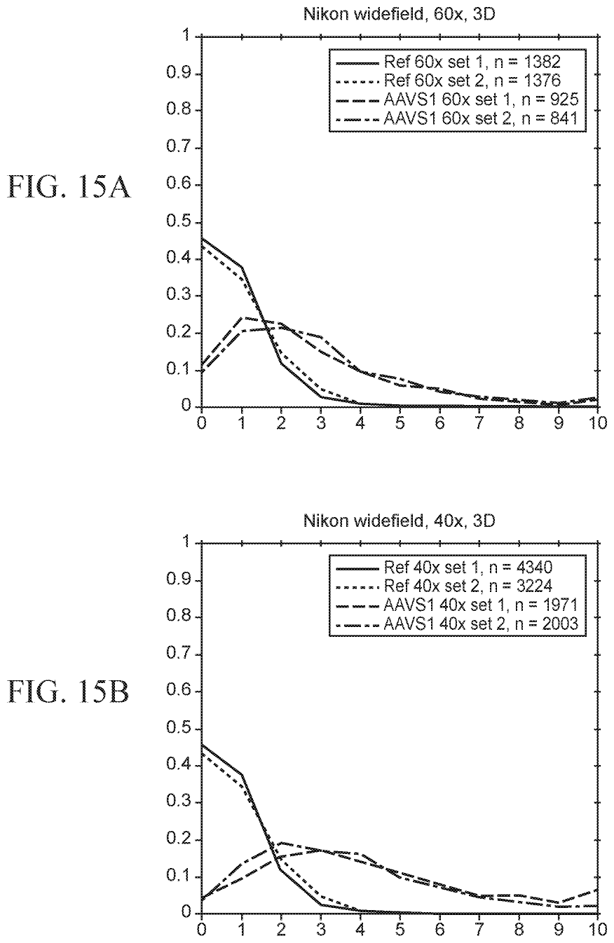

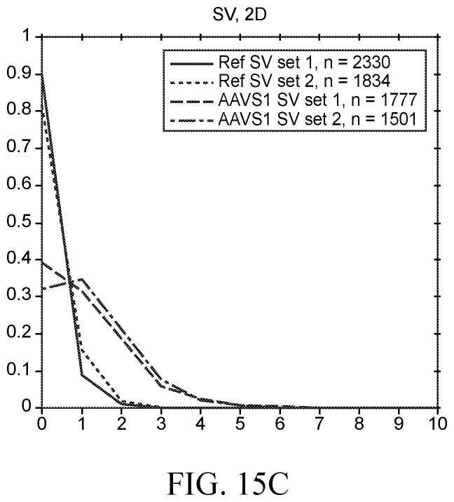

FIG. 15 shows evaluation of nuclease specificity by counting p53BP1 foci in cells transfected with AAVS1-targeting TALENs FIG. 15A illustrates the number of p53BP1 foci on the x axis versus the proportion of cells with p53BP1 foci on the y-axis in cells transfected with AAVS1-targeting TALENs and, in 3D, imaged on a Nikon widefield fluorescence microscope with a 60× magnification lens using oil immersion contact techniques. ‘Ref’ samples indicate control cells that were not transfected with TALENs. Biological replicates are shown for control and transfected cells (indicated by set x). The number of cells analyzed in each sample is indicated by “n.”

FIG. 15B illustrates the number of p53BP1 foci on the x axis versus the proportion of cells with p53BP1 foci on the y-axis in cells transfected with AAVS1-targeting TALENs and imaged, in 3D, on a Nikon widefield fluorescence microscope with a 40× magnification lens using non-contact techniques. “Ref” samples indicate control cells that were not transfected with TALENs. Biological replicates are shown for control and transfected cells. The number of cells analyzed in each sample is indicated by “n.”

FIG. 15C illustrates the number of p53BP1 foci on the x axis versus the proportion of cells with p53BP1 foci on the y-axis in cells transfected with AAVS1-targeting TALENs and imaged on a Stellar-Vision (SV) fluorescence microscope using non-contact techniques. ‘Ref’ samples indicate control cells that were not transfected with TALENs Biological replicates are shown for control and transfected cells. The number of cells analyzed in each sample is indicated by “n.”

TABLE 14 below shows p values from several statistical tests including a t-test, Kolmogorov-Smirnov (KS) test, and Wilcoxon-smith (WS) test comparing of p53BP1 spots in transfected cells and control cells.

TABLE 14

| | Imaging Modality (n = 1000 cells) | | |
|---|---|---|---|
| Test | 60x 3D | 40x 3D | SV |
| t-test | 4e−96 | 2e−203 | 9e−102 |
| KS test | 6e−100 | 6e−225 | 2e−102 |
| WS test | 1e−121 | 1e−233 | 6e−116 |

TABLE 15 below shows p-values from a t-test comparing p53BP1 spots in transfected cells and control cells for different sample sizes. The results below show a high degree of statistical significance even when analyzing a small number of cells across all imaging modalities. These results demonstrated the utility of using p53BP1 analysis for clinically relevant applications that involve the use of small sample sizes to screen nucleases for lead candidates.

TABLE 15

| | t-test for Imaging Modality | | |
|---|---|---|---|
| Sample size | 60x 3D | 40x 3D | SV |
| 1000 | 4e−96 | 2e−203 | 9e−102 |
| 500 | 1e−45 | 4e−95 | 4e−57 |
| 100 | 8e−12 | 2e−23 | 3e−10 |
| 50 | 4e−8 | 4e−11 | 4e−8 |

### Example 13

### Screening of Nucleases for Specificity

This example illustrates screening of nucleases for a nuclease with high specificity using the compositions and methods disclosed herein for staining, imaging, and analyzing a protein (e.g., p53BP1) that accumulates at the site of a double strand break. Several nucleases of various types (e.g., TALENS, Cas9) are screened for nuclease specificity in immortalized cells (e.g., K562, A549) and primary cells (e.g., CD34+ progenitor cells, naïve or stimulated T cells). Nucleases are transfected in immortalized or primary cells, as described in EXAMPLE 2 or EXAMPLE 3. Cells are stained for p53BP1 using the methods as set forth in EXAMPLE 4. Imaging, image analysis, and informatics is carried out using the methods set forth in EXAMPLE 1. p53BP1 foci are automatically counted and plotted against a parameter of interest for each nuclease (dose of nuclease, RVD length, etc.). Nuclease specificity is assessed for each nuclease tested by quantifying the total p53BP1 load (e.g., number of protein foci or total protein content within the nucleus). A high p53BP1 load indicates nucleases with relatively poor specificity. A lower p53BP load indicates nucleases with better specificity.

### Example 14

### Confirming Specificity of Genome Editing with a Nuclease

This example illustrates confirming specificity of genome editing with a nuclease. A genome editing complex comprising a nuclease (e.g., TALENs, zinc finger nucleases (ZFNs), or CRISPR/Cas9) targeting a therapeutic gene of interest for genome editing is transfected in immortalized or primary cells as set forth in EXAMPLE 2 or EXAMPLE 3. The nuclease induces double stranded breaks. Cells are stained and analyzed as described in EXAMPLE 10 with an oligonucleotide Nano-FISH probe set for the particular genomic locus of the therapeutic gene of interest and for p53BP1, indicative of double strand breaks induced by the nuclease. Cells are imaged and analyzed as described in EXAMPLE 1. Co-localization of oligonucleotide Nano-FISH probes and all double strand breaks is observed, indicating a nuclease with high specificity and no off target activity.

### Example 15

### Screening of Epigenomic Repressors for Specificity

This example illustrates screening of repressors for a repressor with high specificity using the compositions and methods disclosed herein for staining, imaging, and analyzing a protein (e.g., KAP1, H3K9me3 or HP1) that accumulates at the site of repression (e.g., by KRAB). Repressors of various types (e.g., KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2) are screened for specificity in immortalized cells (e.g., K562, A549) and primary cells (e.g., CD34+ progenitor cells, naïve or stimulated T cells). Repressors coupled to a binding domain (e.g., RVDs for TALENs, guide RNAs for CRISPR/dCas9 systems) are transfected in immortalized or primary cells, as described in EXAMPLE 2 or EXAMPLE 3. Cells are stained for a protein (e.g., KAP1) using the methods as set forth in EXAMPLE 4 with antibodies specific to the protein. Imaging image analysis, and informatics is carried out using the methods set forth in EXAMPLE 1. Protein (e.g., KAP1) foci are automatically counted and plotted against a parameter of interest for each repressor (e.g., dose of repressor, RVD length, etc.). Repressor specificity is assessed for each repressor tested by counting for protein (e.g., KAP1) foci. A high number of protein (e.g., KAP1) foci indicate repressors with relatively low specificity. A lower number of protein (e.g., KAP1) foci indicate repressors with better specificity. Site-specific detection of proteins such as H3K9me3 or HP1 can be confirmed by combination imaging with Nano-FISH, as described in EXAMPLE 10.

### Example 16

### Detecting Chromosomal Trans Location Events Using p53BP1 Foci Analysis

This example illustrates the detection of translocation events using the image-based analyses of p53BP1 load disclosed herein. A genome editing complex (e.g., TALEN, CRISPR/Cas9, megaTAL, meganuclease) is transfected to an immortalized or primary cell, as described in EXAMPLE 2 or EXAMPLE 3. Cells are stained for p53BP1 as described in EXAMPLE 4 with a first detectable agent and subsequently administered a oligonucleotide Nano-FISH probe set with a second detectable agent for the target genomic locus and a different oligonucleotide Nano-FISH probe set with a third detectable agent for an off-target genomic locus. Samples are imaged as set forth in EXAMPLE 1. Foci of p53BP1 are visualized by signal from the first detectable agent, indicating a double strand break and gene editing with the genome editing complex. Foci of the first oligonucleotide Nano-FISH probe set are visualized by signal from the second detectable agent, indicating the target genomic locus. Foci of the second oligonucleotide Nano-FISH probe set are visualized by signal from the third detectable agent, indicating the off-target genomic locus. In the absence of a translocation event, co-localization of the signal from the first detectable agent and the second detectable agent is observed, indicating co-localization of p53BP1 with the oligonucleotide Nano-FISH probe set for the target genomic locus. When chromosomal translocation occurs, co-localization of the signal from the first detectable agent, the second detectable agent, and the third detectable agent is observed, indicating co-localization of p53BP1 with the oligonucleotide Nano-FISH probe set for the target genomic locus and the oligonucleotide Nano-FISH probe set for the off-target genomic locus.

### Example 17

### Determining Specificity of Genome Editing with a Transthyretin (TTR)-Targeting Nuclease

This example illustrates determining specificity of genome editing with a transthyretin (TTR)-targeting nuclease. A genome editing complex (e.g., TALEN, ZFN, CRISPR/Cas9, megaTAL, meganuclease) targeting TTR is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for TTR and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for TTR and any off-target activity of the nuclease. A nuclease with high specificity for TTR and low to none off-target activity is used to administer in a subject in need thereof. The subject has transthyretin amyloidosis (ATTR).

### Example 18

### Determining Specificity of Genome Editing with a CCR5-Targeting Nuclease

This example illustrates determining specificity of genome editing with a CCR5-targeting nuclease. A genome editing complex (e.g., TALEN, ZFN, CRISPR/Cas9, megaTAL, meganuclease) targeting CCR5 is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for CCR5 and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for CCR5 and any off-target activity of the nuclease. A nuclease with high specificity for CCR5 and low to none off-target activity is used to administer in a subject in need thereof. The subject has HIV.

### Example 19

### Determining Specificity of Genome Editing with a Glucocorticoid Receptor (NR3C1)-Targeting Nuclease

This example illustrates determining specificity of genome editing with a glucocorticoid receptor (NR3C1)-targeting nuclease. A genome editing complex (e.g., TALEN, ZFN, CRISPR/Cas9, megaTAL, meganuclease) targeting NR3C1 is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for NR3C1 and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for NR3C1 and any off-target activity of the nuclease. A nuclease with high specificity for NR3C1 and low to none off-target activity is used to administer in a subject in need thereof. The subject has glioblastoma multiforme.

### Example 20

### Determining Specificity of Genome Editing with a TRA-Targeting Nuclease and/or a CD52-Targeting Nuclease

This example illustrates determining specificity of genome editing with a TRA-targeting nuclease and/or a CD52-targeting nuclease. A genome editing complex (e.g., TALEN, ZFN, CRISPR/Cas9, megaTAL, meganuclease) targeting TRA and a genome editing complex (e.g., TALEN, ZFN, CRISPR/Cas9, megaTAL, meganuclease) targeting CD52 are transfected in immortalized or primary cells as set forth in EXAMPLE 2 or EXAMPLE 3. The nuclease induces double stranded breaks. Cells are stained and analyzed as described in EXAMPLE 1 with an oligonucleotide Nano-FISH probe set for TRA and/or CD52 and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for TRA and/or CD52 and any off-target activity of the nuclease. A nuclease with high specificity for TRA and/or CD52 and low to none off-target activity is used to administer to cells ex vivo to generate a universal T cell therapy, to be administered to a subject in need thereof. The subject has a cancer, such as acute lymphoblastic leukemia or acute myeloid leukemia.

### Example 21

### Determining Specificity of Genome Editing with a Nuclease Targeting the Erythroid Specific Enhancer of BCL11A

This example illustrates determining specificity of genome editing with a nuclease targeting the erythroid specific enhancer of BCL11A. A genome editing complex (e.g., TALEN, ZFN, CRISPR/Cas9, megaTAL, meganuclease) targeting the erythroid specific enhancer of BCL11A is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for the erythroid specific enhancer of BCL11A and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for the erythroid specific enhancer of BCL11A and any off-target activity of the nuclease. A nuclease with high specificity for the erythroid specific enhancer of BCL11A and low to none off-target activity is used to engineer hematopoietic stem cells ex vivo, to be administered to a subject in need thereof. The subject has beta-thalassemia or sickle cell disease.

### Example 22

### Determining Specificity of Genome Editing with a Nuclease to Insert Alpha-L Iduronidase (IDUA)

This example illustrates determining specificity of genome editing with a nuclease disclosed herein to insert alpha-L iduronidase (IDUA). A genome editing complex (e.g., TALEN, ZFN, CRISPR/Cas9, megaTAL, meganuclease) targeting a desired genomic locus for insertion of an ectopic nucleic acid encoding for IDUA is transfected in immortalized or primary cells as set forth in EXAMPLE 2 or EXAMPLE 3. The nuclease induces double stranded breaks to insert a functional IDUA gene. Cells are stained and analyzed as described in EXAMPLE 11 with an oligonucleotide Nano-FISH probe set for IDUA and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease and any off-target activity of the nuclease. A nuclease with high and low to none off-target activity is used to administer in a subject in need thereof. The subject has MPSI.

### Example 23

### Determining Specificity of Genome Editing with a Nuclease to Insert Iduronate-2-Sulfatase (IDS)

This example illustrates determining specificity of genome editing with a nuclease disclosed herein to insert iduronate-2-sulfatase (IDS). A genome editing complex (e.g., TALEN, ZFN, CRISPR/Cas9, megaTAL, meganuclease) targeting a desired genomic locus for insertion of an ectopic nucleic acid encoding for IDS is transfected in immortalized or primary cells as set forth in EXAMPLE 2 or EXAMPLE 3. The nuclease induces double stranded breaks to insert a functional IDS gene. Cells are stained and analyzed as described in EXAMPLE 11 with an oligonucleotide Nano-FISH probe set for IDS and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease and any off-target activity of the nuclease. A nuclease with high specificity and low to none off-target activity is used to administer in a subject in need thereof. The subject has MPSII.

### Example 24

### Determining Specificity of Genome Editing with a Nuclease to Insert Factor IX

This example illustrates determining specificity of genome editing with a nuclease to insert Factor IX. A genome editing complex (e.g., TALEN, ZFN, CRISPR/Cas9, megaTAL, meganuclease) targeting a desired genomic locus for insertion of an ectopic nucleic acid encoding for Factor 9 is transfected in immortalized or primary cells as set forth in EXAMPLE 2 or EXAMPLE 3. The nuclease induces double stranded breaks to insert a functional Factor 9 gene. Cells are stained and analyzed as described in EXAMPLE 11 with an oligonucleotide Nano-FISH probe set for Factor 9 and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease and any off-target activity of the nuclease. A nuclease with high specificity and low to none off-target activity is used to administer in a subject in need thereof. The subject has Hemophilia B.

### Example 25

### Determining Specificity of Genome Editing with a PDCD1-Targeting Nuclease, a TRA-Targeting Nuclease, and/or a TRB-Targeting Nuclease

This example illustrates determining specificity of genome editing with a PDCD1-targeting nuclease, a TRA-target nuclease, and/or a TRB-targeting nuclease. A genome editing complex (e.g., TALEN, ZFN, CRISPR/Cas9, megaTAL, meganuclease) targeting PDCD1, TRA, and/or TRB is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for PDCD1, TRA, and/or TRB and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for PDCD1, TRA, and/or TRB and any off-target activity of the nuclease. A nuclease with high specificity for PDCD1, TRA, and/or TRB and low to none off-target activity is used to administer to engineer CAR T cells ex vivo, to be administered to a subject in need thereof. The subject has cancer, such as multiple myeloma, melanoma, or sarcoma.

### Example 26

### Determining Specificity of Genome Editing with a TRA-Targeting Nuclease, a TRB-Targeting Nuclease, and/or a CS-1-Targeting Nuclease

This example illustrates determining specificity of genome editing with a TRA-targeting nuclease, a TRB-targeting nuclease, and/or a CS-1-targeting nuclease. A genome editing complex (e.g., TALEN, ZFN, CRISPR/Cas9, megaTAL, meganuclease) targeting TRA, TRB, and/or CS-1-1 is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for TRA, TRB, and/or CS-land for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for TRA, TRB, and/or CS-1 and any off-target activity of the nuclease. A nuclease with high specificity for TRA, TRB, and/or CS-1 and low to none off-target activity is used to administer to engineer CAR T cells ex vivo, to be administered to a subject in need thereof. The subject has cancer, such as multiple myeloma.

### Example 27

### Determining Specificity of Genome Editing with a TRA-Targeting Nuclease and/or a TRB-Targeting Nuclease

This example illustrates determining specificity of genome editing with a TRA-targeting nuclease and/or a TRB-targeting nuclease. A genome editing complex (e.g., TALEN, ZFN, CRISPR/Cas9, megaTAL, meganuclease) targeting TRA and/or TRB is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for TRA and/or TRB and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for TRA and/or TRB and any off-target activity of the nuclease. A nuclease with high specificity for TRA and/or TRB and low to none off-target activity is used to administer to engineer CAR T cells ex vivo, to be administered to a subject in need thereof. The subject has cancer, such as acute lymphoblastic leukemia.

### Example 28

### Determining Specificity of Genome Editing with a CEP290-Targeting Nuclease

This example illustrates determining specificity of genome editing with a CEP290-targeting nuclease. A genome editing complex (e.g., TALEN, ZFN, CRISPR/Cas9, megaTAL, meganuclease) targeting CEP290 is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for CEP290 and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for CEP290 and any off-target activity of the nuclease. A nuclease with high specificity for CEP290 and low to none off-target activity is used to administer to a subject in need thereof. The subject has Leber congenital amaurosis (LCA10).

### Example 29

### Determining Specificity of Genome Editing with a TRA-Targeting Nuclease, a TRB-Targeting Nuclease, and/or a B2M-Targeting Nuclease

This example illustrates determining specificity of genome editing with a TRA-targeting nuclease, a TRB-targeting nuclease, and/or a B2M-targeting nuclease. A genome editing complex (e.g., TALEN, ZFN, CRISPR/Cas9, megaTAL, meganuclease) targeting TRA, TRB, and/or B2M is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for TRA, TRB, and/or B2M and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for TRA, TRB, and/or B2M and any off-target activity of the nuclease. A nuclease with high specificity for TRA, TRB, and/or B2M and low to none off-target activity is used to administer to engineer CAR T cells ex vivo, to be administered to a subject in need thereof. The subject has cancer, such as CD19 malignancies or BCMA-related malignancies.

### Example 30

### Multiplexed p53BP1, FLAG, and Nano-FISH Staining for Fine Structural Analysis

This example shows multiplexed p53BP1, FLAG, and Nano-FISH staining and analysis for fine structural analysis of specific genomic loci within the nucleus. Fine structural analysis using Nano-FISH is carried by, for example, probe pools are designed to target a 1.6kb region of chromosome 19 and a 1.4kb region of chromosome 18. Distinct spots are produced by Nano-FISH probes targeting specific loci on these chromosomes. To measure the relative localization of the detected loci, the relative radial distance (RRD), a normalized measure of the position of the detected spot with respect to the nuclear centroid, was calculated. Distributions are obtained across 2,396 chromosome 18 signals and 3,388 chromosome 19 signals. The differences in the distribution of signals with respect to the nuclear centroid are readily apparent in the histograms. Fine structural analysis using Nano-FISH is extended to the multiplexed p53BP1, FLAG, and Nano-FISH staining and analysis disclosed herein to spatially resolve the target genomic locus within the nucleus in 2D or 3D.

### Example 31

### Examination of Enhancer-Promoter Interactions Using Multiplexed p53BP1, FLAG, and Nano-FISH Staining

This example shows multiplexed p53BP1, FLAG, and Nano-FISH staining and analysis for examining the interaction of a gene enhancer with its target gene promoter. The positioning of a known enhancer is examined. Nano-FISH probes targeting the enhancer and promoter are designed and synthesized. The normalized inter-spot distance (NID) between two genomic loci is compared. Small size of genomic regions targeted by Nano-FISH permits fine scale localization of regulatory DNA regions and provides a granular view of their spatial localizations within nuclei. Examination of enhancer-promoter interactions using Nano-FISH is extended to the multiplexed p53BP1, FLAG, and Nano-FISH staining and analysis disclosed herein to examine enhancer-promoter interactions after editing cells with a genome editing complex (e.g., TALEN, ZFN, CRISPR/Cas9, megaTAL, meganuclease).

### Example 32

### Fine Scale Genome Localization Using Multiplexed p53BP1, FLAG, and Nano-FISH Staining and Super-Resolution Microscopy

This example shows multiplexed p53BP1, FLAG, and Nano-FISH staining and analysis super-resolution microscopy to obtain very fine-scale genome localization. Fine scale genome localization using Nano-FISH and super-resolution microscopy is carried out as follows. A custom automated stimulated emission and depletion (STED) microscope is utilized to efficiently acquire multiple measurements of the physical distance between the HS2 and HS3 genomic loci, which are separated by 4.1kb of linear genomic distance. Pairwise measurements of other closely situated genomic segments such as HS1-HS4 (˜12kb) and HS2-HGB2 (˜25kb) are also readily obtained and revealed non-linear compaction of the β-globin locus control region and the surrounding genome which contains its target genes. Importantly, the high-throughput STED microscopy approach enables calculation of the distribution of actual distances between these various loci. Nano-FISH is suitable for super-resolution STED microscopy experiments. Examination of fine scale genome localization using Nano-FISH is extended to the multiplexed p53BP1, FLAG, and Nano-FISH staining and analysis disclosed herein to examine fine scale genome localization after editing cells with a genome editing complex (e.g., TALEN, ZFN, CRISPR/Cas9, megaTAL, meganuclease).

### Example 33

### Determining Specificity of Genome Editing with a CBLB-Targeting Nuclease

This example illustrates determining specificity of genome editing with a CBLB-targeting nuclease. A genome editing complex (e.g., TALEN, ZFN, CRISPR/Cas9, megaTAL, meganuclease) targeting CBLB is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for CBLB and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for CBLB and any off-target activity of the nuclease. A nuclease with high specificity for CBLB and low to none off-target activity is administered to engineer CAR T cells ex vivo, to be administered to a subject in need thereof. The subject has cancer.

### Example 34

### Determining Specificity of Genome Editing with a TGFBR-Targeting Nuclease

This example illustrates determining specificity of genome editing with a TGFbR-targeting nuclease. A genome editing complex (e.g., TALEN, ZFN, CRISPR/Cas9, megaTAL, meganuclease) targeting TGFBR is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for TGFBR and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for TGFBR and any off-target activity of the nuclease. A nuclease with high specificity for TGFBR and low to none off-target activity is administered to engineer CAR T cells ex vivo, to be administered to a subject in need thereof. The subject has multiple myeloma.

### Example 35

### Determining Specificity of Genome Editing with a DMD-Targeting Nuclease

This example illustrates determining specificity of genome editing with a DMD-targeting nuclease. A genome editing complex (e.g., TALEN, ZFN, CRISPR/Cas9, megaTAL, meganuclease) targeting DMD is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for DMD and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for DMD and any off-target activity of the nuclease. A nuclease with high specificity for DMD and low to none off-target activity is administered to a subject in need thereof. The subject has duchenne muscular dystrophy (DMD).

### Example 36

### Determining Specificity of Genome Editing with a CFTR-Targeting Nuclease

This example illustrates determining specificity of genome editing with a CFTR-targeting nuclease. A genome editing complex (e.g., TALEN, ZFN, CRISPR/Cas9, megaTAL, meganuclease) targeting CFTR is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for CFTR and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for CFTR and any off-target activity of the nuclease. A nuclease with high specificity for CFTR and low to none off-target activity is administered to a subject in need thereof. The subject has cystic fibrosis.

### Example 37

### Determining Specificity of Genome Editing with a Serpinal-Targeting Nuclease

This example illustrates determining specificity of genome editing with a serpinal-targeting nuclease. A genome editing complex (e.g., TALEN, ZFN, CRISPR/Cas9, megaTAL, meganuclease) targeting serpinal is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for serpinal and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for serpinal and any off-target activity of the nuclease. A nuclease with high specificity for serpinal and low to none off-target activity is administered to a subject in need thereof. The subject has alpha-1 antitrypsin deficiency (dA1AT def).

### Example 38

### Determining Specificity of Genome Editing with an IL2Rg-Targeting Nuclease

This example illustrates determining specificity of genome editing with an IL2Rg-targeting nuclease. A genome editing complex (e.g., TALEN, ZFN, CRISPR/Cas9, megaTAL, meganuclease) targeting IL2Rg is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for IL2Rg and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for IL2Rg and any off-target activity of the nuclease. A nuclease with high specificity for IL2Rg and low to none off-target activity is administered to a subject in need thereof. The subject has X-linked severe combined immunodeficiency (X-SCID).

### Example 39

### Determining Specificity of Genome Editing with Nuclease Targeting HBV Genomic DNA in Infected Cells

This example illustrates determining specificity of genome editing with a nuclease targeting HBV genomic DNA in infected cells. A genome editing complex (e.g., TALEN, ZFN CRISPR/Cas9, megaTAL, meganuclease) targeting HBV genomic DNA is transfected in immortalized or primary cells as set forth in EXAMPLE 2 or EXAMPLE 3. The nuclease induces double stranded breaks. Cells are stained and analyzed as described in EXAMPLE 11 with an oligonucleotide Nano-FISH probe set for HBV genomic DNA and for p53BP1, indicative of double strand breaks induced by the nuclease. Cells are imaged and analyzed as described in EXAMPLE 1. Co-localization of signal from oligonucleotide Nano-FISH probes and p53BP1 is quantified to determine the specificity of the nuclease for HBV genomic DNA and any off-target activity of the nuclease. A nuclease with high specificity for HBV genomic DNA and low to none off-target activity is administered to a subject in need thereof. The subject has Hepatitis B.

### Example 40

### Calculation of Nuclease Specificity

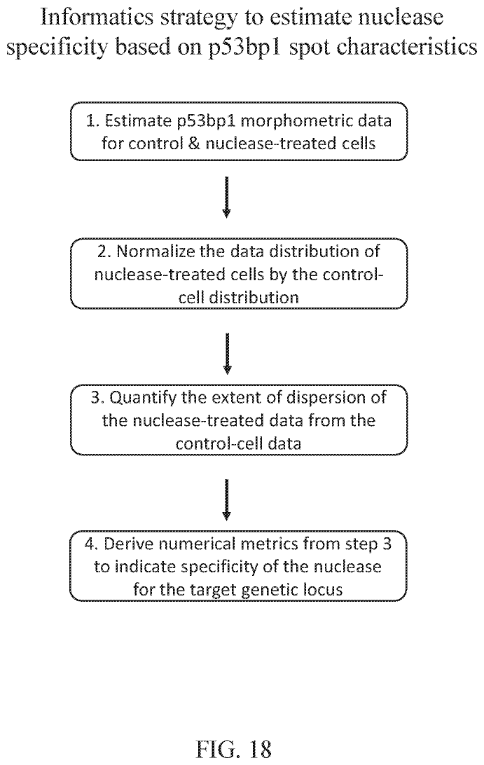

A modular software framework of image processing methods to quantify the amount and localization of proteins (such as p53bp1) on a per-cell basis in response to a perturbant such as a nuclease has been developed. For the protein of interest, morphometric data (such as foci (spot) count, foci size, foci intensity, overall nuclear expression (load), spatial localization patterns of foci, etc) are automatically estimated from the image data on a per-cell basis for the nuclease-treated and mock-treated (control) samples. A generalizable informatics framework of statistical methods to model and analyze the data distributions has also been developed. The informatics framework ultimately yields a numerical estimate ([0,1] or expressed as a percentage) for the specificity of the nuclease. The framework is depicted in FIG. 18. This framework thus provides an objective route for high throughput screening of nucleases to identify lead nucleases against therapeutically useful genomic targets.

### Example 41

### Calculation of Nuclease Specificity Using Per-Cell p53BP1 Foci Counts

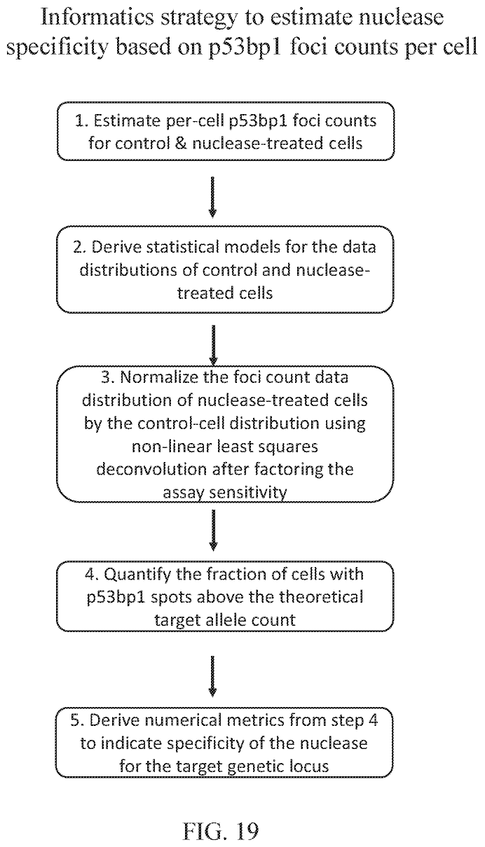

Per-cell spot counts for the p53bp1 protein in control and nuclease-treated cells can be modeled and analyzed using the informatics framework detailed in FIG. 18 to yield numerical estimates of the nuclease specificity. The model incorporates parameters to reflect the sensitivity of the protein marker used, and the ploidy of the target locus that is being edited. The nuclease-treated cell distribution was normalized relative to the distribution of the control sample, and the fraction of cells with p53bp1 foci above the ploidy of the target genomic locus was computed as the promiscuity of the nuclease. Nuclease specificity was estimated to be 1−the promiscuity value. A method for calculation of nuclease specificity based on p53bp1 foci counts is depicted in FIG. 19.

### Example 42

### Calculation of Nuclease Specificity Using Per-Cell p53BP1 Foci Counts Vs. Guide-Seq

Guide-seq is a bulk-cell genomic sequencing-based assay that generally considered as the defacto method to derive the specificity of nucleases. The imaging assay disclosed herein provides a complementary estimate of the nuclease specificity, but within a fraction of the time and expense of the guide-seq assay.

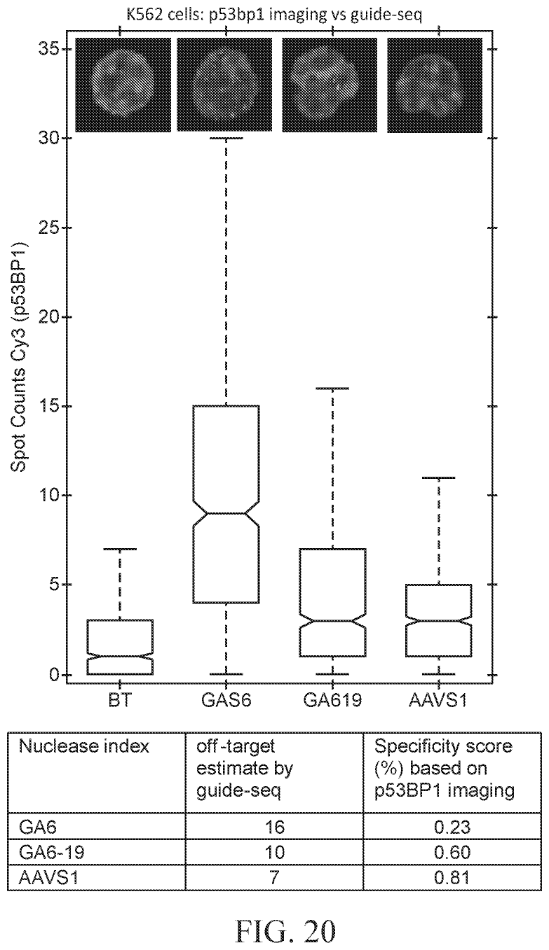

The specificity of p53BP1 imaging assay was compared with guide-seq in K562 cells for 3 nucleases that are considered to have high on-target potency but differing specificities. The p53BP1 imaging-based assay mirrors the specificity profiles provided by guide-seq, but within a fraction of the time and cost of the guide-seq assay. See FIG. 20.

### Example 43

### p53BP1 Imaging Based Optimization of Nuclease Specificity by Altering DNA Binding Domain

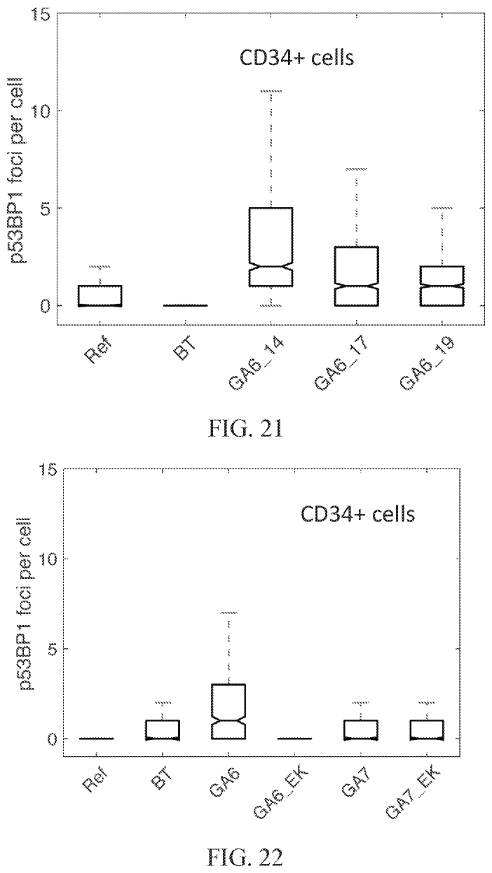

p53BP1 imaging assay was utilized to optimize the specificity of nucleases in primary cells by modifying their design. CD34+ cells were treated with either TALENs featuring homodimeric FokI nuclease domains (GA6_14) or their variants that contained more repeat units (i.e. GA6_17 and GA6_19) in one of the monomers (the left monomer in this case) to enhance specific recognition of their target genomic locus. The assay revealed a dramatic reduction in off-target activity by using longer GA6_L monomers while still providing a comparable on-target editing efficiency (58% for GA6_14, 54% for GA6_17, and 52% for GA6_19). See FIG. 21.

### Example 44

### p53BP1 Imaging Based Optimization of Nuclease Specificity by Altering Nuclease Domain

p53BP1 imaging assay was utilized to optimize the specificity of nuclease action in primary cells. CD34+ cells were treated with either TALENs featuring homodimeric FokI nuclease domains (GA6, GA7) or their variants that contained obligate heterodimeric ETD/KKR FokI nuclease domains (GA6_EK, GA7_EK). The assay revealed a substantial decrease in the off-target nuclease activity of the obligate heterodimer variant of the GA6 talen. The improved specificity does occur with a collateral of lower editing (47% for GA6, 58% for GA7 vs 29% for GA6-EK and 21% for GA7-EK). See FIG. 22.

### Example 45

### p53BP1 Imaging Based Optimization of Nuclease Specificity by Altering Nuclease Domain

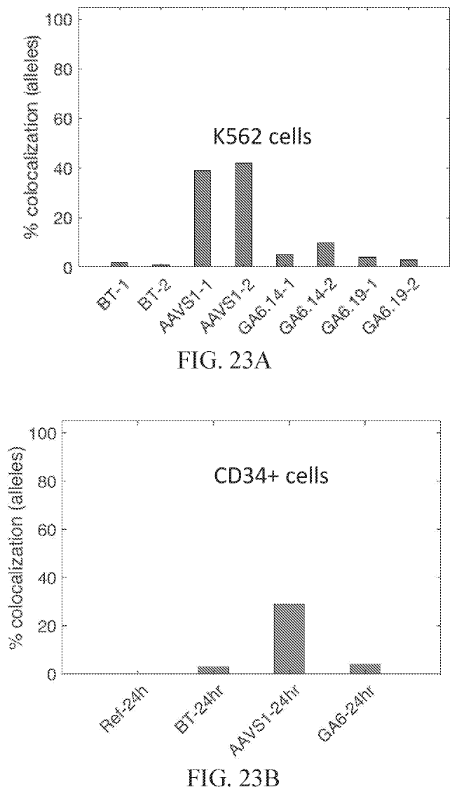

By multiplexing immunofluorescence with NanoFISH, p53BP1 imaging assay can be used to assess both on- and off-target activity on a per-cell basis. K562 cells or CD34+ progenitor cells were treated with AAVS1 and GA6 TALENs that target distinct genomic regions. Untransfected and mock transfected cells were used as controls. An mRNA dose of 2 ug per monomer was used for the TALENs. 24 hours post transfection, all cells were subject to p53BP1/FLAG immunofluorescence and NanoFISH with a pool of 115 oligoprobes that were designed to target the 5 kb genomic region adjacent to AAVS1 TALEN cut site. K562 cell experiments were conducted in duplicate. Colocalization analysis of the AAVS1 FISH probes and the p53BP1 protein foci revealed a significantly higher colocalization of AAVS1 FISH foci with p53BP1 foci in the AAVS1 TALEN treated cells compared to all the other conditions in both cell types. See FIGS. 23A and 23B. These results highlight the utility of the assay for a per-allele per-cell readout of on- and off-target activity of a nuclease.

### Example 46

### Imaging-Based Specificity Screen to Identify Lead Nucleases for Therapeutic Genetic Targets

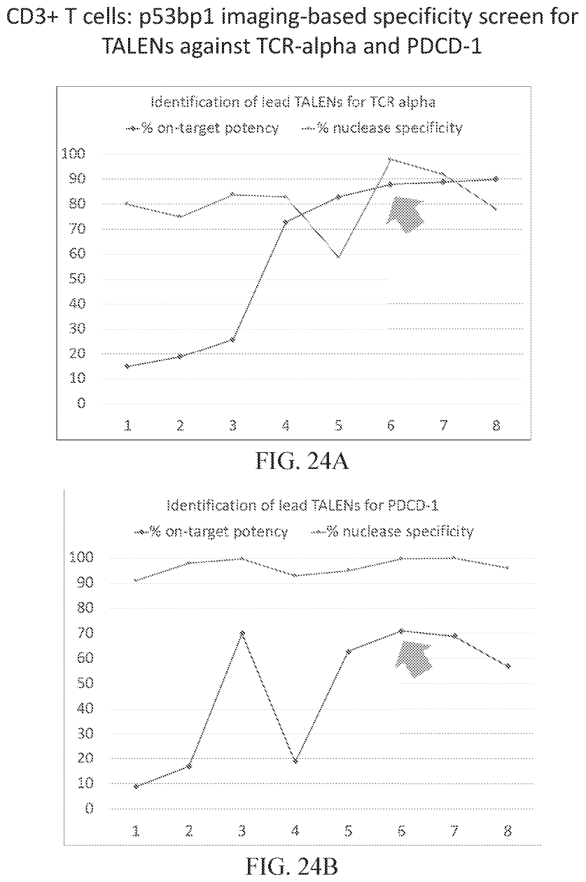

The p53BP1 imaging assay was used to rapidly identify lead nucleases against therapeutically relevant genomic loci. TALENs against the first constant exon of the TCR-alpha gene and the first exon of the PDCD1 gene were designed, and their on-target potency and specificity on primary CD3+ T cells was evaluated. Multiple TALENs provided comparable on-target potency, TALEN #6 had the highest specificity. See FIGS. 24A and 24B. Thus, the p53BP1 imaging assay identified TALEN #6 as the lead nuclease for these genes.

FIGS. 24A-24B: Primary CD3+ T cells were transfected with a set of 8 TALENs against either TCR-alpha (FIG. 24A) or PDCD-1 (FIG. 24B), at a dose of 2 ug per monomer. TALEN mRNA was used for the transfection. Transfected cells were subject to cold shock (30C) for 24 hours, after which they were retrieved, washed with PBS, seeded onto PLL-coated, glass bottom 24-well plates, stained for p53BP1 and FLAG, and imaged in 3D using a Nikon epi fluorescence microscope fitted with an Andor Zyla camera and 60×, 1.4 NA oil objective.

% on-target potency: On target potency is a measure of the cutting efficacy of the nuclease at the intended genomic target site. Genomic DNA is retrieved from cells 72-96 hours post transfection, amplicons generated for the intended target site, and these were sequenced with the miniseq (up to 500,000 reads). The on-target potency value is calculated from the sequencing data as the proportion of reads that contain either insertions or deletions at the edited target genomic locus to the total number of reads sequenced for the sample.

% nuclease specificity is computed from the per-cell p53bp1 foci count data. The data distributions for the nuclease-treated and the corresponding untreated reference (background) cell samples are computed. Given the detection efficiency of the p53BP1 assay (P_{D}) at the target site and the proliferating cell fraction (Fp), a theoretical on-target distribution is calculated for the on-target activity of the nuclease. Subsequently, the distribution of the nuclease-treated sample is normalized by the distribution of the control sample and the theoretical on-target distribution using a process of non-negative least squares deconvolution. Lastly, the specificity is calculated as follows from the distribution of the background-normalized cell population: Given the ploidy (P_{T}) of the editing target, nuclease specificity is the % fraction of background-normalized cells containing p53BP1 foci from 0 to P_{T}. For simplicity in modeling, Fp and P_{D} are set to 0 and 1, respectively.

### Example 47

### Imaging-Based Dose Titration for Identification of Optimal Nuclease Dosing

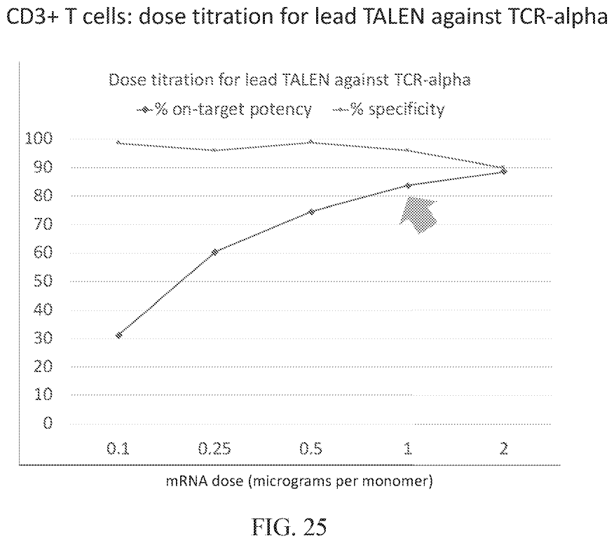

The p53BP1 imaging assay can be used to be used to optimize nuclease doses and thereby further reduce off-target effects of potent nucleases. The lead TALEN against the first constant exon of the TCR-alpha gene was evaluated for the effect of varying its dosage between 0.1 ug to 2 ug per monomer in primary CD3+ T cells. The off-target effects became more pronounced above a dose of 1 ug per monomer, while the on-target potency did not considerably increase. See FIG. 25. Thus, the nuclease dosage for a nuclease against a therapeutically relevant target was optimized using the p53BP1 imaging assay.

FIG. 25: Primary CD3+ T cells were transfected with a high-specificity TALEN against TCR-alpha, at doses of 0, 0.1, 0.25, 0.5, 1, and 2 ug per monomer. TALEN mRNA was used for the transfection. Transfected cells were subject to cold shock (30C) for 24 hours, after which they were retrieved, washed with PBS, seeded onto PLL-coated, glass bottom 24-well plates, stained for p53BP1 and FLAG, and imaged in 3D using a Nikon epi fluorescence microscope fitted with an Andor Zyla camera and 60×, 1.4 NA oil objective. % on-target potency and % nuclease specificity were calculated as detailed above.

### Example 48

### High Throughput Screening of Nucleases for Clinically Relevant Applications

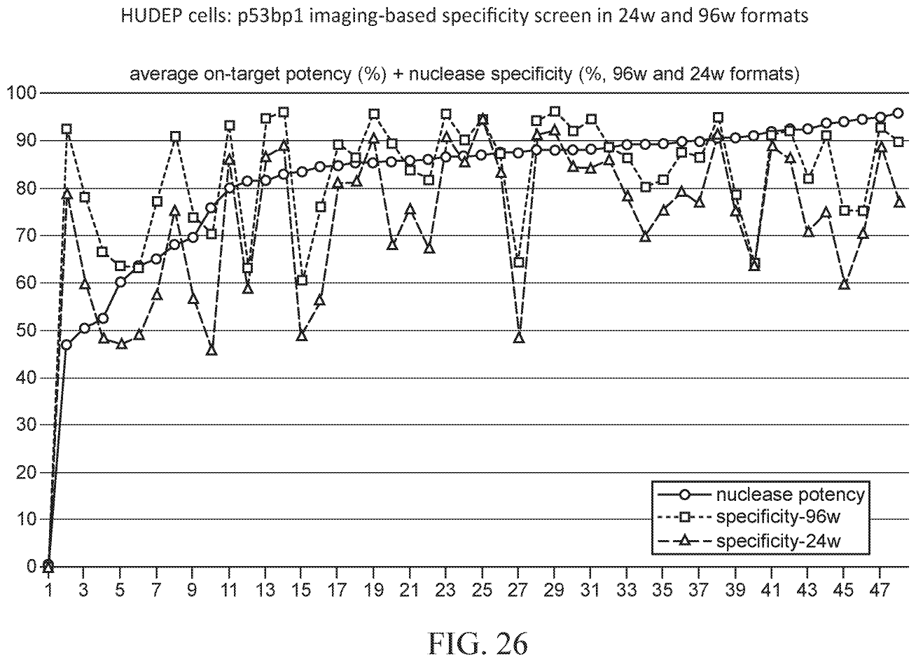

The p53BP1 imaging assay was used to rapidly screen nucleases on the basis of their specificity. 47 TALENs for a clinically relevant genomic target in the vicinity of the human gamma hemoglobin gene were generated, and their specificity evaluated in human erythroid HUDEP2 cells. A subset of TALENs that were highly specific while still being potent were identified. See FIG. 26.

FIG. 26: HUDEP2 cells were transfected with 47 TALENs against the HBG1/2 gene promoter locus, each at dose of 2.5 ug per monomer. TALEN mRNA was used for the transfection. Transfected cells were subject to cold shock (30C) for 24 hours, after which they were retrieved, washed with PBS, seeded onto PLL-coated, glass bottom 24-well plates or 96-well plates, stained for p53BP1 and FLAG, and imaged in 3D using a Nikon epi fluorescence microscope fitted with an Andor Zyla camera and 40×, 0.9 NA air objective. % on-target potency and % nuclease specificity were calculated as detailed above. % indel rates were calculated from cells retrieved 14 days post transfection.

### Example 49

### Analysis of Cellular Perturbation

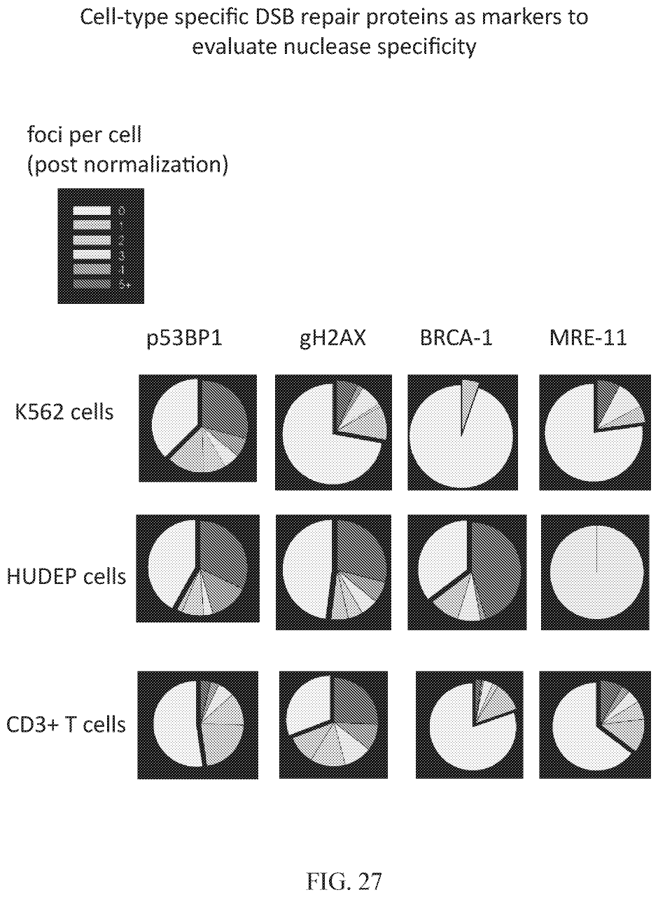

The methods provided herein can be used to evaluate the variation in any protein that responds to an external stimulus or perturbation. The change in foci spot distributions for 4 different DNA repair proteins (p53bp1, gamma-H2AX, BRCA1, and MRE-11) in 3 cell types (K562, HUDFP2, and CD3+ T cells) was analyzed. All of these proteins could be used to estimate nuclease specificity in a cell-type specific manner. FIG. 27.

The examples and embodiments described herein are for illustrative purposes only and various modifications or changes suggested to persons skilled in the art are to be included within the spirit and purview of this application and scope of the appended claims.

For reasons of completeness, certain embodiments of the methods of the present disclosure are set out in the following numbered aspects:

1. A method of quantifying a protein load, the method comprising quantifying a protein that accumulates in a primary cell in response to a cellular perturbation on a per allele per cell basis.

2. A method of quantifying a protein load, the method comprising quantifying a protein that accumulates in a plurality of cells in response to a cellular perturbation in less than 24 hours on a per allele per cell basis.

3. A method of screening a plurality of cell engineering tools for specificity, the method comprising quantifying a protein load in an intact cell in less than 24 hours and determining the specificity of the cell engineering tool for a target genomic locus based on the protein load.

4. A method of producing a potent and specific cell engineering tool, the method comprising:
a) administering a cell engineering tool to a cell;
b) determining specificity, activity, or a combination thereof of the cell engineering tool for a target genomic locus by quantifying a protein load;
c) quantifying potency of the cell engineering tool by measuring gene editing efficiency, activation of gene expression, or repression of gene expression; and
d) adjusting a parameter of the cell engineering tool to increase specificity for the target genomic locus.

5. The method of any one of aspects 3-4, wherein the protein accumulates in response to a cellular perturbation.

6. The method of any one of aspects 3-5, wherein the method further comprises quantifying the protein load on a per allele per cell basis.

7. The method of any one of aspects 3 or 5-6, wherein the intact cell comprises an intact primary cell.

8. The method of any one of aspects 1 or 4-6, wherein the cell or primary cell comprises an intact primary cell.

9. The method of any one of aspects 1 or 5-8, wherein the cellular perturbation comprises administering a cell engineering tool.

10. The method of aspect 9, the method further comprising determining specificity of the cell engineering tool for a target genomic locus.

11. The method of any one of aspects 1-2 or 5-10, the method further comprising quantifying gene editing efficiency, activation of gene expression, or repression or gene expression.

12. The method of aspect 2, wherein the plurality of cells comprises at least 5 cells, at least 10 cells, at least 20 cells, at least 50 cells, at least 100 cells, at least 200 cells, at least 500 cells, or at least 1000 cells.

13. The method of any one of aspects 1-12, wherein the protein indicates a cellular response.

14. The method of aspect 13, wherein the cellular response comprises a double strand break, activation of transcription, repression of transcription, or chromosome translocation.

15. The method of any one of aspects 1-14, wherein the cell or intact cell comprises an immortalized cell.

16. The method of any one of aspects 4 or 9-15, wherein the cell engineering tool comprises a genome editing complex or a gene regulator.

17. The method of aspect 16, wherein the gene regulator comprises a gene activator or a gene repressor.

18. The method of any one of aspects 1-17, wherein the protein comprises phosphorylated p53BP1 (p53BP1), γH2AX, 53BP1, H3K4me1, H3K4me2, H3K27ac, KAP1, H3K9me3, H3K27me3, or HP1.

19. The method of any one of aspects 1-18, wherein the protein comprises p53BP1.

20. The method of any one of aspects 1-19, the method further comprising staining the cell for the protein.

21. The method of aspect 20, wherein the staining the cell for the protein comprises labeling with a primary antibody against the protein and a secondary antibody conjugated to a first fluorophore.

22. The method of aspect 20, wherein the staining the cell for the protein comprises direct labeling with a primary antibody conjugated to a first fluorophore.

23. The method of any one of aspects 21-22, the method further comprising imaging the cell for one or more protein foci comprising the first fluorophore.

24. The method of any one of aspects 21-23, the method further comprising image analysis of the cell for the one or more protein foci comprising the first fluorophore.

25. The method of aspect 24, the method further comprising quantifying the protein load from the one or more protein foci comprising the first fluorophore.

26. The method of any one of aspects 1-25, wherein the protein load comprises a number of protein foci, total protein content within the nucleus, spatial localization pattern, or any combination thereof.

27. The method of any one of aspects 3-26, wherein the cell engineering tool further comprises a polypeptide tag.

28. The method of aspect 27, wherein the polypeptide tag is a FLAG tag.

29. The method of any one of aspects 3-28, the method further comprising staining the cell for the cell engineering tool.

30. The method of aspect 29, wherein the staining the cell for the cell engineering tool comprises staining with a primary antibody against the polypeptide tag and a secondary antibody conjugated to a second fluorophore.

31. The method of aspect 29, wherein the staining the cell for the cell engineering tool comprises direct labeling with a primary antibody conjugated to a second fluorophore.

32. The method of aspect 29, wherein the staining of the cell for the cell engineering tool comprises staining with a primary antibody against the nuclease and a secondary antibody conjugated to a second fluorophore.

33. The method of aspect 29, wherein the staining the cell for the cell engineering tool comprises direct labeling with a primary antibody conjugated to a second fluorophore.

34. The method of aspect 33, further comprising imaging the cell for one or more cell engineering tool foci comprising the second fluorophore.

35. The method of aspect 34, further comprising image analysis of the cell for the one or more cell engineering tool foci comprising the second fluorophore.

36. The method of aspect 35, the method further comprising quantifying cell engineering tool load from the one or more cell engineering tool foci comprising the second fluorophore.

37. The method of aspect 36, wherein the cell engineering tool load comprises a number of cell engineering tool foci, total content of the cell engineering tool within the nucleus, spatial localization pattern, or any combination thereof.

38. The method of any one of aspects 1-37, the method further comprising hybridizing a probe set comprising a plurality of probes to the cell, wherein the probe set targets and binds to a target genomic locus.

39. The method of aspect 38, wherein each probe of the plurality of probes comprises a third fluorophore.

40. The method of any one of aspects 38-39, wherein the probe set comprises an oligonucleotide probe set.

41. The method of aspect 40, further comprising imaging the cell for one or more Nano-FISH foci comprising the third fluorophore.

42. The method of aspect 41, further comprising image analysis of the cell for the one or more Nano-FISH foci comprising the third fluorophore.

43. The method of any one of aspects 39-42, wherein co-localization of signal from the first fluorophore and the third fluorophore indicates that the cellular perturbation occurs at the target genomic locus.

44. The method of any one of aspects 1-43, the method further comprising hybridizing a second probe set comprising a second plurality of probes to the cell, wherein the second probe set targets and binds to an off-target genomic locus.

45. The method of aspect 44, wherein each probe of the second plurality of probes comprises a fourth fluorophore.

46. The method of any one of aspects 44-45, wherein the second probe set comprises a second oligonucleotide probe set.

47. The method of aspect 46, further comprising imaging the cell for one or more Nano-FISH foci comprising the fourth fluorophore.

48. The method of aspect 47, further comprising image analysis of the cell for the one or more Nano-FISH foci comprising the fourth fluorophore.

49. The method of any one of aspects 44-48, wherein co-localization of signal from the first fluorophore, the third fluorophore, and the fourth fluorophore indicates a chromosome translocation.

50. The method of any one of aspects 23-49, wherein imaging the cell comprises acquiring images of the cell by a microscopy mode selected from the group consisting of epifluorescence, widefield, confocal, selective plane illumination, tomography, holography, super-resolution, and synthetic aperture optics (SAO).

51. The method of aspect 50, further comprising processing the acquired images to identify regions of interest (ROIs) comprising cell nuclei, protein marker foci, sites of cell engineering tool localization, or a combination thereof.

52. The method of aspect 51, further comprising processing the ROIs to extract a plurality of features selected from the group consisting of count, spatial location, size (area/volume), shape (circularity/sphericity, eccentricity, irregularity (concavity/convexity), diameter, perimeter/surface area, quantitative measures of image texture that are pixel-based or region-based over a tunable length scale, nuclear diameter, nuclear area, nuclear volume, perimeter, surface area, DNA content, DNA texture measures, number of protein marker foci, size of protein marker foci, shape of protein marker foci, amount of protein marker per cell, spatial location and localization pattern of protein marker foci, number of nuclease per cell, amount of nuclease per cell, nuclease localization or texture, number of cell engineering tool foci, size of cell engineering tool foci, shape of cell engineering tool foci, amount of cell engineering tool foci per cell, spatial location and localization pattern of cell engineering tool foci, number of Nano-FISH foci, size of Nano-FISH foci, shape of Nano-FISH foci, amount of Nano-FISH foci, spatial location of Nano-FISH foci, and localization pattern of Nano-FISH foci.

53. The method of aspect 52, further comprising processing the extracted plurality of features to measure a degree of co-localization between the one or more Nano-FISH foci and the one or more protein marker foci, thereby determining specificity of the genome editing complex or the gene regulator.

54. The method of any one of aspects 52-53, further comprising applying a machine learning predictor to the extracted plurality of features to evaluate performance of cell engineering tools by predicting a distinction capability of nucleases.

55. The method of any one of aspects 16-54, wherein the genome editing complex comprises a DNA binding domain and a nuclease.

56. The method of aspect 55, wherein the genome editing complex further comprises a linker.

57. The method of any one of aspects 17-54, wherein the gene activator comprises a DNA binding domain and an activation domain.

58. The method of aspect 57, wherein the gene activator further comprises a linker.

59. The method of any one of aspects 17-54, wherein the gene repressor comprises a DNA binding domain and a repressor domain.

60. The method of aspect 59, wherein the gene repressor further comprises a linker.

61. The method of any one of aspects 55-60, wherein the DNA binding domain comprises a transcription activator-like effector (TALE) protein, a zinc finger protein (ZFP), or a single guide RNA (sgRNA).

62. The method of any one of aspects 16-54 or 55-56, wherein the genome editing complex is a TALEN, a ZFN, a CRISPR/Cas9, a megaTAL, or a meganuclease.

63. The method of any one of aspects 53-54 or 59-60, wherein the nuclease comprises FokI.

64. The method of aspect 63, wherein FokI has at least 70%, at least 75%, at least 80%, at least 85%, at least 90%, at least 92%, at least 95%, at least 97%, or at least 99% sequence identity to SEQ ID NO: 1062.

65. The method of any one of aspects 56-64, wherein the linker comprises the naturally occurring C-terminus of a TALE protein or any truncation thereof 66. The method of any one of aspects 56-64, wherein the linker comprises 0-15 residues of glycine, methionine, aspartic acid, alanine, lysine, serine, leucine, threonine, tryptophan, or any combination thereof 67. The method of any one of aspects 57-66, wherein the activation domain comprises VP16, VP64, p65, p300 catalytic domain, IET1 catalytic domain, TDG, Ldb1 self-associated domain, SAM activator (VP64, p65, HSF1), VPR (VP64, p65, Rta).

68. The method of any one of aspects 59-66, wherein the repressor domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

69. The method of any one of aspects 16-68 wherein a parameter of the genome editing complex or the gene regulator is adjusted improve specificity.

70. The method of aspect 69, wherein the parameter is a sequence of the DNA binding domain or length of the DNA binding domain.

71. The method of any one of aspects 1-70, the protein load is quantified in at least 50 to 100,000 cells.

72. The method of aspect 71, wherein the protein load is quantified in no more than 1000, no more than 500, no more than 100, or no more than 50 cells. 73. The method of any one of aspects 1-72, wherein the cell comprises a hematopoietic stem cells (HSC), a T cell, a chimeric antigen receptor T cell (CAR T cell).

74. The method of any one of aspects 1-72, wherein the cell is from a normal solid tissue or a tumorigenic solid tissue.

75. The method of any one of aspects 1-74, wherein the target genomic locus is within a PDCD1 gene, a CTLA4 gene, a LAG3 gene, a IET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, an IL2RG gene, or a combination thereof 76. The method of any one of aspects 1-75, wherein a chimeric antigen receptor (CAR), engineered T cell receptor (TCR), alpha-L iduronidase (IDUA), iduronate-2-sulfatase (IDS), IL-12, or Factor 9 (F9) is inserted upon cleavage of a region of the target nucleic acid sequence.

What is claimed is:

1. A method comprising:
contacting a live cell with a cell engineering tool comprising a DNA binding domain and a nuclease domain, a gene repressor, or a gene activator, wherein the live cell comprises genomic DNA comprising a target genomic locus for the DNA binding domain of the cell engineering tool;
fixing the cell and contacting the fixed cell with a plurality of nucleic acid probes complementary to the target genomic locus and assaying for presence of a protein indicative of cellular response to the contacting; and
assaying for colocalization of the probes and the protein, wherein detection of the colocalization indicates activity of the cell engineering tool at the target genomic locus and absence of the colocalization indicates activity of the cell engineering tool at an off-target site.

2. The method of claim 2, wherein assaying for colocalization comprises imaging the cell at 40× or higher magnification.

3. The method of any one of claims 1-3, wherein the fixing of the cell is performed within 24 hours or less of the contacting.

4. The method of any one of claims 1-3, wherein the cell engineering tool comprises a DNA binding domain and a nuclease domain.

5. The method of claim 4, wherein the nuclease domain induces a double strand break in the genomic DNA and wherein the protein indicative of cellular response to the contacting comprises a DNA repair protein.

6. The method of claim 5, wherein DNA repair protein comprises p53BP1, γH2AX, MRE-11, BRCA1, RAD-51, phospho-ATM or MDC1.

7. The method of any one of claims 1-3, wherein the cell engineering tool comprises a DNA binding domain and a gene repressor.

8. The method of claim 7, wherein the gene repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

9. The method of any one of claims 1-3, wherein the cell engineering tool comprises a DNA binding domain and a gene activator.

10. The method of claim 9, wherein the gene activator comprises VP16, VP64, p65, p300 catalytic domain, TET1 catalytic domain, TDG, Ldb1 self-associated domain, SAM activator (VP64, p65, HSF1), VPR (VP64, p65, Rta).

11. The method any one of claims 1-10, wherein the DNA binding domain comprises a transcription activator-like effector (TALE) protein, a zinc finger protein (ZFP), or a single guide RNA (sgRNA).

12. The method of any one of claims 1-11, wherein the cell is a primary cell.

13. The method of any one of claims 1-11, wherein the cell is a hematopoietic stem cell (HSC), a T cell, a chimeric antigen receptor T cell (CAR T cell).

14. The method of any one of claims 1-11, wherein the cell is from a normal solid tissue or a tumorigenic solid tissue.

15. The method of any one of claims 1-11, wherein the cell is an immortalized cell.

16. The method of any one of claims 1-15, wherein the target genomic locus is within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, or an IL2RG gene.

17. The method of any one of claims 1-16, wherein assaying for the colocalization comprises imaging the cell by a microscopy mode selected from the group consisting of epifluorescence, widefield, confocal, selective plane illumination, tomography, holography, super-resolution, and synthetic aperture optics (SAO).

18. The method of any one of claims 1-17, wherein the plurality of nucleic acid probes are 30-60 bases in length.

19. The method of any one of claims 1-18, wherein the plurality of nucleic acid probes comprise 20-200 probes having distinct sequences.

20. The method of any one of claims 1-19, wherein the plurality of nucleic acid probes bind to a 1 kilobase (kb) to 5 kb region comprising the target genomic locus.

21. The method of any one of claim 1-20, wherein when the absence of colocalization is detected, the method further comprises adjusting a parameter of the genome editing tool to improve specificity.

22. The method of claim 21, wherein the parameter is a sequence of the DNA binding domain or length of the DNA binding domain.

23. The method of claim 21, wherein the parameter is an amount of the genome editing tool introduced into the cell.

24. A method comprising:
contacting a live cell with a cell engineering tool comprising a DNA binding domain and a nuclease domain, a gene repressor, or a gene activator, wherein the live cell comprises genomic DNA comprising a target genomic locus for the DNA binding domain of the cell engineering tool;
fixing the cell and assaying for presence of a measurable change in nuclear protein load of a protein indicative of cellular response to the contacting, wherein the measurement reflects the total activity of the cell engineering tool.

25. The method of claim 24, further comprising contacting the fixed cell with a plurality of nucleic acid probes complementary to the target genomic locus; and assaying for colocalization of the probes and the protein indicative of cellular response, wherein detection of the colocalization indicates activity of the cell engineering tool at the target genomic locus and absence of the colocalization indicates activity of the cell engineering tool at an off-target site.

26. The method of claim 24 or 25, wherein assaying for the change in nuclear protein load comprises imaging the cell by a microscopy mode selected from the group consisting of epifluorescence, widefield, confocal, selective plane illumination, tomography, holography, super-resolution, and synthetic aperture optics (SAO) and comparing to nuclear protein load in a reference cell not contacted with the cell engineering tool.

27. The method of any one of claims 24-26, wherein when the measured change in protein load above an application-specific baseline level is detected, the method further comprises adjusting a parameter of the genome editing tool to improve specificity.

28. The method of claim 1, wherein assaying comprises imaging the cell at 40× or higher magnification.

29. The method of any one of claims 24-28, wherein the fixing of the cell is performed within 24 hours or less of the contacting.

30. The method of any one of claims 24-29, wherein the cell engineering tool comprises a DNA binding domain and a nuclease domain.

31. The method of claim 30, wherein the nuclease domain induces a double strand break in the genomic DNA and wherein the protein indicative of cellular response to the contacting comprises a DNA repair protein.

32. The method of claim 31, wherein DNA repair protein comprises p53BP1, γH2AX, MRE-11, BRCA1, RAD-51, phospho-ATM or MDC1.

33. The method of any one of claims 24-28, wherein the cell engineering tool comprises a DNA binding domain and a gene repressor.

34. The method of claim 33, wherein the gene repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

35. The method of any one of claims 24-28, wherein the cell engineering tool comprises a DNA binding domain and a gene activator.

36. The method of claim 35, wherein the gene activator comprises VP16, VP64, p65, p300 catalytic domain, TET1 catalytic domain, TDG, Ldb1 self-associated domain, SAM activator (VP64, p65, HSF1), VPR (VP64, p65, Rta).

37. The method any one of claims 24-36, wherein the DNA binding domain comprises a transcription activator-like effector (TALE) protein, a zinc finger protein (ZFP), or a single guide RNA (sgRNA).

38. The method of any one of claims 24-37, wherein the cell is a primary cell.

39. The method of any one of claims 24-37, wherein the cell is a hematopoietic stem cell (HSC), a T cell, a chimeric antigen receptor T cell (CAR T cell).

40. The method of any one of claims 24-37, wherein the cell is from a normal solid tissue or a tumorigenic solid tissue.

41. The method of any one of claims 24-37, wherein the cell is an immortalized cell.

42. The method of any one of claims 24-41, wherein the target genomic locus is within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, or an IL2RG gene.

43. The method of any one of claims 25-42, wherein the plurality of nucleic acid probes are 30-60 bases in length.

44. The method of any one of claims 25-43, wherein the plurality of nucleic acid probes comprise 20-200 probes having distinct sequences.

45. The method of any one of claims 25-44, wherein the plurality of nucleic acid probes bind to a 1 kilobase (kb) to 5 kb region comprising the target genomic locus.

46. The method of any one of claim 25-45, wherein when the absence of colocalization is detected, the method further comprises adjusting a parameter of the genome editing tool to improve specificity.

47. The method of claim 46, wherein the parameter is a sequence of the DNA binding domain or length of the DNA binding domain.

48. The method of claim 46, wherein the parameter is an amount of the genome editing tool introduced into the cell.
